(19) United States
(12) Patent Application Publication
(10) Pub. No.: US 2022/0380370 A1
(43) Pub. Date: Dec. 1, 2022

(54) XANTHINE CB1 INHIBITORS

(71) Applicant: Goldfinch Bio, Inc., Cambridge, MA (US)

(72) Inventors: Maolin Yu, West Roxbury, MA (US); Brett D. Williams, Boston, MA (US); Mark W. Ledeboer, Acton, MA (US); Jean-Christophe P. Harmange, Andover, MA (US); Sydney E. Carra, Feeding Hills, MA (US)

(21) Appl. No.: 17/763,974

(22) PCT Filed: Sep. 25, 2020
(86) PCT No.: PCT/US20/52627

§ 371 Date: Mar. 25, 2022

Related U.S. Application Data
(60) Provisional application No. 62/905,638, filed on Sep. 25, 2019.

(51) Int. Cl. C07D 473/04; C07D 473/06

(52) U.S. Cl. CPC C07D 473/04; C07D 473/06

(57) ABSTRACT

Disclosed are compounds having structural formula I, and related pharmaceutical compositions. Also disclosed are therapeutic methods, e.g., of treating diseases such as diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease, using the compounds of Formula (I).

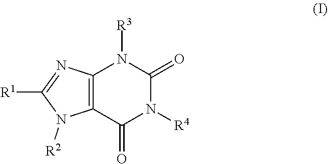

### RELATED APPLICATIONS

This application claims the benefit of priority to U.S. Provisional Application No. 62/905,638, filed Sep. 25, 2019.

### BACKGROUND

The CB1 cannabinoid receptor is one of the most abundant G-protein coupled receptors in the brain; it is highly expressed in the basal ganglia nuclei, hippocampus, cortex, and cerebellum. The distribution of this receptor within the central nervous system (CNS) correlated with its role in the control of motor function, cognition and memory, and analgesia. CB1 receptors are also expressed throughout the periphery, albeit at much lower levels than in the CNS. The receptor has also been detected in a variety of circulating immune cells and numerous peripheral tissues, including the adrenal gland, heart, lung, prostate, liver, bone marrow, and thymus. Endogenous ligands for the CB1 receptor include the arachidonic acid metabolites N-arachidonylethanolamide (anandamide) and 2-arachidonylglycerol (2-AG), and exogenous ligands include phytocannabinoids such as those found in cannabis.

Experimental studies have suggested that stimulation of the CB1 receptor using pharmacologic agents or its natural ligands could have deleterious effects on several different organs. CB1 receptor expression is altered in diabetic kidney disease, and preclinical studies have confirmed that the CB1 receptor is implicated in the pathogenesis of diabetic kidney disease. Several reports have also described the development of acute kidney injury in otherwise healthy patients exposed to synthetic cannabinoids. In the liver, the CB1 and CB2 receptors are faintly expressed under physiological conditions, but induction of these receptors and/or increased levels of cannabinoids are common features of liver injuries such as alcoholic liver disease and non-alcoholic fatty liver disease; the latter of these is characterized by upregulation of adipose tissue and hepatocyte CB1 receptors and increased liver synthesis of anandamide. The CB1 receptor is also implicated in diabetic nephropathy, obesity-related kidney disease, kidney fibrosis, Prader Willi syndrome, focal segmental glomerular sclerosis, IgA nephropathy, nephrotic syndrome, metabolic syndrome, non-alcoholic liver disease, and various gastrointestinal diseases.

As enhanced CB1 expression is associated with pathogenesis of numerous diseases, inhibition of CB1 is a promising therapeutic strategy. Thousands of orthosteric inhibitors of CB1, belonging to many different structural classes, have been synthesized and evaluated. However, this strategy has had only limited success in bringing such leads to the clinic, largely owing to unwanted side effects. Allosteric inhibition strategies have also been of limited value, as promising in vitro activity does not always translate into in vivo potency.

Hence, there is a need for new modulators of CB1 receptor activity.

### SUMMARY

This invention is based, at least in part, on the discovery that inhibition of the CB1 receptor by certain compounds may be useful to treat a disease or condition characterized by aberrant CB1 activity.

One aspect of the invention is compounds that are inhibitors of the CB1 receptor. In some embodiments, the invention relates to compounds having structural formula I:

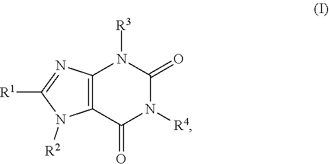

or a pharmaceutically acceptable salt thereof, wherein:

R¹ is aryl, or a 5- to 6-membered heteroaryl that is optionally benzofused, wherein R¹ is optionally substituted;

R² is aryl or a 5- to 6-membered heteroaryl that is optionally benzofused, wherein R² is optionally substituted;

R³ is hydrogen, —(C(R⁵)₂)₀₋₂-carbocyclyl, —(C(R⁵)₂)₀₋₂-heterocyclyl, —(C(R⁵)₂)₁₋₂-pyridinyl, or —(C(R⁵)₂)₁₋₂-phenyl, wherein each R⁵ is independently hydrogen or C₁-C₃ alkyl optionally substituted with one or more substituents independently selected from —OH and halo, and wherein each carbocyclyl, heterocyclyl, pyridinyl and phenyl is optionally substituted with up to two substituents independently selected from halo, —CN, or C₁-C₄ alkyl optionally substituted with halo or hydroxy; and

R⁴ is hydrogen, —C₁-C₄ alkyl optionally substituted with 1 to 3 hydroxyls, —C₁-C₄ alkylene-C(O)—NR⁶R⁷, —C₁-C₄ alkylene-S(O)₂—NR⁶R⁷, —C₁-C₄ alkylene-O—C(O)—C₁-C₄ alkyl, —C₁-C₄ alkylene-O—C₁-C₄ alkyl, —(C(R⁵)₂)₀₋₂-cycloalkyl, or —(C(R⁵)₂)₀₋₂-saturated heterocyclyl, wherein each of R⁶ and R⁷ is independently selected from hydrogen and C₁-C₄ alkyl, and wherein any two methylene units in any alkyl or alkylene portion of R⁴ are optionally taken together with any intervening methylene unit or units to form a cycloalkyl, oxetanyl, tetrahydropyranyl, tetrahydrofuranyl, pyrrolidinyl; and

when R³ is hydrogen, R⁴ is not hydrogen or methyl.

In one aspect, the invention features a composition comprising a compound of the invention and a pharmaceutically acceptable carrier.

In one aspect, the invention relates to a method of treating a disease or condition characterized by aberrant CB1 activity, the method comprising the step of administering to a subject in need thereof an effective amount of a compound or composition of the invention.

In some embodiments, the disease or condition is diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

In some embodiments, the disease or condition is diabetic nephropathy. In some embodiments, the disease or condition is focal segmental glomerular sclerosis. In some embodiments, the disease or condition is nonalcoholic steatohepatitis.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

The invention provides several advantages. The prophylactic and therapeutic methods described herein are effective in treating a disease or condition characterized by aberrant CB1 activity. Further, methods described herein are effective to identify compounds that treat or reduce risk of developing a disease or condition characterized by aberrant CB1 activity.

Other features, objects, and advantages of the invention will be apparent from the detailed description, and from the claims.

### DETAILED DESCRIPTION

### Definitions

Unless otherwise defined, all technical and scientific terms used herein have the same meaning as commonly understood by one of ordinary skill in the art to which this invention belongs. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000). Although methods and materials similar or equivalent to those described herein can be used in the practice or testing of the present invention, suitable methods and materials are described below.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents, published patent applications, or other references referred to in this application are specifically incorporated by reference herein. In case of conflict, the present specification, including its specific definitions, will control. In addition, the materials, methods, and examples are illustrative only and not intended to be limiting.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, sublingually, orally (by ingestion), intranasally (by inhalation), and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless otherwise specified, “alkylene” by itself or as part of another substituent refers to a saturated straight-chain or branched divalent group having the stated number of carbon atoms and derived from the removal of two hydrogen atoms from the corresponding alkane. Examples of straight chained and branched alkylene groups include —CH₂— (methylene), —CH₂—CH₂-(ethylene), —CH₂—CH₂—CH₂— (propylene), —CH(CH₃)—, —C(CH₃)₂—, —CH₂—CH(CH₃)—, —CH₂—CH₂—CH₂—CH₂—, —CH₂—CH₂—CH₂—CH₂—CH₂— (pentylene), —CH₂—CH(CH₃)—CH₂—, and —CH₂—C(CH₃)₂—CH₂—.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. Co alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

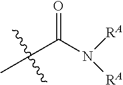

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

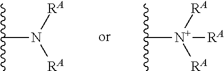

wherein each R^{A} independently represents a hydrogen or a hydrocarbyl group, or two R are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, and/or aryls. Aryl groups include benzene, naphthalene, phenanthrene, phenol, aniline, and the like.

The term “carbamate” is art-recognized and refers to a group

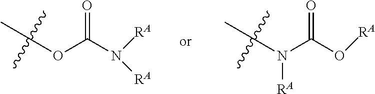

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or non-aromatic unsaturated ring in which each atom of the ring is carbon. Carbocycles include both cycloalkane rings, in which all carbon atoms are saturated, and cycloalkene rings, which contain at least one double bond and have no aromatic character. “Carbocycle” includes 5-7 membered monocyclic and 8-12 membered bicyclic rings. Each ring of a bicyclic carbocycle may be selected from saturated and unsaturated non-aromatic rings in which each atom of each ring is carbon. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated and unsaturated non-aromatic rings. Any combination of saturated and unsaturated non-aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, and adamantane. Exemplary fused carbocycles include decalin, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, and the other cyclic rings can be cycloalkyls, cycloalkenyls, aryls, and/or heteroaryls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, and the other cyclic rings can be cycloalkyls, cycloalkenyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl” or “heterocycloalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

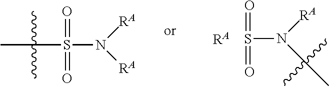

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or —SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

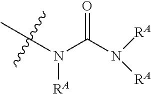

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” or “reduces the risk of developing” a disease, disorder, or condition refers to a compound that, in a statistical sample, reduces the occurrence of the disease, disorder, or condition in the treated sample relative to an untreated control sample, or delays the onset or reduces the severity of one or more symptoms of the disorder or condition relative to the untreated control sample.

The term “treating” includes therapeutic treatments. A treatment is therapeutic, if it is intended to diminish, ameliorate, or stabilize the existing unwanted condition or side effects thereof.

In certain embodiments, compounds of the invention may be used alone or conjointly administered with another therapeutic agent. The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

The term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug, e.g., wherein a hydroxyl in the parent compound is presented as an ester or a carbonate or carboxylic acid present in the parent compound is presented as an ester.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms, An effective amount can be administered in one or more administrations, applications or dosages, A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

Compounds of the Invention

One aspect of the invention provides small molecules that inhibit the CB1 receptor.

In some embodiments, the compound of the invention is a compound of structural formula I:

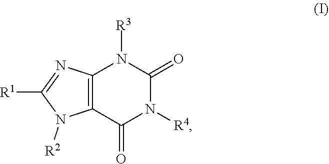

or a pharmaceutically acceptable salt thereof, wherein:

R¹ is aryl, or a 5- to 6-membered heteroaryl that is optionally benzofused, wherein R¹ is optionally substituted;

R² is aryl or a 5- to 6-membered heteroaryl that is optionally benzofused, wherein R² is optionally substituted;

R³ is hydrogen, —(C(R⁵)₂)₀₋₂-carbocyclyl, —(C(R⁵)₂)₀₋₂-heterocyclyl, —(C(R⁵)₂)₁₋₂-pyridinyl, or —(C(R⁵)₂)₁₋₂-phenyl, wherein each R⁵ is independently hydrogen or C₁-C₃ alkyl optionally substituted with one or more substituents independently selected from —OH and halo, and wherein each carbocyclyl, heterocyclyl, pyridinyl and phenyl is optionally substituted with up to two substituents independently selected from halo, —CN, or C₁-C₄ alkyl optionally substituted with halo or hydroxy; and

R⁴ is hydrogen, —C₁-C₄ alkyl optionally substituted with 1 to 3 hydroxyls, —C₁-C₄ alkylene-C(O)—NR⁶R⁷, —C₁-C₄ alkylene-S(O)₂—NR⁶R⁷, —C₁-C₄ alkylene-O—C(O)—C₁-C₄ alkyl, —C₁-C₄ alkylene-O—C₁-C₄ alkyl, —(C(R⁵)₂)₀₋₂-cycloalkyl, or —(C(R⁵)₂)₀₋₂-saturated heterocyclyl, wherein each of R⁶ and R⁷ is independently selected from hydrogen and C₁-C₄ alkyl, and wherein any two methylene units in any alkyl or alkylene portion of R⁴ are optionally taken together with any intervening methylene unit or units to form a cycloalkyl, oxetanyl, tetrahydropyranyl, tetrahydrofuranyl, pyrrolidinyl; and

when R³ is hydrogen, R⁴ is not hydrogen or methyl.

As used herein, a ring that is “benzofused” is fused to a phenyl ring, wherein either ring may be further substituted. The benzofused group is a bicyclic group in which one ring is a phenyl ring and each of the rings shares two adjacent atoms with the other ring.

In certain embodiments, R¹ is optionally substituted with up to 3 substituents independently selected from —CN, —CF₃, halo, or methyl; and R² is optionally substituted with up to 3 substituents independently selected from —CN, —CF₃, halo, or methyl.

In certain embodiments, at least one of R¹ or R² is phenyl optionally substituted with one or more halo.

In certain embodiments, R¹ is phenyl, pyridin-2-yl, pyridin-3-yl, or pyrazol-5-yl, and R¹ is optionally substituted with up to two substituents independently selected from methyl and halo.

In certain embodiments, R¹ is 3-chloropyridin-2-yl, 2-chloropyridin-3-yl, 2-chlorophenyl, 2,4-difluorophenyl, 2-chloro-4-fluorophenyl, or 1-methylpyrazol-5-yl.

In certain embodiments, R² is 4-chlorophenyl or 6-chloropyridin-3-yl.

In certain embodiments, R³ is hydrogen, —(CHR⁵)₀₋₁-piperidin-4-yl, —(CHR⁵)₀₋₁-pyridin-2-yl, —(CHR⁵)₀₋₁-tetrahydropyran-4-yl, —(CHR⁵)₀₋₁-tetrahydrothiopyran-4-yl, —(CHR⁵)₀₋₁-phenyl, —(CHR)₀₋₁-cyclohexyl, —(CHR⁵)₀₋₁-1,4-dioxan-2-yl, —(CHR⁵)₀₋₁-thietan-3-yl, or —(CHR⁵)₀₋₁-tetrahydrothiofuran-3-yl, —(CHR⁵)₀₋₁—, wherein R³ is optionally substituted on any ring with one or more of halo, oxo, —OH, —C₁-C₄ alkyl, —CH₂—O—(CH₂)₂—O—CH₃, —C(═O)—O—C₁-C₄ alkyl, —C(═O)OH, —C(═O)—C₁-C₄ alkyl, —C(═O)N(R⁶)₂, —C(═O)N(R⁶)—CH₂-cyclopropyl, —S(═O)₂N(R⁶)₂, —S(═O)₂—C₁-C₄ alkyl, —S(═O)(═NH)—C₁-C₄ alkyl, 4-methylpiperazin-1-yl, morpholin-4-ylmethyl, 1,4-dioxan-2-yl, 1,4-dioxan-2-ylmethyl, tetrahydropyran-4-ylcarbamyl, or tetrahydrofuran-3-ylcarbamyl.

In certain embodiments, R³ is hydrogen, 1-methylsulfonylpiperidin-4-ylmethyl, 5-chloropyridin-2-ylmethyl, 4-hydroxytetrahydropyran-4-ylmethyl, 5-(tetrahydrofuran-2-ylcarbamyl)pyridin-2-ylmethyl, 5-(2-hydroxy-2-methylpropan-1-ylcarbamyl)pyridin-2-ylmethyl, 5-(tetrahydropyran-4-ylcarbamyl)pyridin-2-ylmethyl, 5-(2-hydroxyethan-1-ylaminosulfonyl)pyridin-2-yl-methyl, 1,1-dioxothiopyran-4-ylmethyl, 5-((1-hydroxycycloprop-1-ylmethyl)carbamyl)pyridin-2-ylmethyl, 5-(3-hydroxypropan-2-ylcarbamyl)pyridin-2-ylmethyl, 5-(aminosulfonyl)pyridin-2-ylmethyl, 4-fluorotetrahydropyran-4-ylmethyl, 4-(methylsulfonimidoyl)phenylmethyl, 4-(methylsulfonyl)phenylmethyl, 5-(methylsulfonyl)pyridin-2-ylmethyl, 4-(aminosulfonyl)phenylmethyl, cyclohexyl, 4-(carbamyl)phenylethan-2-yl, 4-(2,3-dihydroxylpropan-1-yl)phenylmethyl, 4-(1,4-dioxan-2-yl)phenylmethyl, 4-(1,4-dioxan-2-ylmethyl)phenylmethyl, tetrahydropyran-4-ylmethyl, tetrahydropyran-4-yl, 4-(2-hydroxyethan-1-ylcarbamyl)phenylethan-2-yl, 4-(carbamyl)phenylmethyl, 1-acetylpiperidin-4-ylmethyl, 1,4-dioxan-2-ylmethyl, 4-(2-hydroxyethan-1-ylmethylcarbamyl)phenylmethyl, 4-(2-methoxyethan-1-oxymethyl)phenylmethyl, 4-(morpholin-4-ylemthyl)phenylmethyl, 4-(2-hydroxyethan-1-ylmethylaminomethyl)phenylmethyl, 4-chlorophenylmethyl, 4-(4-methylpiperazin-1-ylmethyl)phenylmethyl, 1-(2-hydroxyethan-1-yl)piperidin-4-ylmethyl, 1-methylpiperidin-4-ylmethyl, 1-(carbamylmethyl)piperidin-4-ylmethyl, 1-(2,3-dihydroxypropan-1-yl)piperidin-4-ylmethyl, 4-(2-hydroxyethan-1-ylcarbamyl)phenylmethyl, 4-carboxyphenylmethyl, cyclohexylmethyl, 1-(1,1-dioxotetrahydrothiopyran-4-yl)ethan-1-yl, (1,1-dioxo-4-fluorotetrahydrothiopyran-4-yl)methyl, 1-(5-aminosulfonylpyridin-2-yl)ethan-1-yl, 4-(methylcarboxy)phenylmethyl, 1,1-dioxothietane-3-ylmethyl, 1,1-dioxotetrahydrofuran-3-ylmethyl, 1-(4-aminosulfonylphenyl)ethan-1-yl, (1,1-dioxo-tetrahydrothiopyran-4-yl)methyl, or 1-(5-aminosulfonylpyridin-2-yl)ethan-1-yl.

In certain embodiments, R⁴ is hydrogen, methyl, 2,3-dihydroxypropan-1-yl, 3-hydroxyproan-1-yl, 2-hydroxyethan-1-yl, carbamylmethyl, 1-carbamylcycloprop-1-ylmethyl, aminosulfonylmethyl, 2-(carbamyl)ethan-2-yl, 2-carbamylpropan-1-yl, tetrabutylcarboxymethyl, or 2-methoxyethan-1-yl.

In certain embodiments, the compound is selected from:

| # | Structure |
|---|---|
| 100 | 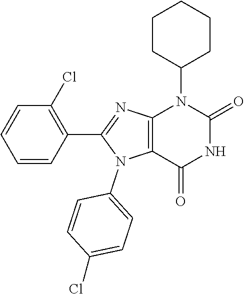 [SMILES: O=c1[nH]c(=O)n(C2CCCCC2)c2nc(-c3ccccc3Cl)n(-c3ccc(Cl)cc3)c12] |
| 101 | 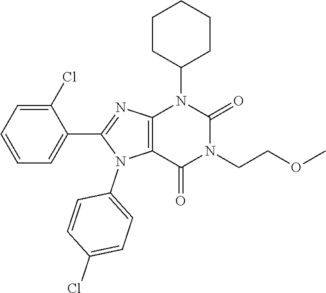 [SMILES: COCCn1c(=O)c2c(nc(-c3ccccc3Cl)n2-c2ccc(Cl)cc2)n(C2CCCCC2)c1=O] |
| 102 | 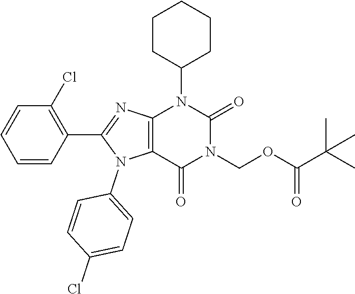 [SMILES: CC(C)(C)C(=O)OCn1c(=O)c2c(nc(-c3ccccc3Cl)n2-c2ccc(Cl)cc2)n(C2CCCCC2)c1=O] |
| 103 | 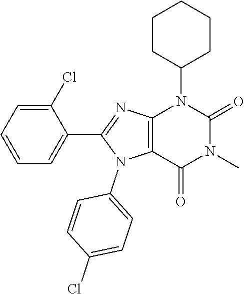 [SMILES: Cn1c(=O)c2c(nc(-c3ccccc3Cl)n2-c2ccc(Cl)cc2)n(C2CCCCC2)c1=O] |
| 104 | 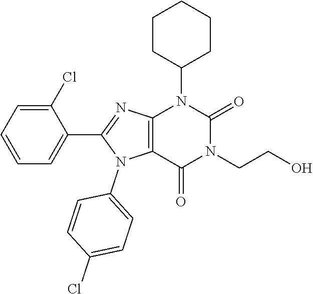 [SMILES: O=c1c2c(nc(-c3ccccc3Cl)n2-c2ccc(Cl)cc2)n(C2CCCCC2)c(=O)n1CCO] |
| 105 | 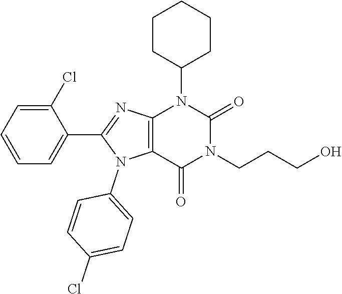 [SMILES: O=c1c2c(nc(-c3ccccc3Cl)n2-c2ccc(Cl)cc2)n(C2CCCCC2)c(=O)n1CCCO] |
| 106 | 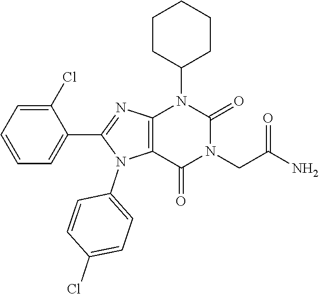 [SMILES: NC(=O)Cn1c(=O)c2c(nc(-c3ccccc3Cl)n2-c2ccc(Cl)cc2)n(C2CCCCC2)c1=O] |
| 107 | 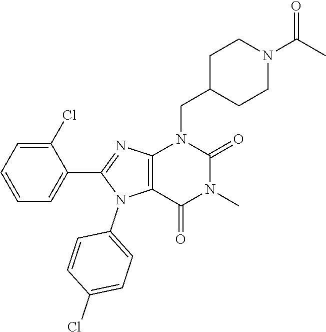 [SMILES: CC(=O)N1CCC(Cn2c(=O)n(C)c(=O)c3c2nc(-c2ccccc2Cl)n3-c2ccc(Cl)cc2)CC1] |
| 108 | 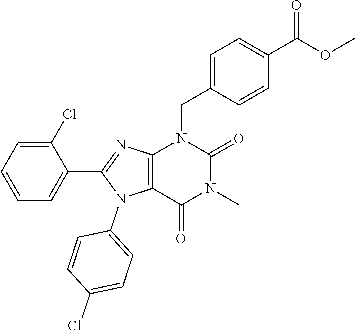 [SMILES: COC(=O)c1ccc(Cn2c(=O)n(C)c(=O)c3c2nc(-c2ccccc2Cl)n3-c2ccc(Cl)cc2)cc1] |
| 109 | 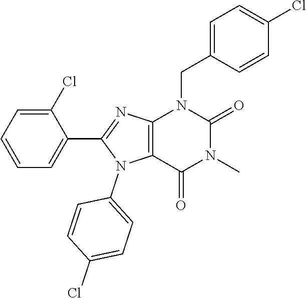 [SMILES: Cn1c(=O)c2c(nc(-c3ccccc3Cl)n2-c2ccc(Cl)cc2)n(Cc2ccc(Cl)cc2)c1=O] |
| 110 | 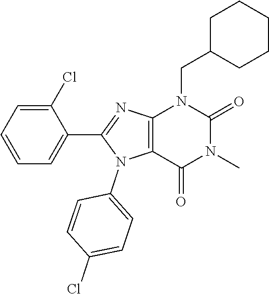 [SMILES: Cn1c(=O)c2c(nc(-c3ccccc3Cl)n2-c2ccc(Cl)cc2)n(CC2CCCCC2)c1=O] |
| 111 | 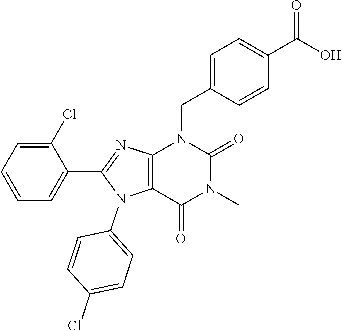 [SMILES: Cn1c(=O)c2c(nc(-c3ccccc3Cl)n2-c2ccc(Cl)cc2)n(Cc2ccc(C(=O)O)cc2)c1=O] |
| 112 | 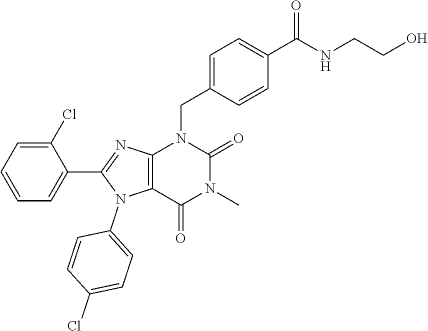 [SMILES: Cn1c(=O)c2c(nc(-c3ccccc3Cl)n2-c2ccc(Cl)cc2)n(Cc2ccc(C(=O)NCCO)cc2)c1=O] |
| 113 | 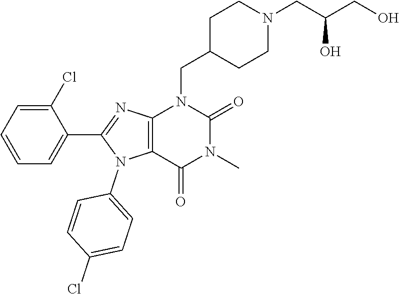 [SMILES: Cn1c(=O)c2c(nc(-c3ccccc3Cl)n2-c2ccc(Cl)cc2)n(CC2CCN(C[C@H](O)CO)CC2)c1=O] |
| 114 | 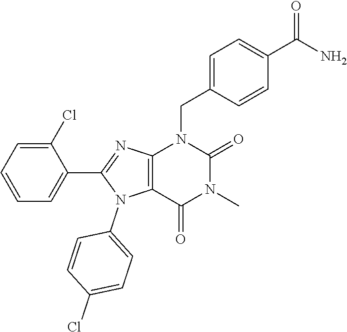 [SMILES: Cn1c(=O)c2c(nc(-c3ccccc3Cl)n2-c2ccc(Cl)cc2)n(Cc2ccc(C(N)=O)cc2)c1=O] |
| 115 | 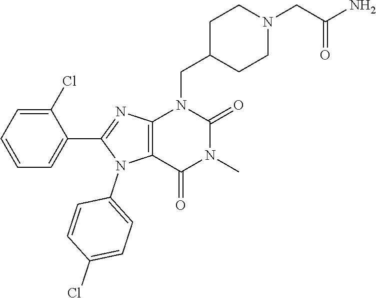 [SMILES: Cn1c(=O)c2c(nc(-c3ccccc3Cl)n2-c2ccc(Cl)cc2)n(CC2CCN(CC(N)=O)CC2)c1=O] |
| 116 | 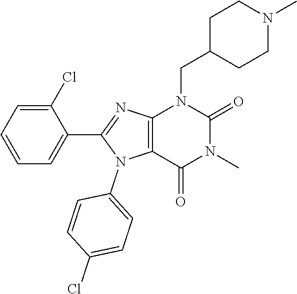 [SMILES: CN1CCC(Cn2c(=O)n(C)c(=O)c3c2nc(-c2ccccc2Cl)n3-c2ccc(Cl)cc2)CC1] |
| 117 | 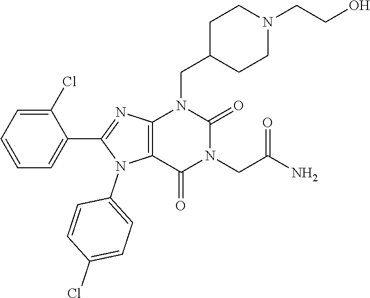 [SMILES: NC(=O)Cn1c(=O)c2c(nc(-c3ccccc3Cl)n2-c2ccc(Cl)cc2)n(CC2CCN(CCO)CC2)c1=O] |
| 118 | 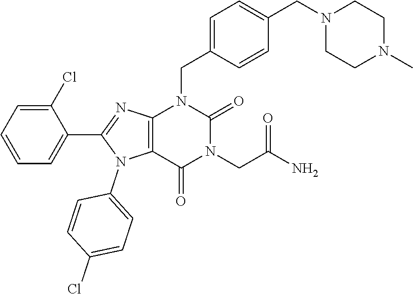 [SMILES: CN1CCN(Cc2ccc(Cn3c(=O)n(CC(N)=O)c(=O)c4c3nc(-c3ccccc3Cl)n4-c3ccc(Cl)cc3)cc2)CC1] |
| 119 | 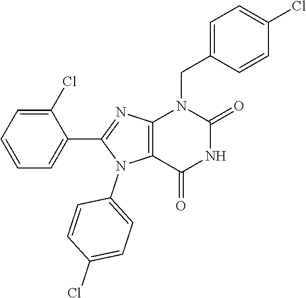 [SMILES: O=c1[nH]c(=O)n(Cc2ccc(Cl)cc2)c2nc(-c3ccccc3Cl)n(-c3ccc(Cl)cc3)c12] |
| 120 | 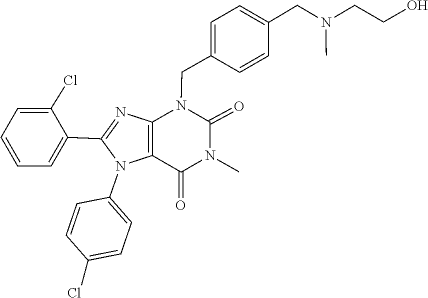 [SMILES: CN(CCO)Cc1ccc(Cn2c(=O)n(C)c(=O)c3c2nc(-c2ccccc2Cl)n3-c2ccc(Cl)cc2)cc1] |
| 121 | 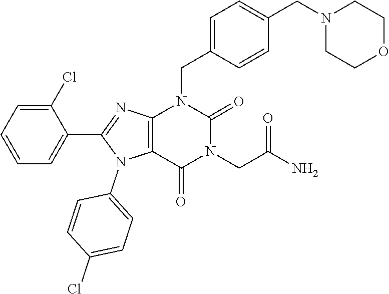 [SMILES: NC(=O)Cn1c(=O)c2c(nc(-c3ccccc3Cl)n2-c2ccc(Cl)cc2)n(Cc2ccc(CN3CCOCC3)cc2)c1=O] |
| 122 | 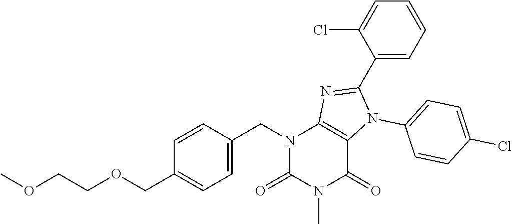 [SMILES: COCCOCc1ccc(Cn2c(=O)n(C)c(=O)c3c2nc(-c2ccccc2Cl)n3-c2ccc(Cl)cc2)cc1] |
| 123 | 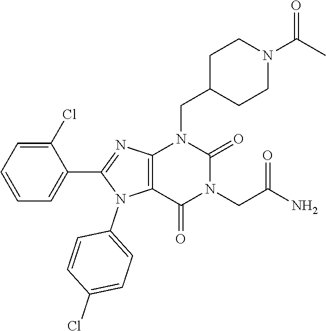 [SMILES: CC(=O)N1CCC(Cn2c(=O)n(CC(N)=O)c(=O)c3c2nc(-c2ccccc2Cl)n3-c2ccc(Cl)cc2)CC1] |
| 124 | 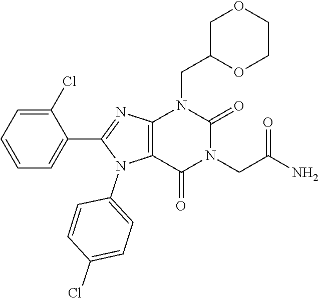 [SMILES: NC(=O)Cn1c(=O)c2c(nc(-c3ccccc3Cl)n2-c2ccc(Cl)cc2)n(CC2COCCO2)c1=O] |
| 125 | 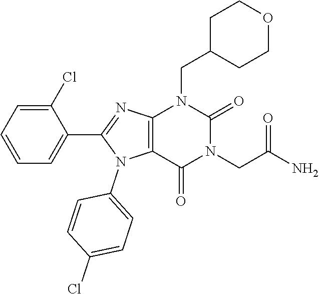 [SMILES: NC(=O)Cn1c(=O)c2c(nc(-c3ccccc3Cl)n2-c2ccc(Cl)cc2)n(CC2CCOCC2)c1=O] |
| 126 | 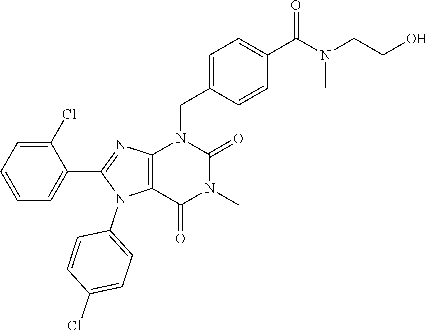 [SMILES: CN(CCO)C(=O)c1ccc(Cn2c(=O)n(C)c(=O)c3c2nc(-c2ccccc2Cl)n3-c2ccc(Cl)cc2)cc1] |
| 127 | 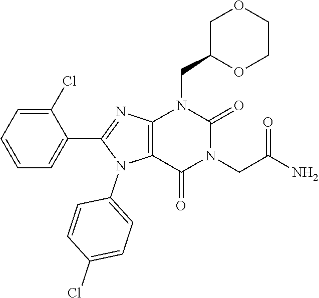 [SMILES: NC(=O)Cn1c(=O)c2c(nc(-c3ccccc3Cl)n2-c2ccc(Cl)cc2)n(C[C@H]2COCCO2)c1=O] |
| 128 | 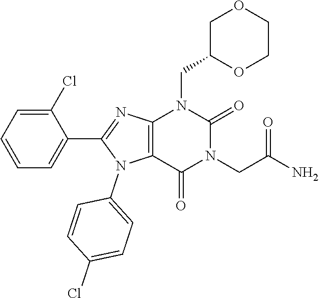 [SMILES: NC(=O)Cn1c(=O)c2c(nc(-c3ccccc3Cl)n2-c2ccc(Cl)cc2)n(C[C@@H]2COCCO2)c1=O] |
| 129 | 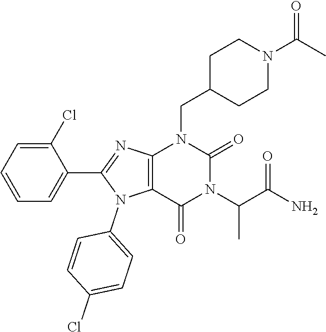 [SMILES: CC(=O)N1CCC(Cn2c(=O)n(C(C)C(N)=O)c(=O)c3c2nc(-c2ccccc2Cl)n3-c2ccc(Cl)cc2)CC1] |
| 130 | 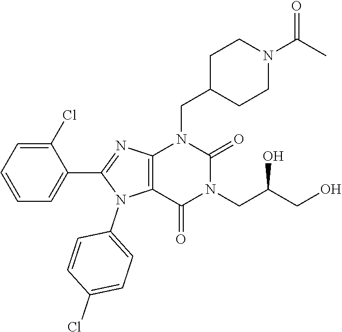 [SMILES: CC(=O)N1CCC(Cn2c(=O)n(C[C@@H](O)CO)c(=O)c3c2nc(-c2ccccc2Cl)n3-c2ccc(Cl)cc2)CC1] |
| 131 | 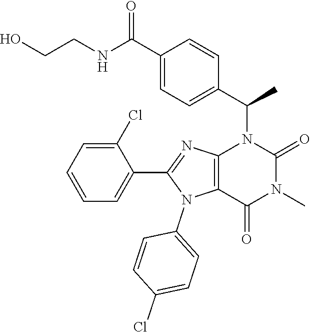 [SMILES: C[C@H](c1ccc(C(=O)NCCO)cc1)n1c(=O)n(C)c(=O)c2c1nc(-c1ccccc1Cl)n2-c1ccc(Cl)cc1] |
| 132 | 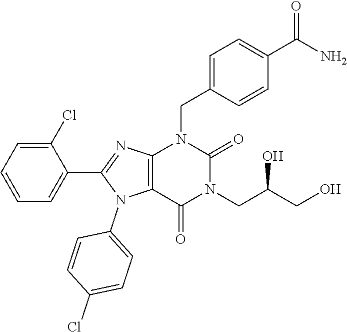 [SMILES: NC(=O)c1ccc(Cn2c(=O)n(C[C@@H](O)CO)c(=O)c3c2nc(-c2ccccc2Cl)n3-c2ccc(Cl)cc2)cc1] |
| 133 | 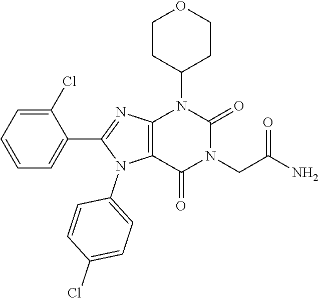 [SMILES: NC(=O)Cn1c(=O)c2c(nc(-c3ccccc3Cl)n2-c2ccc(Cl)cc2)n(C2CCOCC2)c1=O] |
| 134 | 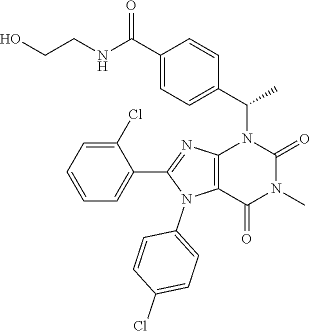 [SMILES: C[C@@H](c1ccc(C(=O)NCCO)cc1)n1c(=O)n(C)c(=O)c2c1nc(-c1ccccc1Cl)n2-c1ccc(Cl)cc1] |
| 135 | 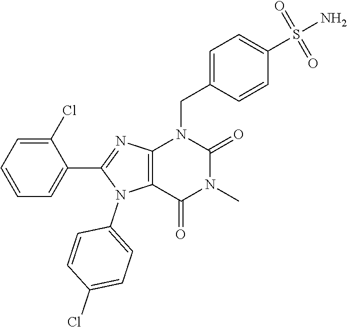 [SMILES: Cn1c(=O)c2c(nc(-c3ccccc3Cl)n2-c2ccc(Cl)cc2)n(Cc2ccc(S(N)(=O)=O)cc2)c1=O] |
| 136 | 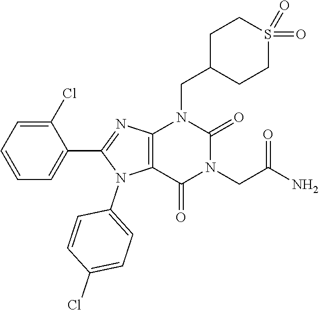 [SMILES: NC(=O)Cn1c(=O)c2c(nc(-c3ccccc3Cl)n2-c2ccc(Cl)cc2)n(CC2CCS(=O)(=O)CC2)c1=O] |
| 137 | 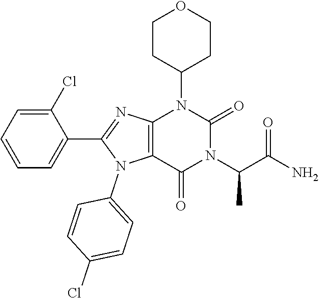 [SMILES: C[C@H](C(N)=O)n1c(=O)c2c(nc(-c3ccccc3Cl)n2-c2ccc(Cl)cc2)n(C2CCOCC2)c1=O] |
| 138 | 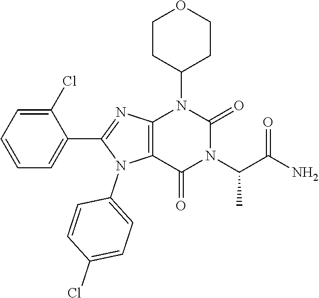 [SMILES: C[C@@H](C(N)=O)n1c(=O)c2c(nc(-c3ccccc3Cl)n2-c2ccc(Cl)cc2)n(C2CCOCC2)c1=O] |
| 139 | 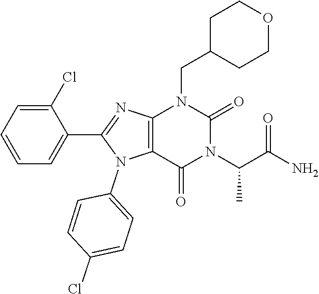 [SMILES: C[C@@H](C(N)=O)n1c(=O)c2c(nc(-c3ccccc3Cl)n2-c2ccc(Cl)cc2)n(CC2CCOCC2)c1=O] |
| 140 | 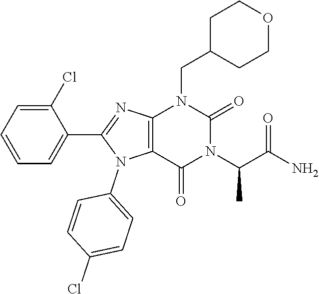 [SMILES: C[C@H](C(N)=O)n1c(=O)c2c(nc(-c3ccccc3Cl)n2-c2ccc(Cl)cc2)n(CC2CCOCC2)c1=O] |
| 141 | 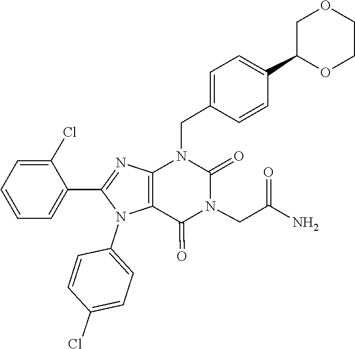 [SMILES: NC(=O)Cn1c(=O)c2c(nc(-c3ccccc3Cl)n2-c2ccc(Cl)cc2)n(Cc2ccc([C@H]3COCCO3)cc2)c1=O] |
| 142 | 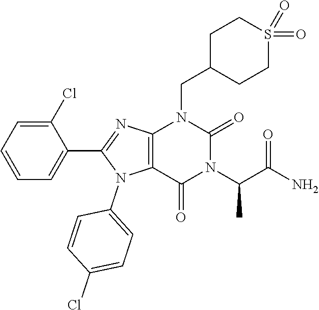 [SMILES: C[C@H](C(N)=O)n1c(=O)c2c(nc(-c3ccccc3Cl)n2-c2ccc(Cl)cc2)n(CC2CCS(=O)(=O)CC2)c1=O] |
| 143 | 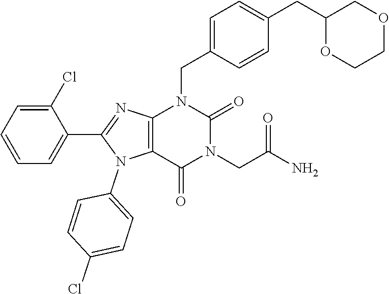 [SMILES: NC(=O)Cn1c(=O)c2c(nc(-c3ccccc3Cl)n2-c2ccc(Cl)cc2)n(Cc2ccc(CC3COCCO3)cc2)c1=O] |
| 144 | 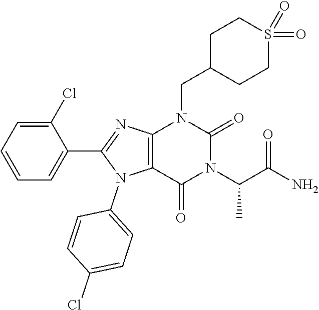 [SMILES: C[C@@H](C(N)=O)n1c(=O)c2c(nc(-c3ccccc3Cl)n2-c2ccc(Cl)cc2)n(CC2CCS(=O)(=O)CC2)c1=O] |
| 145 | 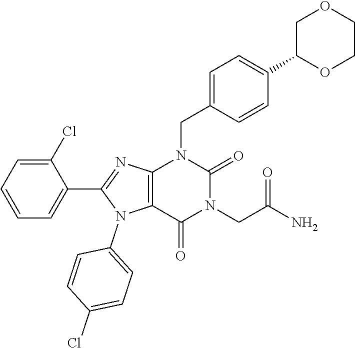 [SMILES: NC(=O)Cn1c(=O)c2c(nc(-c3ccccc3Cl)n2-c2ccc(Cl)cc2)n(Cc2ccc([C@@H]3COCCO3)cc2)c1=O] |
| 146 | 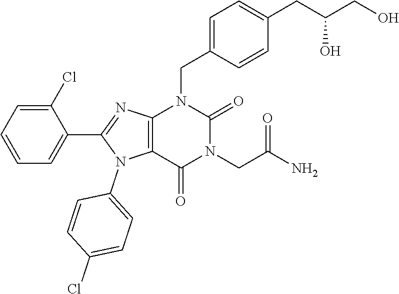 [SMILES: NC(=O)Cn1c(=O)c2c(nc(-c3ccccc3Cl)n2-c2ccc(Cl)cc2)n(Cc2ccc(C[C@@H](O)CO)cc2)c1=O] |
| 147 | 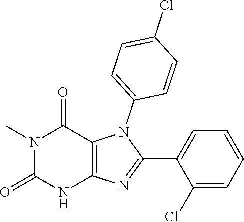 [SMILES: Cn1c(=O)[nH]c2nc(-c3ccccc3Cl)n(-c3ccc(Cl)cc3)c2c1=O] |
| 148 | 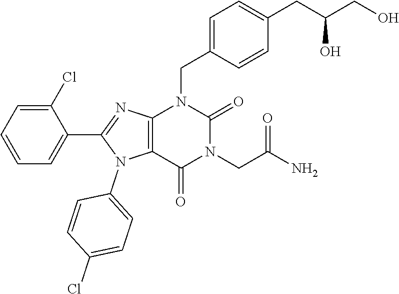 [SMILES: NC(=O)Cn1c(=O)c2c(nc(-c3ccccc3Cl)n2-c2ccc(Cl)cc2)n(Cc2ccc(C[C@H](O)CO)cc2)c1=O] |
| 149 | 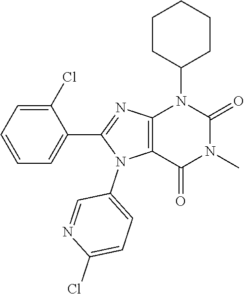 [SMILES: Cn1c(=O)c2c(nc(-c3ccccc3Cl)n2-c2ccc(Cl)nc2)n(C2CCCCC2)c1=O] |
| 150 | 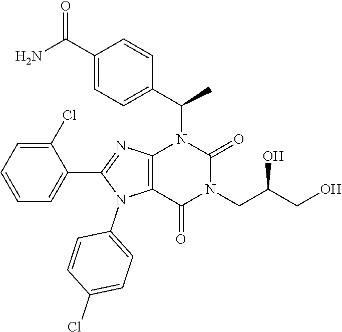 [SMILES: C[C@H](c1ccc(C(N)=O)cc1)n1c(=O)n(C[C@@H](O)CO)c(=O)c2c1nc(-c1ccccc1Cl)n2-c1ccc(Cl)cc1] |
| 151 | 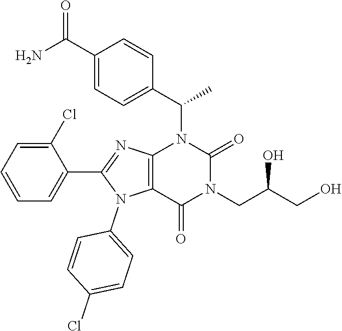 [SMILES: C[C@@H](c1ccc(C(N)=O)cc1)n1c(=O)n(C[C@@H](O)CO)c(=O)c2c1nc(-c1ccccc1Cl)n2-c1ccc(Cl)cc1] |
| 152 | 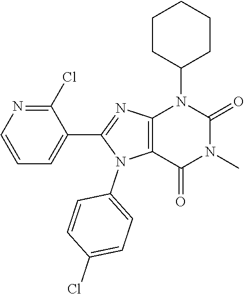 [SMILES: Cn1c(=O)c2c(nc(-c3cccnc3Cl)n2-c2ccc(Cl)cc2)n(C2CCCCC2)c1=O] |
| 153 | 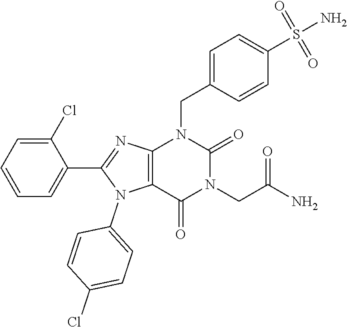 [SMILES: NC(=O)Cn1c(=O)c2c(nc(-c3ccccc3Cl)n2-c2ccc(Cl)cc2)n(Cc2ccc(S(N)(=O)=O)cc2)c1=O] |
| 154 | 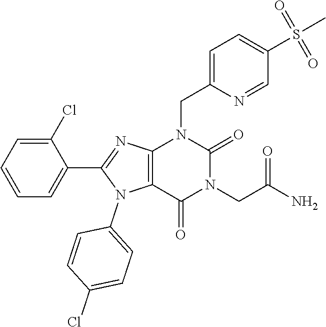 [SMILES: CS(=O)(=O)c1ccc(Cn2c(=O)n(CC(N)=O)c(=O)c3c2nc(-c2ccccc2Cl)n3-c2ccc(Cl)cc2)nc1] |
| 155 | 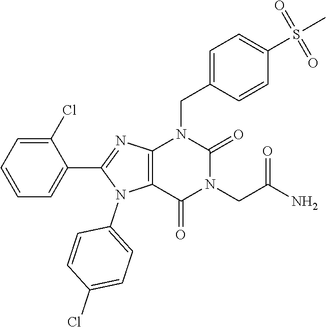 [SMILES: CS(=O)(=O)c1ccc(Cn2c(=O)n(CC(N)=O)c(=O)c3c2nc(-c2ccccc2Cl)n3-c2ccc(Cl)cc2)cc1] |
| 156 | 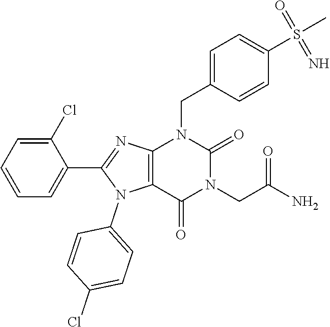 [SMILES: CS(=N)(=O)c1ccc(Cn2c(=O)n(CC(N)=O)c(=O)c3c2nc(-c2ccccc2Cl)n3-c2ccc(Cl)cc2)cc1] |
| 157 | 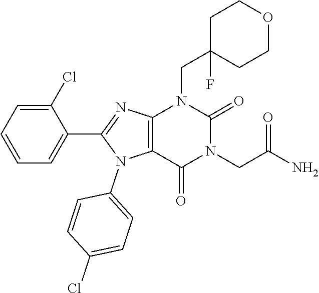 [SMILES: NC(=O)Cn1c(=O)c2c(nc(-c3ccccc3Cl)n2-c2ccc(Cl)cc2)n(CC2(F)CCOCC2)c1=O] |
| 158 | 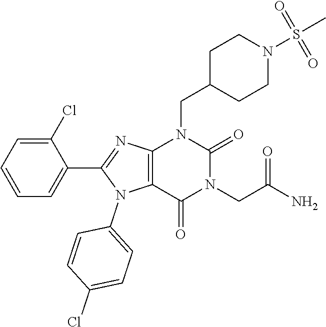 [SMILES: CS(=O)(=O)N1CCC(Cn2c(=O)n(CC(N)=O)c(=O)c3c2nc(-c2ccccc2Cl)n3-c2ccc(Cl)cc2)CC1] |
| 159 | 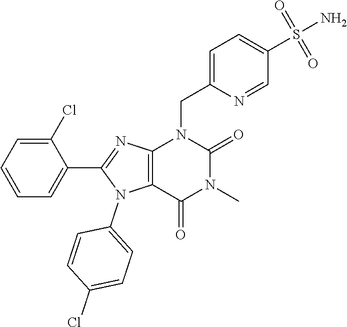 [SMILES: Cn1c(=O)c2c(nc(-c3ccccc3Cl)n2-c2ccc(Cl)cc2)n(Cc2ccc(S(N)(=O)=O)cn2)c1=O] |
| 160 | 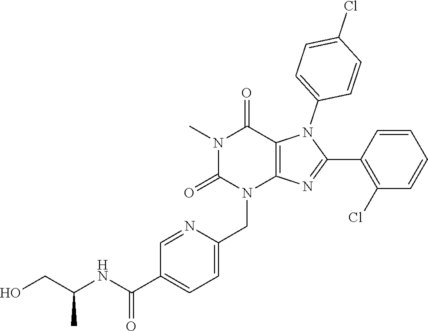 [SMILES: C[C@@H](CO)NC(=O)c1ccc(Cn2c(=O)n(C)c(=O)c3c2nc(-c2ccccc2Cl)n3-c2ccc(Cl)cc2)nc1] |
| 161 | 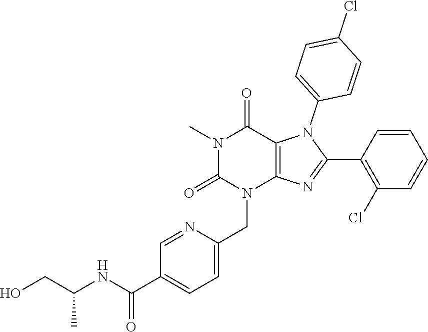 [SMILES: C[C@H](CO)NC(=O)c1ccc(Cn2c(=O)n(C)c(=O)c3c2nc(-c2ccccc2Cl)n3-c2ccc(Cl)cc2)nc1] |
| 162 | 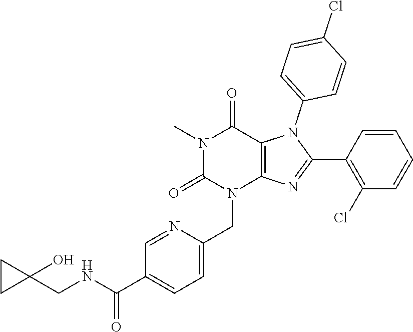 [SMILES: Cn1c(=O)c2c(nc(-c3ccccc3Cl)n2-c2ccc(Cl)cc2)n(Cc2ccc(C(=O)NCC3(O)CC3)cn2)c1=O] |
| 163 | 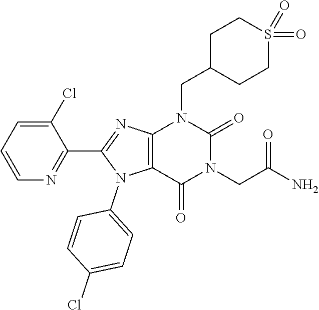 [SMILES: NC(=O)Cn1c(=O)c2c(nc(-c3ncccc3Cl)n2-c2ccc(Cl)cc2)n(CC2CCS(=O)(=O)CC2)c1=O] |
| 164 | 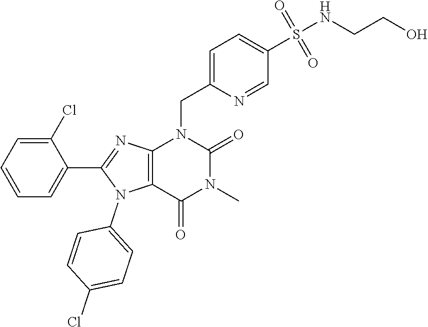 [SMILES: Cn1c(=O)c2c(nc(-c3ccccc3Cl)n2-c2ccc(Cl)cc2)n(Cc2ccc(S(=O)(=O)NCCO)cn2)c1=O] |
| 165 | 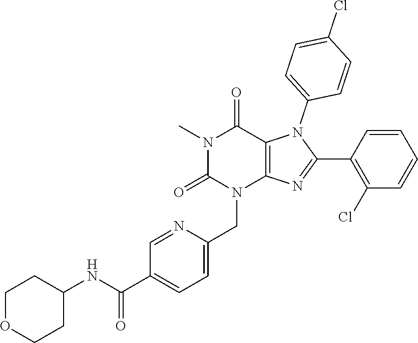 [SMILES: Cn1c(=O)c2c(nc(-c3ccccc3Cl)n2-c2ccc(Cl)cc2)n(Cc2ccc(C(=O)NC3CCOCC3)cn2)c1=O] |
| 166 | 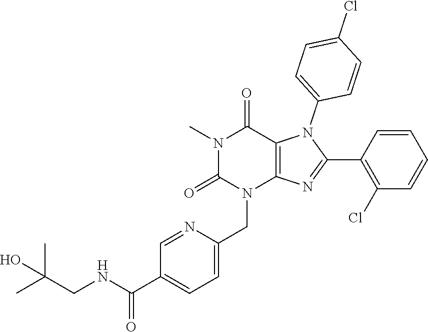 [SMILES: Cn1c(=O)c2c(nc(-c3ccccc3Cl)n2-c2ccc(Cl)cc2)n(Cc2ccc(C(=O)NCC(C)(C)O)cn2)c1=O] |
| 167 | 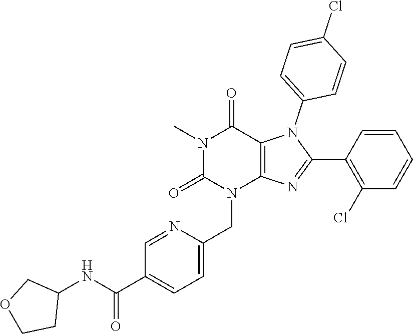 [SMILES: Cn1c(=O)c2c(nc(-c3ccccc3Cl)n2-c2ccc(Cl)cc2)n(Cc2ccc(C(=O)NC3CCOC3)cn2)c1=O] |
| 168 | 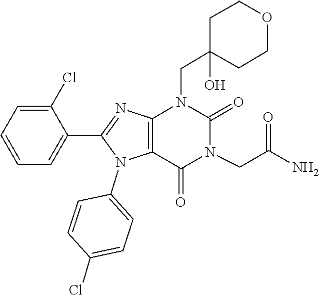 [SMILES: CCC(O)(CCOC)Cn1c(=O)n(CC(N)=O)c(=O)c2c1nc(-c1ccccc1Cl)n2-c1ccc(Cl)cc1] |
| 169 | 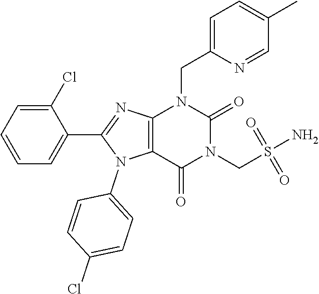 [SMILES: Cc1ccc(Cn2c(=O)n(CS(N)(=O)=O)c(=O)c3c2nc(-c2ccccc2Cl)n3-c2ccc(Cl)cc2)nc1] |
| 170 | 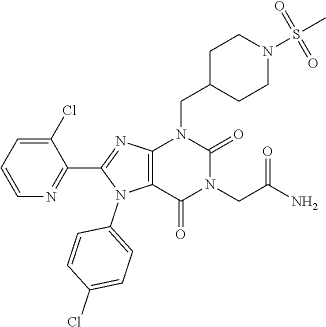 [SMILES: CS(=O)(=O)N1CCC(Cn2c(=O)n(CC(N)=O)c(=O)c3c2nc(-c2ncccc2Cl)n3-c2ccc(Cl)cc2)CC1] |
| 171 | 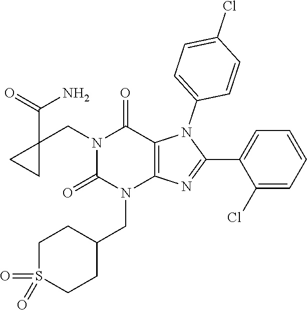 [SMILES: NC(=O)C1(Cn2c(=O)c3c(nc(-c4ccccc4Cl)n3-c3ccc(Cl)cc3)n(CC3CCS(=O)(=O)CC3)c2=O)CC1] |
| 172 | 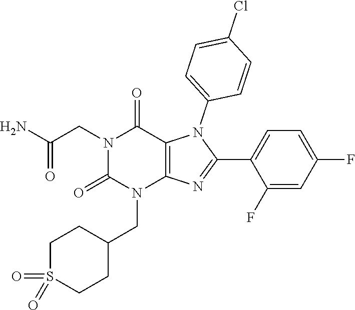 [SMILES: NC(=O)Cn1c(=O)c2c(nc(-c3ccc(F)cc3F)n2-c2ccc(Cl)cc2)n(CC2CCS(=O)(=O)CC2)c1=O] |
| 173 | 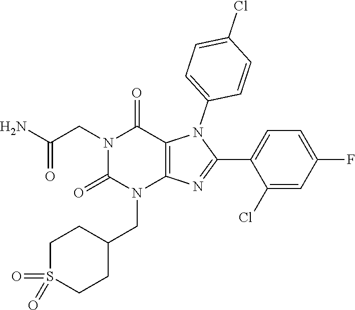 [SMILES: NC(=O)Cn1c(=O)c2c(nc(-c3ccc(F)cc3Cl)n2-c2ccc(Cl)cc2)n(CC2CCS(=O)(=O)CC2)c1=O] |
| 174 | 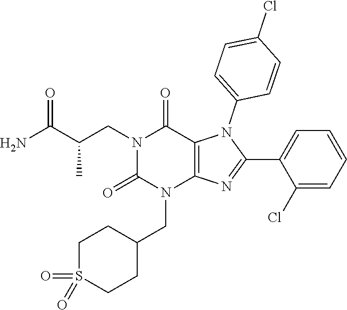 [SMILES: C[C@@H](Cn1c(=O)c2c(nc(-c3ccccc3Cl)n2-c2ccc(Cl)cc2)n(CC2CCS(=O)(=O)CC2)c1=O)C(N)=O] |
| 175 | 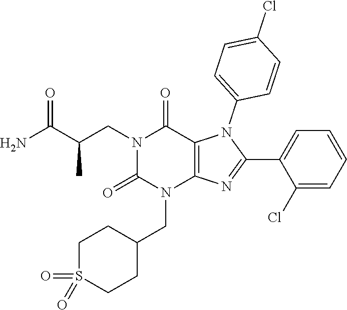 [SMILES: C[C@H](Cn1c(=O)c2c(nc(-c3ccccc3Cl)n2-c2ccc(Cl)cc2)n(CC2CCS(=O)(=O)CC2)c1=O)C(N)=O] |
| 176 | 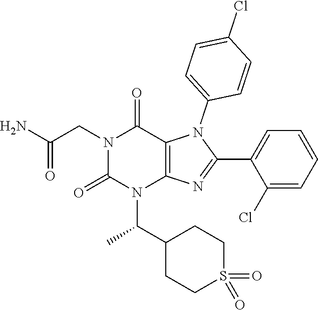 [SMILES: C[C@@H](C1CCS(=O)(=O)CC1)n1c(=O)n(CC(N)=O)c(=O)c2c1nc(-c1ccccc1Cl)n2-c1ccc(Cl)cc1] |
| 177 | 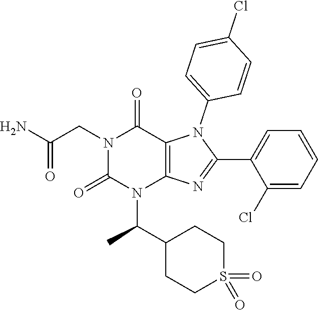 [SMILES: C[C@H](C1CCS(=O)(=O)CC1)n1c(=O)n(CC(N)=O)c(=O)c2c1nc(-c1ccccc1Cl)n2-c1ccc(Cl)cc1] |
| 178 | 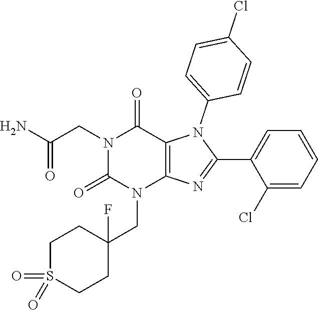 [SMILES: NC(=O)Cn1c(=O)c2c(nc(-c3ccccc3Cl)n2-c2ccc(Cl)cc2)n(CC2(F)CCS(=O)(=O)CC2)c1=O] |
| 179 | 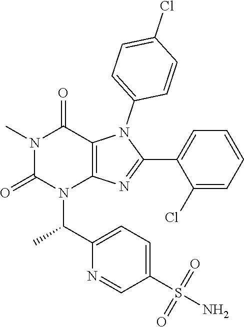 [SMILES: C[C@@H](c1ccc(S(N)(=O)=O)cn1)n1c(=O)n(C)c(=O)c2c1nc(-c1ccccc1Cl)n2-c1ccc(Cl)cc1] |
| 180 | 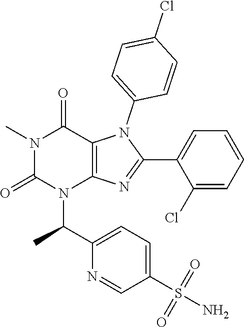 [SMILES: C[C@H](c1ccc(S(N)(=O)=O)cn1)n1c(=O)n(C)c(=O)c2c1nc(-c1ccccc1Cl)n2-c1ccc(Cl)cc1] |
| 181 | 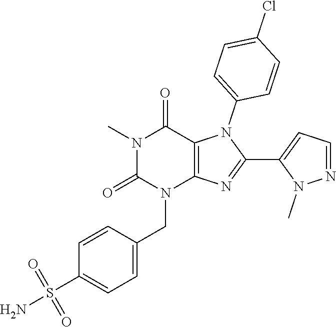 [SMILES: Cn1nccc1-c1nc2c(c(=O)n(C)c(=O)n2Cc2ccc(S(N)(=O)=O)cc2)n1-c1ccc(Cl)cc1] |
| 182 | 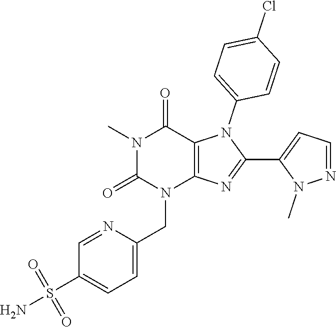 [SMILES: Cn1nccc1-c1nc2c(c(=O)n(C)c(=O)n2Cc2ccc(S(N)(=O)=O)cn2)n1-c1ccc(Cl)cc1] |
| 183 | 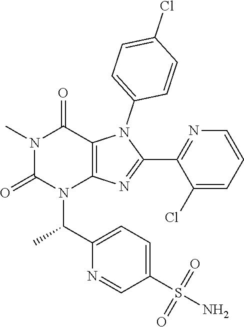 [SMILES: C[C@@H](c1ccc(S(N)(=O)=O)cn1)n1c(=O)n(C)c(=O)c2c1nc(-c1ncccc1Cl)n2-c1ccc(Cl)cc1] |
| 184 | 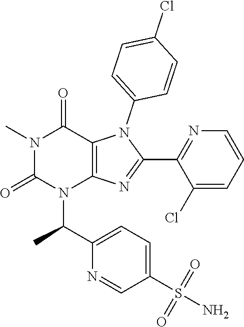 [SMILES: C[C@H](c1ccc(S(N)(=O)=O)cn1)n1c(=O)n(C)c(=O)c2c1nc(-c1ncccc1Cl)n2-c1ccc(Cl)cc1] |
| 185 | 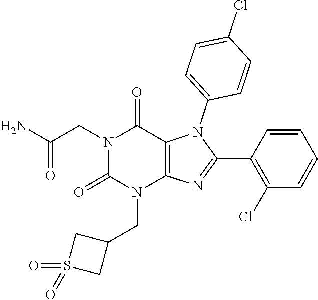 [SMILES: NC(=O)Cn1c(=O)c2c(nc(-c3ccccc3Cl)n2-c2ccc(Cl)cc2)n(CC2CS(=O)(=O)C2)c1=O] |
| 186 | 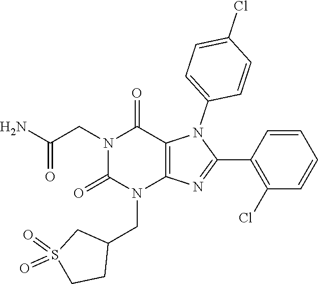 [SMILES: NC(=O)Cn1c(=O)c2c(nc(-c3ccccc3Cl)n2-c2ccc(Cl)cc2)n(CC2CCS(=O)(=O)C2)c1=O] |
| 187 | 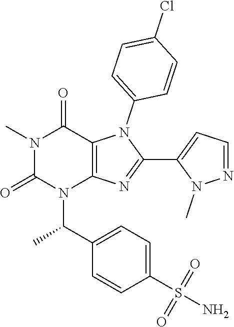 [SMILES: C[C@@H](c1ccc(S(N)(=O)=O)cc1)n1c(=O)n(C)c(=O)c2c1nc(-c1ccnn1C)n2-c1ccc(Cl)cc1] |
| 188 | 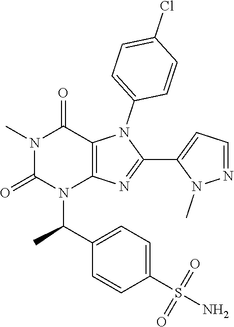 [SMILES: C[C@H](c1ccc(S(N)(=O)=O)cc1)n1c(=O)n(C)c(=O)c2c1nc(-c1ccnn1C)n2-c1ccc(Cl)cc1] |
| 189 | 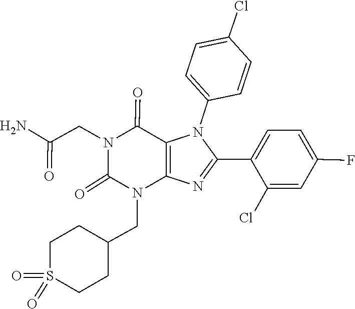 [SMILES: NC(=O)Cn1c(=O)c2c(nc(-c3ccc(F)cc3Cl)n2-c2ccc(Cl)cc2)n(CC2CCS(=O)(=O)CC2)c1=O] |
| 190 | 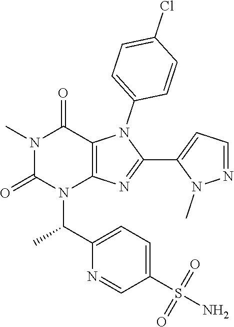 [SMILES: C[C@@H](c1ccc(S(N)(=O)=O)cn1)n1c(=O)n(C)c(=O)c2c1nc(-c1ccnn1C)n2-c1ccc(Cl)cc1] |

In certain embodiments, the compounds of the invention may be racemic. In certain embodiments, the compounds of the invention may be enriched in one enantiomer. For example, a compound of the invention may have greater than 30% ee, 40% ee, 50% ee, 60% ee, 70% ee, 80% ee, 90% ee, or even 95% or greater ee.

The compounds of the invention have more than one stereocenter. Accordingly, the compounds of the invention may be enriched in one or more diastereomers. For example, a compound of the invention may have greater than 30% de, 40% de, 50% de, 60% de, 70% de, 80% de, 90% de, or even 95% or greater de. In certain embodiments, the compounds of the invention have substantially one isomeric configuration at one or more stereogenic centers, and have multiple isomeric configurations at the remaining stereogenic centers.

In certain embodiments, the enantiomeric excess of the stereocenter is at least 40% ee, 50% ee, 60% ee, 70% ee, 80% ee, 90% ee, 92% ee, 94% ee, 95% ee, 96% ee, 98% ee or greater ee.

As used herein, single bonds drawn without stereochemistry do not indicate the stereochemistry of the compound.

As used herein, hashed or bolded non-wedge bonds indicate relative, but not absolute, stereochemical configuration (e.g., do not distinguish between enantiomers of a given diastereomer).

As used herein, hashed or bolded wedge bonds indicate absolute stereochemical configuration.

### Pharmaceutical Compositions

In certain embodiments, the invention relates to a composition comprising a compound of the invention and a pharmaceutically acceptable carrier.

In some embodiments, the invention relates to pharmaceutical composition comprising a compound of the invention and a pharmaceutically acceptable carrier. In certain embodiments, a therapeutic preparation or pharmaceutical composition of the compound of the invention may be enriched to provide predominantly one enantiomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, a therapeutic preparation or pharmaceutical composition may be enriched to provide predominantly one diastereomer of the compound of the invention. A diastereomerically enriched mixture may comprise, for example, at least 60 mol percent of one diastereomer, or more preferably at least 75, 90, 95, or even 99 mol percent.

The compounds of this invention may be used in treating the conditions described herein, in the form of the free base, salts (preferably pharmaceutically acceptable salts), solvates, hydrates, prodrugs, isomers, or mixtures thereof. All forms are within the scope of the disclosure. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the subject organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

Pharmaceutically acceptable salts within the scope of the disclosure include those derived from the following acids: mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

“Pharmaceutically acceptable salt” is used herein to refer to an acid addition salt or a basic addition salt which is suitable for or compatible with the treatment of patients.

The term “pharmaceutically acceptable acid addition salt” as used herein means any non-toxic organic or inorganic salt of the disclosed compounds. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, bitartaric, citric, ascorbic, maleic, benzoic, phenylacetic, cinnamic, salicylic, and sulfosalicylic acids, as well as sulfonic acids such as p-toluene sulfonic and methanesulfonic acids. Mono or di-acid salts can be formed, and such salts may exist in either a hydrated, solvated or substantially anhydrous form. In general, the acid addition salts of compounds disclosed herein are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds disclosed herein for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The compounds of the present invention can be formulated as pharmaceutical compositions and administered to a subject in need of treatment, for example a mammal, such as a human patient, in a variety of forms adapted to the chosen route of administration. A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously, transepithelially, intrapulmonary, or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. Parenteral administration may be by continuous infusion over a selected period of time.

In accordance with the methods of the disclosure, the described compounds may be administered to a patient in a variety of forms depending on the selected route of administration, as will be understood by those skilled in the art. The compositions containing the compounds of the disclosure can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

A composition comprising a compound of the present disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

A person skilled in the art would know how to prepare suitable formulations. Conventional procedures and ingredients for the selection and preparation of suitable formulations are described, for example, in Remington's Pharmaceutical Sciences (1990-18th edition) and in The United States Pharmacopeia: The National Formulary (USP 24 NF19) published in 1999.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Thus, compounds of the invention may be systemically administered, e.g., orally, in combination with a pharmaceutically acceptable vehicle such as an inert diluent or an assimilable edible carrier; or by inhalation or insufflation. They may be enclosed in hard or soft shell gelatin capsules, may be compressed into tablets, or may be incorporated directly with the food of the patient's diet. For oral therapeutic administration, the compounds may be combined with one or more excipients and used in the form of ingestible tablets, buccal tablets, troches, capsules, elixirs, suspensions, syrups, wafers, and the like. The compounds may be combined with a fine inert powdered carrier and inhaled by the subject or insufflated. Such compositions and preparations should contain at least 0.1% of compounds of formula I. The percentage of the compositions and preparations may, of course, be varied and may conveniently be between about 2% to about 60% of the weight of a given unit dosage form. The amount of the compounds in such therapeutically useful compositions is such that an effective dosage level will be obtained.

In certain embodiments of the disclosure, compositions comprising a compound of the present disclosure for oral administration include capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of the compound of the present disclosure as an active ingredient.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

In solid dosage forms for oral administration (capsules, tablets, troches, pills, dragees, powders, granules, and the like), one or more compositions comprising the compound of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, gum tragacanth, corn starch, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the compounds may be incorporated into sustained-release preparations and devices. For example, the compounds may be incorporated into time release capsules, time release tablets, and time release pills.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration include pharmaceutically acceptable emulsions, lyophiles for reconstitution, microemulsions, solutions, suspensions, syrups, and elixirs. In addition to the compound of the present disclosure, the liquid dosage forms may contain inert diluents commonly used in the art, such as water or other solvents, cyclodextrins and derivatives thereof, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, salts and/or prodrugs thereof, may contain suspending agents such as ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, intraocular, subcapsular, subarachnoid, intraspinal and intrasternal injection and infusion.

In certain embodiments, pharmaceutical compositions suitable for parenteral administration comprise the compound of the present disclosure in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compounds may be administered intravenously or intraperitoneally by infusion or injection. Solutions of the compounds or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For topical administration, the compounds may be applied in pure form. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Other solid carriers include nontoxic polymeric nanoparticles or microparticles. Useful liquid carriers include water, alcohols or glycols or water/alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949, which is hereby incorporated by reference.

For example, the concentration of the compounds in a liquid composition, such as a lotion, can be from about 0.1-25% by weight, or from about 0.5-10% by weight. The concentration in a semi-solid or solid composition such as a gel or a powder can be about 0.1-5% by weight, or about 0.5-2.5% by weight.

For use in the methods of this invention, active compounds can be given per se or as a pharmaceutical composition containing, for example, about 0.1 to about 99.5% (more preferably, about 0.5 to about 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The amount of the compounds required for use in treatment will vary not only with the particular salt selected but also with the route of administration, the nature of the condition being treated and the age and condition of the patient and will be ultimately at the discretion of the attendant physician or clinician.

Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms, for example, as two, three, four or more sub-doses per day. The sub-dose itself may be further divided, e.g., into a number of discrete loosely spaced administrations such as multiple inhalations from an insufflator. In certain embodiments of the present invention, the active compound may be administered two or three times daily. In certain embodiments, the active compound will be administered once daily.

The dosage of the compounds and/or compositions of the disclosure can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. The compounds of the disclosure may be administered initially in a suitable dosage that may be adjusted as required, depending on the clinical response. To calculate the human equivalent dose (HED) from a dosage used in the treatment of age-dependent cognitive impairment in rats, the formula HED (mg/kg)=rat dose (mg/kg)×0.16 may be employed (see Estimating the Safe Starting Dose in Clinical Trials for Therapeutics in Adult Healthy Volunteers, December 2002, Center for Biologics Evaluation and Research). For example, using that formula, a dosage of 10 mg/kg in rats is equivalent to 1.6 mg/kg in humans. This conversion is based on a more general formula HED=animal dose in mg/kg×(animal weight in kg/human weight in kg) 0.33. Similarly, to calculate the HED from a dosage used in the treatment in mouse, the formula HED (mg/kg)=mouse dose (mg/kg)×0.08 may be employed (see Estimating the Safe Starting Dose in Clinical Trials for Therapeutics in Adult Healthy Volunteers, December 2002, Center for Biologics Evaluation and Research).

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Treatment

Aberrant activity of the CB1 receptor has been implicated in numerous diseases and conditions, including diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

A. Focal Segmental Glomerulosclerosis (TSGS)

Focal Segmental Glomerulosclerosis (FSGS) is a disease that attacks the kidney's filtering system (glomeruli) causing serious scarring. FSGS is one of the many causes of a disease known as Nephrotic Syndrome, which occurs when protein in the blood leaks into the urine (proteinuria). Primary FSGS, when no underlying cause is found, usually presents as nephrotic syndrome. Secondary FSGS, when an underlying cause is identified, usually presents with kidney failure and proteinuria. FSGS can be genetic; there are currently several known genetic causes of the hereditary forms of FSGS.

Very few treatments are available for patients with FSGS. Many patients are treated with steroid regimens, most of which have very harsh side effects. Some patients have shown to respond positively to immunosuppressive drugs as well as blood pressure drugs which have shown to lower the level of protein in the urine. To date, there is no commonly accepted effective treatment or cure and there are no FDA approved drugs to treat FSGS. Therefore, more effective methods to reduce or inhibit proteinuria are desirable.

B. IgA Nephropathy

IgA nephropathy (also known as IgA nephritis, IgAN, Berger's disease, and synpharyngitic glomerulonephritis) is a form of glomerulonephritis (inflammation of the glomeruli of the kidney). IgA nephropathy is the most common glomerulonephritis throughout the world. Primary IgA nephropathy is characterized by deposition of the IgA antibody in the glomerulus. There are other diseases associated with glomerular IgA deposits, the most common being Henoch-Schönlein purpura (HSP), which is considered by many to be a systemic form of IgA nephropathy. Henoch-Schönlein purpura presents with a characteristic purpuric skin rash, arthritis, and abdominal pain and occurs more commonly in young adults (16-35 yrs old). HSP is associated with a more benign prognosis than IgA nephropathy. In IgA nephropathy there is a slow progression to chronic renal failure in 25-30% of cases during a period of 20 years.

C. Diabetic Nephropathy

Diabetic nephropathy, also known as Kimmelstiel-Wilson syndrome and intercapillary glomerulonephritis, is a progressive kidney disease caused by angiopathy of capillaries in the kidney glomeruli. It is characterized by nephrotic syndrome and diffuse glomerulosclerosis. It is due to longstanding diabetes mellitus and is a prime cause for dialysis. The earliest detectable change in the course of diabetic nephropathy is a thickening in the glomerulus. At this stage, the kidney may start allowing more serum albumin than normal in the urine. As diabetic nephropathy progresses, increasing numbers of glomeruli are destroyed by nodular glomerulosclerosis and the amount of albumin excreted in the urine increases.

D. Nephrotic Syndrome

Nephrotic syndrome is a collection of symptoms due to kidney damage. This includes protein in the urine, low blood albumin levels, high blood lipids, and significant swelling. Other symptoms may include weight gain, feeling tired, and foamy urine. Complications may include blood clots, infections, and high blood pressure. Causes include a number of kidney diseases such as focal segmental glomerulosclerosis, membranous nephropathy, and minimal change disease. It may also occur as a complication of diabetes or lupus. The underlying mechanism typically involves damage to the glomeruli of the kidney. Diagnosis is typically based on urine testing and sometimes a kidney biopsy. It differs from nephritic syndrome in that there are no red blood cells in the urine. Nephrotic syndrome is characterized by large amounts of proteinuria (>3.5 g per 1.73 m2 body surface area per day, or >40 mg per square meter body surface area per hour in children), hypoalbuminemia (<2.5 g/dl), hyperlipidaemia, and edema that begins in the face. Lipiduria (lipids in urine) can also occur, but is not essential for the diagnosis of nephrotic syndrome. Hyponatremia also occur with a low fractional sodium excretion. Genetic forms of nephrotic syndrome are typically resistant to steroid and other immunosuppressive treatment. Goals of therapy are to control urinary protein loss and swelling, provide good nutrition to allow the child to grow, and prevent complications. Early and aggressive treatment are used to control the disorder.

E. Diabetic Kidney Disease

Diabetic kidney disease is a decrease in kidney function that occurs in some diabetes patients. The causes of diabetic kidney disease are complex and most likely related to many factors. Some experts feel that changes in the circulation of blood within the filtering apparatus of the kidney (the glomerulus) may play an important role. In the early stages, there may not be any symptoms. As kidney function decreases further, toxic wastes build up, and patients often feel sick to their stomachs and throw up, lose their appetites, have hiccups and gain weight due to fluid retention. If left untreated, patients can develop heart failure and fluid in their lungs. In patients with Type I (juvenile-onset or insulin-dependent) diabetes, a diagnosis of early kidney disease can be based on the presence of very small amounts of protein in the urine (microalbuminuria). Special methods are needed to measure these small amounts of protein. When the amount of protein in the urine becomes large enough to be detected by standard tests, the patient is said to have “clinical” diabetic kidney disease.

F. Obesity-Related Kidney Disease

Obesity is a potent risk factor for the development of kidney disease. It increases the risk of developing major risk factors for chronic kidney disease (CKD), like diabetes and hypertension, and it has a direct impact on the development of CKD and end-stage renal disease (ESRD). In individuals affected by obesity, a possibly compensatory mechanism of hyperfiltration occurs to meet the heightened metabolic demands of the increased body weight. The increase in intraglomerular pressure can damage the kidney structure and raise the risk of developing CKD in the long term. (Kovesdy, C. P., et al. (2017). Obesity and Kidney Disease: Hidden Consequences of the Epidemic. Canadian Journal of Kidney Health and Disease. doi:10.1177/2054358117698669.)

G. Kidney Fibrosis

Renal fibrosis, characterized by tubulointerstitial fibrosis and gliomerulosclerosis, is the final manifestation of chronic kidney disease. Renal fibrosis is characterized by an excessive accumulation and deposition of extracellular matrix components. This pathologic result usually originates from both underlying complicated cellular activities such as epithelial-to-mesenchymal transition, fibroblast activation, monocyte/macrophage infiltration, and cellular apoptosis and the activation of signaling molecules such as transforming growth factor beta and angiotensin 11. (Cho M-H. Renal fibrosis. Korean J Pediatr. 2010; 53(7):735-740. doi:10.3345/kjp.2010.53.7.735.)

H. Prader Willi Syndrome

Prader-Willi syndrome is a genetic disorder due to loss of function of specific genes and affecting between one in 10,000 and one in 30,000 people. In newborns, symptoms include weak muscles, poor feeding, and slow development. Beginning in childhood, the person becomes constantly hungry, which often leads to obesity and type 2 diabetes. About 74% of cases occur when part of the father's chromosome 15 is deleted. In another 25% of cases, the person has two copies of chromosome 15 from their mother and none from their father. As parts of the chromosome from the mother are turned off, they end up with no working copies of certain genes. Prader-Willi syndrome has no cure. Treatment, however, may improve outcomes, especially if carried out early. In newborns, feeding difficulties may be supported with feeding tubes. Strict food supervision is typically required starting around the age of three in combination with an exercise program. Growth hormone therapy also improves outcomes.

I. Metabolic Syndrome

Metabolic syndrome, sometimes known by other names, is a clustering of at least three of the five following medical conditions: central obesity, high blood pressure, high blood sugar, high serum triglycerides, and low serum high-density lipoprotein (HDL). Metabolic syndrome is associated with the risk of developing cardiovascular disease and type 2 diabetes. In the US about a quarter of the adult population has metabolic syndrome, and the prevalence increases with age, with racial and ethnic minorities being particularly affected. Insulin resistance, metabolic syndrome, and prediabetes are closely related to one another and have overlapping aspects. The syndrome is thought to be caused by an underlying disorder of energy utilization and storage. The key sign of metabolic syndrome is central obesity, also known as visceral, male-pattern or apple-shaped adiposity. It is characterized by adipose tissue accumulation predominantly around the waist and trunk. Other signs of metabolic syndrome include high blood pressure, decreased fasting serum HDL cholesterol, elevated fasting serum triglyceride level, impaired fasting glucose, insulin resistance, or prediabetes. The pathophysiology is very complex and has been only partially elucidated. Most patients are older, obese, sedentary, and have a degree of insulin resistance. Stress can also be a contributing factor. The most important risk factors are diet (particularly sugar-sweetened beverage consumption), genetics, aging, sedentary behavior or low physical activity, disrupted chronobiology/sleep, mood disorders/psychotropic medication use, and excessive alcohol use. Various strategies have been proposed to prevent the development of metabolic syndrome. These include increased physical activity (such as walking 30 minutes every day), and a healthy, reduced calorie diet. Generally, the individual disorders that compose the metabolic syndrome are medically treated separately.

J. Gastrointestinal Diseases

The endocannabinoid system has been implicated in gastrointestinal diseases such as functional dyspepsia (FD) and irritable bowel syndrome (IBS). (Pesce M, D'Alessandro A, Borrelli O, et al. Endocannabinoid-related compounds in gastrointestinal diseases. J Cell Mol Med. 2018; 22(2):706-715. doi:10.1111/jcmm.13359). Impaired gastric accommodation, delayed gastric emptying and visceral hypersensitivity have been suggested as the underlying pathophysiological mechanisms of some FD symptoms, such as nausea, early satiety, post-prandial fullness and pain. Oral administration of dronabinol (Δ9-THC) was able to significantly reduce gastric emptying in human beings. Furthermore, in healthy individuals, administration of a CB1 antagonist (rimonabant) was able to inhibit gastric accommodation, but not affecting gastric sensitivity, suggesting a role of ECS in the control of gastric accommodation. And given the evidence for a role of low-grade inflammation in IBS, endocannabinoids may improve IBS symptoms by decreasing the inflammatory response.

K. Non-Alcoholic Liver Disease and Non-Alcoholic Fatty Liver Disease

Non-alcoholic fatty liver disease (NAFLD) is the build up of extra fat in liver cells that is not caused by alcohol. It is normal for the liver to contain some fat. However, if more than 5%-10% percent of the liver's weight is fat, then it is called a fatty liver (steatosis). The more severe form of non-alcoholic fatty liver disease is called non-alcoholic steatohepatitis (NASH). Non-alcoholic steatohepatitis causes the liver to swell and become damaged. Non-alcoholic steatohepatitis tends to develop in people who are overweight or obese, or have diabetes, high cholesterol or high triglycerides. However, some people have non-alcoholic steatohepatitis even if they do not have any risk factors. Most people with non-alcoholic steatohepatitis are between the ages of 40 and 60 years. It is more common in women than in men. NASH often has no symptoms and people can have non-alcoholic steatohepatitis for years before symptoms occur.

Non-alcoholic steatohepatitis is one of the leading causes of cirrhosis in adults in the United States. Up to 25% of adults with non-alcoholic steatohepatitis may have cirrhosis. (“Non-Alcoholic Fatty Liver Disease,” American Liver Foundation, https://liverfoundation.org/for-patients/about-the-liver/diseases-of-the-liver/non-alcoholic-fatty-liver-disease/#1503448220833-1dc16d27-63ab).

L. Alcoholic Liver Disease

Alcoholic liver disease is a term that encompasses the liver manifestations of alcohol overconsumption, including fatty liver, alcoholic hepatitis, and chronic hepatitis with liver fibrosis or cirrhosis. It is the major cause of liver disease in Western countries. Although steatosis (fatty liver) will develop in any individual who consumes a large quantity of alcoholic beverages over a long period of time, this process is transient and reversible. Of all chronic heavy drinkers, only 15-20% develop hepatitis or cirrhosis, which can occur concomitantly or in succession.

Accordingly, in certain embodiments, the invention provides methods for treating a disease or condition characterized by aberrant CB1 activity comprising the step of administering to a subject in need thereof a compound or composition of the present disclosure. In certain embodiments, the disease or condition is diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

In some embodiments, the disease or condition is diabetic nephropathy.

In some embodiments, the disease or condition is focal segmental glomerular sclerosis.

In some embodiments, the disease or condition is nonalcoholic steatohepatitis.

Subjects to be Treated

In one aspect of the invention, a subject is selected on the basis that they have, or are at risk of developing, a disease or condition characterized by aberrant CB1 activity, such as diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses. In some embodiments, the subject is a mammal. In some embodiments, the subject is a human.

### EXAMPLES

The invention is further described in the following examples, which do not limit the scope of the invention described in the claims.

### Abbreviations

| | |
|---|---|
| Ac | acetyl |
| ACN | acetonitrile |
| aq. | aqueous |
| atm | atmospheres |
| DBU | 1-8-Diazabicyclo[5.4.0]undec-7-ene |
| DCM | Dichloromethane |
| DEAD | Diethyl azodicarboxylate |
| DIAD | Diisopropyl azodicarboxylate |
| DIEA/DIPEA | N,N-Diisopropylethylamine |
| DIPEA | N,N-Diisopropylethylamine |
| DMA | Dimethyl adipate |
| DMAP | |
| DMF | Dimethylformamide |
| DMSO | Dimethylsulfoxide |
| EDTA | Ethylenediamine tetraacetic acid |
| eq(s). | equivalent(s) |
| Et | Ethyl |
| EtOAc/EA | Ethyl acetate |
| EtOH | Ethanol |
| FA | Formic acid |
| g | gram(s) |
| HATU | (Dimethylamino)-N,N-dimethyl(3H- |
| | [1,2,3]triazolo[4,5-b]pyridin-3- |
| | yloxy)methaniminium hexafluorophosphate |
| Hex | Hexanes |
| HPLC | High pressure liquid chromatography |
| L | liter |
| LCMS; LC-MS | liquid chromatography mass spectrometry |
| Me | methyl |
| MeOH | Methanol |
| mg | milligram(s) |
| min | Minute(s) |
| mL; mL | milliliter(s) |
| mM | millimolar |
| MOP/POM | 2,2-dimethylpropanoyl)oxy] methyl |
| MS | mass spectrometry |
| NCS | |
| NMR | Nuclear magnetic resonance |
| PE | Petroleum ether |
| Ph | phenyl |
| RT | Retention time |
| SEMCl/SEM-Cl | 2-(Trimethylsilyl) ethoxymethyl chloride |
| TEA | Triethylamine |
| TFA | Trifluoroacetic acid |
| TLC | Thin layer chromatography |
| Ts | Tosyl |
| UV | Ultraviolet |
| X-Phos/XantPhos | 2-Dicyclohexylphosphino-2′,4′,6′- |
| | triisopropylbiphenyl |
| XantPhos Pd G3/ | (2-Dicyclohexylphosphino-2′,4′,6′- |
| XPhos Pd G3 | triisopropyl-1,1′-biphenyl)[2-(2′-amino-1,1′- |
| | biphenyl)]palladium(II) methanesulfonate |

### Example 1. Preparation of Intermediates

The following chemical intermediates were synthesized and are useful in the production of various compounds of the invention. It will be readily apparent to those of skill in the art that certain of the intermediates described in this Example, as well as in the compound synthesis examples that follow are also compounds within the scope of the invention.

### A. (4-((2-methoxyethoxy)methyl)phenyl)methanol

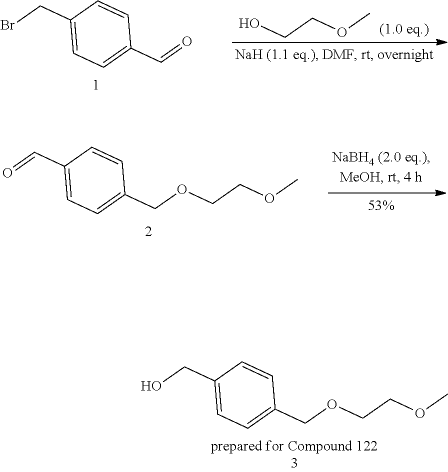

4-[(2-methoxyethoxy)methyl]benzaldehyde. A solution of 2-methoxyethanol (500.00 mg, 6.571 mmol, 1.00 equiv) in DMF (10.00 mL) was treated with NaH (262.80 mg, 6.571 mmol, 1.00 equiv, 60%) for 30 min at room temperature under nitrogen atmosphere followed by the addition of 4-(bromomethyl)benzaldehyde (1.57 g, 7.885 mmol, 1.20 equiv) at room temperature. The resulting mixture was stirred for overnight at room temperature under nitrogen atmosphere. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 60 mL/min; Gradient: 25% B to 45% B in 40 min; 254/220 nm. The fractions containing the desired product were collected at 38% B and concentrated under reduced pressure, to afford 4-[(2-methoxyethoxy)methyl]benzaldehyde (crude) as a yellow oil.

[4-[(2-methoxyethoxy)methyl]phenyl]methanol. To a stirred solution of 4-[(2-methoxyethoxy)methyl]benzaldehyde (170.00 mg, 0.875 mmol, 1.00 equiv) in MeOH (5.00 mL) was added NaBH₄ (66.23 mg, 1.751 mmol, 2.00 equiv) at room temperature. The resulting mixture was stirred for 4 h at room temperature. The reaction was monitored by LCMS. The residue was purified by reverse flash chromatography with the following conditions: Column: C18 Column 120 g; Mobile Phase A: Water (10 mM AcOH), Mobile Phase B: ACN; Flow rate: 60 mL/min; Gradient: 30% B to 50% B in 40 min; 254/220 nm. The fractions containing the desired product were collected at 42% B and concentrated under reduced pressure, to afford [4-[(2-methoxyethoxy)methyl]phenyl]methanol (50 mg, 29.11%) as a light yellow oil.

### B. [4-[([2-[(tert-butyldiphenylsilyl)oxy]ethyl](methyl)amino)methyl]phenyl]methanol [2-[(tert-butyldiphenylsilyl)oxy]ethyl](methyl)amine

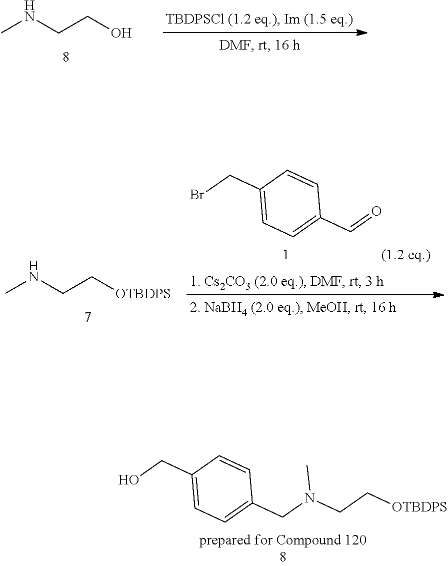

To a stirred solution of 2-(methylamino)ethan-1-ol (500.00 mg, 6.657 mmol, 1.00 equiv) in (10.00 mL) were added 1H-imidazole (679.78 mg, 9.985 mmol, 1.50 equiv) and tert-butyl (chloro)diphenylsilane (2.20 g, 7.988 mmol, 1.20 equiv) at room temperature. The resulting mixture was stirred for 16 h at room temperature. The reaction was monitored by LCMS. The solution was purified by reverse phase flash with the following conditions (Column: silica-CS Column 330 g; Mobile Phase A: PE, Mobile Phase B: EA; Flow rate: 80 mL/min; Gradient: 30% B to 50% B in 40 min; 254/280 nm). The fractions containing the desired product were collected at 38% B and concentrated under reduced pressure to afford [2-[(tert-butyldiphenylsilyl)oxy]ethyl](methyl)amine (1.2 g, 57.50%) as a yellow solid.

4-[([2-[(tert-butyldiphenylsilyl)oxy]ethyl](methyl)amino)methyl]benzaldehyde. To a stirred solution of [2-[(tert-butyldiphenylsilyl)oxy]ethyl](methyl)amine (300.00 mg, 0.957 mmol, 1.00 equiv) in DMF (10.00 mL) were added Cs₂CO₃ (0.62 g, 1.914 mmol, 2.00 equiv) and 4-(bromomethyl)benzaldehyde (228.56 mg, 1.148 mmol, 1.20 equiv) at room temperature. The resulting mixture was stirred for 3 h at room temperature. The resulting mixture was concentrated under reduced pressure. The residue was purified by Prep-TLC (PE/EtOAc 1:1) to afford 4-[([2-[(tert-butyldiphenylsilyl)oxy]ethyl](methyl)amino)methyl]benzaldehyde (200 mg, 48.42%) as a colorless oil.

[4-[([2-[(tert-butyldiphenylsilyl)oxy]ethyl](methyl)amino)methyl]phenyl]methanol. To a stirred solution of 4-[([2-[(tert-butyldiphenylsilyl)oxy]ethyl](methyl)amino)methyl]benzaldehyde (200.00 mg, 0.463 mmol, 1.00 equiv) in MeOH (5.00 mL) was added NaBH₄ (35.06 mg, 0.927 mmol, 2.00 equiv) at room temperature. The resulting mixture was stirred for 16 h at room temperature. The reaction was monitored by LCMS. The resulting mixture was concentrated under reduced pressure. The residue was purified by Prep-TLC (PE/EtOAc 1:1) to afford [4-[([2-[(tert-butyldiphenylsilyl)oxy]ethyl](methyl)amino)methyl]phenyl]methanol (150 mg, 74.65%) as a colorless oil.

### C. (4-((2,2-dimethyl-1,3-dioxolan-4-yl)methyl)phenyl)methanol

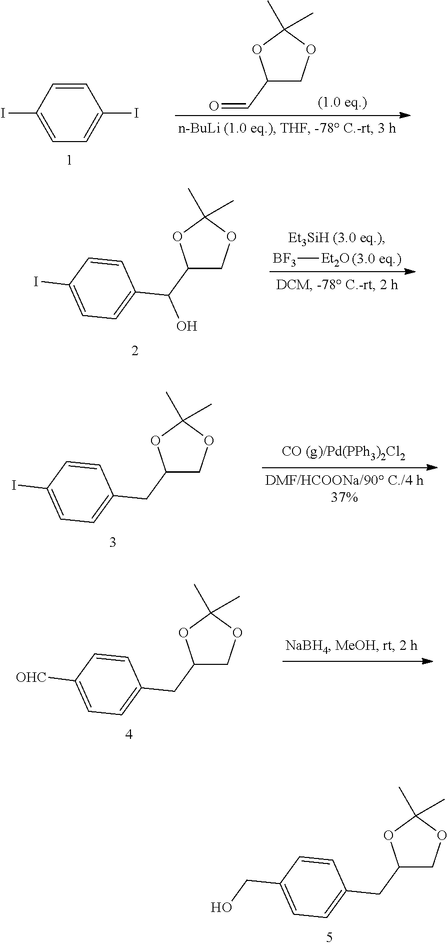

[2,2-dimethyl-1,3-dioxolan-4-yl](4-iodophenyl)methanol. To a stirred solution of 1,4-diiodobenzene (10 g, 30.312 mmol, 1.00 equiv) and n-BuLi (12.12 mL, 30.312 mmol, 1.00 equiv) in THF (150.00 mL) were added (4R)-2,2-dimethyl-1,3-dioxolane-4-carbaldehyde (3.94 g, 30.274 mmol, 1.00 equiv) dropwise at −78° C. under nitrogen atmosphere. The reaction was monitored by LCMS. The mixture was allowed to warm up to room temperature. The reaction was quenched by the addition of Water (70 mL) at room temperature. The resulting mixture was extracted with EtOAc (3×50 mL). The combined organic layers were washed with brine (3×100 mL), dried over anhydrous Na₂SO₄. After filtration, the filtrate was concentrated under reduced pressure. The residue product was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 30% B to 60% B in 40 min; 220/254 nm) to afford [2,2-dimethyl-1,3-dioxolan-4-yl](4-iodophenyl)methanol (2.95 g, 29.13%) as a yellow oil.

4-[(4-iodophenyl)methyl]-2,2-dimethyl-1,3-dioxolane. To a stirred solution of [2,2-dimethyl-1,3-dioxolan-4-yl](4-iodophenyl)methanol (3.1 g, 9.277 mmol, 1.00 equiv) in DCM (50.00 mL) were added Et₃SiH (3.24 g, 27.865 mmol, 3.00 equiv) and BF₃.Et₂O (8.78 g, 27.838 mmol, 3.00 equiv, 45%) dropwise at −78° C. under nitrogen atmosphere. The resulting mixture was stirred for 2 h at room temperature under nitrogen atmosphere. The reaction was monitored by LCMS. The reaction was quenched with sat. NH₄Cl (aq.) at room temperature. The resulting mixture was extracted with EtOAc (3×100 mL), the mixture dried over anhydrous MgSO₄. After filtration, the filtrate was concentrated under reduced pressure. The residue was purified by reverse phase flash chromatography with the following conditions: Column: Spherical C18, 20-40 um, 330 g; Mobile Phase A: Water (plus 5 mM NH₄HCO₃); Mobile Phase B: ACN; Flow rate: 80 mL/min; Gradient: 5% B-5% B, 10 min, 40% B-60% B gradient in 20 min; Detector: 220 nm. The fractions containing the desired product were collected at 51% B and concentrated under reduced pressure to afford 4-[(4-iodophenyl)methyl]-2,2-dimethyl-1,3-dioxolane (1.27 g, 43.03%) as a yellow oil.

4-[2,2-dimethyl-1,3-dioxolan-4-yl]methyl]benzaldehyde. To a stirred solution of 4-[(4-iodophenyl)methyl]-2,2-dimethyl-1,3-dioxolane (1.27 g, 3.992 mmol, 1.00 equiv) and HCOONa (0.54 g, 7.984 mmol, 2.00 equiv) in DMF (20.00 mL) was added Pd(PPh₃)₂Cl₂ (1.12 g, 1.597 mmol, 0.40 equiv) at room temperature under nitrogen atmosphere. The resulting mixture was stirred for 4 h at 90° C. under CO atmosphere. The reaction was monitored by LCMS. The mixture was allowed to cool down to room temperature. The resulting mixture was extracted with EtOAc (3×50 mL). The combined organic layers were washed with brine (3×20 mL), dried over anhydrous MgSO₄. After filtration, the filtrate was concentrated under reduced pressure. The residue was purified by silica gel column chromatography, eluted with PE/EtOAc (10/1 to 1/1) to afford 4-[2,2-dimethyl-1,3-dioxolan-4-yl]methyl]benzaldehyde (300 mg, 34.12%) as a yellow oil.

(4-[2,2-dimethyl-1,3-dioxolan-4-yl]methyl]phenyl)methanol. To a stirred solution of 4-[2,2-dimethyl-1,3-dioxolan-4-yl]methyl]benzaldehyde (259.00 mg, 1.176 mmol, 1.00 equiv) in MeOH (5.00 mL) was added NaBH₄ (88.97 mg, 2.352 mmol, 2.00 equiv) in portions at room temperature under air atmosphere. The resulting mixture was stirred for 2 h at room temperature under nitrogen atmosphere. The reaction was monitored by LCMS. The aqueous layer was extracted with EtOAc (3×50 mL). The resulting mixture was concentrated under reduced pressure. The crude product was purified by reverse phase flash with the following conditions (Column: C18 Column 120 g; Mobile Phase A: Water (10 mM NH₄HCO₃), Mobile Phase B: ACN; Flow rate: 40 mL/min; Gradient: 15% B to 50% B in 40 min; 220/254 nm) to afford (4-[2,2-dimethyl-1,3-dioxolan-4-yl]methyl]phenyl)methanol (155 mg, 59.30%) as a light yellow oil.

### D. (4-((1,4-dioxan-2-yl)methyl)phenyl)methanol

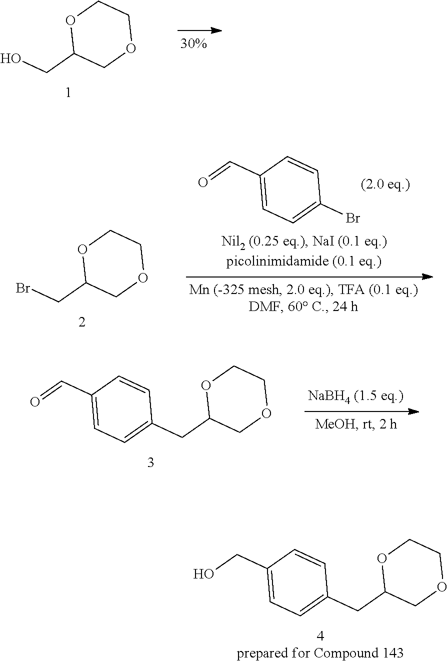 [SMILES: OCC1COCCO1 ; O=Cc1ccc(Br)cc1 ; BrCC1COCCO1 ; O=Cc1ccc(CC2COCCO2)cc1 ; OCc1ccc(CC2COCCO2)cc1]

2-(bromomethyl)-1,4-dioxane. To a stirred solution of 1,4-dioxan-2-ylmethanol (5.00 g, 42.326 mmol, 1.00 equiv) in DCM (50.00 mL) was added PBr₃ (11.46 g, 42.326 mmol, 1.00 equiv) dropwise at −30° C. The resulting mixture was stirred for 24 h at room temperature. The resulting mixture was concentrated under reduced pressure. The residue was purified by silica gel column chromatography, eluted with CH₂Cl₂ (100%) to afford 2-(bromomethyl)-1,4-dioxane (2.3 g, 30.02%) as a yellow oil.

4-(1,4-dioxan-2-ylmethyl)benzaldehyde. To a stirred solution of 2-(bromomethyl)-1,4-dioxane (1.50 g, 8.286 mmol, 1.00 equiv) and 4-bromo-benzaldehyde (3.07 g, 16.572 mmol, 2.00 equiv) in DMA (20.00 mL) were added picolinamidine (130.59 mg, 0.829 mmol, 0.10 equiv), NaI (124.20 mg, 0.829 mmol, 0.10 equiv), NiI₂ (647.34 mg, 2.071 mmol, 0.25 equiv), Mn (910.43 mg, 16.572 mmol, 2.00 equiv) and TFA (94.48 mg, 0.829 mmol, 0.10 equiv) at room temperature under nitrogen atmosphere. The resulting mixture was stirred for 24 h at 60° C. The reaction was monitored by LCMS. The solution was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 20% B to 40% B in 40 min; 254/220 nm) to afford 4-(1,4-dioxan-2-ylmethyl)benzaldehyde (700 mg, 40.96%) as a yellow oil. [4-(1,4-dioxan-2-ylmethyl)phenyl]methanol. To a stirred solution of 4-(1,4-dioxan-2-ylmethyl)benzaldehyde (700.00 mg, 3.394 mmol, 1.00 equiv) in MeOH (10.00 mL) was added NaBH₄ (192.61 mg, 5.091 mmol, 1.50 equiv) at room temperature. The resulting mixture was stirred for 2 h at room temperature. The reaction was monitored by TLC (PE/EA 1/1). The resulting mixture was concentrated under reduced pressure. The residue was purified by reverse phase flash chromatography with the following conditions (Column: C18 Column 120 g; Mobile Phase A: Water (10 mM NH₄HCO₃), Mobile Phase B: ACN; Flow rate: 50 mL/min; Gradient: 5% B to 50% B in 40 min; 254/220 nm) to afford [4-(1,4-dioxan-2-ylmethyl)phenyl]methanol (410 mg, 58.00%) as a yellow oil.

### E. (4-(1,4-dioxan-2-yl)phenyl)methanol

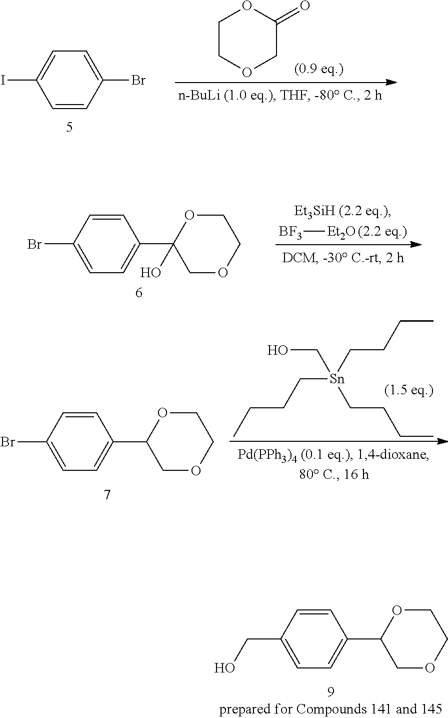

2-(4-bromophenyl)-1,4-dioxan-2-ol. To a stirred solution of 4-bromoiodobenzene (10.00 g, 35.347 mmol, 1.00 equiv) in THF (150.00 mL) was added n-BuLi (14.14 mL, 35.347 mmol, 1.00 equiv) dropwise at −80° C. under nitrogen atmosphere. The resulting mixture was stirred for 1 h at −80° C. under nitrogen atmosphere. To the above mixture was added 1,4-dioxan-2-one (3.60 g, 35.263 mmol, 0.9 equiv) dropwise at −80° C. The resulting mixture was stirred for additional 0.5 h at −80° C. The mixture was allowed to warm up to 0° C. The reaction was quenched by the addition of sat. NH₄Cl (aq.) (20 mL) at 0° C. The resulting mixture was extracted with EtOAc (3×100 mL). The combined organic layers were washed with brine (1×100 mL), dried over anhydrous Na₂SO₄. After filtration, the filtrate was concentrated under reduced pressure to afford 2-(4-bromophenyl)-1,4-dioxan-2-ol (crude) as a yellow oil. The crude product was used in the next step directly without further purification.

2-(4-bromophenyl)-1,4-dioxane. To a stirred solution of crude 2-(4-bromophenyl)-1,4-dioxan-2-ol (9.50 g, 36.666 mmol, 1.00 equiv) in DCM (100.00 mL) were added triethylsilane (9.38 g, 80.664 mmol, 2.20 equiv) and BF₃.Et₂O (10.22 mL, 72.022 mmol, 2.20 equiv) dropwise at −30° C. under nitrogen atmosphere. The resulting mixture was stirred for 2 h at −30° C. under nitrogen atmosphere. The reaction was monitored by LCMS. The mixture was allowed to warm up to 0° C. The reaction was quenched by the addition of sat. NaHCO₃ (aq.) (10 mL) at 0° C. The resulting mixture was extracted with CH₂Cl₂ (1×50 mL). The combined organic layers were washed with water (3×100 mL), dried over anhydrous Na₂SO₄. After filtration, the filtrate was concentrated under reduced pressure. The crude product was re-crystallized from ethanol (20 mL) to afford 2-(4-bromophenyl)-1,4-dioxane (6 g, 67.31%) as a white solid.

[4-(1,4-dioxan-2-yl)phenyl]methanol. To a stirred solution of 2-(4-bromophenyl)-1,4-dioxane (1.00 g, 4.114 mmol, 1.00 equiv) in 1,4-dioxane (10.00 mL) were added Pd(PPh₃)₄ (475.34 mg, 0.411 mmol, 0.10 equiv) and (tributylstannyl)methanol (1.98 g, 6.170 mmol, 1.50 equiv) at room temperature under nitrogen atmosphere. The resulting mixture was stirred for 16 h at 80° C. under nitrogen atmosphere. The reaction was monitored by LCMS. The mixture was allowed to cool down to room temperature. The resulting mixture was concentrated under vacuum. The residue was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 20% B to 50% B in 40 min; 254/220 nm) to afford [4-(1,4-dioxan-2-yl)phenyl]methanol (700 mg, 87.61%) as a yellow solid.

### F. 6-(hydroxymethyl)-N,N-bis[(4-methoxyphenyl)methyl]pyridine-3-sulfonamide

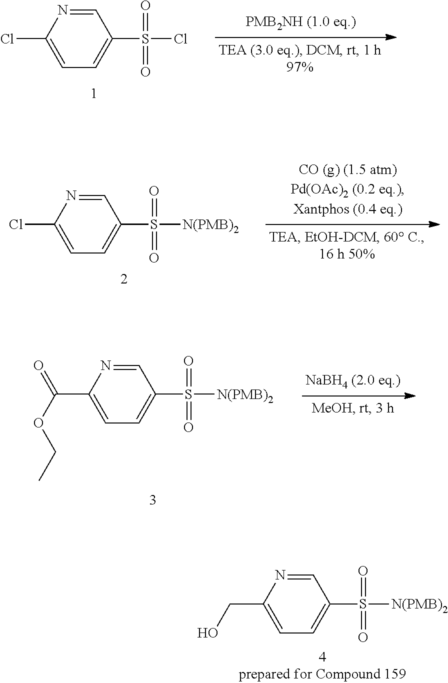

6-chloro-N,N-bis[(4-methoxyphenyl)methyl]pyridine-3-sulfonamide. A solution of 6-chloropyridine-3-sulfonyl chloride (15.00 g, 70.741 mmol, 1.00 equiv) and bis[(4-methoxyphenyl)methyl]amine (18.20 g, 70.725 mmol, 1.00 equiv) and TEA (21.47 g, 212.224 mmol, 3.00 equiv) in DCM (200.00 mL) was stirred for 1 h at room temperature under nitrogen atmosphere. Desired product could be detected by LCMS. The resulting mixture was concentrated under reduced pressure. The crude product was re-crystallized from EtOAc/PE (1:1 200 mL) to afford 6-chloro-N,N-bis[(4-methoxyphenyl)methyl]pyridine-3-sulfonamide (30 g, 97.96%) as a white solid.

Ethyl 5-[bis[(4-methoxyphenyl)methyl]sulfamoyl]pyridine-2-carboxylate. A solution of 6-chloro-N,N-bis[(4-methoxyphenyl)methyl]pyridine-3-sulfonamide (40.00 g, 92.396 mmol, 1.00 equiv) and Pd(AcO)₂ (4.15 g, 18.479 mmol, 0.20 equiv) and XantPhos (21.38 g, 36.958 mmol, 0.40 equiv) and TEA (46.75 g, 461.979 mmol, 5.00 equiv) in EtOH (500.00 mL) and DCM (600.00 mL) was stirred for 16 h at 60° C. under carbon monoxide atmosphere. Desired product could be detected by LCMS. The resulting mixture was filtered, the filter cake was washed with DCM (5×100 mL). The filtrate was concentrated under reduced pressure. The crude product was used in the next step directly without further purification.

6-(hydroxymethyl)-N,N-bis[(4-methoxyphenyl)methyl]pyridine-3-sulfonamide. A solution of ethyl 5-[bis[(4-methoxyphenyl)methyl]sulfamoyl]pyridine-2-carboxylate (30.00 g, 63.757 mmol, 1.00 equiv) and NaBH₄ (3.62 g, 95.684 mmol, 2.0 equiv) in MeOH (50.00 mL) and DCM (50.00 mL, 786.502 mmol, 12.34 equiv) was stirred for 3 h at room temperature under nitrogen atmosphere. Desired product could be detected by LCMS. The reaction was quenched with sat. NH₄Cl (aq.) at room temperature. The resulting mixture was extracted with EtOAc (3×500 mL). The combined organic layers were dried over anhydrous Na₂SO₄. After filtration, the filtrate was concentrated under reduced pressure. The residue was purified by silica gel column chromatography, eluted with PE/EtOAc (1:1) to afford 6-(hydroxymethyl)-N,N-bis[(4-methoxyphenyl)methyl]pyridine-3-sulfonamide (10 g, 36.60%) as an off-white solid.

### G. 6-(1-hydroxyethyl)-N,N-bis[(4-methoxyphenyl)methyl]pyridine-3-sulfonamide

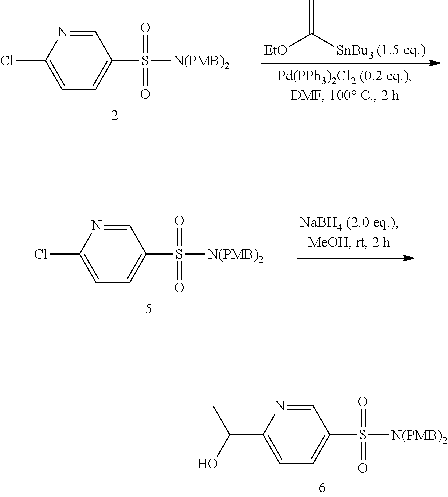

6-acetyl-N,N-bis[(4-methoxyphenyl)methyl]pyridine-3-sulfonamide. To a stirred solution of 6-chloro-N,N-bis[(4-methoxyphenyl)methyl]pyridine-3-sulfonamide (2.00 g, 4.620 mmol, 1.00 equiv) and tributyl-(1-ethoxyethenyl)stannane (2.50 g, 6.930 mmol, 1.50 equiv) in DMF was added Pd(PPh₃)₂Cl₂ (0.65 mg, 0.001 mmol, 0.2 equiv) at room temperature under nitrogen atmosphere. The resulting mixture was stirred for 2 h at 100° C. under nitrogen atmosphere. The reaction was monitored by LCMS. The mixture was allowed to cool down to room temperature. To the above mixture was added HCl (2M) (2.00 mL) at room temperature. The resulting mixture was stirred for additional 2 h at room temperature. The reaction was monitored by LCMS. The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 40% B to 60% B in 40 min; 254/220 nm. The fractions containing the desired product were collected at 48% B and concentrated under reduced pressure to afford 6-acetyl-N,N-bis[(4-methoxyphenyl)methyl]pyridine-3-sulfonamide (1.4 g, 68.79%) as a white solid.

6-(1-hydroxyethyl)-N,N-bis[(4-methoxyphenyl)methyl]pyridine-3-sulfonamide. To a stirred solution of 6-acetyl-N,N-bis[(4-methoxyphenyl)methyl]pyridine-3-sulfonamide (1.40 g, 3.178 mmol, 1.00 equiv) in MeOH (20.00 mL) was added NaBH₄ (180.36 mg, 4.767 mmol, 2.0 equiv) in portions at room temperature. The resulting mixture was stirred for 2 h at room temperature. The reaction was monitored by LCMS. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by reverse flash chromatography with the following conditions: Column: C18 Column 330 g; Mobile Phase A: water (10 mM NH₄HCO₃), Mobile Phase B: ACN; Flow rate: 50 mL/min; Gradient: 35% B to 60% B in 40 min; 254/220 nm. The fractions containing the desired product were collected at 52% B and concentrated under reduced pressure. To afford 6-(1-hydroxyethyl)-N,N-bis[(4-methoxyphenyl)methyl]pyridine-3-sulfonamide (900 mg, 63.99%) as a light yellow solid.

### I. 7-(4-chlorophenyl)-1-methyl-8-(2-methylpyrazol-3-yl)-3H-purine-2,6-dione

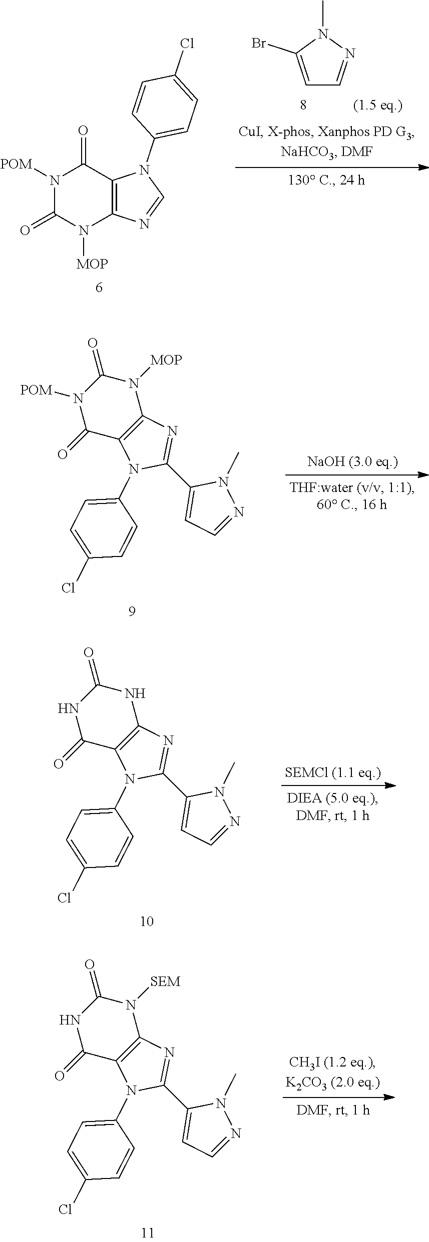

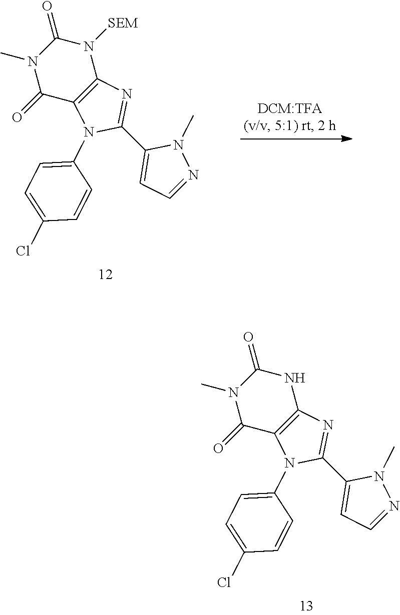

[7-(4-chlorophenyl)-3-[[(2,2-dimethylpropanoyl)oxy]methyl]-8-(2-methylpyrazol-3-yl)-2,6-dioxopurin-1-yl]methyl 2,2-dimethylpropanoate. To a stirred solution of [7-(4-chlorophenyl)-3-[[(2,2-dimethylpropanoyl)oxy]methyl]-2,6-dioxopurin-1-yl]methyl 2,2-dimethylpropanoate (6.00 g, 12.221 mmol, 1.00 equiv) and 5-bromo-1-methylpyrazole (2.95 g, 18.332 mmol, 1.50 equiv) in DMF (50.00 mL) were added X-Phos (0.58 g, 1.222 mmol, 0.10 equiv), XANTPHOS PD G3 (1.16 g, 1.222 mmol, 0.10 equiv), NaHCO₃ (2.57 g, 30.554 mmol, 2.50 equiv) and CuI (6.98 g, 36.664 mmol, 3.00 equiv) at room temperature under nitrogen atmosphere. The resulting mixture was stirred for 24 h at 130° C. under nitrogen atmosphere. The resulting mixture was concentrated under vacuum. The residue was purified by silica gel column chromatography, eluted with PE/EtOAc (3:1) to afford [7-(4-chlorophenyl)-3-[[(2,2-dimethylpropanoyl)oxy]methyl]-8-(2-methylpyrazol-3-yl)-2,6-dioxopurin-1-yl]methyl 2,2-dimethylpropanoate (2 g, 28.66%) as a white solid.

7-(4-chlorophenyl)-8-(2-methylpyrazol-3-yl)-1,3-dihydropurine-2,6-dione. To a stirred solution of [7-(4-chlorophenyl)-3-[[(2,2-dimethylpropanoyl)oxy]methyl]-8-(2-methylpyrazol-3-yl)-2,6-dioxopurin-1-yl]methyl 2,2-dimethylpropanoate (2.00 g, 3.502 mmol, 1.00 equiv) in THF (10.00 mL) and water (10.00 mL) was added NaOH (0.42 g, 10.507 mmol, 3.00 equiv) at room temperature. The resulting mixture was stirred for 16 h at 60° C. The mixture was acidified to pH 6 with 1M HCl (aq.). The resulting mixture was extracted with EtOAc (3×20 mL), dried over anhydrous Na₂SO₄. After filtration, the filtrate was concentrated under reduced pressure to afford 7-(4-chlorophenyl)-8-(2-methylpyrazol-3-yl)-1,3-dihydropurine-2,6-dione (1.1 g, 91.63%) as a white solid.

7-(4-chlorophenyl)-8-(2-methylpyrazol-3-yl)-3-[[2-(trimethylsilyl)ethoxy]methyl]-1H-purine-2,6-dione. To a stirred solution of 7-(4-chlorophenyl)-8-(2-methylpyrazol-3-yl)-1,3-dihydropurine-2,6-dione (1.10 g, 3.209 mmol, 1.00 equiv) and DIEA (2.07 g, 16.047 mmol, 5.00 equiv) in DMF (5.00 mL) was added SEMCl (0.59 g, 3.530 mmol, 1.10 equiv) dropwise at room temperature. The resulting mixture was stirred for 1 h at room temperature. The resulting mixture was diluted with water (30 mL). The resulting mixture was extracted with EtOAc (3×20 mL), dried over anhydrous Na₂SO₄. After filtration, the filtrate was concentrated under reduced pressure. The crude product was used in the next step directly without further purification.

7-(4-chlorophenyl)-1-methyl-8-(2-methylpyrazol-3-yl)-3-[[2-(trimethylsilyl)ethoxy]methyl]purine-2,6-dione. To a stirred solution of 7-(4-chlorophenyl)-8-(2-methylpyrazol-3-yl)-3-[[2-(trimethylsilyl)ethoxy]methyl]-1H-purine-2,6-dione (1.20 g, 2.537 mmol, 1.00 equiv) and K₂CO₃ (1.05 g, 7.611 mmol, 2 equiv) in DMF (5.00 mL) was added Mel (0.72 g, 5.074 mmol, 1.2 equiv) at room temperature. The resulting mixture was stirred for 1 h at room temperature. The resulting mixture was concentrated under vacuum. The residue was purified by silica gel column chromatography, eluted with PE/EtOAc (3:1) to afford 7-(4-chlorophenyl)-1-methyl-8-(2-methylpyrazol-3-yl)-3-[[2-(trimethylsilyl)ethoxy]methyl]purine-2,6-dione (950 mg, 76.89%) as a white solid. 7-(4-chlorophenyl)-1-methyl-8-(2-methylpyrazol-3-yl)-3H-purine-2,6-dione. To a stirred solution of 7-(4-chlorophenyl)-1-methyl-8-(2-methylpyrazol-3-yl)-3-[[2-(trimethylsilyl)ethoxy]methyl]purine-2,6-dione (950.00 mg, 1.951 mmol, 1.00 equiv) in DCM (5 mL) was added TFA (1 mL) at room temperature. The resulting mixture was stirred for 2 h at room temperature. The resulting mixture was concentrated under reduced pressure. The residue was purified by reverse flash chromatography with the following conditions: column, C18 silica gel; mobile phase, ACN in water (10 mM NH₄HCO₃), 25% to 50% gradient in 25 min; detector, UV 254 nm to afford 7-(4-chlorophenyl)-1-methyl-8-(2-methylpyrazol-3-yl)-3H-purine-2,6-dione (490 mg, 70.41%) as a white solid.

### Example 2. Preparation of Compounds 100 and 102

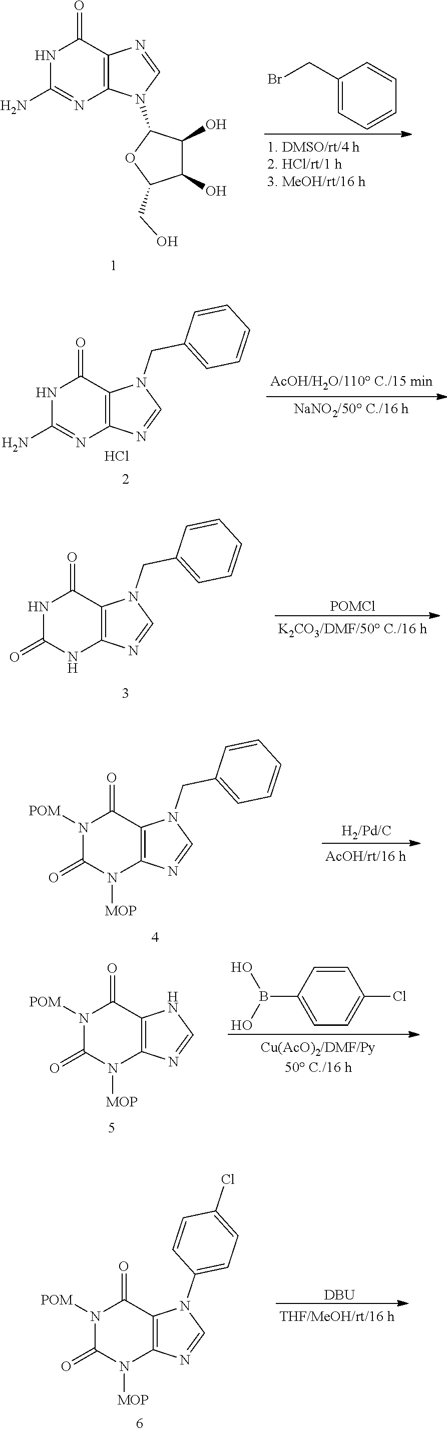

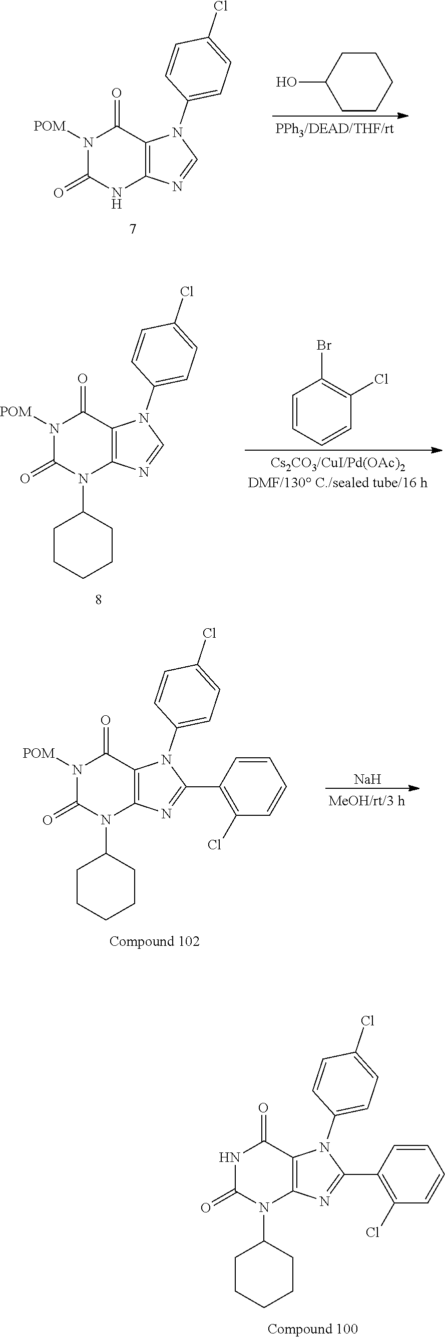

2-Amino-7-benzyl-6,7-dihydro-1H-purin-6-one hydrochloride. To a stirred solution of 2-amino-9-[(2S,3S,4R,5S)-3,4-dihydroxy-5-(hydroxymethyl)oxolan-2-yl]-6,9-dihydro-1H-purin-6-one (100 g, 353.052 mmol, 1 equiv) in DMSO (500 mL) was added (bromomethyl)benzene (144.92 g, 847.326 mmol, 2.4 equiv) dropwise at room temperature. The resulting mixture was stirred for 4 h at room temperature. To the above mixture was added HCl (250 mL, 3000.000 mmol, 8.50 equiv) dropwise at room temperature. The resulting mixture was stirred for additional 1 h at room temperature. To the above mixture was added MeOH (3 L) at room temperature. The resulting mixture was stirred for additional 16 h at room temperature. The reaction was monitored by LCMS. The precipitated solids were collected by filtration and washed with MeOH (3×100 mL) to afford 2-amino-7-benzyl-6,7-dihydro-1H-purin-6-one hydrochloride (85 g, 86.69%) as a white solid.

7-Benzyl-2,3,6,7-tetrahydro-1H-purine-2,6-dione. A mixture of 2-amino-7-benzyl-6,7-dihydro-1H-purin-6-one hydrochloride (85 g, 306.075 mmol, 1 equiv) in AcOH (2 L) and water (200 mL) was stirred for 15 min at 110° C. The solution was allowed to cool down to 50° C. To the above mixture was added solution of NaNO₂ (85 g, 1231.968 mmol, 4.03 equiv) in water (200 mL) dropwise over 2 h at 50° C. The resulting mixture was stirred for additional 16 h at 50° C. The reaction was monitored by LCMS. The mixture was allowed to cool down to room temperature. The precipitated solids were collected by filtration and washed with water (3×50 mL) to afford 7-benzyl-2,3,6,7-tetrahydro-1H-purine-2,6-dione (43 g, 58.00%) as a light yellow solid.

(7-Benzyl-3-[[(2,2-dimethylpropanoyl)oxy]methyl]-2,6-dioxo-2,3,6,7-tetrahydro-1H-purin-1-yl)methyl 2,2-dimethylpropanoate. To a stirred solution of 7-benzyl-2,3,6,7-tetrahydro-1H-purine-2,6-dione (5.5 g, 22.705 mmol, 1 equiv) in DMF (130 mL) were added K₂CO₃ (9.41 g, 68.115 mmol, 3 equiv) and chloromethyl 2,2-dimethylpropanoate (8.55 g, 56.762 mmol, 2.5 equiv) dropwise at room temperature. The resulting mixture was stirred for 16 h at 50° C. The reaction was monitored by LCMS. The resulting mixture was extracted with EtOAc (3×100 mL). The combined organic layers were washed with water (1×100 mL) and HCl (aq 1M), dried over anhydrous Na₂SO₄. After filtration, the filtrate was concentrated under reduced pressure. The residue was purified by silica gel column chromatography, eluted with hexane/EtOAc (5:1 to 3:1) to afford (7-benzyl-3-[[(2,2-dimethylpropanoyl)oxy]methyl]-2,6-dioxo-2,3,6,7-tetrahydro-1H-purin-1-yl)methyl 2,2-dimethylpropanoate (4.5 g, 42.12%) as a white solid.

(3-[[(2,2-dimethylpropanoyl)oxy]methyl]-2,6-dioxo-2,3,6,7-tetrahydro-1H-purin-1-yl)methyl 2,2-dimethylpropanoate To a solution of (7-benzyl-3-[[(2,2-dimethylpropanoyl)oxy]methyl]-2,6-dioxo-2,3,6,7-tetrahydro-1H-purin-1-yl)methyl 2,2-dimethylpropanoate (4.5 g, 9.564 mmol, 1 equiv) in 100 mL AcOH was added Pd/C (1.5 g) under nitrogen atmosphere in a 250 mL round-bottom flask. The mixture was hydrogenated at room temperature for 16 h under hydrogen atmosphere using a hydrogen balloon. The reaction was monitored by LCMS. The solution was filtered through a Celite pad and concentrated under reduced pressure to afford (3-[[(2,2-dimethylpropanoyl)oxy]methyl]-2,6-dioxo-2,3,6,7-tetrahydro-1H-purin-1-yl)methyl 2,2-dimethylpropanoate (2.7 g, 74.22%) as a white solid.

[7-(4-chlorophenyl)-3-[[(2,2-dimethylpropanoyl)oxy]methyl]-2,6-dioxo-2,3,6,7-tetrahydro-1H-purin-1-yl]methyl 2,2-dimethylpropanoate. To a stirred solution of (3-[[(2,2-dimethylpropanoyl)oxy]methyl]-2,6-dioxo-2,3,6,7-tetrahydro-1H-purin-1-yl)methyl 2,2-dimethylpropanoate (2.7 g, 7.098 mmol, 1 equiv) in DMF (50 mL) were added (4-chlorophenyl)boronic acid (2.55 g, 16.325 mmol, 2.3 equiv), Cu(AcO)₂ (644.60 mg, 3.549 mmol, 0.5 equiv) and pyridine (1.68 g, 21.293 mmol, 3 equiv) at room temperature. The resulting mixture was stirred for 16 h at 50° C. The reaction was monitored by LCMS. The mixture was allowed to cool down to room temperature. The resulting mixture was concentrated under reduced pressure. The residue was purified by silica gel column chromatography, eluted with hexane/EtOAc (10:1 to 5:1) to afford [7-(4-chlorophenyl)-3-[[(2,2-dimethylpropanoyl)oxy]methyl]-2,6-dioxo-2,3,6,7-tetrahydro-1H-purin-1-yl]methyl 2,2-dimethylpropanoate (1.3 g, 37.31%) as a white solid.

[7-(4-chlorophenyl)-2,6-dioxo-2,3,6,7-tetrahydro-1H-purin-1-yl]methyl 2,2-dimethylpropanoate. To a stirred solution of [7-(4-chlorophenyl)-3-[[(2,2-dimethylpropanoyl)oxy]methyl]-2,6-dioxo-2,3,6,7-tetrahydro-1H-purin-1-yl]methyl 2,2-dimethylpropanoate (900 mg, 1.833 mmol, 1 equiv) in THF (5 mL) and MeOH (10 mL) was added DBU (279.08 mg, 1.833 mmol, 1 equiv) at room temperature. The resulting mixture was stirred for 16 h at room temperature. The reaction was monitored by LCMS. The reaction was quenched by the addition of HCl (aq. 1M) (5 mL) at room temperature. The resulting mixture was extracted with EtOAc (3×100 mL). The combined organic layers were washed with brine (1×50 mL), dried over anhydrous Na₂SO₄. After filtration, the filtrate was concentrated under reduced pressure. The residue/crude product was purified by reverse phase flash with the following conditions (Column: C18 Column 330 g; Mobile Phase A: Water (10 mM FA), Mobile Phase B: ACN; Flow rate: 80 mL/min; Gradient: 30% B to 50% B in 40 min; 254/220 nm). The fractions containing the desired product were collected at 40% B and concentrated under reduced pressure to afford [7-(4-chlorophenyl)-2,6-dioxo-2,3,6,7-tetrahydro-1H-purin-1-yl]methyl 2,2-dimethylpropanoate (250 mg, 36.19%) as a white solid.

[7-(4-chlorophenyl)-3-cyclohexyl-2,6-dioxo-2,3,6,7-tetrahydro-1H-purin-1-yl]methyl 2,2-dimethylpropanoate. To a stirred solution of [7-(4-chlorophenyl)-2,6-dioxo-2,3,6,7-tetrahydro-1H-purin-1-yl]methyl 2,2-dimethylpropanoate (800 mg, 2.123 mmol, 1 equiv) and PPh₃ (1113.74 mg, 4.246 mmol, 2 equiv) in dry THF (50 mL) were added DEAD (739.51 mg, 4.246 mmol, 2 equiv) and cyclohexanol (212.66 mg, 2.123 mmol, 1 equiv) dropwise at 0° C. The resulting mixture was stirred for 16 h at room temperature. The reaction was monitored by LCMS. The solution was purified by reverse phase flash with the following conditions (Column: C18 Column 330 g; Mobile Phase A: Water (10 mM AcOH), Mobile Phase B: ACN; Flow rate: 80 mL/min; Gradient: 70% B to 90% B in 40 min; 254/220 nm). The fractions containing the desired product were collected at 89% B and concentrated under reduced pressure to afford [7-(4-chlorophenyl)-3-cyclohexyl-2,6-dioxo-2,3,6,7-tetrahydro-1H-purin-1-yl]methyl 2,2-dimethylpropanoate (400 mg, 41.05%) as a white solid.

[8-(2-chlorophenyl)-7-(4-chlorophenyl)-3-cyclohexyl-2,6-dioxo-2,3,6,7-tetrahydro-1H-purin-1-yl]methyl 2,2-dimethylpropanoate. To a stirred solution of [7-(4-chlorophenyl)-3-cyclohexyl-2,6-dioxo-2,3,6,7-tetrahydro-1H-purin-1-yl]methyl 2,2-dimethylpropanoate (300 mg, 0.654 mmol, 1 equiv) in dry DMF (15 mL) were added 1-bromo-2-chlorobenzene (187.72 mg, 0.981 mmol, 1.5 equiv), Cs₂CO₃ (0.53 g, 1.634 mmol, 2.5 equiv), Pd(AcO)₂ (29.35 mg, 0.131 mmol, 0.2 equiv) and CuI (373.48 mg, 1.961 mmol, 3 equiv) at room temperature under nitrogen atmosphere. The resulting mixture was stirred for 16 h at 130° C. under nitrogen atmosphere in sealed tube. The reaction was monitored by LCMS. The mixture was allowed to cool down to room temperature. The resulting mixture was filtered, the filter cake was washed with EtOAc (3×10 mL). The filtrate was concentrated under reduced pressure. The residue/crude product was purified by reverse phase flash with the following conditions (Column: C18 Column 120 g; Mobile Phase A: Water (10 mM TFA), Mobile Phase B: ACN; Flow rate: 70 mL/min; Gradient: 70% B to 95% B in 40 min; 254/220 nm). The fractions containing the desired product were collected at 94% B and concentrated under reduced pressure to afford [8-(2-chlorophenyl)-7-(4-chlorophenyl)-3-cyclohexyl-2,6-dioxo-2,3,6,7-tetrahydro-1H-purin-1-yl]methyl 2,2-dimethylpropanoate (200 mg, 53.73%) as an off-white solid.

8-(2-chlorophenyl)-7-(4-chlorophenyl)-3-cyclohexyl-2,3,6,7-tetrahydro-1H-purine-2,6-dione. To a stirred solution of [8-(2-chlorophenyl)-7-(4-chlorophenyl)-3-cyclohexyl-2,6-dioxo-2,3,6,7-tetrahydro-1H-purin-1-yl]methyl 2,2-dimethylpropanoate (170 mg, 0.299 mmol, 1 equiv) in MeOH (5 mL) was added NaH (23.88 mg, 0.597 mmol, 2 equiv, 60%) at room temperature. The resulting mixture was stirred for 3 h at room temperature. The reaction was monitored by LCMS. The mixture was neutralized to pH 7 with HCl (aq. 1N). The residue was purified by reverse phase flash with the following conditions (Column: C18 Column 120 g; Mobile Phase A: Water (10 mM TFA), Mobile Phase B: ACN; Flow rate: 70 mL/min; Gradient: 60% B to 80% B in 40 min; 254/220 nm). The fractions containing the desired product were collected at 68% B and concentrated under reduced pressure to afford 8-(2-chlorophenyl)-7-(4-chlorophenyl)-3-cyclohexyl-2,3,6,7-tetrahydro-1H-purine-2,6-dione (100 mg, 73.57%) as a white solid.

### Example 3. Preparation of Compound 104

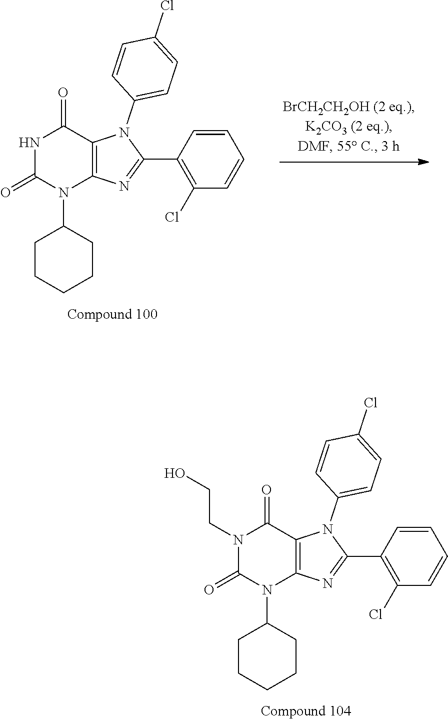 [SMILES: O=c1[nH]c(=O)n(C2CCCCC2)c2nc(-c3ccccc3Cl)n(-c3ccc(Cl)cc3)c12 ; O=c1c2c(nc(-c3ccccc3Cl)n2-c2ccc(Cl)cc2)n(C2CCCCC2)c(=O)n1CCO]

To a stirred solution of 8-(2-chlorophenyl)-7-(4-chlorophenyl)-3-cyclohexyl-2,3,6,7-tetrahydro-1H-purine-2,6-dione (20 mg, 0.044 mmol, 1 equiv) in DMF (3 mL) were added K₂CO₃ (12.14 mg, 0.088 mmol, 2 equiv) and 2-bromoethan-1-ol (10.98 mg, 0.088 mmol, 2 equiv) at room temperature. The resulting mixture was stirred for 3 h at 55° C. The reaction was monitored by LCMS. The mixture was allowed to cool down to room temperature. The solution was purified by reverse phase flash with the following conditions (Column: XBridge Shield RP18 OBD Column, 5 um, 19*150 mm; Mobile Phase A: undefined, Mobile Phase B: undefined; Flow rate: 25 mL/min; Gradient: 45% B to 60% B in 12 min; 220 nm; Rt: 11.38 min) to afford 8-(2-chlorophenyl)-7-(4-chlorophenyl)-3-cyclohexyl-1-(2-hydroxyethyl)-2,3,6,7-tetrahydro-1H-purine-2,6-dione (5 mg, 22.79%) as an off-white solid.

Compounds 101, 103, 105 and 106 were prepared by the methods and scheme described above for Compound 104 using the appropriate reagent.

### Example 4. Preparation of Compound 147

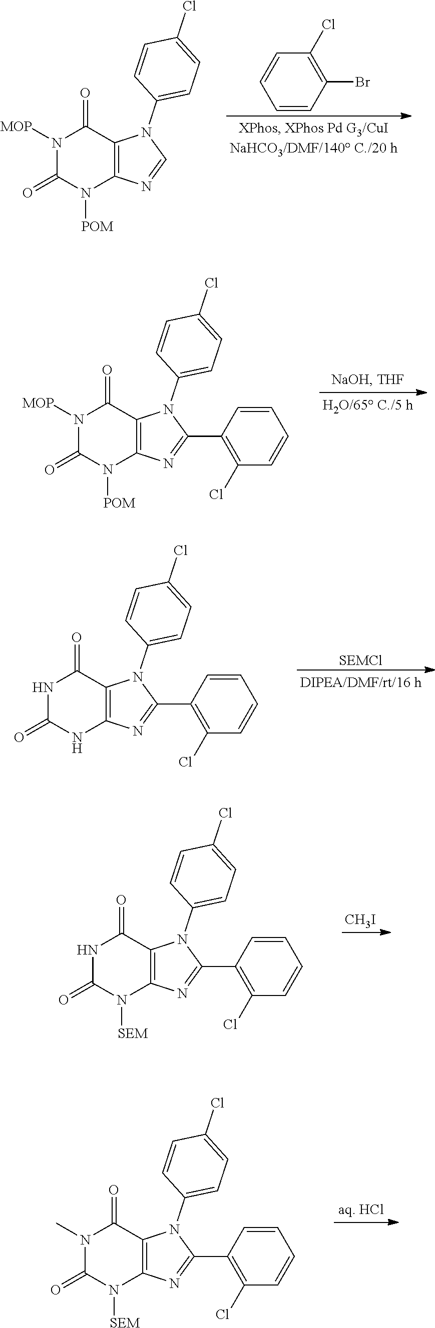

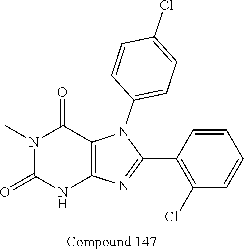 [SMILES: Cn1c(=O)[nH]c2nc(-c3ccccc3Cl)n(-c3ccc(Cl)cc3)c2c1=O]

[8-(2-chlorophenyl)-7-(4-chlorophenyl)-3-[[(2,2-dimethylpropanoyl)oxy]methyl]-2,6-dioxopurin-1-yl]methyl 2,2-dimethylpropanoate. To a stirred solution of [7-(4-chlorophenyl)-3-[[(2,2-dimethylpropanoyl)oxy]methyl]-2,6-dioxopurin-1-yl]methyl 2,2-dimethylpropanoate (40.00 g, 81.476 mmol, 1.00 equiv) in DMF (2.00 L) were added 2-bromochlorobenzene (23.40 g, 122.215 mmol, 1.50 equiv), XPhos (3.88 g, 8.148 mmol, 0.10 equiv), NaHCO₃ (17.11 g, 203.691 mmol, 2.50 equiv), XPhos Pd G3 (6.90 g, 8.148 mmol, 0.10 equiv) and CuI (31.03 g, 162.953 mmol, 2.00 equiv) at room temperature under nitrogen atmosphere. The resulting mixture was stirred for 20 h at 140° C. under nitrogen atmosphere in sealed tube. The reaction was monitored by LCMS. The mixture was allowed to cool down to room temperature. The resulting mixture was extracted with EtOAc (3×1 L). The combined organic layers were washed with water (1×1 L), dried over anhydrous Na₂SO₄. After filtration, the filtrate was concentrated under reduced pressure. The residue was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 70% B to 95% B in 40 min; 254/220 nm). The fractions containing the desired product were collected at 87% B and concentrated under reduced pressure to afford [8-(2-chlorophenyl)-7-(4-chlorophenyl)-3-[[(2,2-dimethylpropanoyl)oxy]methyl]-2,6-dioxopurin-1-yl]methyl 2,2-dimethylpropanoate (32 g, 65.30%) as a white solid.

8-(2-chlorophenyl)-7-(4-chlorophenyl)-1,3-dihydropurine-2,6-dione. To a stirred solution of [8-(2-chlorophenyl)-7-(4-chlorophenyl)-3-[[(2,2-dimethylpropanoyl)oxy]methyl]-2,6-dioxopurin-1-yl]methyl 2,2-dimethylpropanoate (32.00 g, 53.202 mmol, 1.00 equiv) and in THF (200.00 mL) and water (200.00 mL) was added NaOH (6.38 g, 159.606 mmol, 3.00 equiv) at room temperature. The resulting mixture was stirred for 5 h at 65° C. The reaction was monitored by LCMS. The mixture was allowed to cool down to room temperature. The mixture was acidified to pH 5 with HCl (aq. 1N). The precipitated solids were collected by filtration and washed with water (3×300 mL) to afford 8-(2-chlorophenyl)-7-(4-chlorophenyl)-1,3-dihydropurine-2,6-dione (17 g, 85.62%) as a white solid.

8-(2-chlorophenyl)-7-(4-chlorophenyl)-3-[[2-(trimethylsilyl)ethoxy]methyl]-1H-purine-2,6-dione. To a stirred solution of 8-(2-chlorophenyl)-7-(4-chlorophenyl)-1,3-dihydropurine-2,6-dione (1.80 g, 4.823 mmol, 1.00 equiv) in DMF (30.00 mL) were added DIPEA (3.12 g, 24.116 mmol, 5.00 equiv) and SEMCl (804.14 mg, 4.823 mmol, 1.00 equiv) at room temperature. The resulting mixture was stirred for 16 h at room temperature. The reaction was monitored by LCMS. The solution was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 60% B to 80% B in 40 min; 254/220 nm). The fractions containing the desired product were collected at 65% B and concentrated under reduced pressure to afford 8-(2-chlorophenyl)-7-(4-chlorophenyl)-3-[[2-(trimethylsilyl)ethoxy]methyl]-1H-purine-2,6-dione (2.3 g, 94.72%) as a yellow oil.

8-(2-chlorophenyl)-7-(4-chlorophenyl)-1-methyl-3-[[2-(trimethylsilyl)ethoxy]methyl]purine-2,6-dione. To a stirred solution of 8-(2-chlorophenyl)-7-(4-chlorophenyl)-3-[[2-(trimethylsilyl)ethoxy]methyl]-1H-purine-2,6-dione (2.30 g, 4.568 mmol, 1.00 equiv) in DMF (20.00 mL) were added K₂CO₃ (1262.75 mg, 9.137 mmol, 2.00 equiv) and Mel (778.12 mg, 5.482 mmol, 1.20 equiv) at room temperature. The resulting mixture was stirred for 16 h at room temperature. The reaction was monitored by LCMS. The resulting mixture was extracted with EtOAc (3×50 mL). The combined organic layers were washed with brine (1×50 mL), dried over anhydrous Na₂SO₄. After filtration, the filtrate was concentrated under reduced pressure. The reside was used in the next step directly without further purification.

8-(2-chlorophenyl)-7-(4-chlorophenyl)-1-methyl-3H-purine-2,6-dione. To a stirred solution of 8-(2-chlorophenyl)-7-(4-chlorophenyl)-1-methyl-3-[[2-(trimethylsilyl)ethoxy]methyl]purine-2,6-dione (2.00 g, 3.865 mmol, 1.00 equiv) in 1,4-dioxane (10.00 mL) were added HCl (12M) (20.00 mL, 658.238 mmol) at room temperature. The resulting mixture was stirred for 16 h at room temperature. The reaction was monitored by LCMS. The mixture was basified to pH 7 with saturated NaHCO₃ (aq.). The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18 Column 330 g; Mobile Phase A: Water (10 mM NH₄HCO₃), Mobile Phase B: ACN; Flow rate: 70 mL/min; Gradient: 35% B to 45% B in 40 min; 254/220 nm). The fractions containing the desired product were collected at 40% B and concentrated under reduced pressure to afford 8-(2-chlorophenyl)-7-(4-chlorophenyl)-1-methyl-3H-purine-2,6-dione (1.1 g, 73.50%) as an off-white solid.

### Example 5. Preparation of Compound 110

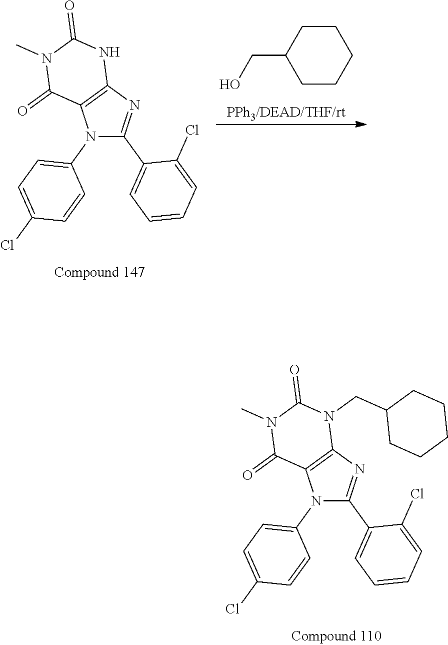 [SMILES: Cn1c(=O)[nH]c2nc(-c3ccccc3Cl)n(-c3ccc(Cl)cc3)c2c1=O ; OCC1CCCCC1 ; Cn1c(=O)c2c(nc(-c3ccccc3Cl)n2-c2ccc(Cl)cc2)n(CC2CCCCC2)c1=O]

To a stirred solution of 8-(2-chlorophenyl)-7-(4-chlorophenyl)-1-methyl-2,3,6,7-tetrahydro-1H-purine-2,6-dione (30.00 mg, 0.077 mmol, 1.00 equiv) and PPh₃ (60.96 mg, 0.232 mmol, 3.00 equiv) in THF (2.00 mL) was added DEAD (40.48 mg, 0.232 mmol, 3.00 equiv) and cyclohexylmethanol (10.62 mg, 0.093 mmol, 1.20 equiv) at 0° C. under nitrogen atmosphere. The resulting mixture was stirred for 2 h at room temperature under nitrogen atmosphere. The reaction was monitored by LCMS. The crude product was purified by Prep-HPLC with the following conditions (Column: Xselect CSH OBD Column 30*150 mm 5 um; Mobile Phase A: Water (0.05% TFA), Mobile Phase B: ACN; Flow rate: 60 mL/min; Gradient: 55% B to 93% B in 7 min, then from 93% B to 0% B, From 7 to 0 min; 220 nm; RT1: 6.73) to afford 8-(2-chlorophenyl)-7-(4-chlorophenyl)-3-(cyclohexylmethyl)-1-methyl-2,3,6,7-tetrahydro-1H-purine-2,6-dione (12 mg, 32.04%) as an off-white solid.

Compounds 120 and 122 were prepared by the methods and scheme described for Compound 110 using the appropriate reagents.

### Example 6. Preparation of Compound 109

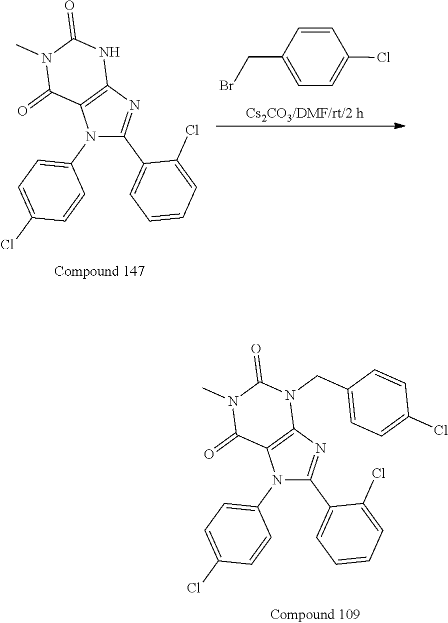 [SMILES: Cn1c(=O)[nH]c2nc(-c3ccccc3Cl)n(-c3ccc(Cl)cc3)c2c1=O ; Clc1ccc(CBr)cc1 ; Cn1c(=O)c2c(nc(-c3ccccc3Cl)n2-c2ccc(Cl)cc2)n(Cc2ccc(Cl)cc2)c1=O]

To a stirred solution of 8-(2-chlorophenyl)-7-(4-chlorophenyl)-1-methyl-2,3,6,7-tetrahydro-1H-purine-2,6-dione (30.00 mg, 0.077 mmol, 1.00 equiv) and 1-(bromomethyl)-4-chlorobenzene (19.10 mg, 0.093 mmol, 1.20 equiv) in DMF (2.00 mL) was added Cs₂CO₃ (50.49 mg, 0.155 mmol, 2.00 equiv) at room temperature. The resulting mixture was stirred for 2 h at room temperature. The reaction was monitored by LCMS. The crude product (20 mg) was purified by Prep-HPLC with the following conditions (Column: Xselect CSH OBD Column 30*150 mm 5 um n; Mobile Phase A: Water (0.05% TFA), Mobile Phase B: ACN; Flow rate: 60 mL/min; Gradient: 55% B to 90% B in 7 min; 220 nm; Rt: 6.35 min) to afford 8-(2-chlorophenyl)-7-(4-chlorophenyl)-3-[(4-chlorophenyl)methyl]-1-methyl-2,3,6,7-tetrahydro-1H-purine-2,6-dione (10 mg, 25.22%) as an off-white solid.

Compound 108 was prepared by the methods and scheme described for Compound 109 using the appropriate reagents.

### Example 7. Preparation of Compounds 111, 112 and 114

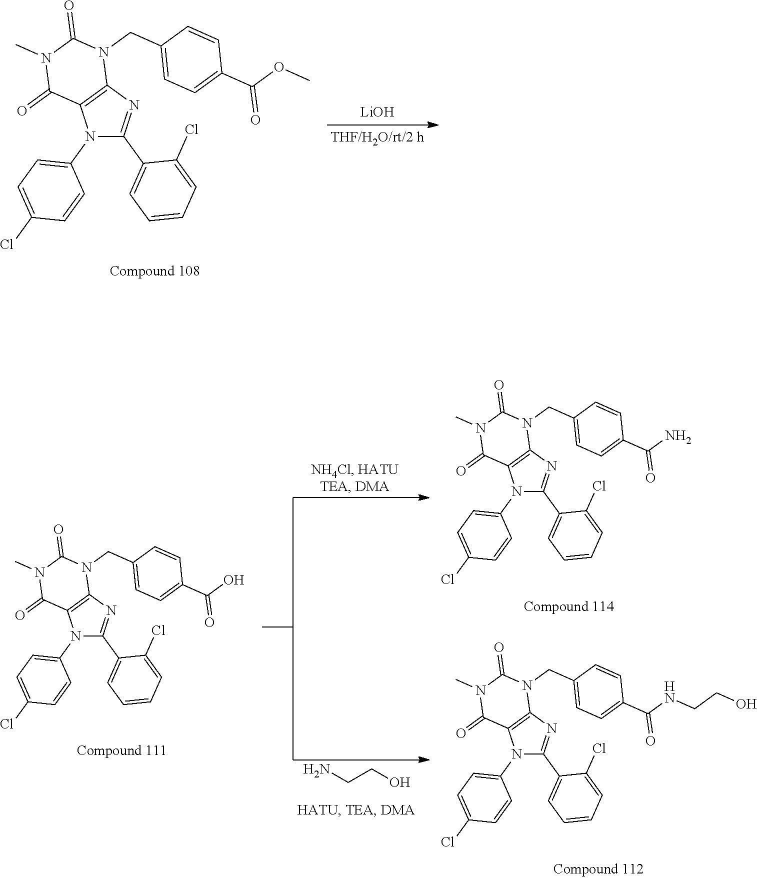

4-[[8-(2-chlorophenyl)-7-(4-chlorophenyl)-1-methyl-2,6-dioxo-2,3,6,7-tetrahydro-1H-purin-3-yl]methyl]benzoic acid. A solution/mixture of methyl 4-[[8-(2-chlorophenyl)-7-(4-chlorophenyl)-1-methyl-2,6-dioxo-2,3,6,7-tetrahydro-1H-purin-3-yl]methyl]benzoate (60.00 mg, 0.112 mmol, 1.00 equiv) and LiOH (26.84 mg, 1.121 mmol, 10.00 equiv) in THF (2.50 mL) and H₂O (2.50 mL) was stirred for 2 h at room temperature. The reaction was monitored by LCMS. The mixture was basified to pH 5 with acetic acid. The residue was purified by reverse flash chromatography with the following conditions: Column: C18 Column 120 g; Mobile Phase A: Water (10 mM AcOH), Mobile Phase B: ACN; Flow rate: 60 mL/min; Gradient: 55% B to 80% B in 40 min; 254/220 nm. The fractions containing the desired product were collected at 64% B and concentrated under reduced pressure, to afford 4-[[8-(2-chlorophenyl)-7-(4-chlorophenyl)-1-methyl-2,6-dioxo-2,3,6,7-tetrahydro-1H-purin-3-yl]methyl]benzoic acid (30 mg, 51.35%) as a white solid.

4-[[8-(2-chlorophenyl)-7-(4-chlorophenyl)-1-methyl-2,6-dioxo-2,3,6,7-tetrahydro-1H-purin-3-yl]methyl]benzamide. A solution of 4-[[8-(2-chlorophenyl)-7-(4-chlorophenyl)-1-methyl-2,6-dioxo-2,3,6,7-tetrahydro-1H-purin-3-yl]methyl]benzoic acid (120.00 mg, 0.230 mmol, 1.00 equiv) and HATU (131.28 mg, 0.345 mmol, 1.50 equiv) in DMA (1.50 mL) was stirred for 30 min at room temperature followed by the addition of NH₄Cl (36.94 mg, 0.691 mmol, 3.00 equiv) at room temperature. To the above mixture was added TEA (232.91 mg, 2.302 mmol, 10.00 equiv) for 30 min at room temperature. The resulting mixture was stirred for additional 16 h at room temperature. The reaction was monitored by LCMS. The crude product was purified by Prep-HPLC with the following conditions (Column: XBridge Shield RP18 OBD Column, 5 um, 19*150 mm; Mobile Phase A: Water (0.05% TFA), Mobile Phase B: ACN; Flow rate: 25 mL/min; Gradient: 25% B to 46% B in 7 min; 220 nm; Rt: 6.92 min) to afford 4-[[8-(2-chlorophenyl)-7-(4-chlorophenyl)-1-methyl-2,6-dioxo-2,3,6,7-tetrahydro-1H-purin-3-yl]methyl]benzamide (100 mg, 83.49%) as a white solid.

4-[[8-(2-chlorophenyl)-7-(4-chlorophenyl)-1-methyl-2,6-dioxo-2,3,6,7-tetrahydro-1H-purin-3-yl]methyl]-N-(2-hydroxyethyl)benzamide. A solution/mixture of 4-[[8-(2-chlorophenyl)-7-(4-chlorophenyl)-1-methyl-2,6-dioxo-2,3,6,7-tetrahydro-1H-purin-3-yl]methyl]benzoic acid (100.00 mg, 0.192 mmol, 1.00 equiv) and HATU (109.40 mg, 0.288 mmol, 1.50 equiv) in DMA (2.00 mL) was stirred for 30 min at room temperature followed by the addition of 2-aminoethan-1-ol (35.15 mg, 0.575 mmol, 3.00 equiv) at room temperature. To the above mixture was added TEA (194.09 mg, 1.918 mmol, 10.00 equiv) over 30 min at room temperature. The resulting mixture was stirred for additional 8 h at room temperature. The reaction was monitored by LCMS. The crude product was purified by Prep-HPLC with the following conditions (Column: XBridge Prep C18 OBD Column, 5 um, 19*150 mm; Mobile Phase A: Water (0.05% TFA), Mobile Phase B: ACN; Flow rate: 25 mL/min; Gradient: 36% B to 56% B in 7 min; 220 nm; Rt: 6.8 min) to afford 4-[[8-(2-chlorophenyl)-7-(4-chlorophenyl)-1-methyl-2,6-dioxo-2,3,6,7-tetrahydro-1H-purin-3-yl]methyl]-N-(2-hydroxyethyl)benzamide (80 mg, 73.90%) as a white solid

Compounds 126, 160, 161, 162, 165, 166 and 167 were prepared by the methods and scheme described above for Compound 112 using the appropriate reagents.

### Example 8. Preparation of Compounds 131 and 134

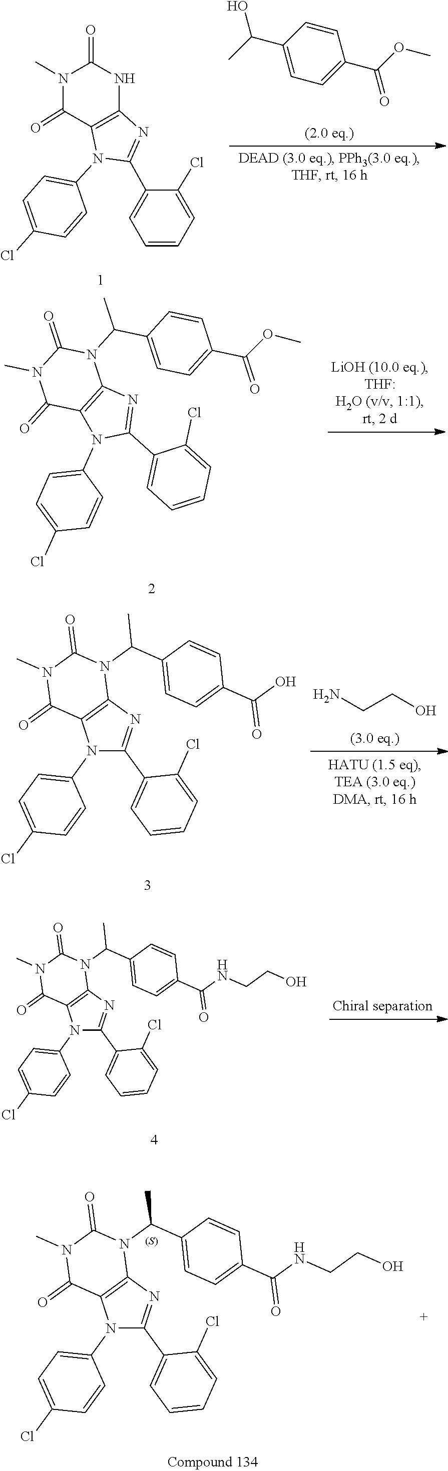

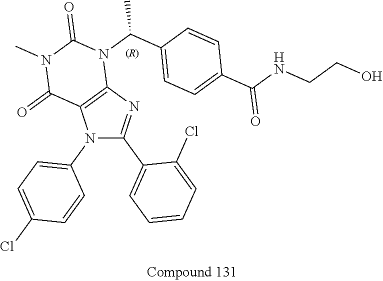

Methyl 4-[1-[8-(2-chlorophenyl)-7-(4-chlorophenyl)-1-methyl-2,6-dioxopurin-3-yl]ethyl]benzoate. To a stirred solution of 8-(2-chlorophenyl)-7-(4-chlorophenyl)-1-methyl-3H-purine-2,6-dione (150.00 mg, 0.387 mmol, 1.00 equiv) and PPh₃ (304.81 mg, 1.162 mmol, 3.00 equiv) in THF (5.00 mL) were added DEAD (202.39 mg, 1.162 mmol, 3.00 equiv) and methyl 4-(1-hydroxyethyl)benzoate (139.61 mg, 0.775 mmol, 2.00 equiv) at 0° C. under nitrogen atmosphere. The resulting mixture was stirred for 16 h at room temperature under nitrogen atmosphere. The reaction was monitored by LCMS. The residue was purified by reverse flash chromatography with the following conditions: Column: C18 Column 80 g; Mobile Phase A: Water (10 mM AcOH), Mobile Phase B: ACN; Flow rate: 50 mL/min; Gradient: 60% B to 80% B in 40 min; 254/220 nm. The fractions containing the desired product were collected at 73% B and concentrated under reduced pressure. to afford methyl 4-[1-[8-(2-chlorophenyl)-7-(4-chlorophenyl)-1-methyl-2,6-dioxopurin-3-yl]ethyl]benzoate (150 mg, 70.48%) as an off-white solid.

4-[1-[8-(2-chlorophenyl)-7-(4-chlorophenyl)-1-methyl-2,6-dioxopurin-3-yl]ethyl]benzoic acid. To a stirred solution of methyl 4-[1-[8-(2-chlorophenyl)-7-(4-chlorophenyl)-1-methyl-2,6-dioxopurin-3-yl]ethyl]benzoate (150.00 mg, 0.273 mmol, 1.00 equiv) and LiOH (65.38 mg, 2.730 mmol, 10.00 equiv) in THF (3.00 mL) was added H₂O (3.00 mL) at room temperature. The resulting mixture was stirred for 2 days at room temperature. The reaction was monitored by LCMS. The mixture was acidified to pH 6 with HCl (aq.). The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 55% B to 75% B in 40 min; 254/220 nm. The fractions containing the desired product were collected at 65% B and concentrated under reduced pressure. To afford 4-[1-[8-(2-chlorophenyl)-7-(4-chlorophenyl)-1-methyl-2,6-dioxopurin-3-yl]ethyl]benzoic acid (110 mg, 75.26%) as a white solid.

4-[(1S)-1-[8-(2-chlorophenyl)-7-(4-chlorophenyl)-1-methyl-2,6-dioxopurin-3-yl]ethyl]-N-(2-hydroxyethyl)benzamide and 4-[(1R)-1-[8-(2-chlorophenyl)-7-(4-chlorophenyl)-1-methyl-2,6-dioxopurin-3-yl]ethyl]-N-(2-hydroxyethyl)benzamide. A solution of 4-[1-[8-(2-chlorophenyl)-7-(4-chlorophenyl)-1-methyl-2,6-dioxopurin-3-yl]ethyl]benzoic acid (110.00 mg, 0.205 mmol, 1.00 equiv) in DMA (5.00 mL) was treated with HATU (117.18 mg, 0.308 mmol, 1.50 equiv) for 30 min at room temperature followed by the addition of ethanolamine (37.65 mg, 0.616 mmol, 3.00 equiv) at room temperature. To the above mixture was added TEA (62.37 mg, 0.616 mmol, 3.00 equiv) for 30 min at room temperature. The resulting mixture was stirred for additional overnight at room temperature. The reaction was monitored by LCMS. The crude product was purified by Prep-HPLC with the following conditions (Column: XBridge Shield RP18 OBD Column, 5 um, 19×150 mm; Flow rate: 25 mL/min; Gradient: 37 B to 42 B in 7 min; 220/254 nm; RT1: 6.5; RT2:; Injection Volume: mL) to afford 4-[(1S)-1-[8-(2-chlorophenyl)-7-(4-chlorophenyl)-1-methyl-2,6-dioxopurin-3-yl]ethyl]-N-(2-hydroxyethyl)benzamide (40 mg, 33.66%) and 4-[(1R)-1-[8-(2-chlorophenyl)-7-(4-chlorophenyl)-1-methyl-2,6-dioxopurin-3-yl]ethyl]-N-(2-hydroxyethyl)benzamide (40 mg, 33.66%) as a white solid.

### Example 9. Preparation of Compound 107

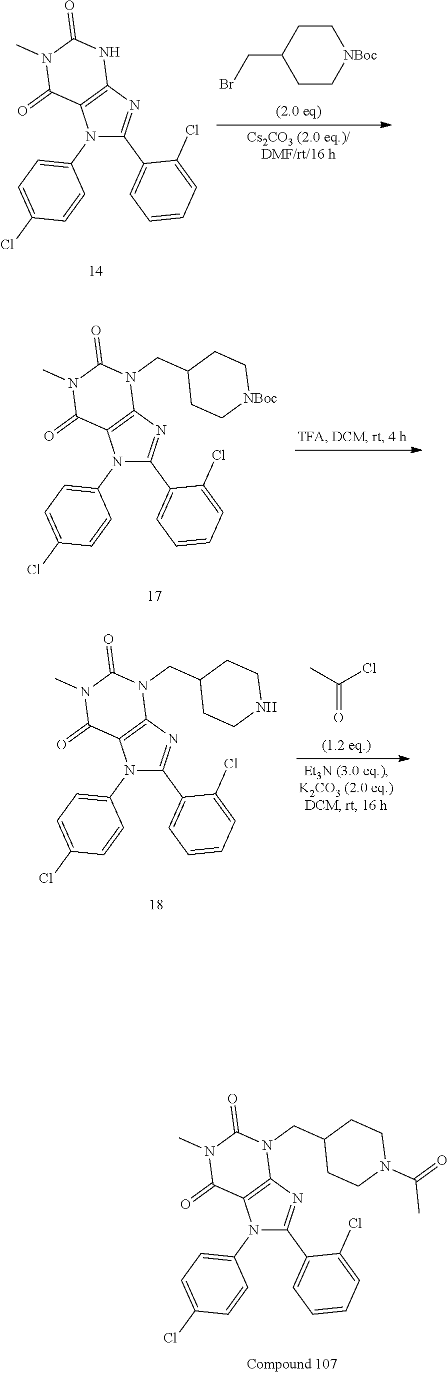

tert-butyl 4-[[8-(2-chlorophenyl)-7-(4-chlorophenyl)-1-methyl-2,6-dioxopurin-3-yl]methyl]piperidine-1-carboxylate. To a stirred solution/mixture of 8-(2-chlorophenyl)-7-(4-chlorophenyl)-1-methyl-3H-purine-2,6-dione (100.00 mg, 0.258 mmol, 1.00 equiv) and Cs₂CO₃ (168.29 mg, 0.517 mmol, 2.00 equiv) in DMF (5.00 mL) was added tert-butyl 4-(bromomethyl)piperidine-1-carboxylate (143.69 mg, 0.517 mmol, 2.00 equiv) at room temperature. The resulting mixture was stirred for 16 h at room temperature. The reaction was monitored by LCMS. The residue was purified by reverse flash chromatography with the following conditions: Column: C18 Column 40 g; Mobile Phase A: Water (10 mM AcOH), Mobile Phase B: ACN; Flow rate: 50 mL/min; Gradient: 60% B to 80% B in 40 min; 254/220 nm. The fractions containing the desired product were collected at 77% B and concentrated under reduced pressure. To afford tert-butyl 4-[[8-(2-chlorophenyl)-7-(4-chlorophenyl)-1-methyl-2,6-dioxopurin-3-yl]methyl]piperidine-1-carboxylate (125 mg, 82.81%) as an off-white solid.

8-(2-chlorophenyl)-7-(4-chlorophenyl)-1-methyl-3-(piperidin-4-ylmethyl)purine-2,6-dione. To a stirred solution/mixture of tert-butyl 4-[[8-(2-chlorophenyl)-7-(4-chlorophenyl)-1-methyl-2,6-dioxopurin-3-yl]methyl]piperidine-1-carboxylate (125.00 mg, 1 equiv) in DCM (4.00 mL) was added TFA (1.00 mL) at room temperature. The resulting mixture was stirred for 4 h at room temperature. The reaction was monitored by LCMS. The resulting mixture was extracted with EtOAc (3×20 mL). The combined organic layers were washed with brine (3×20 mL), dried over anhydrous Na₂SO₄. After filtration, the filtrate was concentrated under reduced pressure. to afford 8-(2-chlorophenyl)-7-(4-chlorophenyl)-1-methyl-3-(piperidin-4-ylmethyl)purine-2,6-dione (80 mg, 77.23%) as a light yellow oil.

3-[(1-acetylpiperidin-4-yl)methyl]-8-(2-chlorophenyl)-7-(4-chlorophenyl)-1-methylpurine-2,6-dione. To a stirred solution of 8-(2-chlorophenyl)-7-(4-chlorophenyl)-1-methyl-3-(piperidin-4-ylmethyl)purine-2,6-dione (80.00 mg, 0.165 mmol, 1.00 equiv) and TEA (50.14 mg, 0.495 mmol, 3 equiv) in DCM (5.00 mL) was added acetyl chloride (15.56 mg, 0.198 mmol, 1.2 equiv) for 16 h at room temperature. The reaction was monitored by LCMS. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 60 mL/min; Gradient: 30% B to 60% B in 40 min; 254/220 nm. The fractions containing the desired product were collected at 49% B and concentrated under reduced pressure. To afford 3-[(1-acetylpiperidin-4-yl)methyl]-8-(2-chlorophenyl)-7-(4-chlorophenyl)-1-methylpurine-2,6-dione (40 mg, 46.01%) as a white solid.

### Example 10. Preparation of Compound 115

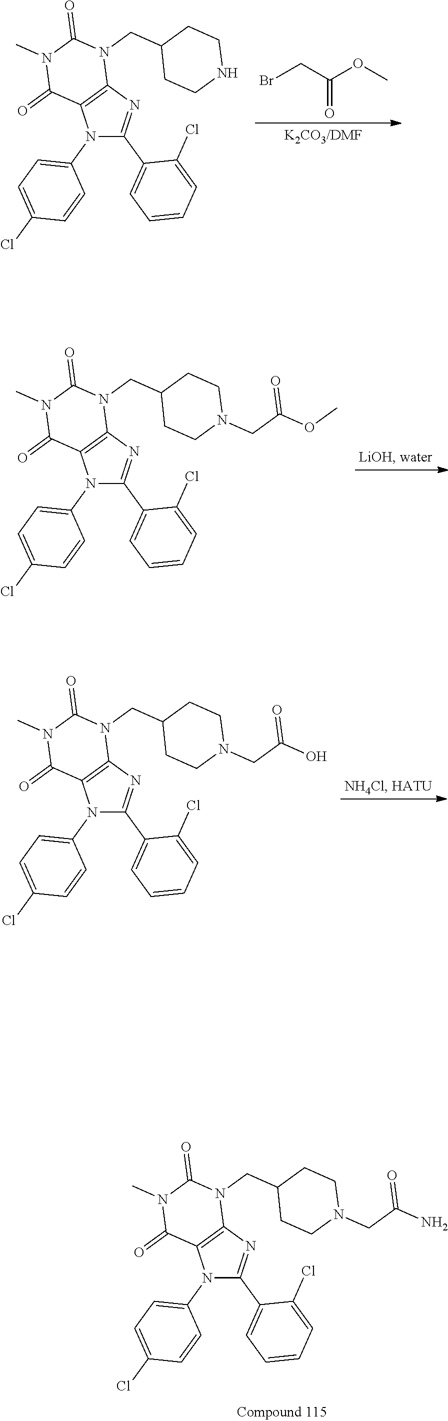

Methyl 2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetate. To a stirred solution of 8-(2-chlorophenyl)-7-(4-chlorophenyl)-1-methyl-3-[(piperidin-4-yl)methyl]-2,3,6,7-tetrahydro-1H-purine-2,6-dione (100.00 mg, 0.206 mmol, 1.00 equiv) in DMF (5.00 mL) were added K₂CO₃ (57.06 mg, 0.413 mmol, 2.00 equiv) and methyl 2-bromoacetate (37.90 mg, 0.248 mmol, 1.20 equiv) at room temperature. The resulting mixture was stirred for 2 h at room temperature. The residue was purified by silica gel column chromatography, eluted with CH₂Cl₂/MeOH (20:1) to afford methyl 2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetate (60 mg, 52.23%) as a yellow solid. 2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetic acid. To a stirred solution of methyl 2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetate (70.00 mg, 0.126 mmol, 1.00 equiv) in THF (5.00 mL) and H₂O (5.00 mL) was added LiOH (30.13 mg, 1.258 mmol, 10.00 equiv) at room temperature. The resulting mixture was stirred for 16 h at room temperature. The mixture was acidified to pH 6 with AcOH. The resulting mixture was concentrated under reduced pressure. The residue was purified by reverse phase flash with the following conditions (Column: C18 Column 80 g; Mobile Phase A: Water (10 mM NH₄HCO₃), Mobile Phase B: ACN; Flow rate: 50 mL/min; Gradient: 20% B to 40% B in 40 min; 254/220 nm). The fractions containing the desired product were collected at 34% B and concentrated under reduced pressure to afford 2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetic acid (40 mg, 58.62%) as an off-white solid.

2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetamide. To a stirred solution of 2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetic acid (20.00 mg, 0.037 mmol, 1.00 equiv) and HATU (21.03 mg, 0.055 mmol, 1.50 equiv) in DMA (3.00 mL) for 30 min at room temperature followed by the addition of NH₄Cl (5.92 mg, 0.111 mmol, 3.00 equiv) at room temperature. To the above mixture was added TEA (37.31 mg, 0.369 mmol, 10.00 equiv) over 30 min at room temperature. The resulting mixture was stirred for additional 16 h at room temperature. The reaction was monitored by LCMS. The crude product was purified by Prep-HPLC with the following conditions (Column: XBridge Prep C18 OBD Column, 5 um, 19×150 mm; Mobile Phase A: Water (0.05% TFA), Mobile Phase B: ACN; Flow rate: 25 mL/min; Gradient: 35% B to 60% B in 7 min; 220 nm; Rt: 7.22 min) to afford 2-(4-[[8-(2-chlorophenyl)-7-(4-chlorophenyl)-1-methyl-2,6-dioxo-2,3,6,7-tetrahydro-1H-purin-3-yl]methyl]piperidin-1-yl)acetamide (16 mg, 80.15%) as an off-white solid.

### Example 11. Preparation of Compound 113

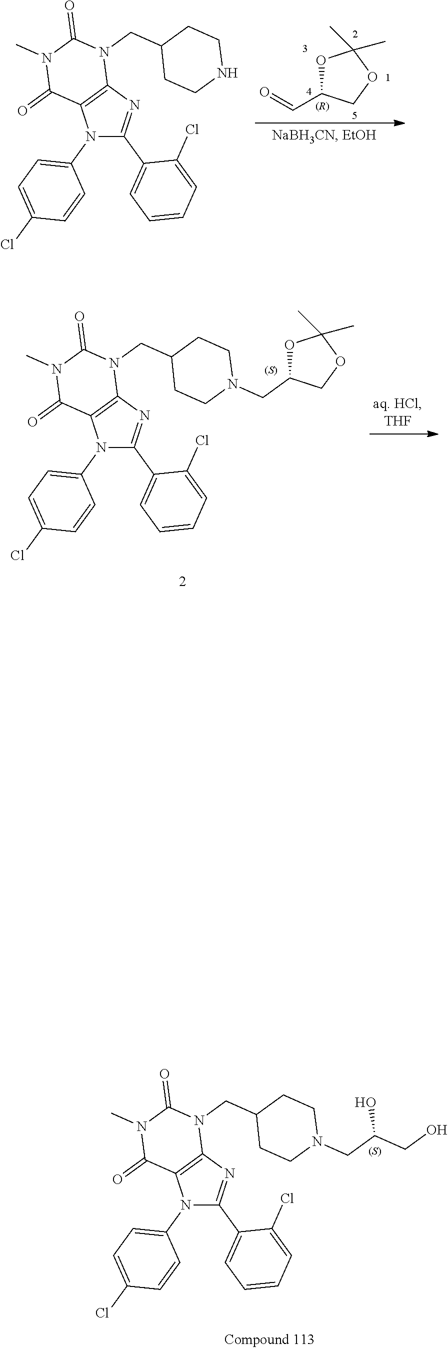

8-(2-chlorophenyl)-7-(4-chlorophenyl)-3-[(1-[[(4S)-2,2-dimethyl-1,3-dioxolan-4-yl]methyl]piperidin-4-yl)methyl]-1-methyl-2,3,6,7-tetrahydro-1H-purine-2,6-dione. To a stirred solution of 8-(2-chlorophenyl)-7-(4-chlorophenyl)-1-methyl-3-[(piperidin-4-yl)methyl]-2,3,6,7-tetrahydro-1H-purine-2,6-dione (50.00 mg, 0.103 mmol, 1.00 equiv) in EtOH (3.00 mL) was added (R)-2,2-dimethyl-1,3-dioxolane-4-carbaldehyde (26.87 mg, 0.206 mmol, 2.00 equiv) at room temperature. The resulting mixture was stirred for 0.5 h at room temperature. To the above mixture was added NaBH₃CN (12.97 mg, 0.206 mmol, 2.00 equiv) at room temperature.

The resulting mixture was stirred for additional 16 h at room temperature. The resulting mixture was extracted with EtOAc (3×10 mL). The combined organic layers were washed with brine (1×10 mL), dried over anhydrous Na₂SO₄. After filtration, the filtrate was concentrated under reduced pressure. The crude product was used in the next step directly without further purification.

8-(2-chlorophenyl)-7-(4-chlorophenyl)-3-([1-[(2S)-2,3-dihydroxypropyl]piperidin-4-yl]methyl)-1-methyl-2,3,6,7-tetrahydro-1H-purine-2,6-dione. To a stirred solution of 8-(2-chlorophenyl)-7-(4-chlorophenyl)-3-[(1-[[(4S)-2,2-dimethyl-1,3-dioxolan-4-yl]methyl]piperidin-4-yl)methyl]-1-methyl-2,3,6,7-tetrahydro-1H-purine-2,6-dione (50.00 mg, 0.084 mmol, 1.00 equiv) in THF (3.00 mL) was added HCl (3.00 mL, 2N) at room temperature. The reaction was monitored by LCMS. The mixture was basified to pH 8 with saturated NaHCO₃ (aq.). The resulting mixture was concentrated under vacuum. The residue was purified by Prep-HPLC with the following conditions (Column: XBridge Shield RP18 OBD Column, 5 um, 19*150 mm; Mobile Phase A: undefined, Mobile Phase B: undefined; Flow rate: 25 mL/min; Gradient: 50% B to 58% B in 8 min; 220 nm; Rt: 5.30 min) to afford 8-(2-chlorophenyl)-7-(4-chlorophenyl)-3-([1-[(2S)-2,3-dihydroxypropyl]piperidin-4-yl]methyl)-1-methyl-2,3,6,7-tetrahydro-1H-purine-2,6-dione (20 mg, 42.87%) as an off-white solid.

### Example 12. Preparation of Compound 116

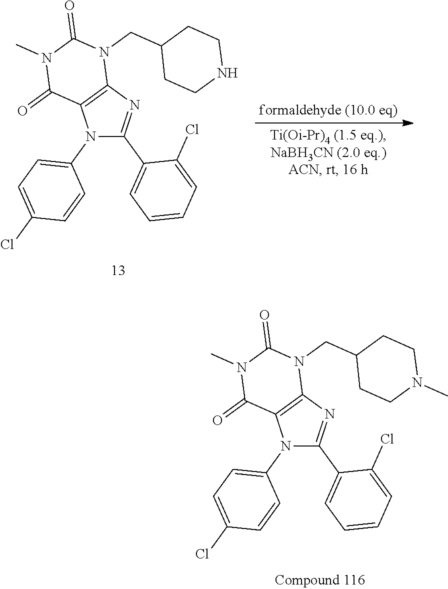 [SMILES: Cn1c(=O)c2c(nc(-c3ccccc3Cl)n2-c2ccc(Cl)cc2)n(CC2CCNCC2)c1=O ; CN1CCC(Cn2c(=O)n(C)c(=O)c3c2nc(-c2ccccc2Cl)n3-c2ccc(Cl)cc2)CC1]

8-(2-chlorophenyl)-7-(4-chlorophenyl)-1-methyl-3-[(1-methylpiperidin-4-yl)methyl]-2,3,6,7-tetrahydro-1H-purine-2,6-dione. To a stirred solution of 8-(2-chlorophenyl)-7-(4-chlorophenyl)-1-methyl-3-[(piperidin-4-yl)methyl]-2,3,6,7-tetrahydro-1H-purine-2,6-dione (30.00 mg, 0.062 mmol) in acetonitrile (5.00 mL) were added formaldehyde (aq. 37%, 50.26 mg, 0.619 mmol) and tetraisopropoxytitanium (26.40 mg, 0.093 mmol) followed by sodium cyanoborohydride (7.78 mg, 0.124 mmol) at ambient temperature. After stirring at ambient temperature for 16 h, the reaction mixture was purified directly by reversed phase flash chromatography with the following conditions: Column: XBridge Prep C18 OBD Column, 5 μm, 19×150 mm; Mobile Phase A: Water (plus 0.05% TFA); Mobile Phase B: ACN; Flow rate: 25 mL/min; Gradient (B): 25% to 47% in 7 min; Detector: UV 220 nm/254 nm. The fractions containing desired product were collected at 39% B and concentrated under reduced pressure to afford 8-(2-chlorophenyl)-7-(4-chlorophenyl)-1-methyl-3-[(1-methylpiperidin-4-yl)methyl]-2,3,6,7-tetrahydro-1H-purine-2,6-dione (5.0 mg, 16%) as an off-white solid

### Example 13. Preparation of Compound 119

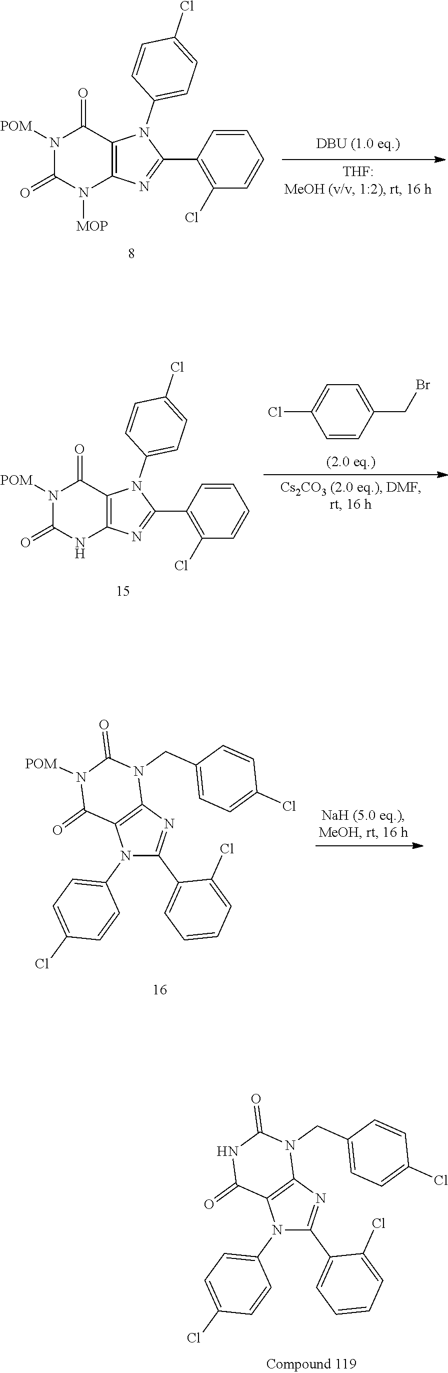

[8-(2-chlorophenyl)-7-(4-chlorophenyl)-2,6-dioxo-2,3,6,7-tetrahydro-1H-purin-1-yl]methyl 2,2-dimethylpropanoate. To a stirred solution of [8-(2-chlorophenyl)-7-(4-chlorophenyl)-3-[[(2,2-dimethylpropanoyl)oxy]methyl]-2,6-dioxo-2,3,6,7-tetrahydro-1H-purin-1-yl]methyl 2,2-dimethylpropanoate (8.00 g, 13.301 mmol, 1.00 equiv) in MeOH (100.00 mL) and THF (50.00 mL) was added DBU (2.02 g, 13.301 mmol, 1.00 equiv) at room temperature, and the resulting reaction mixture was stirred for 16 h. The mixture was acidified to pH 6 with HCl (aq. 2 N). The resulting mixture was extracted with EtOAc (3×30 mL). The combined organic layers were washed with brine (1×100 mL), dried over anhydrous Na₂SO₄. After filtration, the filtrate was concentrated under reduced pressure. The residue was purified by reverse phase flash with the following conditions (Column: silica-CS Column 330 g; Mobile Phase A: PE, Mobile Phase B: EA; Flow rate: 80 mL/min; Gradient: 50% B to 70% B in 40 min; 254/280 nm). The fractions containing the desired product were collected at 60% B and concentrated under reduced pressure to afford [8-(2-chlorophenyl)-7-(4-chlorophenyl)-2,6-dioxo-2,3,6,7-tetrahydro-1H-purin-1-yl]methyl 2,2-dimethylpropanoate (2.3 g, 35.48%) as a off-white solid. [8-(2-chlorophenyl)-7-(4-chlorophenyl)-3-[(4-chlorophenyl)methyl]-2,6-dioxo-2,3,6,7-tetrahydro-1H-purin-1-yl]methyl 2,2-dimethylpropanoate. To a stirred solution of [8-(2-chlorophenyl)-7-(4-chlorophenyl)-2,6-dioxo-2,3,6,7-tetrahydro-1H-purin-1-yl]methyl 2,2-dimethylpropanoate (400.00 mg, 0.821 mmol, 1.00 equiv) and Cs₂CO₃ (534.85 mg, 1.642 mmol, 2.00 equiv) in DMF (8.00 mL) was added 1-(bromomethyl)-4-chlorobenzene (337.31 mg, 1.642 mmol, 2.00 equiv) at room temperature. The resulting mixture was stirred for 16 h at room temperature. The reaction was monitored by LCMS. The residue was purified by reverse flash chromatography with the following conditions: Column: C18 Column 120 g; Mobile Phase A: Water (10 mM AcOH), Mobile Phase B: ACN; Flow rate: 60 mL/min; Gradient: 75% B to 95% B in 40 min; 254/220 nm. The fractions containing the desired product were collected at 89% B and concentrated under reduced pressure, to afford [8-(2-chlorophenyl)-7-(4-chlorophenyl)-3-[(4-chlorophenyl)methyl]-2,6-dioxo-2,3,6,7-tetrahydro-1H-purin-1-yl]methyl 2,2-dimethylpropanoate (400 mg, 79.64%) as an off-white solid.

8-(2-chlorophenyl)-7-(4-chlorophenyl)-3-[(4-chlorophenyl)methyl]-2,3,6,7-tetrahydro-1H-purine-2,6-dione. To a stirred solution of [8-(2-chlorophenyl)-7-(4-chlorophenyl)-3-[(4-chlorophenyl)methyl]-2,6-dioxo-2,3,6,7-tetrahydro-1H-purin-1-yl]methyl 2,2-dimethylpropanoate (400.00 mg, 0.654 mmol, 1.00 equiv) in MeOH (10.00 mL) was added sodium hydride (130.73 mg, 3.269 mmol, 5.00 equiv, 60%) at 0° C. The resulting mixture was stirred for 16 h at room temperature. The reaction was monitored by LCMS. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 65% B to 90% B in 40 min; 254/220 nm. The fractions containing the desired product were collected at 84% B and concentrated under reduced pressure. To afford 8-(2-chlorophenyl)-7-(4-chlorophenyl)-3-[(4-chlorophenyl)methyl]-2,3,6,7-tetrahydro-1H-purine-2,6-dione (50 mg, 15.37%) as a white solid.

### Example 14. Preparation of Compound 121

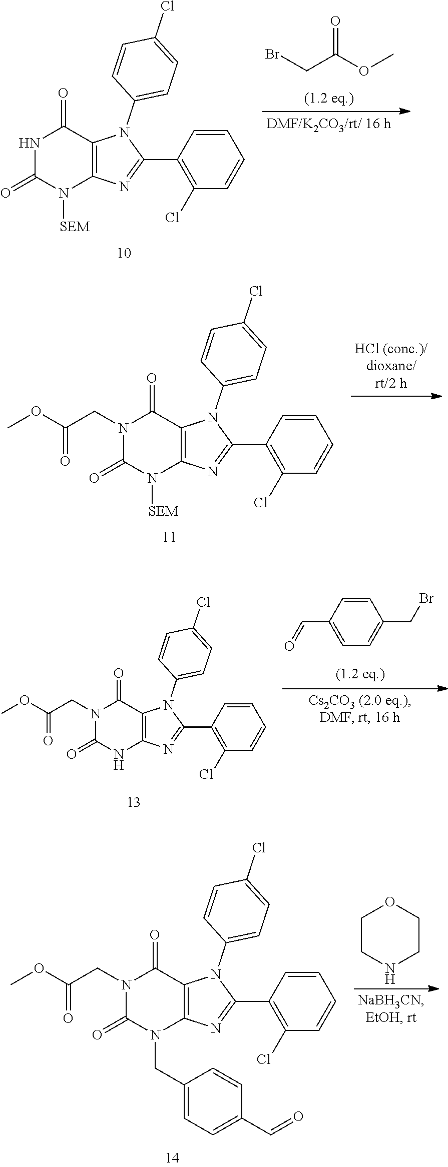

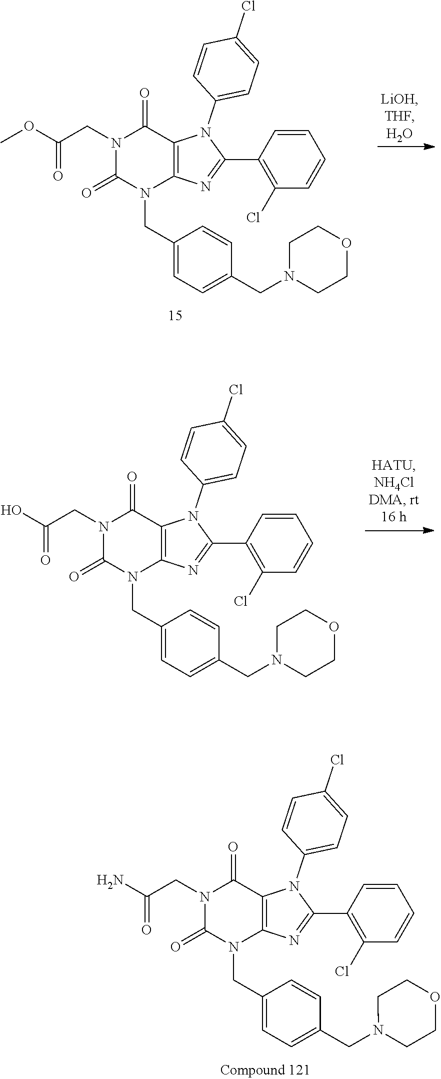 [SMILES: COC(=O)Cn1c(=O)c2c(nc(-c3ccccc3Cl)n2-c2ccc(Cl)cc2)n(Cc2ccc(CN3CCOCC3)cc2)c1=O ; O=C(O)Cn1c(=O)c2c(nc(-c3ccccc3Cl)n2-c2ccc(Cl)cc2)n(Cc2ccc(CN3CCOCC3)cc2)c1=O ; NC(=O)Cn1c(=O)c2c(nc(-c3ccccc3Cl)n2-c2ccc(Cl)cc2)n(Cc2ccc(CN3CCOCC3)cc2)c1=O]

Methyl 2-[8-(2-chlorophenyl)-7-(4-chlorophenyl)-2,6-dioxo-3-[[2-(trimethylsilyl)ethoxy]methyl]-2,3,6,7-tetrahydro-1H-purin-1-yl]acetate. To a stirred solution of 8-(2-chlorophenyl)-7-(4-chlorophenyl)-3-[[2-(trimethylsilyl)ethoxy]methyl]-2,3,6,7-tetrahydro-1H-purine-2,6-dione (200.00 mg, 0.397 mmol, 1.00 equiv) in DMF (5.00 mL) were added K₂CO₃ (109.80 mg, 0.795 mmol, 2.00 equiv) and methyl 2-bromoacetate (72.92 mg, 0.477 mmol, 1.20 equiv) at room temperature. The resulting mixture was stirred for 16 h at room temperature. The reaction was monitored by LCMS. The resulting mixture was extracted with EtOAc (3×20 mL). The combined organic layers were washed with brine (1×50 mL), dried over anhydrous Na₂SO₄. After filtration, the filtrate was concentrated under reduced pressure. The residue was purified by Prep-TLC (hexane/EtOAc 1:1) to afford methyl 2-[8-(2-chlorophenyl)-7-(4-chlorophenyl)-2,6-dioxo-3-[[2-(trimethylsilyl)ethoxy]methyl]-2,3,6,7-tetrahydro-1H-purin-1-yl]acetate (200 mg, 87.48%) as a light yellow oil.

Methyl 2-[8-(2-chlorophenyl)-7-(4-chlorophenyl)-2,6-dioxo-2,3,6,7-tetrahydro-1H-purin-1-yl]acetate. To a stirred solution of 1,4-dioxane (10.00 mL) and HCl (12M) (20.00 mL) was added methyl 2-[8-(2-chlorophenyl)-7-(4-chlorophenyl)-2,6-dioxo-3-[[2-(trimethylsilyl)ethoxy]methyl]-2,3,6,7-tetrahydro-1H-purin-1-yl]acetate (200.00 mg, 0.348 mmol, 1.00 equiv) in portions at room temperature under air atmosphere. The resulting mixture was stirred for 2 h at room temperature under air atmosphere. The reaction was monitored by LCMS. The mixture was basified to pH 6 with saturated NaHCO₃ (aq.). The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE/EtOAc (1:3) to afford methyl 2-[8-(2-chlorophenyl)-7-(4-chlorophenyl)-2,6-dioxo-2,3,6,7-tetrahydro-1H-purin-1-yl]acetate (100 mg, 64.63%) as an off-white solid.

Methyl 2-[8-(2-chlorophenyl)-7-(4-chlorophenyl)-3-[(4-formylphenyl)methyl]-2,6-dioxo-2,3,6,7-tetrahydro-1H-purin-1-yl]acetate. To a stirred solution of methyl 2-[8-(2-chlorophenyl)-7-(4-chlorophenyl)-2,6-dioxo-2,3,6,7-tetrahydro-1H-purin-1-yl]acetate (100.00 mg, 0.225 mmol, 1.00 equiv) in DMF (5.00 mL) were added Cs₂CO₃ (146.35 mg, 0.449 mmol, 2.00 equiv) and 4-(bromomethyl)benzaldehyde (53.64 mg, 0.270 mmol, 1.20 equiv) at room temperature. The resulting mixture was stirred for 16 h at room temperature. The reaction was monitored by LCMS. The resulting mixture was concentrated under reduced pressure. The residue was purified by Prep-TLC (PE/EtOAc 1:1) to afford methyl 2-[8-(2-chlorophenyl)-7-(4-chlorophenyl)-3-[(4-formylphenyl)methyl]-2,6-dioxo-2,3,6,7-tetrahydro-1H-purin-1-yl]acetate (60 mg, 47.42%) as an off-white solid.

Methyl 2-[8-(2-chlorophenyl)-7-(4-chlorophenyl)-3-[[4-(morpholin-4-ylmethyl)phenyl]methyl]-2,6-dioxopurin-1-yl]acetate. To a stirred solution of methyl 2-[8-(2-chlorophenyl)-7-(4-chlorophenyl)-3-[(4-formylphenyl)methyl]-2,6-dioxopurin-1-yl]acetate (60.00 mg, 0.106 mmol, 1.00 equiv) in EtOH (5.00 mL) was added morpholine (18.56 mg, 0.213 mmol, 2.00 equiv) at room temperature. The resulting mixture was stirred for 16 h at 50° C. To the above mixture was added NaBH₃CN (8.03 mg, 0.128 mmol, 1.20 equiv) at room temperature. The resulting mixture was stirred for additional 16 h at room temperature. The reaction was monitored by LCMS. The resulting mixture was concentrated under reduced pressure. The residue was purified by Prep-TLC (PE/EtOAc 1:1) to afford methyl 2-[8-(2-chlorophenyl)-7-(4-chlorophenyl)-3-[[4-(morpholin-4-ylmethyl)phenyl]methyl]-2,6-dioxopurin-1-yl]acetate (45 mg, 66.59%) as a colorless oil.

2-[8-(2-chlorophenyl)-7-(4-chlorophenyl)-3-([4-[(morpholin-4-yl)methyl]phenyl]methyl)-2,6-dioxo-2,3,6,7-tetrahydro-1H-purin-1-yl]acetic acid. To a stirred solution of methyl 2-[8-(2-chlorophenyl)-7-(4-chlorophenyl)-3-[[4-(morpholin-4-ylmethyl)phenyl]methyl]-2,6-dioxopurin-1-yl]acetate (45.00 mg, 0.071 mmol, 1.00 equiv) in THF (3.00 mL) and water (3.00 mL) was added LiOH (16.98 mg, 0.709 mmol, 10.00 equiv) at room temperature. The resulting mixture was stirred for 16 h at room temperature. The reaction was monitored by LCMS. The resulting mixture was filtered, the filter cake was washed with EtOAc (3×10 mL). The filtrate was concentrated under reduced pressure. The crude product was used in the next step directly without further purification.

2-[8-(2-chlorophenyl)-7-(4-chlorophenyl)-3-([4-[(morpholin-4-yl)methyl]phenyl]methyl)-2,6-dioxo-2,3,6,7-tetrahydro-1H-purin-1-yl]acetamide. To a stirred solution of 2-[8-(2-chlorophenyl)-7-(4-chlorophenyl)-3-([4-[(morpholin-4-yl)methyl]phenyl]methyl)-2,6-dioxo-2,3,6,7-tetrahydro-1H-purin-1-yl]acetic acid (12.00 mg, 0.019 mmol, 1.00 equiv) in DMA (3.00 mL) was added HATU (11.03 mg, 0.029 mmol, 1.50 equiv) at room temperature. The resulting mixture was stirred for 0.5 h at room temperature. To the above mixture was added NH₄Cl (1.24 mg, 0.023 mmol, 1.20 equiv) at room temperature. The resulting mixture was stirred for additional 0.5 h at room temperature. To the above mixture was added TEA (5.87 mg, 0.058 mmol, 3.00 equiv) at room temperature. The resulting mixture was stirred for additional 16 h at room temperature. The reaction was monitored by LCMS. The solution was purified by reverse phase flash with the following conditions (Column: XBridge Prep C18 OBD Column, 5 um, 19*150 mm; Mobile Phase A: Water (0.05% TFA), Mobile Phase B: ACN; Flow rate: 25 mL/min; Gradient: 23% B to 45% B in 7 min; 220 nm; Rt: 6.78 min) to afford 2-[8-(2-chlorophenyl)-7-(4-chlorophenyl)-3-([4-[(morpholin-4-yl)methyl]phenyl]methyl)-2,6-dioxo-2,3,6,7-tetrahydro-1H-purin-1-yl]acetamide (3 mg, 25.04%) as an off-white solid.

Compound 118 was prepared by the methods and scheme described for Compound 121 using the appropriate reagents.

### Example 15. Preparation of Compound 117

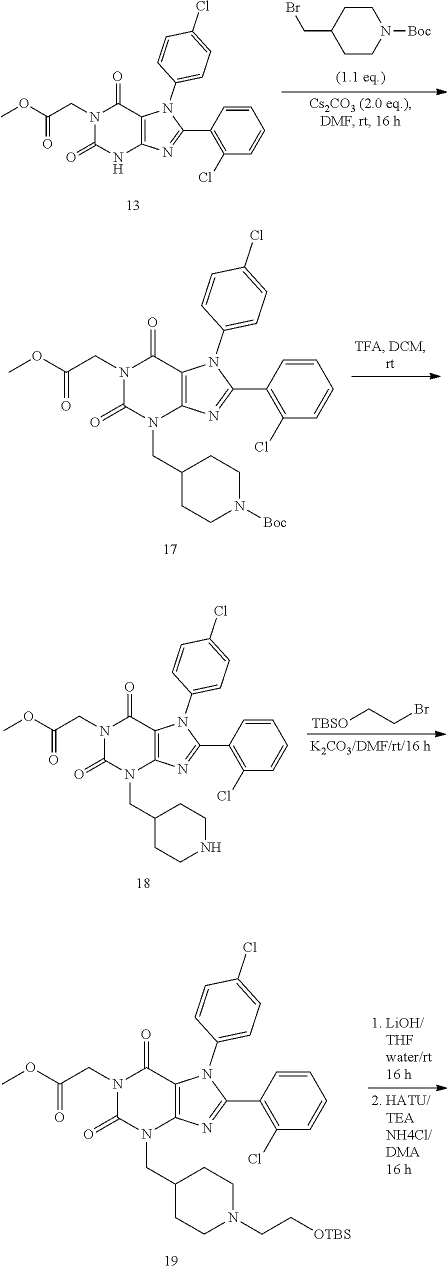 [SMILES: COC(=O)Cn1c(=O)[nH]c2nc(-c3ccccc3Cl)n(-c3ccc(Cl)cc3)c2c1=O ; CC(C)(C)OC(=O)N1CCC(CBr)CC1 ; COC(=O)Cn1c(=O)c2c(nc(-c3ccccc3Cl)n2-c2ccc(Cl)cc2)n(CC2CCN(C(=O)OC(C)(C)C)CC2)c1=O ; COC(=O)Cn1c(=O)c2c(nc(-c3ccccc3Cl)n2-c2ccc(Cl)cc2)n(CC2CCNCC2)c1=O ; CC(C)(C)[Si](C)(C)OCCBr ; COC(=O)Cn1c(=O)c2c(nc(-c3ccccc3Cl)n2-c2ccc(Cl)cc2)n(CC2CCN(CCO[Si](C)(C)C(C)(C)C)CC2)c1=O]

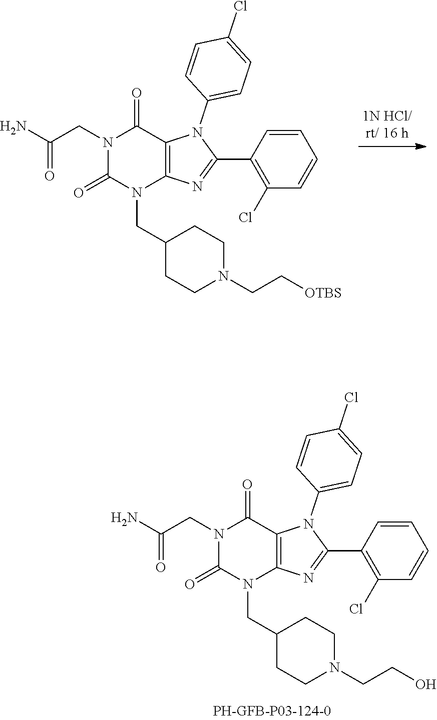 [SMILES: CC(C)(C)[Si](C)(C)OCCN1CCC(Cn2c(=O)n(CC(N)=O)c(=O)c3c2nc(-c2ccccc2Cl)n3-c2ccc(Cl)cc2)CC1 ; NC(=O)Cn1c(=O)c2c(nc(-c3ccccc3Cl)n2-c2ccc(Cl)cc2)n(CC2CCN(CCO)CC2)c1=O]

tert-butyl 4-[[8-(2-chlorophenyl)-7-(4-chlorophenyl)-1-(2-methoxy-2-oxoethyl)-2,6-dioxo-2,3,6,7-tetrahydro-1H-purin-3-yl]methyl]piperidine-1-carboxylate. To a stirred solution/mixture of methyl 2-[8-(2-chlorophenyl)-7-(4-chlorophenyl)-2,6-dioxo-2,3,6,7-tetrahydro-1H-purin-1-yl]acetate (50.00 mg, 0.112 mmol, 1.00 equiv) and Cs₂CO₃ (73.18 mg, 0.225 mmol, 2.00 equiv) in DMF (2.00 mL) was added tert-butyl 4-(bromomethyl)piperidine-1-carboxylate (46.86 mg, 0.168 mmol, 1.50 equiv) at room temperature. The resulting mixture was stirred for 16 h at room temperature. The reaction was monitored by LCMS. The resulting mixture was filtered, the filter cake was washed with EtOAc (2×20 mL). The filtrate was concentrated under reduced pressure. The residue was purified by silica gel column chromatography, eluted with PE/EtOAc (1:1) to afford tert-butyl 4-[[8-(2-chlorophenyl)-7-(4-chlorophenyl)-1-(2-methoxy-2-oxoethyl)-2,6-dioxo-2,3,6,7-tetrahydro-1H-purin-3-yl]methyl]piperidine-1-carboxylate (80 mg, 92.40%) as an light yellow oil.

Methyl 2-[8-(2-chlorophenyl)-7-(4-chlorophenyl)-2,6-dioxo-3-[(piperidin-4-yl)methyl]-2,3,6,7-tetrahydro-1H-purin-1-yl]acetate. To a stirred solution of tert-butyl 4-[[8-(2-chlorophenyl)-7-(4-chlorophenyl)-1-(2-methoxy-2-oxoethyl)-2,6-dioxo-2,3,6,7-tetrahydro-1H-purin-3-yl]methyl]piperidine-1-carboxylate (80.00 mg) in DCM (4.00 mL) was added TFA (1.00 mL) at room temperature. The resulting mixture was stirred for 2 h at room temperature. The mixture was basified to pH 8 with saturated NaHCO₃ (aq.). The resulting mixture was extracted with EtOAc (3×20 mL). The combined organic layers were washed with brine (2×20 mL), dried over anhydrous Na₂SO₄. After filtration, the filtrate was concentrated under reduced pressure. to afford methyl 2-[8-(2-chlorophenyl)-7-(4-chlorophenyl)-2,6-dioxo-3-[(piperidin-4-yl)methyl]-2,3,6,7-tetrahydro-1H-purin-1-yl]acetate (60 mg, 88.84%) as a light yellow oil.

Methyl 2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetate. To a stirred solution/mixture of methyl 2-[8-(2-chlorophenyl)-7-(4-chlorophenyl)-2,6-dioxo-3-[(piperidin-4-yl)methyl]-2,3,6,7-tetrahydro-1H-purin-1-yl]acetate (60.00 mg, 0.111 mmol, 1.00 equiv) and K₂CO₃ (30.58 mg, 0.221 mmol, 2.00 equiv) in DMF (2.00 mL) was added (2-bromoethoxy)(tert-butyl)dimethylsilane (39.69 mg, 0.166 mmol, 1.50 equiv) at room temperature. The resulting mixture was stirred for 16 h at room temperature. The reaction was monitored by LCMS. The residue was purified by silica gel column chromatography, eluted with PE/EtOAc (1:1) to afford methyl 2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetate (41 mg, 52.90%) as a light yellow solid.

2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetic acid. To a stirred solution of methyl 2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetate (41.00 mg, 0.059 mmol, 1.00 equiv) and LiOH (14.01 mg, 0.585 mmol, 10 equiv) in THF (2.00 mL) was added H₂O (2.00 mL) at room temperature. The resulting mixture was stirred for 16 h at room temperature. The reaction was monitored by LCMS. The mixture was acidified to pH 6 with HCl (2M)(aq.). The resulting mixture was extracted with EtOAc (3×10 mL). The combined organic layers were washed with brine (3×10 mL), dried over anhydrous Na₂SO₄. After filtration, the filtrate was concentrated under reduced pressure. to afford 2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetic acid (25 mg, 62.22%) as an off-white solid.

2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetamide. To a stirred solution of 2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetic acid (25.00 mg, 0.036 mmol, 1.00 equiv) in DMA (3.00 mL) was added HATU (20.76 mg, 0.055 mmol, 1.50 equiv) at room temperature. The resulting mixture was stirred for 30 min at room temperature. To the above mixture was added NH₄Cl (5.84 mg, 0.109 mmol, 3.00 equiv). The resulting mixture was stirred for 30 min at room temperature. To the above mixture was added TEA (36.84 mg, 0.364 mmol, 10.00 equiv) at room temperature. The resulting mixture was stirred for 16 h at room temperature. The reaction was monitored by LCMS. The residue was purified by silica gel column chromatography, eluted with PE/EtOAc (1:1) to afford 2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetamide (20 mg, 80.12%) as a light yellow solid.

2-[8-(2-chlorophenyl)-7-(4-chlorophenyl)-3-[[1-(2-hydroxyethyl)piperidin-4-yl]methyl]-2,6-dioxo-2,3,6,7-tetrahydro-1H-purin-1-yl]acetamide. A solution of 2-[3-[(1-[2-[(tert-butyldimethylsilyl)oxy]ethyl]piperidin-4-yl)methyl]-8-(2-chlorophenyl)-7-(4-chlorophenyl)-2,6-dioxo-2,3,6,7-tetrahydro-1H-purin-1-yl]acetamide (20.00 mg, 0.029 mmol, 1.00 equiv) and HCl (2M) (2.00 mL) in THF (2.00 mL) was stirred for 16 h at room temperature. The reaction was monitored by LCMS. The mixture was basified to pH 8 with saturated NaHCO₃ (aq.). The resulting mixture was extracted with EtOAc (3×10 mL). The combined organic layers were washed with brine (3×10 mL), dried over anhydrous Na₂SO₄. After filtration, the filtrate was concentrated under reduced pressure. The crude product was purified by Prep-HPLC with the following conditions (Column: Xselect CSH OBD Column 30×150 mm 5 um; Mobile Phase A: Water (0.05% TFA), Mobile Phase B: ACN; Flow rate: 60 mL/min; Gradient: 15% B to 39% B in 7 min; 220 nm; Rt: 6.02 min) to afford 2-[8-(2-chlorophenyl)-7-(4-chlorophenyl)-3-[[1-(2-hydroxyethyl)piperidin-4-yl]methyl]-2,6-dioxo-2,3,6,7-tetrahydro-1H-purin-1-yl]acetamide (8 mg, 48.00%) as an off-white solid.

### Example 16. Preparation of Compound 125

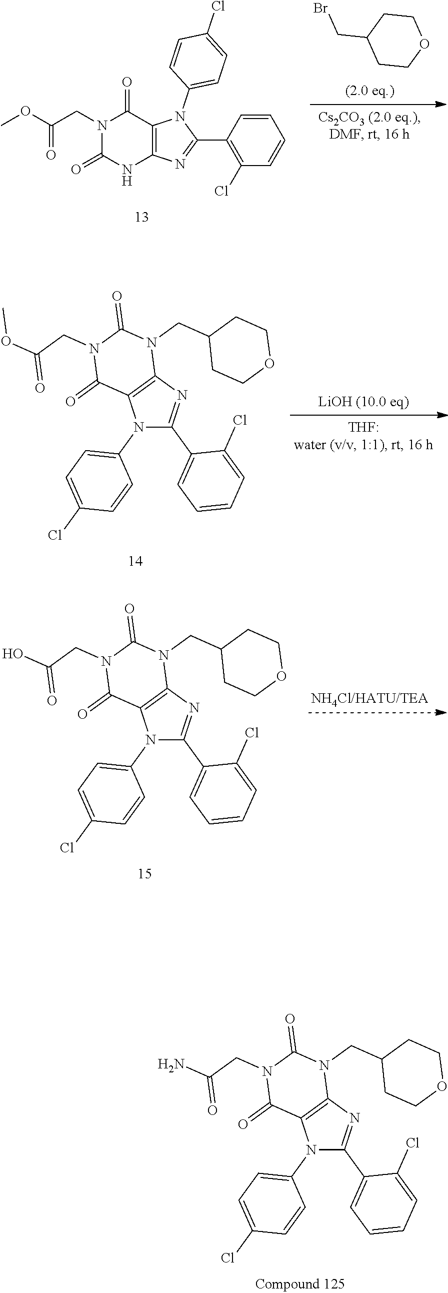 [SMILES: COC(=O)Cn1c(=O)[nH]c2nc(-c3ccccc3Cl)n(-c3ccc(Cl)cc3)c2c1=O ; BrCC1CCOCC1 ; COC(=O)Cn1c(=O)c2c(nc(-c3ccccc3Cl)n2-c2ccc(Cl)cc2)n(CC2CCOCC2)c1=O ; O=C(O)Cn1c(=O)c2c(nc(-c3ccccc3Cl)n2-c2ccc(Cl)cc2)n(CC2CCOCC2)c1=O ; NC(=O)Cn1c(=O)c2c(nc(-c3ccccc3Cl)n2-c2ccc(Cl)cc2)n(CC2CCOCC2)c1=O]

Methyl 2-[8-(2-chlorophenyl)-7-(4-chlorophenyl)-3-(oxan-4-ylmethyl)-2,6-dioxopurin-1-yl]acetate. To a stirred solution of methyl 2-[8-(2-chlorophenyl)-7-(4-chlorophenyl)-2,6-dioxo-3H-purin-1-yl]acetate (30.00 mg, 0.067 mmol, 1.00 equiv) and Cs₂CO₃ (43.91 mg, 0.135 mmol, 2.00 equiv) in DMF (5.00 mL) was added 4-(bromomethyl)oxane (24.13 mg, 0.135 mmol, 2.00 equiv) at room temperature. The resulting mixture was stirred for overnight at room temperature. The reaction was monitored by LCMS. The resulting mixture was extracted with EtOAc (3×20 mL). The combined organic layers were washed with brine (3×60 mL), dried over anhydrous Na₂SO₄. After filtration, the filtrate was concentrated under reduced pressure. The residue was purified by silica gel column chromatography, eluted with PE/EtOAc (1:1) to afford methyl 2-[8-(2-chlorophenyl)-7-(4-chlorophenyl)-3-(oxan-4-ylmethyl)-2,6-dioxopurin-1-yl]acetate (30 mg, 81.94%) as a light yellow oil.

[8-(2-chlorophenyl)-7-(4-chlorophenyl)-3-(oxan-4-ylmethyl)-2,6-dioxopurin-1-yl]acetic acid. To a stirred solution of methyl 2-[8-(2-chlorophenyl)-7-(4-chlorophenyl)-3-(oxan-4-ylmethyl)-2,6-dioxopurin-1-yl]acetate (30.00 mg, 0.055 mmol, 1.00 equiv) in THF (2.00 mL) was added LiOH (13.22 mg, 0.552 mmol, 10 equiv) in H₂O (2.00 mL) at room temperature. The resulting mixture was stirred for overnight at room temperature. The reaction was monitored by LCMS. The mixture/residue was acidified to pH 6 with HCl (aq.). The resulting mixture was extracted with EtOAc (3×20 mL). The combined organic layers were washed with brine (3×20 mL), dried over anhydrous Na₂SO₄. After filtration, the filtrate was concentrated under reduced pressure. to afford [8-(2-chlorophenyl)-7-(4-chlorophenyl)-3-(oxan-4-ylmethyl)-2,6-dioxopurin-1-yl]acetic acid (crude) as a light yellow oil.

2-[8-(2-chlorophenyl)-7-(4-chlorophenyl)-3-(oxan-4-ylmethyl)-2,6-dioxopurin-1-yl]acetamide. A solution/mixture of [8-(2-chlorophenyl)-7-(4-chlorophenyl)-3-(oxan-4-ylmethyl)-2,6-dioxopurin-1-yl]acetic acid (30.00 mg, 0.057 mmol, 1.00 equiv) and HATU (32.32 mg, 0.085 mmol, 1.5 equiv) in DMA (5.00 mL) was stirred for 30 min at room temperature. followed by the addition of NH₄Cl (9.09 mg, 0.170 mmol, 3 equiv) at room temperature. To the above mixture was added TEA (17.20 mg, 0.170 mmol, 3 equiv) over 30 min at room temperature. The resulting mixture was stirred for additional overnight at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC with the following conditions (Column: XBridge Shield RP18 OBD Column, 5 um, 19*150 mm; Flow rate: 25 mL/min; Gradient: 35% B to 50% B in 7 min; 220/254 nm; Rt: 6.5 min) to afford 2-[8-(2-chlorophenyl)-7-(4-chlorophenyl)-3-(oxan-4-ylmethyl)-2,6-dioxopurin-1-yl]acetamide (15 mg, 50.09%) as a white solid.

### Example 17. Preparation of Compounds 124, 127, and 128

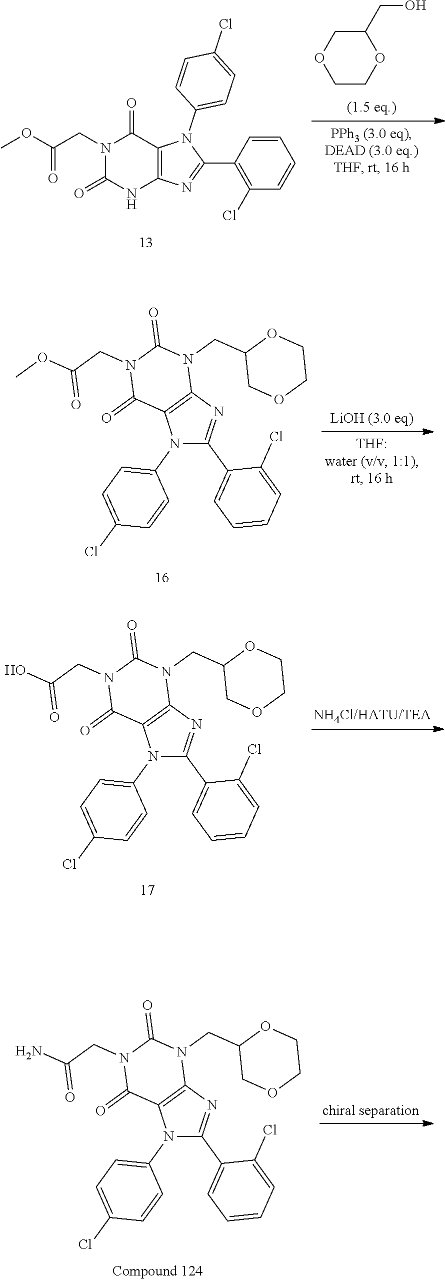 [SMILES: COC(=O)Cn1c(=O)[nH]c2nc(-c3ccccc3Cl)n(-c3ccc(Cl)cc3)c2c1=O ; OCC1COCCO1 ; COC(=O)Cn1c(=O)c2c(nc(-c3ccccc3Cl)n2-c2ccc(Cl)cc2)n(CC2COCCO2)c1=O ; O=C(O)Cn1c(=O)c2c(nc(-c3ccccc3Cl)n2-c2ccc(Cl)cc2)n(CC2COCCO2)c1=O ; NC(=O)Cn1c(=O)c2c(nc(-c3ccccc3Cl)n2-c2ccc(Cl)cc2)n(CC2COCCO2)c1=O]

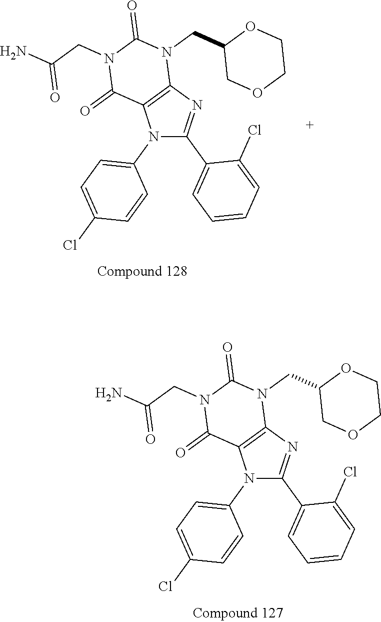

Methyl 2-[8-(2-chlorophenyl)-7-(4-chlorophenyl)-3-(1,4-dioxan-2-ylmethyl)-2,6-dioxopurin-1-yl]acetate. To a stirred solution/mixture of methyl 2-[8-(2-chlorophenyl)-7-(4-chlorophenyl)-2,6-dioxo-3H-purin-1-yl]acetate (30.00 mg, 0.067 mmol, 1.00 equiv) and PPh₃ (53.02 mg, 0.202 mmol, 3 equiv) in THF (5.00 mL) was added DEAD (35.20 mg, 0.202 mmol, 3.00 equiv) dropwise at 0° C. under nitrogen atmosphere. To the above mixture was added 1,4-dioxan-2-ylmethanol (11.94 mg, 0.101 mmol, 1.5 equiv) at room temperature. The resulting mixture was stirred for additional overnight at room temperature under nitrogen atmosphere. The reaction was monitored by LCMS. The residue was purified by silica gel column chromatography, eluted with PE/EtOAc (1:1) to afford methyl 2-[8-(2-chlorophenyl)-7-(4-chlorophenyl)-3-(1,4-dioxan-2-ylmethyl)-2,6-dioxopurin-1-yl]acetate (30 mg, 81.64%) as a light yellow oil.

[8-(2-chlorophenyl)-7-(4-chlorophenyl)-3-(1,4-dioxan-2-ylmethyl)-2,6-dioxopurin-1-yl]acetic acid. To a stirred solution of methyl 2-[8-(2-chlorophenyl)-7-(4-chlorophenyl)-3-(1,4-dioxan-2-ylmethyl)-2,6-dioxopurin-1-yl]acetate (30.00 mg, 0.055 mmol, 1.00 equiv) in THF (2.00 mL) was added LiOH (3.95 mg, 0.165 mmol, 3 equiv) in H₂O (2.00 mL) at room temperature. The resulting mixture was stirred for 16 h at room temperature. The mixture was acidified to pH 6 with HCl (aq.). The crude product was used in the next step directly without further purification.

2-[8-(2-chlorophenyl)-7-(4-chlorophenyl)-3-(1,4-dioxan-2-ylmethyl)-2,6-dioxopurin-1-yl]acetamide. To a stirred solution of [8-(2-chlorophenyl)-7-(4-chlorophenyl)-3-(1,4-dioxan-2-ylmethyl)-2,6-dioxopurin-1-yl]acetic acid (25.00 mg, 0.047 mmol, 1.00 equiv) in DMA (3.00 mL) was added HATU (26.83 mg, 0.071 mmol, 1.5 equiv) at room temperature. The resulting mixture was stirred for 30 min at room temperature. To the above mixture was added NH₄Cl (7.55 mg, 0.141 mmol, 3 equiv) at room temperature. The resulting mixture was stirred for 30 min at room temperature. To the above mixture was added TEA (14.28 mg, 0.141 mmol, 3 equiv) at room temperature. The resulting mixture was stirred for 16 h at room temperature. The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC with the following conditions (Column: XBridge Shield RP18 OBD Column, 5 um, 19*150 mm; Flow rate: 25 mL/min; Gradient: 35% B to 45% B in 7 min; 220/254 nm; Rt: 6.5 min) to afford 2-[8-(2-chlorophenyl)-7-(4-chlorophenyl)-3-(1,4-dioxan-2-ylmethyl)-2,6-dioxopurin-1-yl]acetamide (10 mg, 40.07%) as a white solid.

2-[8-(2-chlorophenyl)-7-(4-chlorophenyl)-3-[(2R)-1,4-dioxan-2-ylmethyl]-2,6-dioxopurin-1-yl]acetamide and 2-[8-(2-chlorophenyl)-7-(4-chlorophenyl)-3-[(2S)-1,4-dioxan-2-ylmethyl]-2,6-dioxopurin-1-yl]acetamide. The racemic (scale up batch) was purified by reverse phase flash with the following conditions (Column: CHIRALPAK IE, 2*25 cm, 5 um; Mobile Phase A: Hex (0.2% IPA)—HPLC, Mobile Phase B: EtOH—HPLC; Flow rate: 17 mL/min; Gradient: 50 B to 50 B in 39 min; 220/254 nm; RT1: 26.099) to afford 2-[8-(2-chlorophenyl)-7-(4-chlorophenyl)-3-[(2R)-1,4-dioxan-2-ylmethyl]-2,6-dioxopurin-1-yl]acetamide (30 mg, 16.22%) and 2-[8-(2-chlorophenyl)-7-(4-chlorophenyl)-3-[(2S)-1,4-dioxan-2-ylmethyl]-2,6-dioxopurin-1-yl]acetamide (30 mg, 16.22%) as white solid.

### Example 18. Preparation of Compound 123

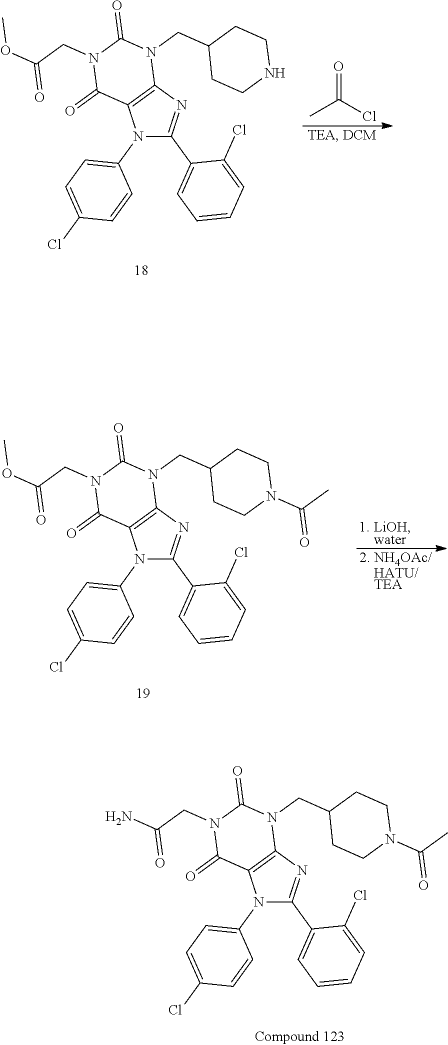

Methyl 2-[3-[(1-acetylpiperidin-4-yl)methyl]-8-(2-chlorophenyl)-7-(4-chlorophenyl)-2,6-dioxopurin-1-yl]acetate. To a stirred solution of methyl 2-[8-(2-chlorophenyl)-7-(4-chlorophenyl)-2,6-dioxo-3-(piperidin-4-ylmethyl)purin-1-yl]acetate (70.00 mg, 0.129 mmol, 1.00 equiv) in DCM (3.00 mL) were added TEA (39.18 mg, 0.387 mmol, 3.00 equiv) and acetyl chloride (20.26 mg, 0.258 mmol, 2.00 equiv) at room temperature. The resulting mixture was stirred for 16 h at room temperature. The reaction was monitored by LCMS. The resulting mixture was concentrated under vacuum. The residue was purified by Prep-TLC (PE/EtOAc 1:1) to afford methyl 2-[3-[(1-acetylpiperidin-4-yl)methyl]-8-(2-chlorophenyl)-7-(4-chlorophenyl)-2,6-dioxopurin-1-yl]acetate (50 mg, 66.29%) as a colorless oil.

[3-[(1-acetylpiperidin-4-yl)methyl]-8-(2-chlorophenyl)-7-(4-chlorophenyl)-2,6-dioxopurin-1-yl]acetic acid. To a stirred mixture of methyl 2-[3-[(1-acetylpiperidin-4-yl)methyl]-8-(2-chlorophenyl)-7-(4-chlorophenyl)-2,6-dioxopurin-1-yl]acetate (50.00 mg, 0.086 mmol, 1.00 equiv) in THF (2.00 mL) and H₂O (2.00 mL) was added LiOH (20.49 mg, 0.856 mmol, 10.00 equiv) at room temperature. The resulting mixture was stirred for 16 h at room temperature. The reaction was monitored by LCMS. The mixture was acidified to pH 6 with HCl (aq. 2N). The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by Prep-TLC (CH₂Cl₂/MeOH 10:1) to afford [3-[(1-acetylpiperidin-4-yl)methyl]-8-(2-chlorophenyl)-7-(4-chlorophenyl)-2,6-dioxopurin-1-yl]acetic acid (30 mg).

2-[3-[(1-acetylpiperidin-4-yl)methyl]-8-(2-chlorophenyl)-7-(4-chlorophenyl)-2,6-dioxopurin-1-yl]acetamide. To a stirred solution of [3-[(1-acetylpiperidin-4-yl)methyl]-8-(2-chlorophenyl)-7-(4-chlorophenyl)-2,6-dioxopurin-1-yl]acetic acid (30.00 mg, 0.053 mmol, 1.00 equiv) in DMA (5.00 mL) was added HATU (30.00 mg, 0.079 mmol, 1.50 equiv) at room temperature. The resulting mixture was stirred for 0.5 h at room temperature. To the above mixture was added NH₄OAc (4.86 mg, 0.063 mmol, 1.20 equiv) at room temperature. The resulting mixture was stirred for additional 0.5 h at room temperature. To the above mixture was added TEA (15.97 mg, 0.158 mmol, 3.00 equiv) at room temperature. The resulting mixture was stirred for additional 16 h at room temperature. The solution was purified by reverse phase flash with the following conditions (Column: XBridge Shield RP18 OBD Column, 5 um, 19*150 mm; Flow rate: 25 mL/min; Gradient: 30% B to 50% B in 7 min; 220/254 nm; Rt: 6.5 min) to afford 2-[3-[(1-acetylpiperidin-4-yl)methyl]-8-(2-chlorophenyl)-7-(4-chlorophenyl)-2,6-dioxopurin-1-yl]acetamide (20 mg, 66.78%) as a white solid.

### Example 19. Preparation of Compound 122

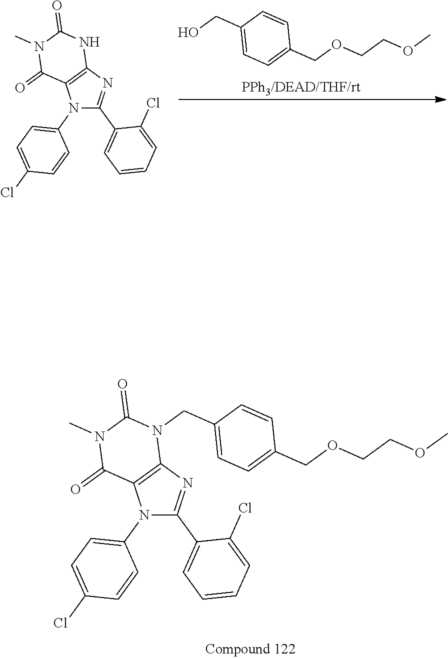

To a stirred solution of 8-(2-chlorophenyl)-7-(4-chlorophenyl)-1-methyl-3H-purine-2,6-dione (20.00 mg, 0.052 mmol, 1.00 equiv) and PPh₃ (40.64 mg, 0.155 mmol, 3 equiv) in THF (5.00 mL) were added DEAD (26.99 mg, 0.155 mmol, 3 equiv) and [4-[(2-methoxyethoxy)methyl]phenyl]methanol (15.20 mg, 0.077 mmol, 1.5 equiv) at 0° C. under nitrogen atmosphere. The resulting mixture was stirred for 3 h at room temperature under nitrogen atmosphere. The reaction was monitored by LCMS. The resulting mixture was extracted with EtOAc (3×10 mL). The combined organic layers were washed with brine (3×10 mL), dried over anhydrous Na₂SO₄. After filtration, the filtrate was concentrated under reduced pressure. The crude product was purified by Prep-HPLC with the following conditions (Column: Xselect CSH OBD Column 30*150 mm 5 um n; Mobile Phase A: Water (0.05% TFA), Mobile Phase B: ACN; Flow rate: 60 mL/min; Gradient: 43% B to 80% B in 7 min; 220 nm; Rt: 6.50 min) to afford 8-(2-chlorophenyl)-7-(4-chlorophenyl)-3-([4-[(2-methoxyethoxy)methyl]phenyl]methyl)-1-methylpurine-2,6-dione (15 mg, 51.36%) as a white solid.

### Example 20. Preparation of Compound 130

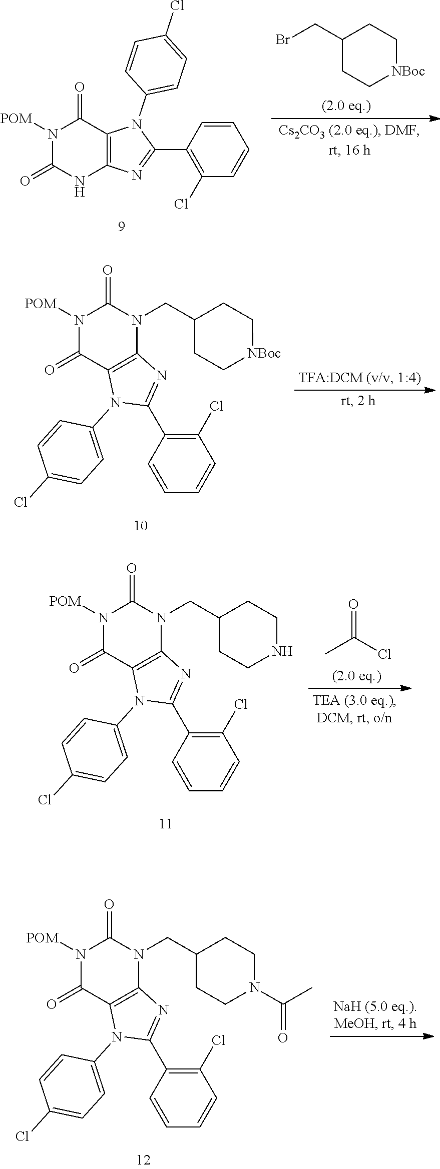

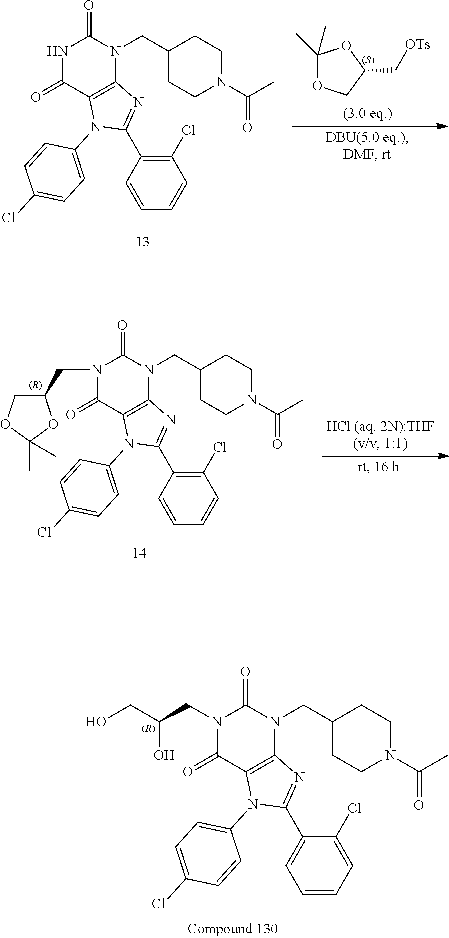

tert-butyl 4-[[8-(2-chlorophenyl)-7-(4-chlorophenyl)-1-[[(2,2-dimethylpropanoyl)oxy]methyl]-2,6-dioxopurin-3-yl]methyl]piperidine-1-carboxylate. To a stirred solution of [8-(2-chlorophenyl)-7-(4-chlorophenyl)-2,6-dioxo-3H-purin-1-yl]methyl 2,2-dimethylpropanoate (300.00 mg, 0.616 mmol, 1.00 equiv) and Cs₂CO₃ (401.14 mg, 1.231 mmol, 2 equiv) in DMF (5.00 mL) was added tert-butyl 4-(bromomethyl)piperidine-1-carboxylate (342.50 mg, 1.231 mmol, 2 equiv) at room temperature. The resulting mixture was stirred for 20 h at room temperature. The reaction was monitored by LCMS. The residue was purified by reverse flash chromatography with the following conditions: Column: C18 Column 120 g; Mobile Phase A: Water (10 mM AcOH), Mobile Phase B: ACN; Flow rate: 50 mL/min; Gradient: 80% B to 95% B in 40 min; 254/220 nm. The fractions containing the desired product were collected at 90% B and concentrated under reduced pressure. To afford tert-butyl 4-[[8-(2-chlorophenyl)-7-(4-chlorophenyl)-1-[[(2,2-dimethylpropanoyl)oxy]methyl]-2,6-dioxopurin-3-yl]methyl]piperidine-1-carboxylate (190 mg, 45.08%) as an off-white solid. [8-(2-chlorophenyl)-7-(4-chlorophenyl)-2,6-dioxo-3-(piperidin-4-ylmethyl)purin-1-yl]methyl 2,2-dimethylpropanoate. To a stirred solution of tert-butyl 4-[[8-(2-chlorophenyl)-7-(4-chlorophenyl)-1-[[(2,2-dimethylpropanoyl)oxy]methyl]-2,6-dioxopurin-3-yl]methyl]piperidine-1-carboxylate (190.00 mg, 0.278 mmol, 1 equiv) in DCM (4.00 mL, 62.920 mmol, 172.31 equiv) was added TFA (1.00 mL, 12.343 mmol, 33.80 equiv) at room temperature. The resulting mixture was stirred for 2 h at room temperature. The reaction was monitored by LCMS. The mixture was acidified to pH 6 with saturated NaHCO₃ (aq.). The resulting mixture was extracted with EtOAc (3×20 mL). The combined organic layers were washed with brine (3×20 mL), dried over anhydrous Na₂SO₄. After filtration, the filtrate was concentrated under reduced pressure. to afford [8-(2-chlorophenyl)-7-(4-chlorophenyl)-2,6-dioxo-3-(piperidin-4-ylmethyl)purin-1-yl]methyl 2,2-dimethylpropanoate (crude) as an off-white solid.

[3-[(1-acetylpiperidin-4-yl)methyl]-8-(2-chlorophenyl)-7-(4-chlorophenyl)-2,6-dioxopurin-1-yl]methyl 2,2-dimethylpropanoate. To a stirred solution of [8-(2-chlorophenyl)-7-(4-chlorophenyl)-2,6-dioxo-3-(piperidin-4-ylmethyl)purin-1-yl]methyl 2,2-dimethylpropanoate (190.00 mg, 0.325 mmol, 1.00 equiv) and acetyl chloride (51.04 mg, 0.650 mmol, 2.00 equiv) in DCM (5.00 mL) was added TEA (98.68 mg, 0.975 mmol, 3.00 equiv) at room temperature. The resulting mixture was stirred for overnight at room temperature. The reaction was monitored by LCMS. The residue was purified by silica gel column chromatography, eluted with PE/EtOAc (1:1) to afford [3-[(1-acetylpiperidin-4-yl)methyl]-8-(2-chlorophenyl)-7-(4-chlorophenyl)-2,6-dioxopurin-1-yl]methyl 2,2-dimethylpropanoate (150 mg, 73.65%) as an off-white solid. 3-[(1-acetylpiperidin-4-yl)methyl]-8-(2-chlorophenyl)-7-(4-chlorophenyl)-1H-purine-2,6-dione. To a stirred solution/mixture of [3-[(1-acetylpiperidin-4-yl)methyl]-8-(2-chlorophenyl)-7-(4-chlorophenyl)-2,6-dioxopurin-1-yl]methyl 2,2-dimethylpropanoate (150.00 mg, 0.239 mmol, 1.00 equiv) in MeOH (5.00 mL) was added NaH (47.88 mg, 1.197 mmol, 5 equiv, 60%) at room temperature. The resulting mixture was stirred for 4 h at room temperature. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 55% B to 70% B in 40 min; 254/220 nm. The fractions containing the desired product were collected at 65% B and concentrated under reduced pressure. To afford 3-[(1-acetylpiperidin-4-yl)methyl]-8-(2-chlorophenyl)-7-(4-chlorophenyl)-1H-purine-2,6-dione (88 mg, 71.74%) as an off-white solid.

(R)-3-[(1-acetylpiperidin-4-yl)methyl]-8-(2-chlorophenyl)-7-(4-chlorophenyl)-1-[(2,2-dimethyl-1,3-dioxolan-4-yl)methyl]purine-2,6-dione. To a stirred solution of 3-[(1-acetylpiperidin-4-yl)methyl]-8-(2-chlorophenyl)-7-(4-chlorophenyl)-1H-purine-2,6-dione (40.00 mg, 0.078 mmol, 1.00 equiv) and (2,2-dimethyl-1,3-dioxolan-4-yl)methyl 4-methylbenzenesulfonate (67.06 mg, 0.234 mmol, 3.00 equiv) in DMF (3.00 mL) was added DBU (59.42 mg, 0.390 mmol, 5.00 equiv) at room temperature. The resulting mixture was stirred for 30 h at room temperature. The reaction was monitored by LCMS. The residue was purified by silica gel column chromatography, eluted with CH₂Cl₂/MeOH (20:1) to afford 3-[(1-acetylpiperidin-4-yl)methyl]-8-(2-chlorophenyl)-7-(4-chlorophenyl)-1-[(2,2-dimethyl-1,3-dioxolan-4-yl)methyl]purine-2,6-dione (crude) as a light yellow oil.

(R)-3-[(1-acetylpiperidin-4-yl)methyl]-8-(2-chlorophenyl)-7-(4-chlorophenyl)-1-[(2R)-2,3-dihydroxypropyl]purine-2,6-dione. To a stirred solution/mixture of (R)-3-[(1-acetylpiperidin-4-yl)methyl]-8-(2-chlorophenyl)-7-(4-chlorophenyl)-1-[[2,2-dimethyl-1,3-dioxolan-4-yl]methyl]purine-2,6-dione (40.00 mg, 0.064 mmol, 1.00 equiv) and HCl (2M) (2.00 mL) in THF (2.00 mL) at room temperature. The resulting mixture was stirred for 1 h at room temperature. The reaction was monitored by LCMS. The mixture was acidified to pH 7 with saturated NaHCO₃ (aq.). The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC with the following conditions (Column: XBridge Shield RP18 OBD Column, 30*150 mm, 5 um; Mobile Phase A: Water (0.1% FA), Mobile Phase B: ACN; Flow rate: 60 mL/min; Gradient: 35 B to 65 B in 7 min; 254/220 nm; RT1: 6.5; RT2:; Injection Volume: mL) to afford (R)-3-[(1-acetylpiperidin-4-yl)methyl]-8-(2-chlorophenyl)-7-(4-chlorophenyl)-1-[2,3-dihydroxypropyl]purine-2,6-dione (20 mg, 53.42%) as a white solid.

### Example 21. Preparation of Compounds 139 and 140

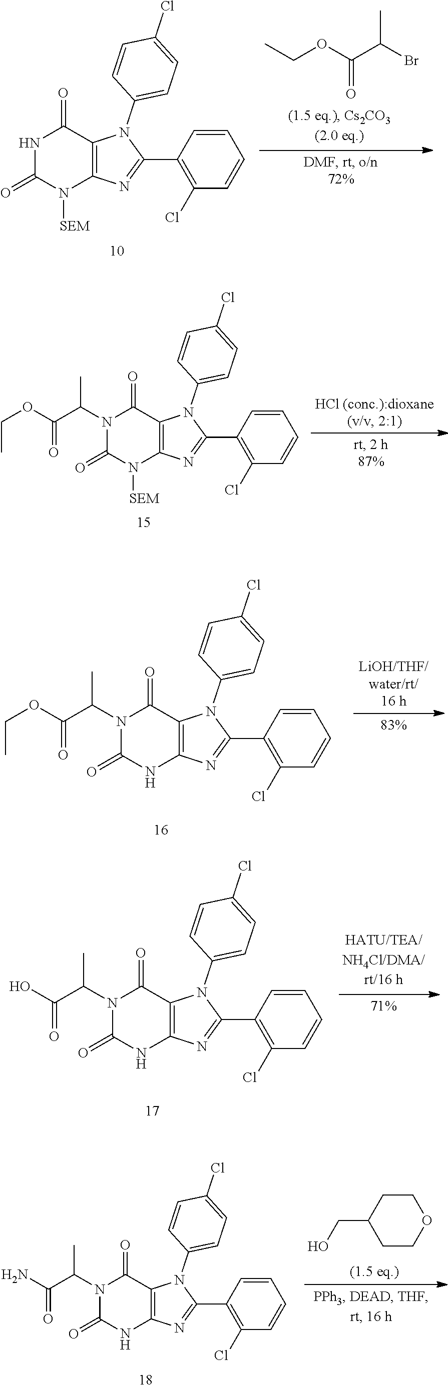

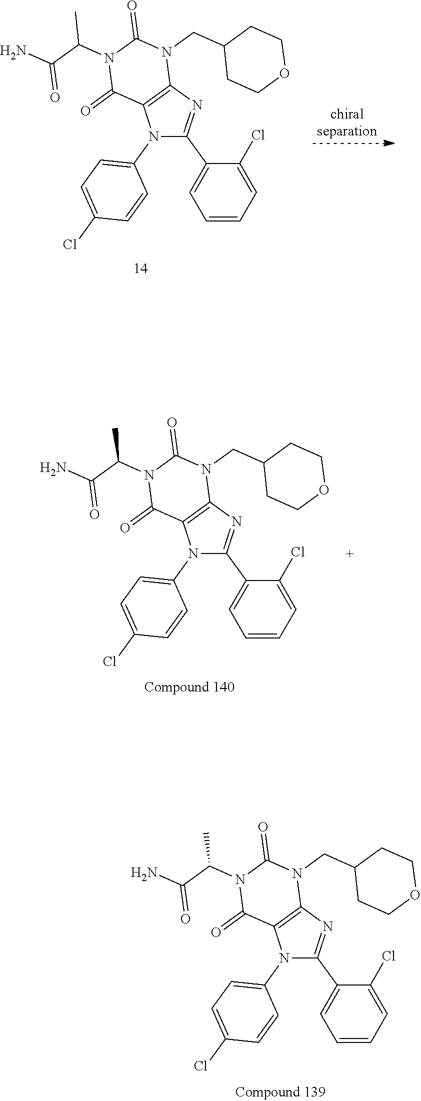 [SMILES: CC(C(N)=O)n1c(=O)c2c(nc(-c3ccccc3Cl)n2-c2ccc(Cl)cc2)n(CC2CCOCC2)c1=O ; C[C@H](C(N)=O)n1c(=O)c2c(nc(-c3ccccc3Cl)n2-c2ccc(Cl)cc2)n(CC2CCOCC2)c1=O ; C[C@@H](C(N)=O)n1c(=O)c2c(nc(-c3ccccc3Cl)n2-c2ccc(Cl)cc2)n(CC2CCOCC2)c1=O]

Ethyl 2-[8-(2-chlorophenyl)-7-(4-chlorophenyl)-2,6-dioxo-3-[[2-(trimethylsilyl)ethoxy]methyl]purin-1-yl]propanoate. To a stirred solution of 8-(2-chlorophenyl)-7-(4-chlorophenyl)-3-[[2-(trimethylsilyl)ethoxy]methyl]-1H-purine-2,6-dione (800.00 mg, 1.589 mmol, 1.00 equiv) and Cs₂CO₃ (1.04 g, 3.178 mmol, 2.00 equiv) in DMF (8.00 mg) was added ethyl a-bromopropionate (431.48 mg, 2.384 mmol, 1.50 equiv) at room temperature. The resulting mixture was stirred for overnight at room temperature. The reaction was monitored by LCMS. The residue was purified by reverse flash chromatography with the following conditions: Column: C18 Column 120 g; Mobile Phase A: Water (10 mM AcOH), Mobile Phase B: ACN; Flow rate: 50 mL/min; Gradient: 80% B to 98% B in 40 min; 254/220 nm. The fractions containing the desired product were collected at 93% B and concentrated under reduced pressure, to afford ethyl 2-[8-(2-chlorophenyl)-7-(4-chlorophenyl)-2,6-dioxo-3-[[2-(trimethylsilyl)ethoxy]methyl]purin-1-yl]propanoate (870 mg, 90.71%) as a yellow oil.

Ethyl 2-[8-(2-chlorophenyl)-7-(4-chlorophenyl)-2,6-dioxo-3H-purin-1-yl]propanoate. To a stirred solution of ethyl 2-[8-(2-chlorophenyl)-7-(4-chlorophenyl)-2,6-dioxo-3-[[2-(trimethylsilyl)ethoxy]methyl]purin-1-yl]propanoate (870.00 mg, 1 equiv) and HCl (12M) (8.00 mL) in 1,4-dioxane (4.00 mL) at room temperature. The resulting mixture was stirred for 4 h at room temperature. The mixture was acidified to pH 6 with saturated NaHCO₃ (aq.). The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM AcOH), Mobile Phase B: ACN; Flow rate: 60 mL/min; Gradient: 80% B to 98% B in 40 min; 254/220 nm. The fractions containing the desired product were collected at 95% B and concentrated under reduced pressure, to afford ethyl 2-[8-(2-chlorophenyl)-7-(4-chlorophenyl)-2,6-dioxo-3H-purin-1-yl]propanoate (560 mg, 82.08%) as a white solid.

2-[8-(2-chlorophenyl)-7-(4-chlorophenyl)-2,6-dioxo-3H-purin-1-yl]propanoic acid. To a stirred solution of ethyl 2-[8-(2-chlorophenyl)-7-(4-chlorophenyl)-2,6-dioxo-3H-purin-1-yl]propanoate (560.00 mg, 1.183 mmol, 1.00 equiv) and LiOH (283.34 mg, 11.832 mmol, 10.00 equiv) in THF (6.00 mL) was added H₂O (3.00 mL) at room temperature. The resulting mixture was stirred for 16 h at room temperature. The reaction was monitored by LCMS. The mixture was acidified to pH 6 with HCl (aq.). The residue was purified by reverse phase flash chromatography with the following conditions: Column: C18 Column 80 g; Mobile Phase A: Water (10 mM AcOH), Mobile Phase B: ACN; Flow rate: 60 mL/min; Gradient: 60% B to 80% B in 40 min; 254/220 nm. The fractions containing the desired product were collected at 70% B and concentrated under reduced pressure. To afford 2-[8-(2-chlorophenyl)-7-(4-chlorophenyl)-2,6-dioxo-3H-purin-1-yl]propanoic acid (450 mg, 85.42%) as a light yellow oil.

2-[8-(2-chlorophenyl)-7-(4-chlorophenyl)-2,6-dioxo-3H-purin-1-yl]propanamide. A solution of 2-[8-(2-chlorophenyl)-7-(4-chlorophenyl)-2,6-dioxo-3H-purin-1-yl]propanoic acid (450.00 mg, 1.011 mmol, 1.00 equiv) in DMA (8.00 mL) was treated with HATU (576.42 mg, 1.516 mmol, 1.50 equiv) for 30 min at room temperature followed by the addition of NH₄Cl (162.18 mg, 3.032 mmol, 3.00 equiv) at room temperature. To the above mixture was added TEA (306.80 mg, 3.032 mmol, 3.00 equiv) for 30 min at room temperature. The resulting mixture was stirred for additional overnight at room temperature. The residue was purified by reverse phase flash chromatography with the following conditions: Column: C18 Column 120 g; Mobile Phase A: Water (10 mM AcOH), Mobile Phase B: ACN; Flow rate: 50 mL/min; Gradient: 55% B to 75% B in 40 min; 254/220 nm. The fractions containing the desired product were collected at 64% B and concentrated under reduced pressure to afford 2-[8-(2-chlorophenyl)-7-(4-chlorophenyl)-2,6-dioxo-3H-purin-1-yl]propanamide (320 mg, 71.27%) as a white solid.

(2R)-2-[8-(2-chlorophenyl)-7-(4-chlorophenyl)-3-(oxan-4-ylmethyl)-2,6-dioxopurin-1-yl]propanamide. To a stirred solution of 2-[8-(2-chlorophenyl)-7-(4-chlorophenyl)-2,6-dioxo-3H-purin-1-yl]propanamide (120.00 mg, 0.270 mmol, 1.00 equiv) and PPh₃ (212.53 mg, 0.810 mmol, 3 equiv) in THF (3.00 mL) were added DEAD (141.12 mg, 0.810 mmol, 3 equiv) and oxan-4-ylmethanol (47.06 mg, 0.405 mmol, 1.5 equiv) dropwise at 0° C. under nitrogen atmosphere. The resulting mixture was stirred for overnight at room temperature under nitrogen atmosphere. The reaction was monitored by LCMS. The resulting mixture was extracted with EtOAc (3×10 mL). The combined organic layers were washed with brine (3×10 mL), dried over anhydrous Na₂SO₄. After filtration, the filtrate was concentrated under reduced pressure. The crude product was purified by Prep-HPLC with the following conditions (Column: XBridge Prep OBD C18 Column, 30×150 mm 5 um; Flow rate: 60 mL/min; Gradient: 30 B to 50 B in 8 min; 220 nm; RT1: 7.19; RT2:; Injection Volume: mL) to afford (2R)-2-[8-(2-chlorophenyl)-7-(4-chlorophenyl)-3-(oxan-4-ylmethyl)-2,6-dioxopurin-1-yl]propanamide (30 mg, 20.48%) and (2S)-2-[8-(2-chlorophenyl)-7-(4-chlorophenyl)-3-(oxan-4-ylmethyl)-2,6-dioxopurin-1-yl]propanamide (35 mg, 23.89%) as white solid.

### Example 22. Preparation of Compounds 137 and 138

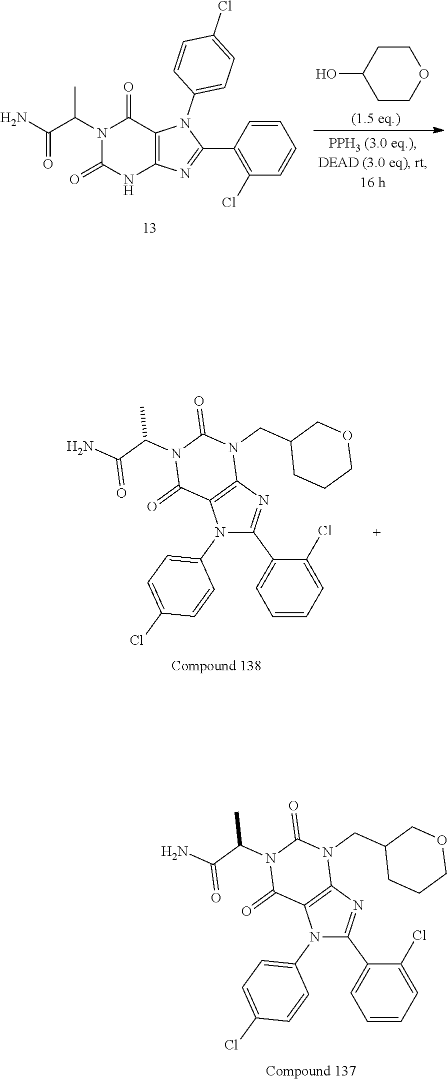 [SMILES: CC(C(N)=O)n1c(=O)[nH]c2nc(-c3ccccc3Cl)n(-c3ccc(Cl)cc3)c2c1=O ; OC1CCOCC1 ; C[C@@H](C(N)=O)n1c(=O)c2c(nc(-c3ccccc3Cl)n2-c2ccc(Cl)cc2)n(CC2CCCOC2)c1=O ; C[C@H](C(N)=O)n1c(=O)c2c(nc(-c3ccccc3Cl)n2-c2ccc(Cl)cc2)n(CC2CCCOC2)c1=O]

(2S)-2-[8-(2-chlorophenyl)-7-(4-chlorophenyl)-3-(oxan-4-yl)-2,6-dioxopurin-1-yl]propanamide and (2R)-2-[8-(2-chlorophenyl)-7-(4-chlorophenyl)-3-(oxan-4-yl)-2,6-dioxopurin-1-yl]propanamide. To a stirred solution of 2-[8-(2-chlorophenyl)-7-(4-chlorophenyl)-2,6-dioxo-3H-purin-1-yl]propanamide (120.00 mg, 0.270 mmol, 1.00 equiv) and tetrahydro-2H-pyran-4-ol (41 mg, 0.41 mmol, 1.5 eq) in THF (3.00 mL) were added DEAD (141.12 mg, 0.810 mmol, 3.00 equiv) and PPh₃ (212.53 mg, 0.810 mmol, 3.00 equiv) dropwise at 0° C. under nitrogen atmosphere. The resulting mixture was stirred for overnight at room temperature under nitrogen atmosphere. The reaction was monitored by LCMS. The resulting mixture was extracted with EtOAc (3×10 mL). The combined organic layers were washed with brine (3×10 mL), dried over anhydrous Na₂SO₄. After filtration, the filtrate was concentrated under reduced pressure. The crude product was purified by Prep-HPLC with the following conditions (Column: XBridge Prep OBD C18 Column, 30×150 mm 5 um; Flow rate: 60 mL/min; Gradient: 30 B to 50 B in 8 min; 220 nm; RT1: 7.19; RT2:; Injection Volume: mL) to afford (2S)-2-[8-(2-chlorophenyl)-7-(4-chlorophenyl)-3-(oxan-4-yl)-2,6-dioxopurin-1-yl]propanamide (15 mg, 10.49%) as a white solid and (2R)-2-[8-(2-chlorophenyl)-7-(4-chlorophenyl)-3-(oxan-4-yl)-2,6-dioxopurin-1-yl]propanamide (15 mg, 10.49%) as a white solid.

### Example 23. Preparation of Compound 132

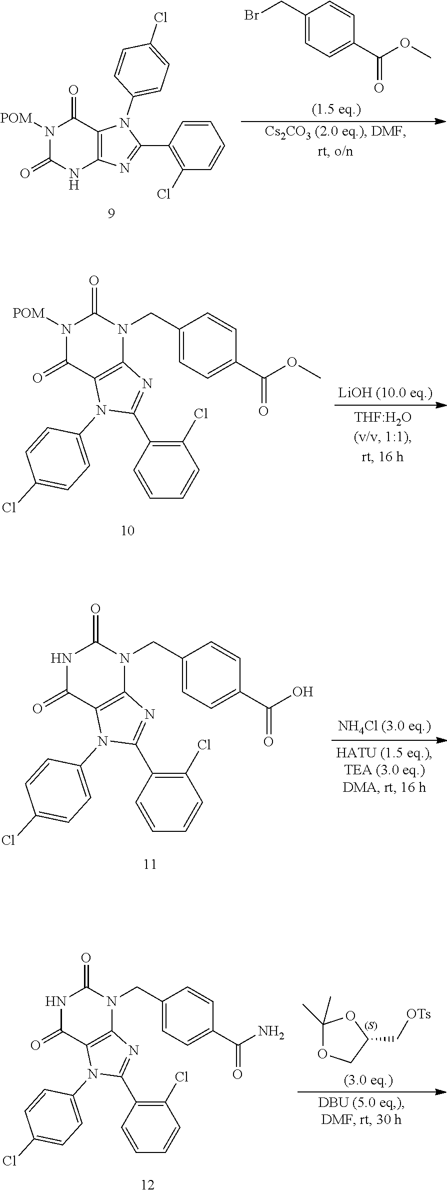

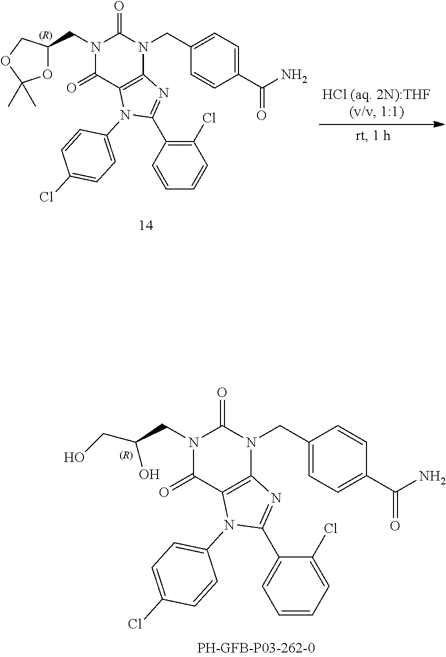

Methyl 4-[[8-(2-chlorophenyl)-7-(4-chlorophenyl)-1-[[(2,2-dimethylpropanoyl)oxy]methyl]-2,6-dioxopurin-3-yl]methyl]benzoate. To a stirred solution/mixture of [8-(2-chlorophenyl)-7-(4-chlorophenyl)-2,6-dioxo-3H-purin-1-yl]methyl 2,2-dimethylpropanoate (300.00 mg, 0.616 mmol, 1.00 equiv) and Cs₂CO₃ (401.14 mg, 1.231 mmol, 2 equiv) in DMF (5.00 mL) was added methyl 4-(bromomethyl)benzoate (211.52 mg, 0.923 mmol, 1.50 equiv) at room temperature. The resulting mixture was stirred for overnight at room temperature. The reaction was monitored by LCMS. The residue was purified by reverse flash chromatography with the following conditions: Column: C18 Column 80 g; Mobile Phase A: Water (10 mM AcOH), Mobile Phase B: ACN; Flow rate: 50 mL/min; Gradient: 60% B to 80% B in 40 min; 254/220 nm. The fractions containing the desired product were collected at 71% B and concentrated under reduced pressure, to afford methyl 4-[[8-(2-chlorophenyl)-7-(4-chlorophenyl)-1-[[(2,2-dimethylpropanoyl)oxy]methyl]-2,6-dioxopurin-3-yl]methyl]benzoate (280 mg, 71.57%) as a white solid.

4-[[8-(2-chlorophenyl)-7-(4-chlorophenyl)-2,6-dioxo-1H-purin-3-yl]methyl]benzoic acid. To a stirred solution of methyl 4-[[8-(2-chlorophenyl)-7-(4-chlorophenyl)-1-[[(2,2-dimethylpropanoyl)oxy]methyl]-2,6-dioxopurin-3-yl]methyl]benzoate (600.00 mg, 1.151 mmol, 1.00 equiv) and LiOH (275.61 mg, 11.509 mmol, 10 equiv) in THF (10.00 mL) was added H₂O (10.00 mL) at room temperature. The resulting mixture was stirred for overnight at room temperature. The reaction was monitored by LCMS. The mixture/residue was acidified to pH 6 with HCl (aq.). The residue was purified by reverse flash chromatography with the following conditions: Column: C18 Column 120 g; Mobile Phase A: Water (10 mM AcOH), Mobile Phase B: ACN; Flow rate: 50 mL/min; Gradient: 50% B to 70% B in 40 min; 254/220 nm. The fractions containing the desired product were collected at 61% B and concentrated under reduced pressure, to afford 4-[[8-(2-chlorophenyl)-7-(4-chlorophenyl)-2,6-dioxo-1H-purin-3-yl]methyl]benzoic acid (400 mg, 82.21%) as a white solid.

4-[[8-(2-chlorophenyl)-7-(4-chlorophenyl)-2,6-dioxo-1H-purin-3-yl]methyl]benzamide. A solution of 4-[[8-(2-chlorophenyl)-7-(4-chlorophenyl)-2,6-dioxo-1H-purin-3-yl]methyl]benzoic acid (500.00 mg, 0.986 mmol, 1.00 equiv) in DMA (10.00 mL) was treated with HATU (562.10 mg, 1.478 mmol, 1.5 equiv) for 30 min at room temperature followed by the addition of NH₄Cl (158.15 mg, 2.957 mmol, 3 equiv) at room temperature. The resulting mixture was stirred for 30 min at room temperature. To the above mixture was added TEA (299.18 mg, 2.957 mmol, 3 equiv) for 30 min at room temperature. The resulting mixture was stirred for additional 6 h at room temperature. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 40% B to 80% B in 40 min; 254/220 nm. The fractions containing the desired product were collected at 53% B and concentrated under reduced pressure, to afford 4-[[8-(2-chlorophenyl)-7-(4-chlorophenyl)-2,6-dioxo-1H-purin-3-yl]methyl]benzamide (350 mg, 70.14%) as a white solid.

4-[[8-(2-chlorophenyl)-7-(4-chlorophenyl)-1-[(2,2-dimethyl-1,3-dioxolan-4-yl)methyl]-2,6-dioxopurin-3-yl]methyl]benzamide. To a stirred solution of 4-[[8-(2-chlorophenyl)-7-(4-chlorophenyl)-2,6-dioxo-1H-purin-3-yl]methyl]benzamide (70.00 mg, 0.138 mmol, 1.00 equiv) and (S)-(2,2-dimethyl-1,3-dioxolan-4-yl)methyl 4-methylbenzenesulfonate (118.76 mg, 0.415 mmol, 3.00 equiv) in DMF (5.00 mL) was added DBU (105.23 mg, 0.691 mmol, 5.00 equiv) at room temperature. The resulting mixture was stirred for 30 h at room temperature. The reaction was monitored by LCMS. The residue was purified by silica gel column chromatography, eluted with CH₂Cl₂/MeOH (20:1) to afford (R)-4-[[8-(2-chlorophenyl)-7-(4-chlorophenyl)-1-[(2,2-dimethyl-1,3-dioxolan-4-yl)methyl]-2,6-dioxopurin-3-yl]methyl]benzamide (crude) as a light yellow oil.

4-[[8-(2-chlorophenyl)-7-(4-chlorophenyl)-1-[(2R)-2,3-dihydroxypropyl]-2,6-dioxopurin-3-yl]methyl]benzamide. To a stirred solution of 4-[[8-(2-chlorophenyl)-7-(4-chlorophenyl)-1-[[(4R)-2,2-dimethyl-1,3-dioxolan-4-yl]methyl]-2,6-dioxopurin-3-yl]methyl]benzamide (70.00 mg, 0.113 mmol, 1.00 equiv) and HCl (2M) (4.00 mL) in THF (4.00 mL) at room temperature. The resulting mixture was stirred for 1 h at room temperature. The reaction was monitored by LCMS. The mixture was acidified to pH 7 with saturated NaHCO₃ (aq.). The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC with the following conditions (Column: XBridge Shield RP18 OBD Column, 30*150 mm, 5 um; Mobile Phase A: Water (10 mM NH₄HCO₃), Mobile Phase B: ACN; Flow rate: 60 mL/min; Gradient: 25 B to 42 B in 7 min; 254/220 nm; RT1: 6.5) to afford 4-[[8-(2-chlorophenyl)-7-(4-chlorophenyl)-1-[(2R)-2,3-dihydroxypropyl]-2,6-dioxopurin-3-yl]methyl]benzamide (35 mg, 53.45%) as a white solid.

### Example 24. Preparation of Compound 135

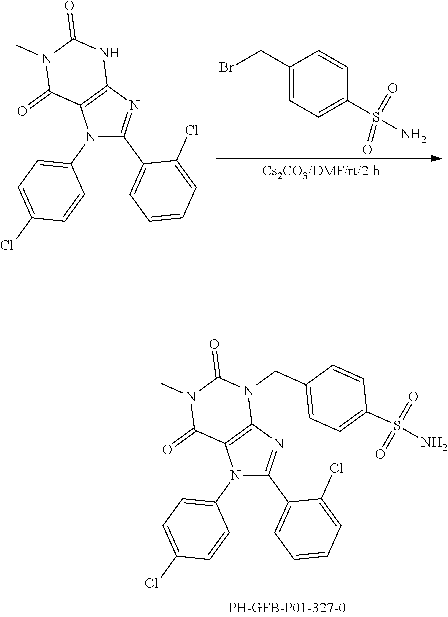 [SMILES: Cn1c(=O)[nH]c2nc(-c3ccccc3Cl)n(-c3ccc(Cl)cc3)c2c1=O ; NS(=O)(=O)c1ccc(CBr)cc1 ; Cn1c(=O)c2c(nc(-c3ccccc3Cl)n2-c2ccc(Cl)cc2)n(Cc2ccc(S(N)(=O)=O)cc2)c1=O]

To a stirred solution of 8-(2-chlorophenyl)-7-(4-chlorophenyl)-1-methyl-3H-purine-2,6-dione (100.00 mg, 0.258 mmol, 1.00 equiv) and Cs₂CO₃ (168.29 mg, 0.517 mmol, 2.00 equiv) in DMF (5.00 mL) was added 4-(bromomethyl)benzenesulfonamide (96.89 mg, 0.387 mmol, 1.50 equiv) at room temperature. The resulting mixture was stirred for overnight at room temperature. The reaction was monitored by LCMS. The residue was purified by reverse flash chromatography with the following conditions: Column: C18 Column 120 g; Mobile Phase A: Water (10 mM AcOH), Mobile Phase B: ACN; Flow rate: 50 mL/min; Gradient: 50% B to 70% B in 40 min; 254/220 nm. The fractions containing the desired product were collected at 66% B and concentrated under reduced pressure. The crude product was purified by Prep-HPLC with the following conditions (Column: XBridge Prep OBD C18 Column, 30×150 mm 5 um; Flow rate: 60 mL/min; Gradient: 40% B to 55% B in 11 min; 220 nm; RT1: 9.68; RT2:; Injection Volume: mL) to afford 4-[[8-(2-chlorophenyl)-7-(4-chlorophenyl)-1-methyl-2,6-dioxopurin-3-yl]methyl]benzenesulfonamide (45 mg, 31.32%) as an off-white solid.

### Example 25. Preparation of Compounds 150 and 151

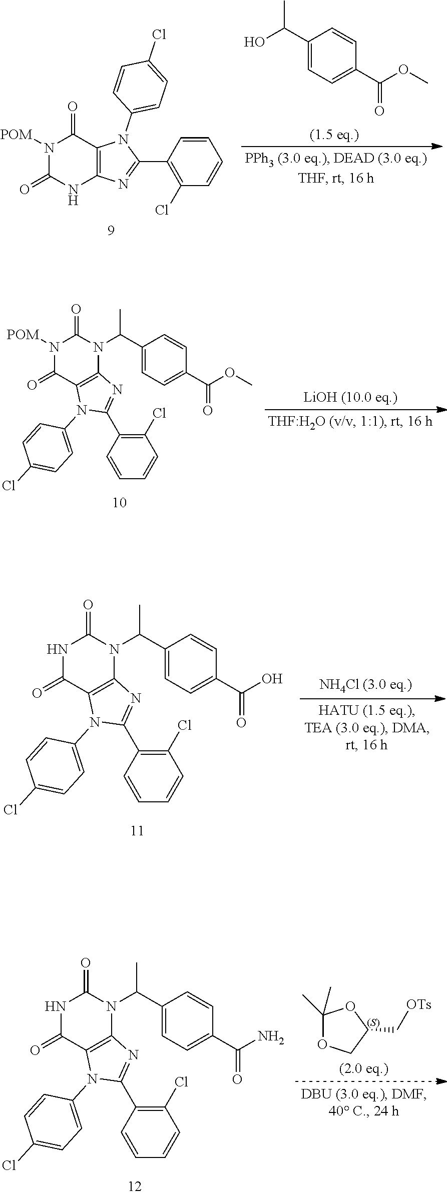

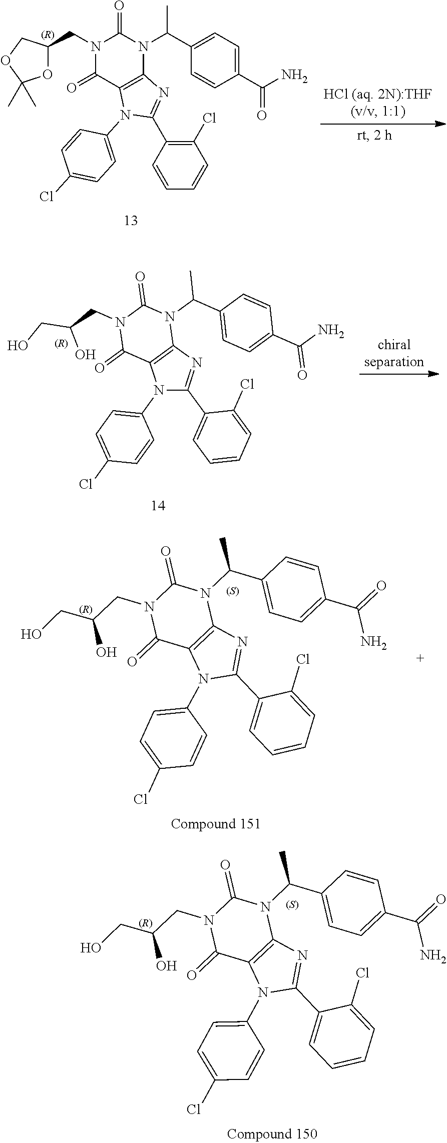

Methyl 4-[1-[8-(2-chlorophenyl)-7-(4-chlorophenyl)-1-[[(2,2-dimethylpropanoyl)oxy]methyl]-2,6-dioxopurin-3-yl]ethyl]benzoate. To a stirred solution of [8-(2-chlorophenyl)-7-(4-chlorophenyl)-2,6-dioxo-3H-purin-1-yl]methyl 2,2-dimethylpropanoate (900.00 mg, 1.847 mmol, 1.00 equiv) and PPh₃ (1453.14 mg, 5.540 mmol, 3.00 equiv) in THF (10.00 mL) were added DEAD (964.87 mg, 5.540 mmol, 3.00 equiv) and methyl 4-(1-hydroxyethyl)benzoate (499.19 mg, 2.770 mmol, 1.50 equiv) at 0° C. under nitrogen atmosphere. The resulting mixture was stirred for 16 h at room temperature under nitrogen atmosphere. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 5% B to 50% B in 40 min; 254/220 nm. The fractions containing the desired product were collected at 40% B and concentrated under reduced pressure. This resulted in methyl 4-[1-[8-(2-chlorophenyl)-7-(4-chlorophenyl)-1-[[(2,2-dimethylpropanoyl)oxy]methyl]-2,6-dioxopurin-3-yl]ethyl]benzoate (600 mg, 50%) as an off-white solid.

4-[1-[8-(2-chlorophenyl)-7-(4-chlorophenyl)-2,6-dioxo-1H-purin-3-yl]ethyl]benzoic acid. To a stirred solution of methyl 4-[1-[8-(2-chlorophenyl)-7-(4-chlorophenyl)-1-[[(2,2-dimethylpropanoyl)oxy]methyl]-2,6-dioxopurin-3-yl]ethyl]benzoate (600.00 mg, 0.924 mmol, 1.00 equiv) and LiOH (221.22 mg, 9.237 mmol, 10.00 equiv) in THF (5.00 mL) was added H₂O (5.00 mL) at room temperature. The resulting mixture was stirred for 16 h at room temperature.

The reaction was monitored by LCMS. The mixture was acidified to pH 6 with HCl (aq.). The residue was purified by reverse flash chromatography with the following conditions: Column: C18 Column 120 g; Mobile Phase A: Water (10 mM AcOH), Mobile Phase B: ACN; Flow rate: 50 mL/min; Gradient: 45% B to 70% B in 40 min; 254/220 nm. The fractions containing the desired product were collected at 58% B and concentrated under reduced pressure. to afford 4-[1-[8-(2-chlorophenyl)-7-(4-chlorophenyl)-2,6-dioxo-1H-purin-3-yl]ethyl]benzoic acid (330 mg, 68.52%) as a white solid.

4-[1-[8-(2-chlorophenyl)-7-(4-chlorophenyl)-2,6-dioxo-1H-purin-3-yl]ethyl]benzamide. To a stirred solution of 4-[1-[8-(2-chlorophenyl)-7-(4-chlorophenyl)-2,6-dioxo-1H-purin-3-yl]ethyl]benzoic acid (330.00 mg, 0.633 mmol, 1.00 equiv) in DMA (6.00 mL) was added HATU (361.01 mg, 0.949 mmol, 1.50 equiv) at room temperature. The resulting mixture was stirred for 30 min at room temperature. To the above mixture was added NH₄Cl (101.57 mg, 1.899 mmol, 3.00 equiv) at room temperature. The resulting mixture was stirred for 30 min at room temperature. To the above mixture was added TEA (192.15 mg, 1.899 mmol, 3.00 equiv) at room temperature. The resulting mixture was stirred for 16 h at room temperature. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 50% B to 80% B in 40 min; 254/220 nm. The fractions containing the desired product were collected at 63% B and concentrated under reduced pressure, to afford 4-[1-[8-(2-chlorophenyl)-7-(4-chlorophenyl)-2,6-dioxo-1H-purin-3-yl]ethyl]benzamide (150 mg, 45.54%) as a white solid.

4-[1-[8-(2-chlorophenyl)-7-(4-chlorophenyl)-1-[[(4R)-2,2-dimethyl-1,3-dioxolan-4-yl]methyl]-2,6-dioxopurin-3-yl]ethyl]benzamide. To a stirred solution of 4-[1-[8-(2-chlorophenyl)-7-(4-chlorophenyl)-2,6-dioxo-1H-purin-3-yl]ethyl]benzamide (150.00 mg, 0.288 mmol, 1.00 equiv) and DBU (131.65 mg, 0.865 mmol, 3.00 equiv) in DMF (5.00 mL) was added [(4S)-2,2-dimethyl-1,3-dioxolan-4-yl]methyl 4-methylbenzenesulfonate (165.08 mg, 0.577 mmol, 2.00 equiv) at room temperature. The resulting mixture was stirred for 24 h at 40° C. The reaction was monitored by LCMS. The residue was purified by silica gel column chromatography, eluted with PE/EtOAc (1:1) to afford 4-[1-[8-(2-chlorophenyl)-7-(4-chlorophenyl)-1-[[(4R)-2,2-dimethyl-1,3-dioxolan-4-yl]methyl]-2,6-dioxopurin-3-yl]ethyl]benzamide (120 mg, 65.61%) as a white solid.

4-[(1S)-1-[8-(2-chlorophenyl)-7-(4-chlorophenyl)-1-[(2R)-2,3-dihydroxypropyl]-2,6-dioxopurin-3-yl]ethyl]benzamide and 4-[(1R)-1-[8-(2-chlorophenyl)-7-(4-chlorophenyl)-1-[(2R)-2,3-dihydroxypropyl]-2,6-dioxopurin-3-yl]ethyl]benzamide. To a stirred solution of 4-[1-[8-(2-chlorophenyl)-7-(4-chlorophenyl)-1-[[(4R)-2,2-dimethyl-1,3-dioxolan-4-yl]methyl]-2,6-dioxopurin-3-yl]ethyl]benzamide (70.00 mg, 1 equiv) and HCl (2M) (3.00 mL) in THF (3.00 mL) at room temperature. The resulting mixture was stirred for 2 h at room temperature. The reaction was monitored by LCMS. The mixture was basified to pH 7 with saturated NaHCO₃ (aq.). The resulting mixture was extracted with EtOAc (3×10 mL). The combined organic layers were washed with brine (3×10 mL), dried over anhydrous Na₂SO₄. After filtration, the filtrate was concentrated under reduced pressure. The crude product was purified by Prep-HPLC with the following conditions (Column: CHIRALPAK IA, 2*25 cm, 5 um; Mobile Phase A: MTBE (10 mM NH₃-MeOH)—HPLC, Mobile Phase B: EtOH—HPLC; Flow rate: 14 mL/min; Gradient: 30 B to 30 B in 12 min; 220/254 nm; RT1: 7.012; RT2: 9.03; Injection Volume: 0.5 mL; Number Of Runs: 7) to afford 4-[(1S)-1-[8-(2-chlorophenyl)-7-(4-chlorophenyl)-1-[(2R)-2,3-dihydroxypropyl]-2,6-dioxopurin-3-yl]ethyl]benzamide (15 mg, 22.87%) and 4-[(1R)-1-[8-(2-chlorophenyl)-7-(4-chlorophenyl)-1-[(2R)-2,3-dihydroxypropyl]-2,6-dioxopurin-3-yl]ethyl]benzamide (15 mg, 22.87%) as white solid.

### Example 26. Preparation of Compound 152

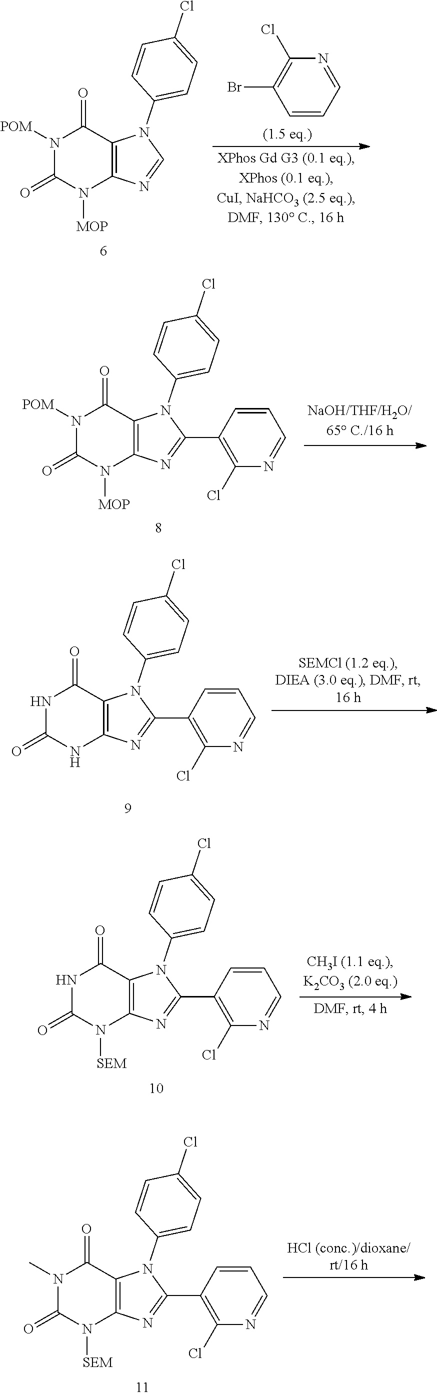

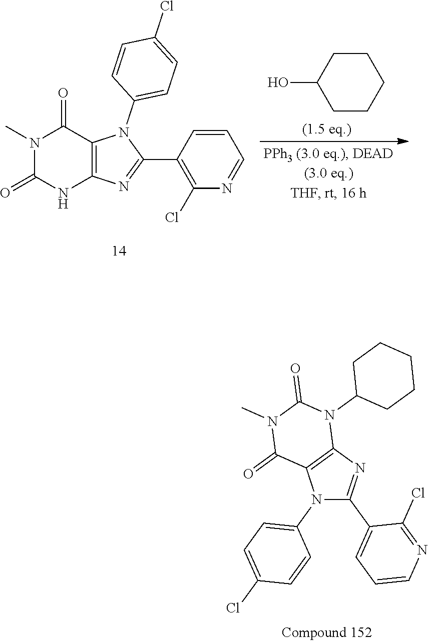 [SMILES: Cn1c(=O)[nH]c2nc(-c3cccnc3Cl)n(-c3ccc(Cl)cc3)c2c1=O ; OC1CCCCC1 ; Cn1c(=O)c2c(nc(-c3cccnc3Cl)n2-c2ccc(Cl)cc2)n(C2CCCCC2)c1=O]

[7-(4-chlorophenyl)-8-(2-chloropyridin-3-yl)-3-[[(2,2-dimethylpropanoyl)oxy]methyl]-2,6-dioxopurin-1-yl]methyl 2,2-dimethylpropanoate. To a stirred solution of [7-(4-chlorophenyl)-3-[[(2,2-dimethylpropanoyl)oxy]methyl]-2,6-dioxopurin-1-yl]methyl 2,2-dimethylpropanoate (1.70 g, 3.463 mmol, 1.00 equiv) and 3-bromo-2-chloropyridine (999.56 mg, 5.194 mmol, 1.50 equiv) in DMF (20.00 mL, 258.435 mmol) were added NaHCO₃ (727.23 mg, 8.657 mmol, 2.50 equiv), XPhos (165.07 mg, 0.346 mmol, 0.10 equiv), XPhos Pd G3 (293.10 mg, 0.346 mmol, 0.10 equiv) and CuI (1.32 g, 6.925 mmol, 2.00 equiv) at room temperature under nitrogen atmosphere. The resulting mixture was stirred for 16 h at 130° C. under nitrogen atmosphere in sealed tube. The reaction was monitored by LCMS. The mixture was allowed to cool down to room temperature. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 70% B to 90% B in 40 min; 254/220 nm). The fractions containing the desired product were collected at 85% B and concentrated under reduced pressure to afford [7-(4-chlorophenyl)-8-(2-chloropyridin-3-yl)-3-[[(2,2-dimethylpropanoyl)oxy]methyl]-2,6-dioxopurin-1-yl]methyl 2,2-dimethylpropanoate (1.1 g, 52.73%) as a white solid

7-(4-chlorophenyl)-8-(2-chloropyridin-3-yl)-1,3-dihydropurine-2,6-dione. To a stirred solution of [7-(4-chlorophenyl)-8-(2-chloropyridin-3-yl)-3-[[(2,2-dimethylpropanoyl)oxy]methyl]-2,6-dioxopurin-1-yl]methyl 2,2-dimethylpropanoate (1.10 g, 1.826 mmol, 1.00 equiv) and NaOH (219.08 mg, 5.477 mmol, 3.00 equiv) in THF (20.00 mL) was added H₂O (20.00 mL) at room temperature. The resulting mixture was stirred for overnight at 65° C. The reaction was monitored by LCMS. The mixture was allowed to cool down to room temperature. The mixture was acidified to pH 6 with HCl (aq.). The residue was purified by reverse flash chromatography with the following conditions: Column: C18 Column 330 g; Mobile Phase A: Water (10 mM NH₄HCO₃), Mobile Phase B: ACN; Flow rate: 50 mL/min; Gradient: 25% B to 50% B in 40 min; 254/220 nm. The fractions containing the desired product were collected at 45% B and concentrated under reduced pressure. to afford 7-(4-chlorophenyl)-8-(2-chloropyridin-3-yl)-1,3-dihydropurine-2,6-dione (160 mg, 23.42%) as a white solid.

7-(4-chlorophenyl)-8-(2-chloropyridin-3-yl)-3-[[2-(trimethylsilyl)ethoxy]methyl]-1H-purine-2,6-dione. To a stirred solution of 7-(4-chlorophenyl)-8-(2-chloropyridin-3-yl)-1,3-dihydropurine-2,6-dione (160.00 mg, 0.428 mmol, 1.00 equiv) and [2-(chloromethoxy)ethyl]trimethylsilane (85.55 mg, 0.513 mmol, 1.20 equiv) in DMF (6.00 mL) was added DIEA (165.79 mg, 1.283 mmol, 3.00 equiv) at room temperature. The resulting mixture was stirred for overnight at room temperature. The reaction was monitored by LCMS. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 45% B to 75% B in 40 min; 254/220 nm. The fractions containing the desired product were collected at 63% B and concentrated under reduced pressure, to afford 7-(4-chlorophenyl)-8-(2-chloropyridin-3-yl)-3-[[2-(trimethylsilyl)ethoxy]methyl]-1H-purine-2,6-dione (165 mg, 76.50%) as a light yellow solid.

7-(4-chlorophenyl)-8-(2-chloropyridin-3-yl)-1-methyl-3-[[2-(trimethylsilyl)ethoxy]methyl]purine-2,6-dione. To a stirred solution of 7-(4-chlorophenyl)-8-(2-chloropyridin-3-yl)-3-[[2-(trimethylsilyl)ethoxy]methyl]-1H-purine-2,6-dione (165.00 mg, 0.327 mmol, 1.00 equiv) and K₂CO₃ (90.41 mg, 0.654 mmol, 2.00 equiv) in DMF (5.00 mL) was added CH₃I (51.07 mg, 0.360 mmol, 1.10 equiv) at room temperature. The resulting mixture was stirred for 4 h at room temperature. The reaction was monitored by LCMS. The resulting mixture was extracted with EtOAc (3×50 mL). The combined organic layers were washed with brine (3×50 mL), dried over anhydrous Na₂SO₄. After filtration, the filtrate was concentrated under reduced pressure. The crude resulting mixture was used in the next step directly without further purification.

7-(4-chlorophenyl)-8-(2-chloropyridin-3-yl)-1-methyl-3H-purine-2,6-dione. To a stirred solution of 7-(4-chlorophenyl)-8-(2-chloropyridin-3-yl)-1-methyl-3-[[2-(trimethylsilyl)ethoxy]methyl]purine-2,6-dione (150.00 mg, 0.289 mmol, 1.00 equiv) in dioxane (3.00 mL) was added HCl (12M) (6.00 mL) at room temperature. The resulting mixture was stirred for overnight at room temperature. The reaction was monitored by LCMS. The mixture was basified to pH 7 with saturated NaHCO₃ (aq.). The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 35% B to 55% B in 40 min; 254/220 nm. The fractions containing the desired product were collected at 47% B and concentrated under reduced pressure to afford 7-(4-chlorophenyl)-8-(2-chloropyridin-3-yl)-1-methyl-3H-purine-2,6-dione (100 mg, 89.04%) as a white solid.

7-(4-chlorophenyl)-8-(2-chloropyridin-3-yl)-3-cyclohexyl-1-methylpurine-2,6-dione. To a stirred solution of 7-(4-chlorophenyl)-8-(2-chloropyridin-3-yl)-1-methyl-3H-purine-2,6-dione (100.00 mg, 0.258 mmol, 1.00 equiv) and PPh₃ (0.20 g, 0.773 mmol, 3.00 equiv) in THF (6.00 mL) were added DEAD (134.58 mg, 0.773 mmol, 3.00 equiv) and cyclohexanol (38.70 mg, 0.386 mmol, 1.50 equiv) at 0° C. under nitrogen atmosphere. The resulting mixture was stirred for overnight at room temperature under nitrogen atmosphere. The reaction was monitored by LCMS. The resulting mixture was extracted with EtOAc (3×30 mL). The combined organic layers were washed with brine (3×30 mL), dried over anhydrous Na₂SO₄. After filtration, the filtrate was concentrated under reduced pressure. The crude product was purified by Prep-HPLC with the following conditions (Column: Sunfire Prep C18 OBD Column, 10 um, 19*250 mm; Mobile Phase A: undefined, Mobile Phase B: undefined; Flow rate: 25 mL/min; Gradient: 70 B to 95 B in 7 min; 254 nm; RT1: 5.9; RT2:; Injection Volume: mL) to afford 7-(4-chlorophenyl)-8-(2-chloropyridin-3-yl)-3-cyclohexyl-1-methylpurine-2,6-dione (40 mg, 33.01%) as a white solid.

### Example 27. Preparation of Compounds 141 and 145

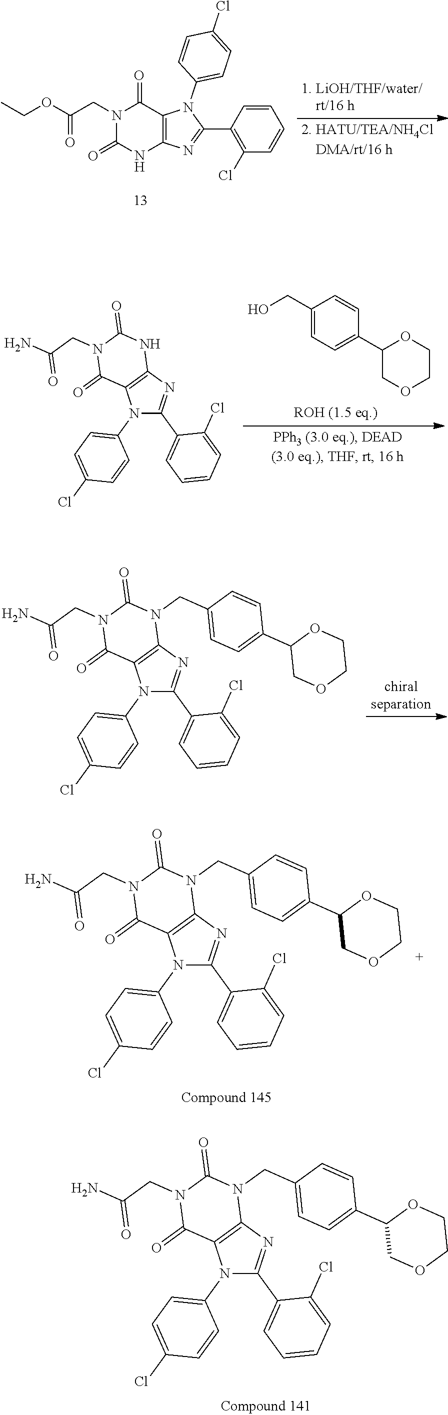 [SMILES: CCOC(=O)Cn1c(=O)[nH]c2nc(-c3ccccc3Cl)n(-c3ccc(Cl)cc3)c2c1=O ; NC(=O)Cn1c(=O)[nH]c2nc(-c3ccccc3Cl)n(-c3ccc(Cl)cc3)c2c1=O ; OCc1ccc(C2COCCO2)cc1 ; NC(=O)Cn1c(=O)c2c(nc(-c3ccccc3Cl)n2-c2ccc(Cl)cc2)n(Cc2ccc(C3COCCO3)cc2)c1=O ; NC(=O)Cn1c(=O)c2c(nc(-c3ccccc3Cl)n2-c2ccc(Cl)cc2)n(Cc2ccc([C@@H]3COCCO3)cc2)c1=O ; NC(=O)Cn1c(=O)c2c(nc(-c3ccccc3Cl)n2-c2ccc(Cl)cc2)n(Cc2ccc([C@H]3COCCO3)cc2)c1=O]

[8-(2-chlorophenyl)-7-(4-chlorophenyl)-2,6-dioxo-3H-purin-1-yl]acetic acid. To a stirred solution of ethyl 2-[8-(2-chlorophenyl)-7-(4-chlorophenyl)-2,6-dioxo-3H-purin-1-yl]acetate (300.00 mg, 0.653 mmol, 1.00 equiv) and LiOH (156.43 mg, 6.532 mmol, 10.00 equiv) in THF (3.00 mL) was added H₂O (3.00 mL) at room temperature. The resulting mixture was stirred for 16 h at room temperature. The reaction was monitored by LCMS. The mixture was acidified to pH 6 with HCl (aq.). The residue was purified by reverse flash chromatography with the following conditions: Column: C18 Column 120 g; Mobile Phase A: Water (10 mM AcOH), Mobile Phase B: ACN; Flow rate: 50 mL/min; Gradient: 45% B to 65% B in 40 min; 254/220 nm. The fractions containing the desired product were collected at 53% B and concentrated under reduced pressure, to afford [8-(2-chlorophenyl)-7-(4-chlorophenyl)-2,6-dioxo-3H-purin-1-yl]acetic acid (200 mg, 71.00%) as a white solid.

2-[8-(2-chlorophenyl)-7-(4-chlorophenyl)-2,6-dioxo-3H-purin-1-yl]acetamide. To a stirred solution of [8-(2-chlorophenyl)-7-(4-chlorophenyl)-2,6-dioxo-3H-purin-1-yl]acetic acid (200.00 mg, 0.464 mmol, 1.00 equiv) in DMA (4.00 mL) was added HATU (264.52 mg, 0.696 mmol, 1.50 equiv) at room temperature. The resulting mixture was stirred for 30 min at room temperature. To the above mixture was added NH₄Cl (74.43 mg, 1.391 mmol, 3.00 equiv) at room temperature. The resulting mixture was stirred for 30 min at room temperature. To the above mixture was added TEA (140.79 mg, 1.391 mmol, 3.00 equiv) at room temperature. The resulting mixture was stirred for 16 h at room temperature. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 40% B to 70% B in 40 min; 254/220 nm. The fractions containing the desired product were collected at 59% B and concentrated under reduced pressure, to afford 2-[8-(2-chlorophenyl)-7-(4-chlorophenyl)-2,6-dioxo-3H-purin-1-yl]acetamide (150 mg, 75.17%) as an off-white solid.

2-[8-(2-chlorophenyl)-7-(4-chlorophenyl)-3-([4-[(2S)-1,4-dioxan-2-yl]phenyl]methyl)-2,6-dioxopurin-1-yl]acetamide (20 mg, 10.91%) and 2-[8-(2-chlorophenyl)-7-(4-chlorophenyl)-3-([4-[(2R)-1,4-dioxan-2-yl]phenyl]methyl)-2,6-dioxopurin-1-yl]acetamide. To a stirred solution of 2-[8-(2-chlorophenyl)-7-(4-chlorophenyl)-2,6-dioxo-3H-purin-1-yl]acetamide (130.00 mg, 0.302 mmol, 1.00 equiv) and PPh₃ (237.75 mg, 0.906 mmol, 3.00 equiv) in THF (3.00 mg) were added DEAD (157.86 mg, 0.906 mmol, 3.00 equiv) and [4-(1,4-dioxan-2-yl)phenyl]methanol (88.03 mg, 0.453 mmol, 1.50 equiv) dropwise at 0° C. under nitrogen atmosphere. The resulting mixture was stirred for overnight at room temperature under nitrogen atmosphere. The reaction was monitored by LCMS. The crude product was purified by Prep-HPLC to afford 2-[8-(2-chlorophenyl)-7-(4-chlorophenyl)-3-([4-[(2S)-1,4-dioxan-2-yl]phenyl]methyl)-2,6-dioxopurin-1-yl]acetamide (20 mg, 10.91%) as a light yellow solid and 2-[8-(2-chlorophenyl)-7-(4-chlorophenyl)-3-([4-[(2R)-1,4-dioxan-2-yl]phenyl]methyl)-2,6-dioxopurin-1-yl]acetamide (20 mg, 10.91%) as a light yellow solid.

### Example 28. Preparation of Compounds 146 and 148

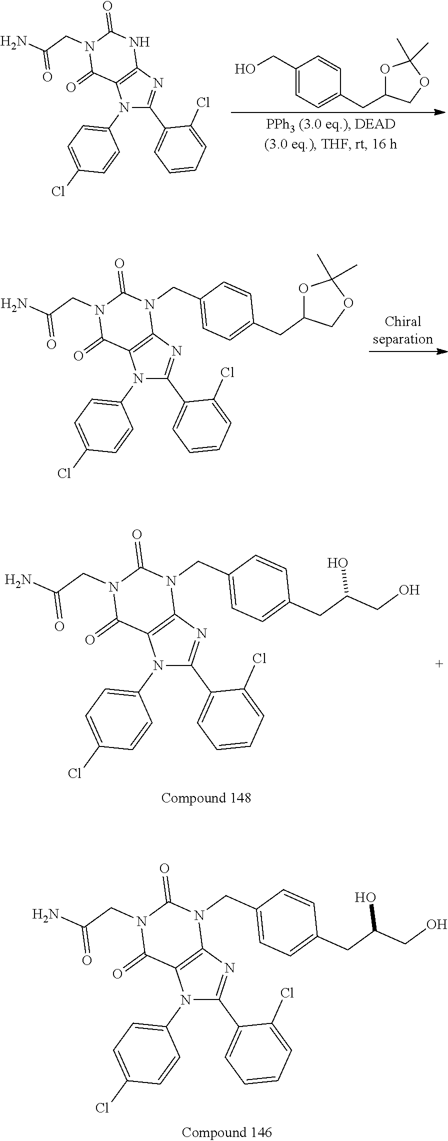 [SMILES: NC(=O)Cn1c(=O)[nH]c2nc(-c3ccccc3Cl)n(-c3ccc(Cl)cc3)c2c1=O ; CC1(C)OCC(Cc2ccc(CO)cc2)O1 ; CC1(C)OCC(Cc2ccc(Cn3c(=O)n(CC(N)=O)c(=O)c4c3nc(-c3ccccc3Cl)n4-c3ccc(Cl)cc3)cc2)O1 ; NC(=O)Cn1c(=O)c2c(nc(-c3ccccc3Cl)n2-c2ccc(Cl)cc2)n(Cc2ccc(C[C@H](O)CO)cc2)c1=O ; NC(=O)Cn1c(=O)c2c(nc(-c3ccccc3Cl)n2-c2ccc(Cl)cc2)n(Cc2ccc(C[C@@H](O)CO)cc2)c1=O]

2-[8-(2-chlorophenyl)-7-(4-chlorophenyl)-3-([4-[(2,2-dimethyl-1,3-dioxolan-4-yl)methyl]phenyl]methyl)-2,6-dioxopurin-1-yl]acetamide. To a stirred solution of 2-[8-(2-chlorophenyl)-7-(4-chlorophenyl)-2,6-dioxo-3H-purin-1-yl]acetamide (150.00 mg, 0.349 mmol, 1.00 equiv) and PPh₃ (274.33 mg, 1.046 mmol, 3.00 equiv) in THF (5.00 mL) were added DEAD (182.15 mg, 1.046 mmol, 3.00 equiv) and [4-[(2,2-dimethyl-1,3-dioxolan-4-yl)methyl]phenyl]methanol (116.24 mg, 0.523 mmol, 1.50 equiv) at 0° C. under nitrogen atmosphere. The resulting mixture was stirred for overnight at room temperature under nitrogen atmosphere. The reaction was monitored by LCMS. The resulting mixture was extracted with EtOAc (3×10 mL). The combined organic layers were washed with brine (3×20 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 5% B to 50% B in 40 min; 254/220 nm. The fractions containing the desired product were collected at 40% B and concentrated under reduced pressure. To afford 2-[8-(2-chlorophenyl)-7-(4-chlorophenyl)-3-([4-[(2,2-dimethyl-1,3-dioxolan-4-yl)methyl]phenyl]methyl)-2,6-dioxopurin-1-yl]acetamide (180 mg, 81.37%) as a white solid.

2-[8-(2-chlorophenyl)-7-(4-chlorophenyl)-3-([4-[(2S)-2,3-dihydroxypropyl]phenyl]methyl)-2,6-dioxopurin-1-yl]acetamide and 2-[8-(2-chlorophenyl)-7-(4-chlorophenyl)-3-([4-[(2R)-2,3-dihydroxypropyl]phenyl]methyl)-2,6-dioxopurin-1-yl]acetamide. To a stirred solution of 2-[8-(2-chlorophenyl)-7-(4-chlorophenyl)-3-([4-[(2,2-dimethyl-1,3-dioxolan-4-yl)methyl]phenyl]methyl)-2,6-dioxopurin-1-yl]acetamide (180.00 mg, 0.284 mmol, 1.00 equiv) and HCl (2M) (4.00 mL) in THF (4.00 mL) at room temperature. The resulting mixture was stirred for 1 h at room temperature. The reaction was monitored by LCMS. The mixture was basified to pH 7 with saturated NaHCO₃ (aq.). The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC with the following conditions (Column: CHIRALPAK IC, 2*25 cm, 5 um; Mobile Phase A: Hex:DCM=3:1 (10 mM NH₃-MEOH)—HPLC, Mobile Phase B: EtOH:DCM=1:1-HPLC; Flow rate: 20 mL/min; Gradient: 40 B to 40 B in 19 min; 220/254 nm; RT1: 13.46; RT2: 16.075; Injection Volume: 0.4 mL; Number Of Runs: 7) to afford 2-[8-(2-chlorophenyl)-7-(4-chlorophenyl)-3-([4-[(2S)-2,3-dihydroxypropyl]phenyl]methyl)-2,6-dioxopurin-1-yl]acetamide (40 mg, 23.72%) and 2-[8-(2-chlorophenyl)-7-(4-chlorophenyl)-3-([4-[(2R)-2,3-dihydroxypropyl]phenyl]methyl)-2,6-dioxopurin-1-yl]acetamide (40 mg, 23.72%) as a white solid.

Compounds 133 and 143 were prepared by the methods and scheme described for Compounds 146 and 148 above using the appropriate reagents.

### Example 29. Preparation of Compound 129

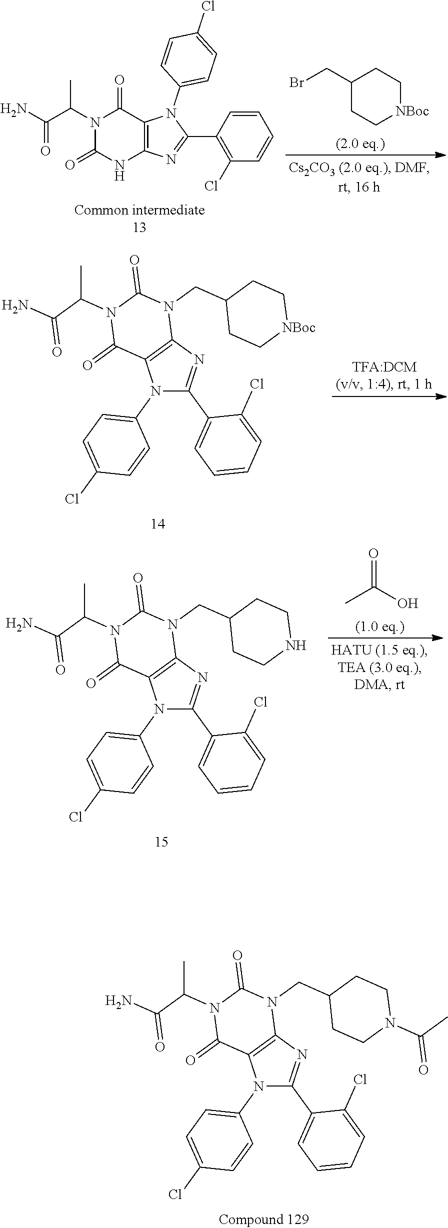

tert-butyl 4-[[1-(1-carbamoylethyl)-8-(2-chlorophenyl)-7-(4-chlorophenyl)-2,6-dioxopurin-3-yl]methyl]piperidine-1-carboxylate. To a stirred solution of 2-[8-(2-chlorophenyl)-7-(4-chlorophenyl)-2,6-dioxo-3H-purin-1-yl]propanamide (80.00 mg, 0.180 mmol, 1.00 equiv) and Cs₂CO₃ (117.34 mg, 0.360 mmol, 2.00 equiv) in DMF (5.00 mL) were added tert-butyl 4-(bromomethyl)piperidine-1-carboxylate (100.19 mg, 0.360 mmol, 2.00 equiv) at room temperature. The resulting mixture was stirred for 16 h at room temperature. The reaction was monitored by LCMS. The residue was purified by reverse flash chromatography with the following conditions: Column: C18 Column 120 g; Mobile Phase A: Water (10 mM FA), Mobile Phase B: ACN; Flow rate: 50 mL/min; Gradient: 65% B to 85% B in 40 min; 254/220 nm. The fractions containing the desired product were collected at 73% B and concentrated under reduced pressure. To afford tert-butyl 4-[[1-(1-carbamoylethyl)-8-(2-chlorophenyl)-7-(4-chlorophenyl)-2,6-dioxopurin-3-yl]methyl]piperidine-1-carboxylate (60 mg, 51.94%) as a light yellow oil.

2-[8-(2-chlorophenyl)-7-(4-chlorophenyl)-2,6-dioxo-3-(piperidin-4-ylmethyl)purin-1-yl]propanamide. To a stirred solution of tert-butyl 4-[[1-(1-carbamoylethyl)-8-(2-chlorophenyl)-7-(4-chlorophenyl)-2,6-dioxopurin-3-yl]methyl]piperidine-1-carboxylate (60.00 mg, 1 equiv) in DCM (4.00 mL) was added TFA (1.00 mL) at room temperature. The resulting mixture was stirred for 1 h at room temperature. The reaction was monitored by LCMS. The mixture was acidified to pH 6 with saturated NaHCO₃ (aq.). The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 60 mL/min; Gradient: 60% B to 80% B in 40 min; 254/220 nm. The fractions containing the desired product were collected at 71% B and concentrated under reduced pressure to afford 2-[8-(2-chlorophenyl)-7-(4-chlorophenyl)-2,6-dioxo-3-(piperidin-4-ylmethyl)purin-1-yl]propanamide (40 mg, 78.99%) as a colorless oil.

2-[3-[(1-acetylpiperidin-4-yl)methyl]-8-(2-chlorophenyl)-7-(4-chlorophenyl)-2,6-dioxopurin-1-yl]propanamide. To a stirred solution of acetic acid (4.7 mg, 0.078 mmol, 1.0 equiv) in DMA (2.00 mL) was added HATU (42 mg, 0.11 mmol, 1.5 equiv) at room temperature. To the above mixture was added 2-[8-(2-chlorophenyl)-7-(4-chlorophenyl)-2,6-dioxo-3-(piperidin-4-ylmethyl)purin-1-yl]propanamide (40.00 mg, 0.074 mmol, 1.00 equiv) at room temperature. The resulting mixture was stirred for 30 min at room temperature. To the above mixture was added TEA (22 mg, 0.22 mmol, 3 equiv) at room temperature. The resulting mixture was stirred for 16 h at room temperature. The reaction was monitored by LCMS. The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC with the following conditions (Column: XBridge Shield RP18 OBD Column, 5 um, 19*150 mm; Flow rate: 25 mL/min; Gradient: 37 B to 42 B in 7 min; 220/254 nm; RT1: 6.5) to afford 2-[3-[(1-acetylpiperidin-4-yl)methyl]-8-(2-chlorophenyl)-7-(4-chlorophenyl)-2,6-dioxopurin-1-yl]propanamide (15 mg, 34.80%) as a white solid.

### Example 30. Preparation of Compound 136

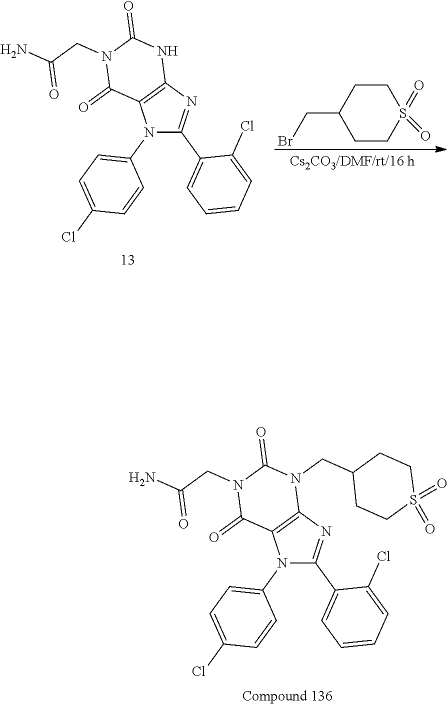 [SMILES: NC(=O)Cn1c(=O)[nH]c2nc(-c3ccccc3Cl)n(-c3ccc(Cl)cc3)c2c1=O ; O=S1(=O)CCC(CBr)CC1 ; NC(=O)Cn1c(=O)c2c(nc(-c3ccccc3Cl)n2-c2ccc(Cl)cc2)n(CC2CCS(=O)(=O)CC2)c1=O]

To a stirred solution of 2-[8-(2-chlorophenyl)-7-(4-chlorophenyl)-2,6-dioxo-3H-purin-1-yl]acetamide (30.00 mg, 0.070 mmol, 1.00 equiv) and Cs₂CO₃ (45.44 mg, 0.139 mmol, 2 equiv) in DMF (2.00 mL) was added 4-(bromomethyl)-1λ⁶-thiane-1,1-dione (19.00 mg, 0.084 mmol, 1.2 equiv) at room temperature. The resulting mixture was stirred for 16 h at room temperature. The reaction was monitored by LCMS. The crude product was purified by Prep-HPLC with the following conditions (Column: XSelect CSH Prep C18 OBD Column, 5 um, 19*150 mm; Flow rate: 25 mL/min; Gradient: 25 B to 50 B in 8 min; 220 nm; RT1: 7.19) to afford 2-[8-(2-chlorophenyl)-7-(4-chlorophenyl)-3-[(1,1-dioxo-1λ⁶-thian-4-yl)methyl]-2,6-dioxopurin-1-yl]acetamide (20 mg, 49.76%) as a white solid.

Compounds 153, 185, and 186 were prepared by the methods and scheme described for Compound 136 using the appropriate reagents.

### Example 31. Preparation of Compound 155

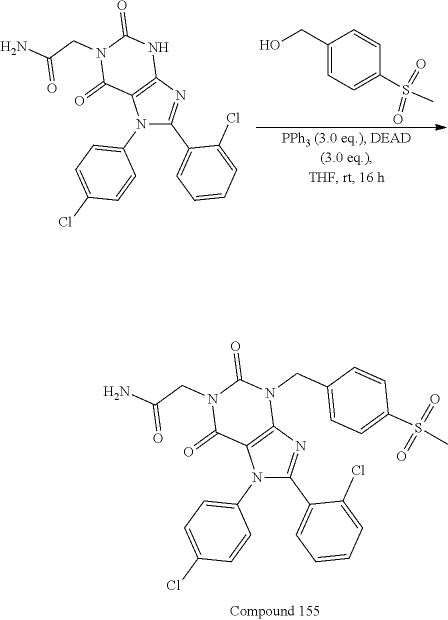 [SMILES: NC(=O)Cn1c(=O)[nH]c2nc(-c3ccccc3Cl)n(-c3ccc(Cl)cc3)c2c1=O ; CS(=O)(=O)c1ccc(CO)cc1 ; CS(=O)(=O)c1ccc(Cn2c(=O)n(CC(N)=O)c(=O)c3c2nc(-c2ccccc2Cl)n3-c2ccc(Cl)cc2)cc1]

To a stirred solution of 2-[8-(2-chlorophenyl)-7-(4-chlorophenyl)-2,6-dioxo-3H-purin-1-yl]acetamide (40.00 mg, 0.093 mmol, 1.00 equiv) and PPh₃ (73.15 mg, 0.279 mmol, 3.00 equiv) in THF (5.00 mL) were added DEAD (48.57 mg, 0.279 mmol, 3.00 equiv) and (4-methanesulfonylphenyl)methanol (25.97 mg, 0.139 mmol, 1.50 equiv) at 0° C. under nitrogen atmosphere. The resulting mixture was stirred for overnight at room temperature under nitrogen atmosphere. The reaction was monitored by LCMS. The resulting mixture was extracted with EtOAc (3×30 mL). The combined organic layers were washed with brine (3×30 mL), dried over anhydrous Na₂SO₄. After filtration, the filtrate was concentrated under reduced pressure. The crude product was purified by Prep-HPLC with the following conditions (Column: Sunfire Prep C18 OBD Column, 10 um, 19*250 mm; Flow rate: 25 mL/min; Gradient: 45 B to 53 B in 11 min; 254 nm; RT1: 10.2) to afford 2-[8-(2-chlorophenyl)-7-(4-chlorophenyl)-3-[(4-methanesulfonylphenyl)methyl]-2,6-dioxopurin-1-yl]acetamide (30 mg, 53.92%) as a white solid.

Compounds 154, 157, and 158 were prepared by the methods and scheme described for Compound 155 using the appropriate reagents.

### Example 32. Preparation of Compounds 142 and 144

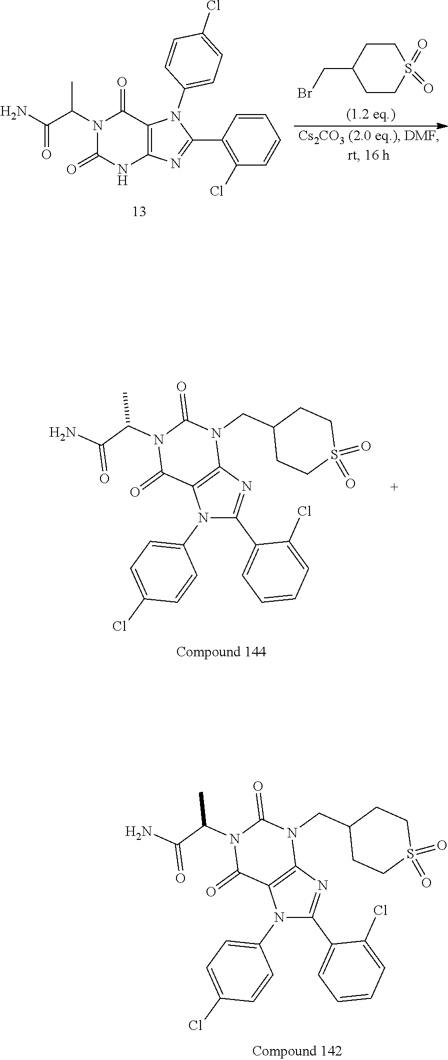 [SMILES: CC(C(N)=O)n1c(=O)[nH]c2nc(-c3ccccc3Cl)n(-c3ccc(Cl)cc3)c2c1=O ; O=S1(=O)CCC(CBr)CC1 ; C[C@@H](C(N)=O)n1c(=O)c2c(nc(-c3ccccc3Cl)n2-c2ccc(Cl)cc2)n(CC2CCS(=O)(=O)CC2)c1=O ; C[C@H](C(N)=O)n1c(=O)c2c(nc(-c3ccccc3Cl)n2-c2ccc(Cl)cc2)n(CC2CCS(=O)(=O)CC2)c1=O]

To a stirred solution of 2-[8-(2-chlorophenyl)-7-(4-chlorophenyl)-2,6-dioxo-3H-purin-1-yl]propanamide (80.00 mg, 0.180 mmol, 1.00 equiv) and Cs₂CO₃ (117.34 mg, 0.360 mmol, 2 equiv) in DMF (3.00 mL) was added 4-(bromomethyl)-1λ⁶-thiane-1,1-dione (49.08 mg, 0.216 mmol, 1.20 equiv) at room temperature. The resulting mixture was stirred for overnight at room temperature. The reaction was monitored by LCMS. The resulting mixture was extracted with EtOAc (3×10 mL). The combined organic layers were washed with brine (3×20 mL), dried over anhydrous Na₂SO₄. After filtration, the filtrate was concentrated under reduced pressure. The crude product was purified by Prep-HPLC to afford (2S)-2-[8-(2-chlorophenyl)-7-(4-chlorophenyl)-3-[(1,1-dioxo-1 lambda6-thian-4-yl)methyl]-2,6-dioxopurin-1-yl]propanamide (30 mg, 28.21%) as a white solid and (2R)-2-[8-(2-chlorophenyl)-7-(4-chlorophenyl)-3-[(1,1-dioxo-1 lambda6-thian-4-yl)methyl]-2,6-dioxopurin-1-yl]propanamide (30 mg, 28.21%) as a white solid.

### Example 33. Preparation of Compound 159

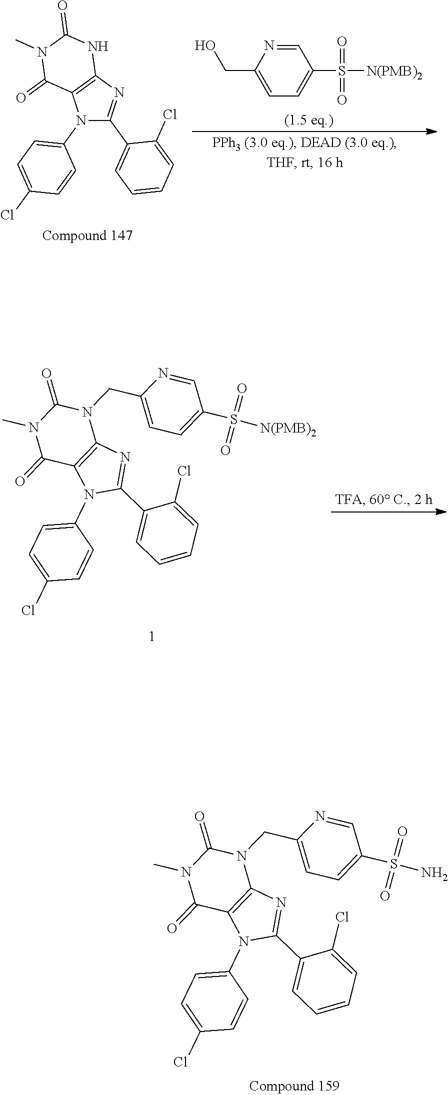

6-[[8-(2-chlorophenyl)-7-(4-chlorophenyl)-1-methyl-2,6-dioxopurin-3-yl]methyl]-N,N-bis[(4-methoxyphenyl)methyl]pyridine-3-sulfonamide. To a stirred solution of 8-(2-chlorophenyl)-7-(4-chlorophenyl)-1-methyl-3H-purine-2,6-dione (1.20 g, 3.099 mmol, 1.00 equiv) and PPh₃ (2.44 g, 9.297 mmol, 3.00 equiv) in THF (20.00 mL) were added 6-(hydroxymethyl)-N,N-bis[(4-methoxyphenyl)methyl]pyridine-3-sulfonamide (1.99 g, 4.649 mmol, 1.50 equiv) and DEAD (1.62 g, 9.297 mmol, 3.00 equiv) at 0° C. under nitrogen atmosphere. The resulting mixture was stirred for 16 h at room temperature under nitrogen atmosphere. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 50% B to 80% B in 40 min; 254/220 nm. The fractions containing the desired product were collected at 71% B and concentrated under reduced pressure. To afford 6-[[8-(2-chlorophenyl)-7-(4-chlorophenyl)-1-methyl-2,6-dioxopurin-3-yl]methyl]-N,N-bis[(4-methoxyphenyl)methyl]pyridine-3-sulfonamide (1.8 g, 72.81%) as a white solid.

6-[[8-(2-chlorophenyl)-7-(4-chlorophenyl)-1-methyl-2,6-dioxopurin-3-yl]methyl]pyridine-3-sulfonamide. To a stirred solution of 6-[[8-(2-chlorophenyl)-7-(4-chlorophenyl)-1-methyl-2,6-dioxopurin-3-yl]methyl]-N,N-bis[(4-methoxyphenyl)methyl]pyridine-3-sulfonamide (1.80 g, 2.256 mmol, 1.00 equiv) in TFA (7.00 mL) at room temperature. The resulting mixture was stirred for 2 h at 60° C. The reaction was monitored by LCMS. The mixture was allowed to cool down to room temperature. The mixture was basified to pH 6 with saturated NaHCO₃ (aq.). The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 30% B to 50% B in 40 min; 254/220 nm. The fractions containing the desired product were collected at 42% B and concentrated under reduced pressure. To afford 6-[[8-(2-chlorophenyl)-7-(4-chlorophenyl)-1-methyl-2,6-dioxopurin-3-yl]methyl]pyridine-3-sulfonamide (1.02 g, 81.10%) as a white solid.

### Example 34. Preparation of Compound 164

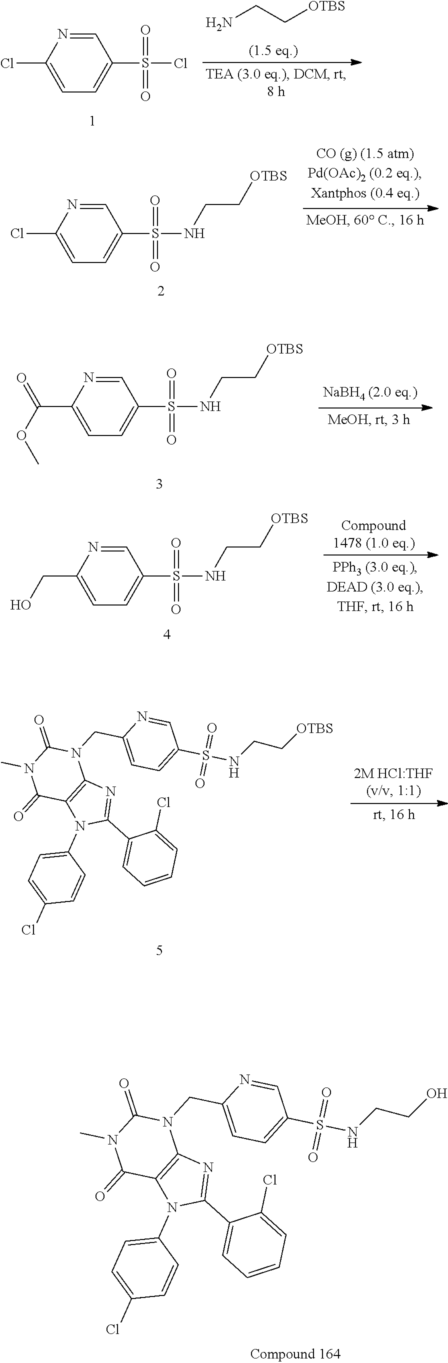

N-[2-[(tert-butyldimethylsilyl)oxy]ethyl]-6-chloropyridine-3-sulfonamide. To a stirred solution of (2-aminoethoxy)(tert-butyl)dimethylsilane (2.48 g, 14.148 mmol, 1.50 equiv) in DCM (50.00 mL) were added TEA (2.86 g, 28.297 mmol, 3.00 equiv) and 6-chloropyridine-3-sulfonyl chloride (2.00 g, 9.432 mmol, 1.00 equiv) at room temperature. The resulting mixture was stirred for 8 h at room temperature. The resulting mixture was concentrated under reduced pressure. The residue was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 60% B to 80% B in 30 min; 254/220 nm). The fractions containing the desired product were collected at 70% B and concentrated under reduced pressure to afford N-[2-[(tert-butyldimethylsilyl)oxy]ethyl]-6-chloropyridine-3-sulfonamide (3 g, 90.63%) as a light blue solid.

Methyl 5-([2-[(tert-butyldimethylsilyl)oxy]ethyl]sulfamoyl)pyridine-2-carboxylate. To a stirred solution of N-[2-[(tert-butyldimethylsilyl)oxy]ethyl]-6-chloropyridine-3-sulfonamide (1.00 g, 2.850 mmol, 1.00 equiv) in MeOH (50.00 mL) were added TEA (865.04 mg, 8.549 mmol, 3.00 equiv), XantPhos (659.53 mg, 1.140 mmol, 0.40 equiv) and Pd(AcO)₂ (127.95 mg, 0.570 mmol, 0.20 equiv) at room temperature under nitrogen atmosphere. The resulting mixture was stirred for 16 h at 60° C. under CO atmosphere. The reaction was monitored by LCMS. The mixture was allowed to cool down to room temperature. The resulting mixture was filtered, the filter cake was washed with MeOH (3×50 mL). The filtrate was concentrated under reduced pressure. The residue was purified by reverse phase flash with the following conditions (Column: C18 Column 330 g; Mobile Phase A: Water (10 mM NH₄HCO₃), Mobile Phase B: ACN; Flow rate: 50 mL/min; Gradient: 50% B to 70% B in 25 min; 254/220 nm). The fractions containing the desired product were collected at 64% B and concentrated under reduced pressure to afford methyl 5-([2-[(tert-butyldimethylsilyl)oxy]ethyl]sulfamoyl)pyridine-2-carboxylate (500 mg, 46.85%) as a white solid.

N-[2-[(tert-butyldimethylsilyl)oxy]ethyl]-6-(hydroxymethyl)pyridine-3-sulfonamide. To a stirred solution of methyl 5-([2-[(tert-butyldimethylsilyl)oxy]ethyl]sulfamoyl)pyridine-2-carboxylate (500.00 mg, 1.335 mmol, 1.00 equiv) in MeOH (20.00 mL) was added NaBH₄ (101.01 mg, 2.670 mmol, 2.00 equiv) at room temperature. The resulting mixture was stirred for 3 h at room temperature. The solution was purified by reverse phase flash with the following conditions (Column: C18 Column 330 g; Mobile Phase A: Water (10 mM AcOH), Mobile Phase B: ACN; Flow rate: 80 mL/min; Gradient: 50% B to 70% B in 25 min; 254/220 nm). The fractions containing the desired product were collected at 64% B and concentrated under reduced pressure to afford N-[2-[(tert-butyldimethylsilyl)oxy]ethyl]-6-(hydroxymethyl)pyridine-3-sulfonamide (330 mg, 71.33%) as a white solid.

N-[2-[(tert-butyldimethylsilyl)oxy]ethyl]-6-[[8-(2-chlorophenyl)-7-(4-chlorophenyl)-1-methyl-2,6-dioxopurin-3-yl]methyl]pyridine-3-sulfonamide. To a stirred solution of 8-(2-chlorophenyl)-7-(4-chlorophenyl)-1-methyl-3H-purine-2,6-dione (50.00 mg, 0.129 mmol, 1.00 equiv), N-[2-[(tert-butyldimethylsilyl)oxy]ethyl]-6-(hydroxymethyl)pyridine-3-sulfonamide (44.74 mg, 0.129 mmol, 1.00 equiv) and PPh₃ (101.60 mg, 0.387 mmol, 3.00 equiv) in THF (5.00 mL) was added DEAD (67.46 mg, 0.387 mmol, 3.00 equiv) at 0° C. under nitrogen atmosphere. The resulting mixture was stirred for 16 h at room temperature under nitrogen atmosphere. The reaction was monitored by LCMS. The solution was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 60% B to 80% B in 40 min; 254/220 nm). The fractions containing the desired product were collected at 69% B and concentrated under reduced pressure to afford N-[2-[(tert-butyldimethylsilyl)oxy]ethyl]-6-[[8-(2-chlorophenyl)-7-(4-chlorophenyl)-1-methyl-2,6-dioxopurin-3-yl]methyl]pyridine-3-sulfonamide (70 mg, 75.74%) as a white solid.

6-[[8-(2-chlorophenyl)-7-(4-chlorophenyl)-1-methyl-2,6-dioxopurin-3-yl]methyl]-N-(2-hydroxyethyl)pyridine-3-sulfonamide. To a stirred solution of N-[2-[(tert-butyldimethylsilyl)oxy]ethyl]-6-[[8-(2-chlorophenyl)-7-(4-chlorophenyl)-1-methyl-2,6-dioxopurin-3-yl]methyl]pyridine-3-sulfonamide (100.00 mg, 0.140 mmol, 1.00 equiv) in THF (3.00 mL) was added HCl (2M) (3.00 mL, 98.736 mmol, 706.67 equiv) at room temperature. The resulting mixture was stirred for 16 h at room temperature. The reaction was monitored by LCMS. The mixture was neutralized to pH 7 with saturated NaHCO₃ (aq.). The solution was purified by reverse phase flash with the following conditions (Column: C18 Column 120 g; Mobile Phase A: Water (10 mM NH₄HCO₃), Mobile Phase B: ACN; Flow rate: 60 mL/min; Gradient: 35% B to 60% B in 30 min; 254/220 nm). The fractions containing the desired product were collected at 45% B and concentrated under reduced pressure to afford 6-[[8-(2-chlorophenyl)-7-(4-chlorophenyl)-1-methyl-2,6-dioxopurin-3-yl]methyl]-N-(2-hydroxyethyl)pyridine-3-sulfonamide (50 mg, 59.50%) as an off-white solid.

### Example 35. Preparation of Compound 156

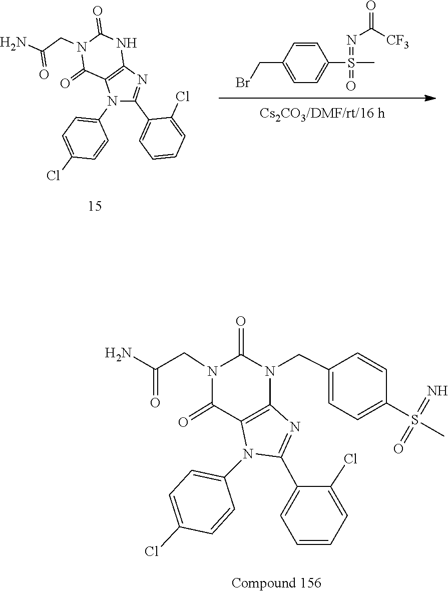 [SMILES: NC(=O)Cn1c(=O)[nH]c2nc(-c3ccccc3Cl)n(-c3ccc(Cl)cc3)c2c1=O ; CS(=O)(=NC(=O)C(F)(F)F)c1ccc(CBr)cc1 ; CS(=N)(=O)c1ccc(Cn2c(=O)n(CC(N)=O)c(=O)c3c2nc(-c2ccccc2Cl)n3-c2ccc(Cl)cc2)cc1]

To a stirred solution of 2-[8-(2-chlorophenyl)-7-(4-chlorophenyl)-2,6-dioxo-3H-purin-1-yl]acetamide (20.00 mg, 0.046 mmol, 1.00 equiv) and K₂CO₃ (32.12 mg, 0.232 mmol, 5.00 equiv) in DMF (3.00 mL) were added N-[[4-(bromomethyl)phenyl](methyl)oxo-lambda6-sulfanylidene]-2,2,2-trifluoroacetamide (19.20 mg, 0.056 mmol, 1.20 equiv) at room temperature. The resulting mixture was stirred for overnight at room temperature. To the above mixture was added MeOH (2.00 mL) at room temperature. The resulting mixture was stirred for additional 1 h at room temperature. The reaction was monitored by LCMS. The crude product was purified by Prep-HPLC to afford 2-[8-(2-chlorophenyl)-7-(4-chlorophenyl)-3-([4-[imino(methyl)oxo-lambda6-sulfanyl]phenyl]methyl)-2,6-dioxopurin-1-yl]acetamide (15 mg) as a white solid.

### Example 36. Preparation of Compound 163

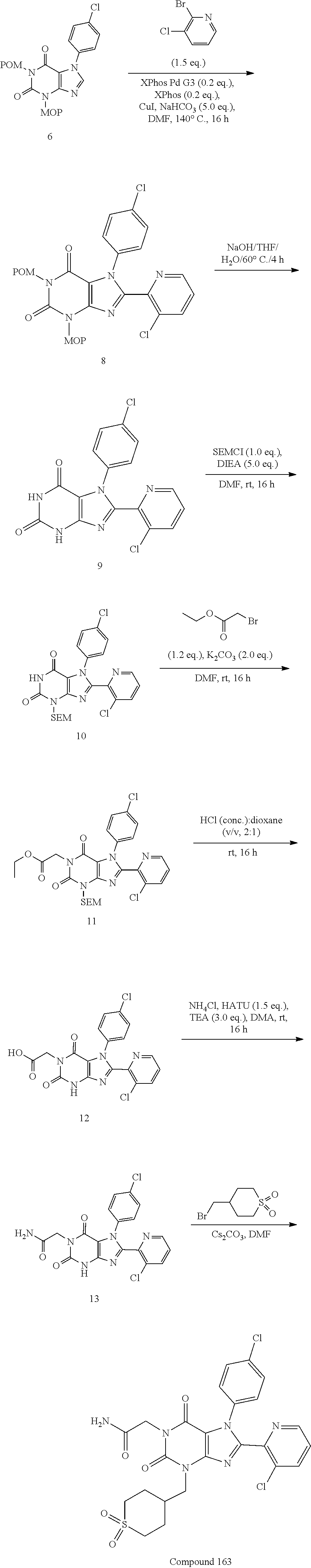

[7-(4-chlorophenyl)-8-(3-chloropyridin-2-yl)-3-[[(2,2-dimethylpropanoyl)oxy]methyl]-2,6-dioxopurin-1-yl]methyl 2,2-dimethylpropanoate. To a stirred solution of [7-(4-chlorophenyl)-3-[[(2,2-dimethylpropanoyl)oxy]methyl]-2,6-dioxopurin-1-yl]methyl 2,2-dimethylpropanoate (2.00 g, 4.074 mmol, 1.00 equiv) and NaHCO₃ (1.71 g, 20.369 mmol, 5.00 equiv) in DMF (50.00 mL) were added 2-bromo-3-chloropyridine (1.18 g, 6.111 mmol, 1.50 equiv), XPhos (388.41 mg, 0.815 mmol, 0.20 equiv). XPhos Pd G3 (689.66 mg, 0.815 mmol, 0.20 equiv) and CuI (3.10 g, 16.295 mmol, 4.00 equiv) at room temperature under nitrogen atmosphere. The resulting mixture was stirred for 16 h at 140° C. under nitrogen atmosphere. The reaction was monitored by LCMS. The mixture was allowed to cool down to room temperature. The resulting mixture was extracted with EtOAc (3×200 mL). The combined organic layers were washed with brine (3×500 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 55% B to 85% B in 40 min; 254/220 nm. The fractions containing the desired product were collected at 71% B and concentrated under reduced pressure. To afford [7-(4-chlorophenyl)-8-(3-chloropyridin-2-yl)-3-[[(2,2-dimethylpropanoyl)oxy]methyl]-2,6-dioxopurin-1-yl]methyl 2,2-dimethylpropanoate (1 g, 40.74%) as a brown oil.

7-(4-chlorophenyl)-8-(3-chloropyridin-2-yl)-1,3-dihydropurine-2,6-dione. To a stirred solution of [7-(4-chlorophenyl)-8-(3-chloropyridin-2-yl)-3-[[(2,2-dimethylpropanoyl)oxy]methyl]-2,6-dioxopurin-1-yl]methyl 2,2-dimethylpropanoate (1.00 g, 1.660 mmol, 1.00 equiv) and NaOH (199.17 mg, 4.980 mmol, 3.00 equiv) in THF (5.00 mL) was added H₂O (5.00 mL) at room temperature. The resulting mixture was stirred for 4 h at 60° C. The reaction was monitored by LCMS. The mixture was acidified to pH 6 with HCl (aq.). The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 30% B to 50% B in 40 min; 254/220 nm. The fractions containing the desired product were collected at 45% B and concentrated under reduced pressure. To afford 7-(4-chlorophenyl)-8-(3-chloropyridin-2-yl)-1,3-dihydropurine-2,6-dione (500 mg, 80.51%) as a light green solid.

7-(4-chlorophenyl)-8-(3-chloropyridin-2-yl)-3-[[2-(trimethylsilyl)ethoxy]methyl]-1H-purine-2,6-dione. To a stirred solution of 7-(4-chlorophenyl)-8-(3-chloropyridin-2-yl)-1,3-dihydropurine-2,6-dione (500.00 mg, 1.336 mmol, 1.00 equiv) in DMF (30.00 mL) were added DIEA (863.51 mg, 6.681 mmol, 5.00 equiv) and SEMCl (222.78 mg, 1.336 mmol, 1.00 equiv) at room temperature. The resulting mixture was stirred for 16 h at room temperature. The reaction was monitored by LCMS. The solution was purified by reverse phase flash with the following conditions (Column: C18 Column 330 g; Mobile Phase A: Water (10 mM NH₄HCO₃), Mobile Phase B: ACN; Flow rate: 80 mL/min; Gradient: 65% B to 85% B in 25 min; 254/220 nm). The fractions containing the desired product were collected at 69% B and concentrated under reduced pressure to afford 7-(4-chlorophenyl)-8-(3-chloropyridin-2-yl)-3-[[2-(trimethylsilyl)ethoxy]methyl]-1H-purine-2,6-dione (350 mg, 51.92%) as a yellow solid.

Ethyl 2-[7-(4-chlorophenyl)-8-(3-chloropyridin-2-yl)-2,6-dioxo-3-[[2-(trimethylsilyl)ethoxy]methyl]purin-1-yl]acetate. To a stirred solution of 7-(4-chlorophenyl)-8-(3-chloropyridin-2-yl)-3-[[2-(trimethylsilyl)ethoxy]methyl]-1H-purine-2,6-dione (350.00 mg, 0.694 mmol, 1.00 equiv) in DMF (10.00 mL) were added K₂CO₃ (191.78 mg, 1.388 mmol, 2.00 equiv) and ethyl bromoacetate (139.05 mg, 0.833 mmol, 1.20 equiv) at room temperature. The resulting mixture was stirred for 16 h at room temperature. The reaction was monitored by LCMS. The resulting mixture was extracted with EtOAc (3×200 mL). The combined organic layers were washed with brine (1×100 mL), dried over anhydrous Na₂SO₄. After filtration, the filtrate was concentrated under reduced pressure. The resulting mixture was used in the next step directly without further purification.

[7-(4-chlorophenyl)-8-(3-chloropyridin-2-yl)-2,6-dioxo-3H-purin-1-yl]acetic acid. To a stirred solution of ethyl 2-[7-(4-chlorophenyl)-8-(3-chloropyridin-2-yl)-2,6-dioxo-3-[[2-(trimethylsilyl)ethoxy]methyl]purin-1-yl]acetate (400.00 mg) in 1,4-dioxane (5.00 mL) was added HCl (12M) (10.00 mL) at room temperature. The resulting mixture was stirred for 16 h at room temperature. The reaction was monitored by LCMS. The residue was purified by reverse flash chromatography with the following conditions: Column: C18 Column 120 g; Mobile Phase A: Water (10 mM AcOH), Mobile Phase B: ACN; Flow rate: 50 mL/min; Gradient: 40% B to 60% B in 40 min; 254/220 nm. The fractions containing the desired product were collected at 55% B and concentrated under reduced pressure to afford [7-(4-chlorophenyl)-8-(3-chloropyridin-2-yl)-2,6-dioxo-3H-purin-1-yl]acetic acid (200 mg, 51.24%) as a light yellow solid.

2-[7-(4-chlorophenyl)-8-(3-chloropyridin-2-yl)-2,6-dioxo-3H-purin-1-yl]acetamide. To a stirred solution of [7-(4-chlorophenyl)-8-(3-chloropyridin-2-yl)-2,6-dioxo-3H-purin-1-yl]acetic acid (200.00 mg, 0.463 mmol, 1.00 equiv) and HATU (197.94 mg, 0.521 mmol, 1.50 equiv) in DMA (8.00 mL) at room temperature. The resulting mixture was stirred for 30 min at room temperature. To the above mixture was added NH₄Cl (55.69 mg, 1.041 mmol, 3.00 equiv) at room temperature. The resulting mixture was stirred for additional 30 min at room temperature. To the above mixture was added TEA (105.35 mg, 1.041 mmol, 3.00 equiv) at room temperature. The resulting mixture was stirred for additional overnight at room temperature. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 20% B to 40% B in 40 min; 254/220 nm. The fractions containing the desired product were collected at 33% B and concentrated under reduced pressure. To afford 2-[7-(4-chlorophenyl)-8-(3-chloropyridin-2-yl)-2,6-dioxo-3H-purin-1-yl]acetamide (150 mg, 75.17%) as a white solid.

2-[7-(4-chlorophenyl)-8-(3-chloropyridin-2-yl)-3-[(1,1-dioxo-1λ⁶-thian-4-yl)methyl]-2,6-dioxopurin-1-yl]acetamide. To a stirred solution of 2-[7-(4-chlorophenyl)-8-(3-chloropyridin-2-yl)-2,6-dioxo-3H-purin-1-yl]acetamide (70.00 mg, 0.162 mmol, 1.00 equiv) and Cs₂CO₃ (105.78 mg, 0.325 mmol, 2.00 equiv) in DMF (5.00 mL) was added 4-(bromomethyl)-1λ⁶-thiane-1,1-dione (44.24 mg, 0.195 mmol, 1.20 equiv) at room temperature. The resulting mixture was stirred for 24 h at room temperature. The reaction was monitored by LCMS. The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC to afford 2-[7-(4-chlorophenyl)-8-(3-chloropyridin-2-yl)-3-[(1,1-dioxo-1λ⁶-thian-4-yl)methyl]-2,6-dioxopurin-1-yl]acetamide (50 mg, 53.34%) as a white solid.

### Example 37. Preparation of Compound 170

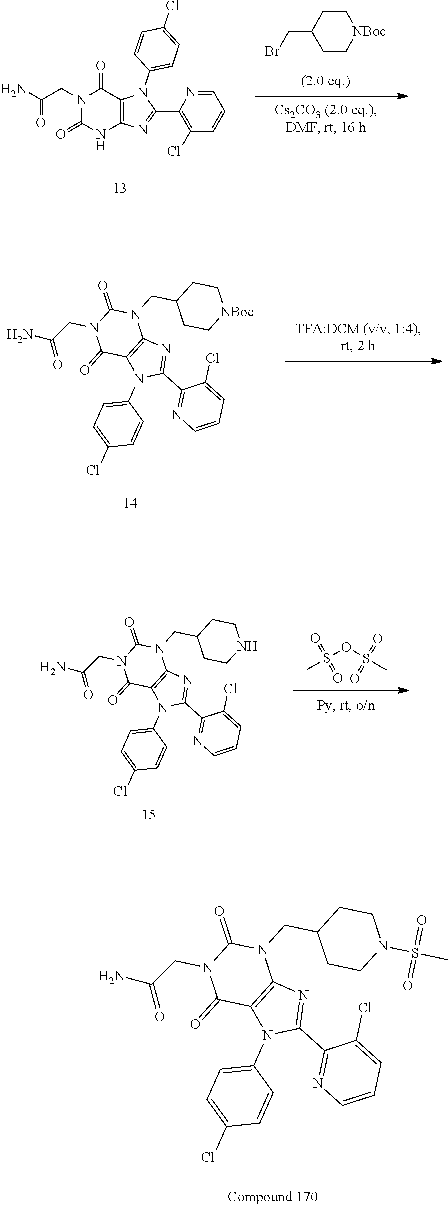

tert-butyl 4-[[1-(carbamoylmethyl)-7-(4-chlorophenyl)-8-(3-chloropyridin-2-yl)-2,6-dioxopurin-3-yl]methyl]piperidine-1-carboxylate. To a stirred solution of 2-[7-(4-chlorophenyl)-8-(3-chloropyridin-2-yl)-2,6-dioxo-3H-purin-1-yl]acetamide (100.00 mg, 0.232 mmol, 1.00 equiv) and Cs₂CO₃ (151.11 mg, 0.464 mmol, 2.00 equiv) in DMF (5.00 mL) was added tert-butyl 4-(bromomethyl)piperidine-1-carboxylate (129.02 mg, 0.464 mmol, 2.00 equiv) at room temperature. The resulting mixture was stirred for 16 h at room temperature. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 40% B to 60% B in 40 min; 254/220 nm. The fractions containing the desired product were collected at 56% B and concentrated under reduced pressure to afford tert-butyl 4-[[1-(carbamoylmethyl)-7-(4-chlorophenyl)-8-(3-chloropyridin-2-yl)-2,6-dioxopurin-3-yl]methyl]piperidine-1-carboxylate (110 mg, 75.47%) as a white solid.

2-[7-(4-chlorophenyl)-8-(3-chloropyridin-2-yl)-2,6-dioxo-3-(piperidin-4-ylmethyl)purin-1-yl]acetamide. To a stirred solution of tert-butyl 4-[[1-(carbamoylmethyl)-7-(4-chlorophenyl)-8-(3-chloropyridin-2-yl)-2,6-dioxopurin-3-yl]methyl]piperidine-1-carboxylate (110.00 mg, 0.175 mmol, 1.00 equiv) in DCM (6.00 mL) was added TFA (1.50 mL) at room temperature. The resulting mixture was stirred for 2 h at room temperature. The reaction was monitored by LCMS. The mixture was basified to pH 7 with saturated NaHCO₃ (aq.). The resulting mixture was extracted with EtOAc (3×30 mL). The combined organic layers were washed with brine (3×50 mL), dried over anhydrous Na₂SO₄. After filtration, the filtrate was concentrated under reduced pressure. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 40% B to 60% B in 40 min; 254/220 nm. The fractions containing the desired product were collected at 49% B and concentrated under reduced pressure. To afford 2-[7-(4-chlorophenyl)-8-(3-chloropyridin-2-yl)-2,6-dioxo-3-(piperidin-4-ylmethyl)purin-1-yl]acetamide (80 mg, 86.51%) as a light yellow solid.

2-[7-(4-chlorophenyl)-8-(3-chloropyridin-2-yl)-3-[(1-methanesulfonylpiperidin-4-yl)methyl]-2,6-dioxopurin-1-yl]acetamide. To a stirred solution of 2-[7-(4-chlorophenyl)-8-(3-chloropyridin-2-yl)-2,6-dioxo-3-(piperidin-4-ylmethyl)purin-1-yl]acetamide (70.00 mg, 0.132 mmol, 1.00 equiv) and methanesulfonic anhydride (65.93 mg, 0.379 mmol, 2.00 equiv) in Pyridine (5.00 mg) at room temperature under nitrogen atmosphere. The resulting mixture was stirred for overnight at room temperature under nitrogen atmosphere. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 30% B to 60% B in 40 min; 254/220 nm. The fractions containing the desired product were collected at 55% B and concentrated under reduced pressure. To afford 2-[7-(4-chlorophenyl)-8-(3-chloropyridin-2-yl)-3-[(1-methanesulfonylpiperidin-4-yl)methyl]-2,6-dioxopurin-1-yl]acetamide (10 mg, 12.45%) as a yellow solid.

### Example 38. Preparation of Compound 168

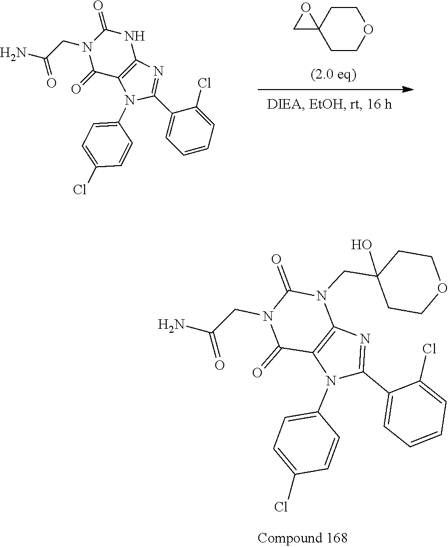 [SMILES: NC(=O)Cn1c(=O)[nH]c2nc(-c3ccccc3Cl)n(-c3ccc(Cl)cc3)c2c1=O ; C1CC2(CCO1)CO2 ; NC(=O)Cn1c(=O)c2c(nc(-c3ccccc3Cl)n2-c2ccc(Cl)cc2)n(CC2(O)CCOCC2)c1=O]

To a stirred solution of 2-[8-(2-chlorophenyl)-7-(4-chlorophenyl)-2,6-dioxo-3H-purin-1-yl]acetamide (50.00 mg, 0.116 mmol, 1.00 equiv) and DIEA (45.06 mg, 0.349 mmol, 3.00 equiv) in EtOH (3.00 mL) was added 1,6-dioxaspiro[2.5]octane (26.53 mg, 0.232 mmol, 2.00 equiv) at room temperature. The resulting mixture was stirred for 16 h at room temperature. The reaction was monitored by LCMS. The solution was purified by reverse phase flash chromatography with the following conditions (Column: C18 Column 120 g; Mobile Phase A: Water (10 mM NH₄HCO₃), Mobile Phase B: ACN; Flow rate: 60 mL/min; Gradient: 35% B to 50% B in 40 min; 254/220 nm). The fractions containing the desired product were collected at 42% B and concentrated under reduced pressure to afford 2-[8-(2-chlorophenyl)-7-(4-chlorophenyl)-3-[(4-hydroxyoxan-4-yl)methyl]-2,6-dioxopurin-1-yl]acetamide (30 mg, 47.42%) as a white solid.

### Example 39. Preparation of Compound 169

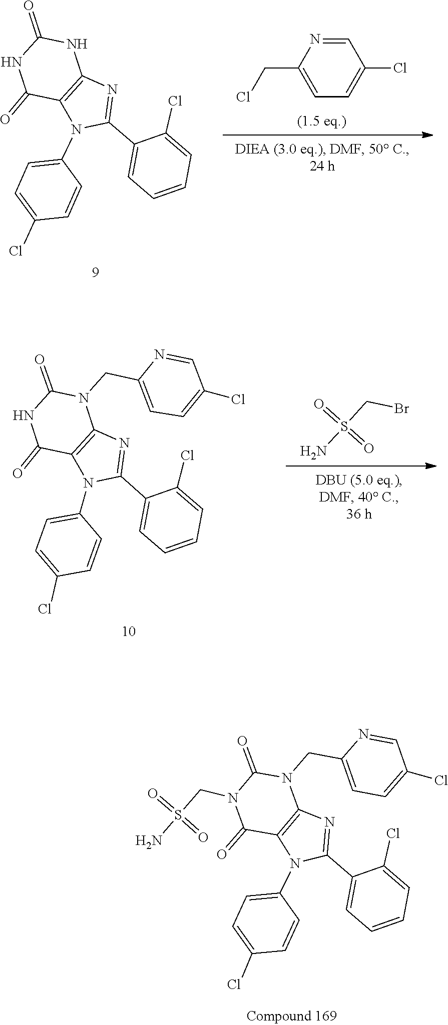 [SMILES: O=c1[nH]c(=O)c2c(nc(-c3ccccc3Cl)n2-c2ccc(Cl)cc2)[nH]1 ; ClCc1ccc(Cl)cn1 ; O=c1[nH]c(=O)n(Cc2ccc(Cl)cn2)c2nc(-c3ccccc3Cl)n(-c3ccc(Cl)cc3)c12 ; NS(=O)(=O)CBr ; NS(=O)(=O)Cn1c(=O)c2c(nc(-c3ccccc3Cl)n2-c2ccc(Cl)cc2)n(Cc2ccc(Cl)cn2)c1=O]

8-(2-chlorophenyl)-7-(4-chlorophenyl)-3-[(5-chloropyridin-2-yl)methyl]-1H-purine-2,6-dione. To a stirred solution of 8-(2-chlorophenyl)-7-(4-chlorophenyl)-1,3-dihydropurine-2,6-dione (70.00 mg, 0.188 mmol, 1.00 equiv) and 5-chloro-2-(chloromethyl)pyridine (45.58 mg, 0.281 mmol, 1.50 equiv) in DMF (7.00 mL) was added DIEA (72.73 mg, 0.563 mmol, 3.00 equiv) at room temperature. The resulting mixture was stirred for 24 h at 50° C. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 50 mL/min; Gradient: 40% B to 80% B in 40 min; 254/220 nm. The fractions containing the desired product were collected at 56% B and concentrated under reduced pressure. to afford 8-(2-chlorophenyl)-7-(4-chlorophenyl)-3-[(5-chloropyridin-2-yl)methyl]-1H-purine-2,6-dione (50 mg, 53.45%) as a white solid.

[8-(2-chlorophenyl)-7-(4-chlorophenyl)-3-[(5-chloropyridin-2-yl)methyl]-2,6-dioxopurin-1-yl]methanesulfonamide. To a stirred solution of 8-(2-chlorophenyl)-7-(4-chlorophenyl)-3-[(5-chloropyridin-2-yl)methyl]-1H-purine-2,6-dione (60.00 mg, 0.120 mmol, 100 equiv) and DBU (91.57 mg, 0.602 mmol, 5.00 equiv) in DMF (7.00 mL) was added bromomethanesulfonamide (41.87 mg, 0.241 mmol, 2.00 equiv) at room temperature. The resulting mixture was stirred for 36 h at 40° C. The reaction was monitored by LCMS. The crude product was purified by Prep-HPLC to afford [8-(2-chlorophenyl)-7-(4-chlorophenyl)-3-[(5-chloropyridin-2-yl)methyl]-2,6-dioxopurin-1-yl]methanesulfonamide (5 mg, 7.02%) as a white solid.

### Example 40. Preparation of Compound 174

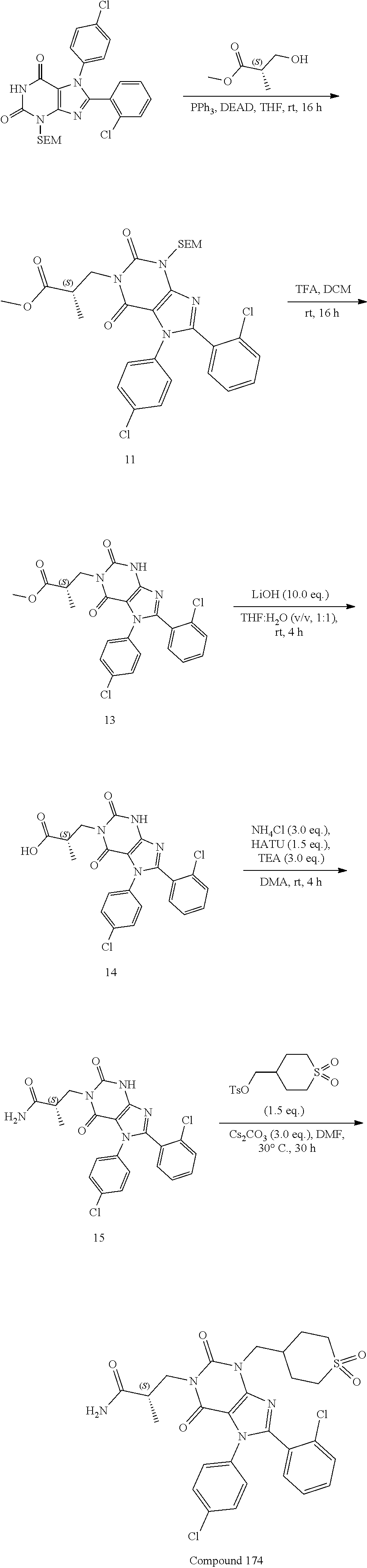

Methyl (2S)-3-[8-(2-chlorophenyl)-7-(4-chlorophenyl)-2,6-dioxo-3-[[2-(trimethylsilyl)ethoxy]methyl]purin-1-yl]-2-methylpropanoate. To a stirred solution of 8-(2-chlorophenyl)-7-(4-chlorophenyl)-3-[[2-(trimethylsilyl)ethoxy]methyl]-1H-purine-2,6-dione (150.00 mg, 0.298 mmol, 1.00 equiv) and PPh₃ (234.43 mg, 0.894 mmol, 3.00 equiv) in THF (5.00 mL) were added methyl (2S)-3-hydroxy-2-methylpropanoate (52.79 mg, 0.447 mmol, 1.50 equiv) and DEAD (155.66 mg, 0.894 mmol, 3.00 equiv) at 0° C. under nitrogen atmosphere. The resulting mixture was stirred for overnight at room temperature under nitrogen atmosphere. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 50% B to 80% B in 40 min; 254/220 nm. The fractions containing the desired product were collected at 65% B and concentrated under reduced pressure. To afford methyl (2S)-3-[8-(2-chlorophenyl)-7-(4-chlorophenyl)-2,6-dioxo-3-[[2-(trimethylsilyl)ethoxy]methyl]purin-1-yl]-2-methylpropanoate (140 mg, 77.85%) as a light yellow solid.

Methyl (2S)-3-[8-(2-chlorophenyl)-7-(4-chlorophenyl)-2,6-dioxo-3H-purin-1-yl]-2-methylpropanoate. To a stirred solution/mixture of methyl (2S)-3-[8-(2-chlorophenyl)-7-(4-chlorophenyl)-2,6-dioxo-3-[[2-(trimethylsilyl)ethoxy]methyl]purin-1-yl]-2-methylpropanoate (140.00 mg, 0.232 mmol, 1.00 equiv) in DCM (5.00 mL) was added TFA (1.00 mL) at room temperature. The resulting mixture was stirred for 16 h at room temperature. The reaction was monitored by LCMS. The mixture was basified to pH 7 with saturated NaHCO₃ (aq.). The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 40% B to 70% B in 40 min; 254/220 nm. The fractions containing the desired product were collected at 55% B and concentrated under reduced pressure. To afford methyl (2S)-3-[8-(2-chlorophenyl)-7-(4-chlorophenyl)-2,6-dioxo-3H-purin-1-yl]-2-methylpropanoate (100 mg, 91.09%) as a white solid.

(2S)-3-[8-(2-chlorophenyl)-7-(4-chlorophenyl)-2,6-dioxo-3H-purin-1-yl]-2-methylpropanoic acid. To a stirred solution/mixture of methyl (2S)-3-[8-(2-chlorophenyl)-7-(4-chlorophenyl)-2,6-dioxo-3H-purin-1-yl]-2-methylpropanoate (100.00 mg, 0.211 mmol, 1.00 equiv) and LiOH (50.60 mg, 2.113 mmol, 10.00 equiv) in THF (4.00 mL) was added H₂O (4.00 mL) at room temperature. The resulting mixture was stirred for 4 h at room temperature. The reaction was monitored by LCMS. The mixture was acidified to pH 6 with HCl (aq.). The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 30% B to 60% B in 40 min; 254/220 nm. The fractions containing the desired product were collected at 45% B and concentrated under reduced pressure. To afford (2S)-3-[8-(2-chlorophenyl)-7-(4-chlorophenyl)-2,6-dioxo-3H-purin-1-yl]-2-methylpropanoic acid (85 mg, 87.60%) as a white solid.

(2S)-3-[8-(2-chlorophenyl)-7-(4-chlorophenyl)-2,6-dioxo-3H-purin-1-yl]-2-methylpropanamide. A solution of (2S)-3-[8-(2-chlorophenyl)-7-(4-chlorophenyl)-2,6-dioxo-3H-purin-1-yl]-2-methylpropanoic acid (90.00 mg, 0.196 mmol, 1.00 equiv) and HATU (111.76 mg, 0.294 mmol, 1.5 equiv) in DMA (4.00 mL) was stirred for 30 min at room temperature. To the above mixture was added NH₄Cl (31.45 mg, 0.588 mmol, 3 equiv) at room temperature. The resulting mixture was stirred for 30 min at room temperature. To the above mixture was added TEA (59.49 mg, 0.588 mmol, 3 equiv) at room temperature. The resulting mixture was stirred for additional 4 h at room temperature. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 30% B to 60% B in 40 min; 254/220 nm. The fractions containing the desired product were collected at 45% B and concentrated under reduced pressure to afford (2S)-3-[8-(2-chlorophenyl)-7-(4-chlorophenyl)-2,6-dioxo-3H-purin-1-yl]-2-methylpropanamide (80 mg, 89.08%) as a white solid.

(2S)-3-[8-(2-chlorophenyl)-7-(4-chlorophenyl)-3-[(1,1-dioxo-1λ⁶-thian-4-yl)methyl]-2,6-dioxopurin-1-yl]-2-methylpropanamide. To a stirred solution of (2S)-3-[8-(2-chlorophenyl)-7-(4-chlorophenyl)-2,6-dioxo-3H-purin-1-yl]-2-methylpropanamide (80.00 mg, 0.175 mmol, 1.00 equiv) and Cs₂CO₃ (170.62 mg, 0.524 mmol, 3 equiv) in DMF (5.00 mL) was added (1,1-dioxo-1λ⁶-thian-4-yl)methyl 4-methylbenzenesulfonate (83.37 mg, 0.262 mmol, 1.5 equiv) at room temperature. The resulting mixture was stirred for 30 h at 30° C. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 40% B to 70% B in 40 min; 254/220 nm. The fractions containing the desired product were collected at 61% B and concentrated under reduced pressure. To afford (2S)-3-[8-(2-chlorophenyl)-7-(4-chlorophenyl)-3-[(1,1-dioxo-1λ⁶-thian-4-yl)methyl]-2,6-dioxopurin-1-yl]-2-methylpropanamide (65 mg, 61.60%) as a white solid.

Compound 175 was prepared by the methods and scheme described for Compound 174 using the appropriate reagents.

### Example 41. Preparation of Compounds 183 and 184

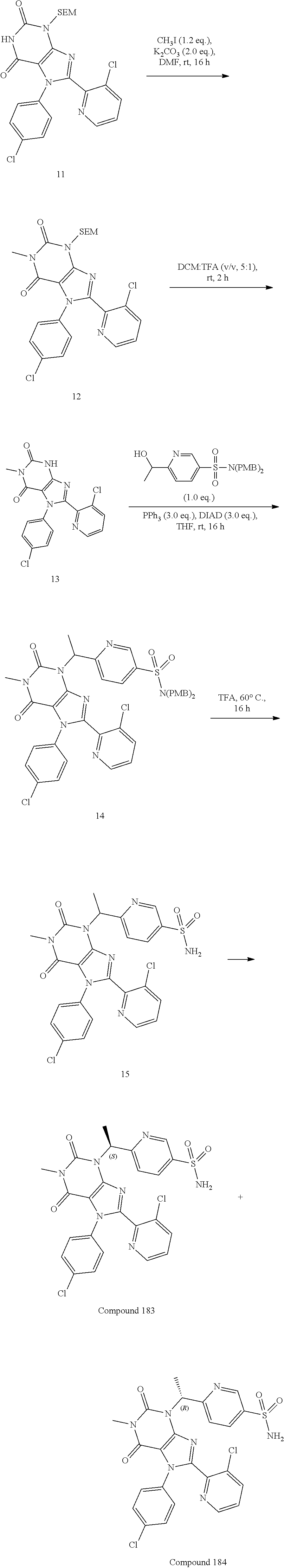

7-(4-chlorophenyl)-8-(3-chloropyridin-2-yl)-1-methyl-3-[[2-(trimethylsilyl)ethoxy]methyl]purine-2,6-dione. To a stirred solution of 7-(4-chlorophenyl)-8-(3-chloropyridin-2-yl)-3-[[2-(trimethylsilyl)ethoxy]methyl]-1H-purine-2,6-dione (1.80 g, 3.568 mmol, 1.00 equiv) and K₂CO₃ (0.99 g, 7.137 mmol, 2.00 equiv) in DMF (20.00 mL) was added CH₃I (0.61 g, 4.282 mmol, 1.20 equiv) at room temperature under air atmosphere. The resulting mixture was stirred for 16 h at room temperature under air atmosphere. The resulting mixture was concentrated under reduced pressure. The residue was purified by silica gel column chromatography, eluted with PE/EtOAc (4:1) to afford 7-(4-chlorophenyl)-8-(3-chloropyridin-2-yl)-1-methyl-3-[[2-(trimethylsilyl)ethoxy]methyl]purine-2,6-dione (1.2 g, 64.86%) as an off-white solid.

7-(4-chlorophenyl)-8-(3-chloropyridin-2-yl)-1-methyl-3H-purine-2,6-dione. To a stirred solution of 7-(4-chlorophenyl)-8-(3-chloropyridin-2-yl)-1-methyl-3-[[2-(trimethylsilyl)ethoxy]methyl]purine-2,6-dione (1.20 g, 2.315 mmol, 1.00 equiv) in DCM (5.00 mL) was added TFA (1.00 mL, 13.463 mmol, 5.82 equiv) at room temperature under air atmosphere. The resulting mixture was stirred for 2 h at room temperature under air atmosphere. The resulting mixture was concentrated under reduced pressure. The residue was purified by silica gel column chromatography, eluted with PE/EtOAc (1:1) to afford 7-(4-chlorophenyl)-8-(3-chloropyridin-2-yl)-1-methyl-3H-purine-2,6-dione (800 mg, 89.04%) as an off-white solid.

6-[1-[7-(4-chlorophenyl)-8-(3-chloropyridin-2-yl)-1-methyl-2,6-dioxopurin-3-yl]ethyl]-N,N-bis[(4-methoxyphenyl)methyl]pyridine-3-sulfonamide. To a stirred solution of 7-(4-chlorophenyl)-8-(3-chloropyridin-2-yl)-1-methyl-3H-purine-2,6-dione (800.00 mg, 2.061 mmol, 1.00 equiv) and 6-(1-hydroxyethyl)-N,N-bis[(4-methoxyphenyl)methyl]pyridine-3-sulfonamide (911.94 mg, 2.061 mmol, 1.00 equiv) in THF (10.00 mL) were added DIAD (799.01 mg, 6.182 mmol, 3.00 equiv) and PPh₃ (1621.51 mg, 6.182 mmol, 3.00 equiv) at room temperature under nitrogen atmosphere. The resulting mixture was stirred for 16 h at room temperature under nitrogen atmosphere. The resulting mixture was concentrated under vacuum. The residue was purified by silica gel column chromatography, eluted with PE/EtOAc (2:1) to afford 6-[1-[7-(4-chlorophenyl)-8-(3-chloropyridin-2-yl)-1-methyl-2,6-dioxopurin-3-yl]ethyl]-N,N-bis[(4-methoxyphenyl)methyl]pyridine-3-sulfonamide (1 g, 59.71%) as an off-white solid. 6-[(1R)-1-[7-(4-chlorophenyl)-8-(3-chloropyridin-2-yl)-1-methyl-2,6-dioxopurin-3-yl]ethyl]pyridine-3-sulfonamide. Into a 50 mL round-bottom flask were added 6-[1-[7-(4-chlorophenyl)-8-(3-chloropyridin-2-yl)-1-methyl-2,6-dioxopurin-3-yl]ethyl]-N,N-bis[(4-methoxyphenyl)methyl]pyridine-3-sulfonamide (1.00 g, 1.230 mmol, 1.00 equiv) and TFA (20.00 mL) at room temperature. The resulting mixture was stirred for 16 h at 60° C. under air atmosphere. The resulting mixture was concentrated under vacuum. The residue was purified by reverse flash chromatography with the following conditions: column, C18 silica gel; mobile phase, ACN in water (10 mM NH₄HCO₃), 20% to 50% gradient in 25 min; detector, UV 254 nm to afford a crude product. The crude product (500 mg) was purified by Prep-Chiral HPLC with the following conditions (Column: CHIRAL IC, 2*25 cm, 5 um; Mobile Phase A: Hex:DCM=3:1 (10 mM NH₃-MEOH)—HPLC, Mobile Phase B: EtOH:DCM=1:1—HPLC; Flow rate: 20 mL/min; Gradient: 60 B to 60 B in 13.5 min; 220/254 nm; RT1: 5.557; RT2: 11.872; Injection Volume: 2.96 mL; Number Of Runs: 5) to afford 6-[(1S)-1-[7-(4-chlorophenyl)-8-(3-chloropyridin-2-yl)-1-methyl-2,6-dioxopurin-3-yl]ethyl]pyridine-3-sulfonamide (175.2 mg) as an off-white solid and 6-[(1R)-1-[7-(4-chlorophenyl)-8-(3-chloropyridin-2-yl)-1-methyl-2,6-dioxopurin-3-yl]ethyl]pyridine-3-sulfonamide (160.6 mg) as an off-white solid.

### Example 42. Preparation of Compounds 179 and 180

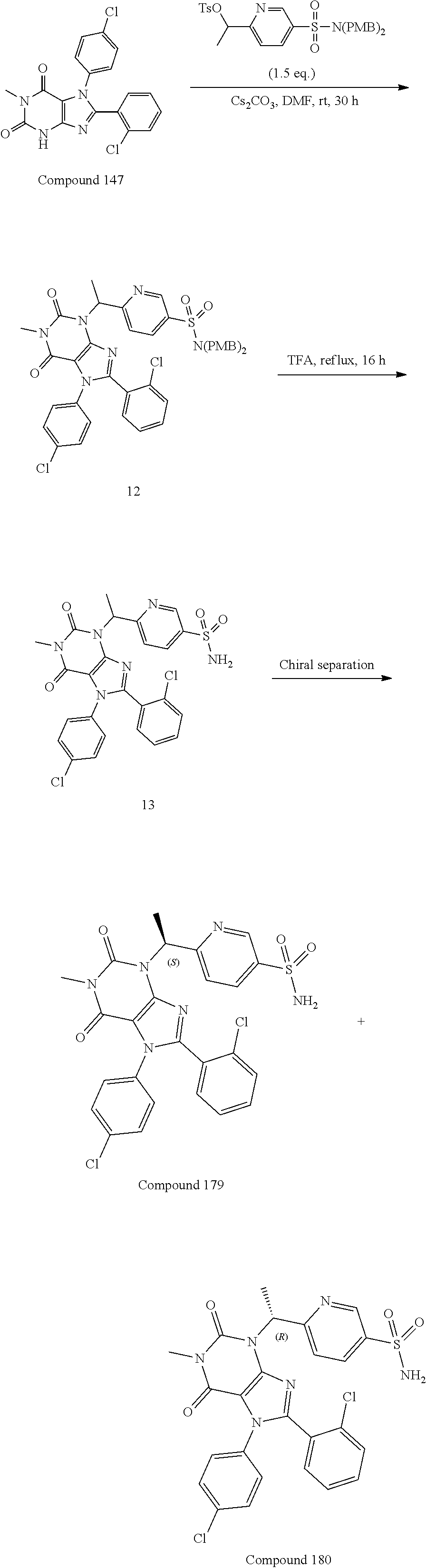

6-[1-[8-(2-chlorophenyl)-7-(4-chlorophenyl)-1-methyl-2,6-dioxopurin-3-yl]ethyl]-N,N-bis[(4-methoxyphenyl)methyl]pyridine-3-sulfonamide. To a stirred solution of 8-(2-chlorophenyl)-7-(4-chlorophenyl)-1-methyl-3H-purine-2,6-dione (100.00 mg, 0.258 mmol, 1.00 equiv) and Cs₂CO₃ (252.43 mg, 0.775 mmol, 3.00 equiv) in DMF (5.00 mL) was added 1-(5-[bis[(4-methoxyphenyl)methyl]sulfamoyl]pyridin-2-yl)ethyl 4-methylbenzenesulfonate (231.15 mg, 0.387 mmol, 1.50 equiv) at room temperature. The resulting mixture was stirred for 30 h at room temperature. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 40% B to 70% B in 40 min; 254/220 nm. The fractions containing the desired product were collected at 60% B and concentrated under reduced pressure. To afford 6-[1-[8-(2-chlorophenyl)-7-(4-chlorophenyl)-1-methyl-2,6-dioxopurin-3-yl]ethyl]-N,N-bis[(4-methoxyphenyl)methyl]pyridine-3-sulfonamide (150 mg, 71.55%) as a white solid.

6-[(1S)-1-[8-(2-chlorophenyl)-7-(4-chlorophenyl)-1-methyl-2,6-dioxopurin-3-yl]ethyl]pyridine-3-sulfonamide and 6-[(1R)-1-[8-(2-chlorophenyl)-7-(4-chlorophenyl)-1-methyl-2,6-dioxopurin-3-yl]ethyl]pyridine-3-sulfonamide. To a stirred solution of 6-[1-[8-(2-chlorophenyl)-7-(4-chlorophenyl)-1-methyl-2,6-dioxopurin-3-yl]ethyl]-N,N-bis[(4-methoxyphenyl)methyl]pyridine-3-sulfonamide (350.00 mg, 0.431 mmol, 1.00 equiv) in TFA (10.00 mL) at 60° C. The resulting mixture was stirred for 16 h at reflux. The reaction was monitored by LCMS. The mixture/residue was basified to pH 6 with saturated NaHCO₃ (aq.). The resulting mixture was extracted with EtOAc (3×20 mL). The combined organic layers were washed with brine (3×20 mL), dried over anhydrous Na₂SO₄. After filtration, the filtrate was concentrated under reduced pressure. The crude product was purified by Prep-HPLC with the following conditions (Column: XBridge Shield RP18 OBD Column, 30*150 mm, 5 um; Flow rate: 60 mL/min; Gradient: 40 B to 60 B in 7 min; 254 nm; RT1: 5.9; RT2:; Injection Volume: mL) to afford 6-[(1S)-1-[8-(2-chlorophenyl)-7-(4-chlorophenyl)-1-methyl-2,6-dioxopurin-3-yl]ethyl]pyridine-3-sulfonamide (115 mg, 46.67%) and 6-[(1R)-1-[8-(2-chlorophenyl)-7-(4-chlorophenyl)-1-methyl-2,6-dioxopurin-3-yl]ethyl]pyridine-3-sulfonamide (115 mg, 46.67%) as a white solid.

### Example 43. Preparation of Compounds 187 and 188

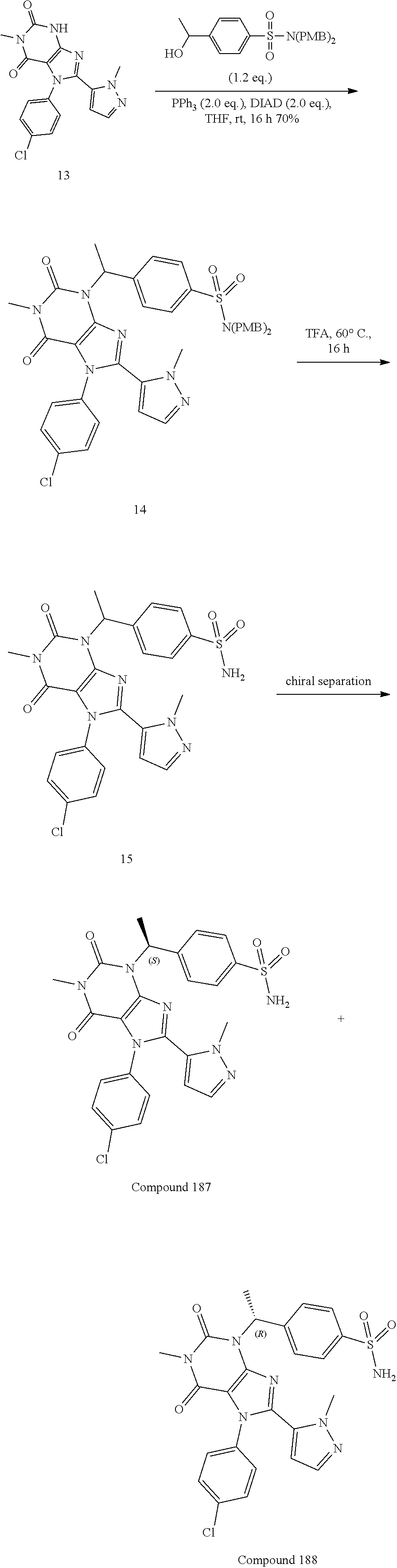

6-[1-[7-(4-chlorophenyl)-1-methyl-8-(2-methylpyrazol-3-yl)-2,6-dioxopurin-3-yl]ethyl]-N,N-bis[(4-methoxyphenyl)methyl]pyridine-3-sulfonamide. To a stirred solution of 7-(4-chlorophenyl)-1-methyl-8-(2-methylpyrazol-3-yl)-3H-purine-2,6-dione (360.00 mg, 1.009 mmol, 1.00 equiv) and 6-(1-hydroxyethyl)-N,N-bis[(4-methoxyphenyl)methyl]pyridine-3-sulfonamide (535.84 mg, 1.211 mmol, 1.20 equiv) in THF (5.00 mL) were added DIAD (408.08 mg, 2.018 mmol, 2.00 equiv) and PPh₃ (529.32 mg, 2.018 mmol, 2.00 equiv) at room temperature under nitrogen atmosphere. The resulting mixture was stirred for 16 h at room temperature under nitrogen atmosphere. The resulting mixture was concentrated under reduced pressure to afford 6-[1-[7-(4-chlorophenyl)-1-methyl-8-(2-methylpyrazol-3-yl)-2,6-dioxopurin-3-yl]ethyl]-N,N-bis[(4-methoxyphenyl)methyl]pyridine-3-sulfonamide (420 mg, crude) as a white solid.

4-[(1S)-1-[7-(4-chlorophenyl)-1-methyl-8-(2-methylpyrazol-3-yl)-2,6-dioxopurin-3-yl]ethyl]benzenesulfonamide and 4-[(1R)-1-[7-(4-chlorophenyl)-1-methyl-8-(2-methylpyrazol-3-yl)-2,6-dioxopurin-3-yl]ethyl]benzenesulfonamide. Into a 25 mL round-bottom flask were added 4-[1-[7-(4-chlorophenyl)-1-methyl-8-(2-methylpyrazol-3-yl)-2,6-dioxopurin-3-yl]ethyl]-N,N-bis[(4-methoxyphenyl)methyl]benzenesulfonamide (800.00 mg) and TFA (10.00 mL) at room temperature. The resulting mixture was stirred for 16 h at 60° C. under air atmosphere. The resulting mixture was concentrated under reduced pressure. The residue was purified by reverse flash chromatography with the following conditions: column, C18 silica gel; mobile phase, ACN in water (10 mM NH₄HCO₃), 20% to 45% gradient in 25 min; detector, UV 254 nm to afford a crude product (350 mg). The crude product (350 mg) was purified by Prep-Chiral-HPLC with the following conditions (Column: CHIRALPAK IG, 2*25 cm, 5 um; Mobile Phase A: Hex (10 mM NH₃), Mobile Phase B: EtOH:DCM=1:1—HPLC; Flow rate: 20 mL/min; Gradient: 35 B to 35 B in 17 min; 220/254 nm; RT1: 12.837; RT2: 15.325; Injection Volume: 0.9 mL; Number Of Runs: 17) to afford 4-[(1S)-1-[7-(4-chlorophenyl)-1-methyl-8-(2-methylpyrazol-3-yl)-2,6-dioxopurin-3-yl]ethyl]benzenesulfonamide (125.8 mg) as an off-white solid and 4-[(1R)-1-[7-(4-chlorophenyl)-1-methyl-8-(2-methylpyrazol-3-yl)-2,6-dioxopurin-3-yl]ethyl]benzenesulfonamide (128.6 mg) as an off-white solid.

### Example 44. Preparation of Compound 182

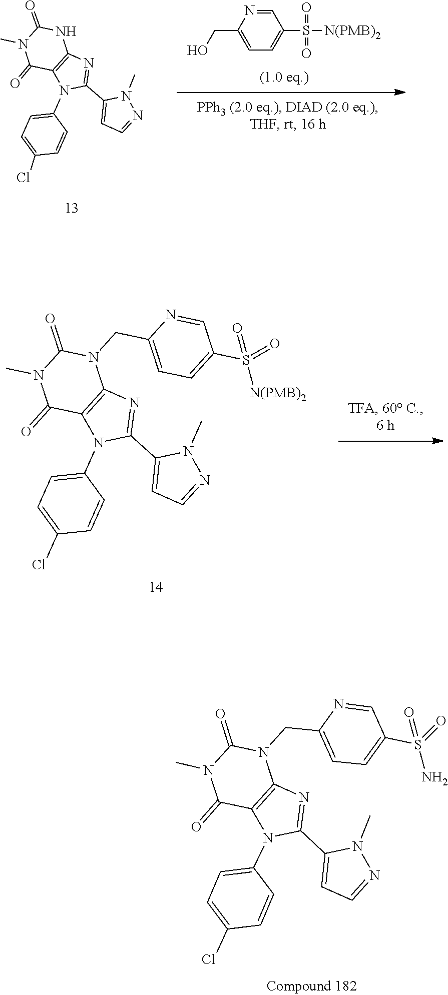

6-[[7-(4-chlorophenyl)-1-methyl-8-(2-methylpyrazol-3-yl)-2,6-dioxopurin-3-yl]methyl]-N,N-bis[(4-methoxyphenyl)methyl]pyridine-3-sulfonamide. To a stirred solution of 7-(4-chlorophenyl)-1-methyl-8-(2-methylpyrazol-3-yl)-3H-purine-2,6-dione (60.00 mg, 0.168 mmol, 1.00 equiv) and 6-(hydroxymethyl)-N,N-bis[(4-methoxyphenyl)methyl]pyridine-3-sulfonamide (86.48 mg, 0.202 mmol, 1.20 equiv) in THF (2.00 mL) were added DIAD (68.01 mg, 0.336 mmol, 2.00 equiv) and PPh₃ (88.22 mg, 0.336 mmol, 2.00 equiv) at room temperature under nitrogen atmosphere. The resulting mixture was stirred for 16 h at room temperature under nitrogen atmosphere. The resulting mixture was concentrated under reduced pressure. The residue was purified by silica gel column chromatography, eluted with PE/EtOAc (1:1) to afford 6-[[7-(4-chlorophenyl)-1-methyl-8-(2-methylpyrazol-3-yl)-2,6-dioxopurin-3-yl]methyl]-N,N-bis[(4-methoxyphenyl)methyl]pyridine-3-sulfonamide (90 mg) as a white solid.

6-[[7-(4-chlorophenyl)-1-methyl-8-(2-methylpyrazol-3-yl)-2,6-dioxopurin-3-yl]methyl]pyridine-3-sulfonamide. Into a 50 mL round-bottom flask were added 6-[[7-(4-chlorophenyl)-1-methyl-8-(2-methylpyrazol-3-yl)-2,6-dioxopurin-3-yl]methyl]-N,N-bis[(4-methoxyphenyl)methyl]pyridine-3-sulfonamide (90.00 mg, 0.117 mmol, 1.00 equiv) and TFA (5.00 mL) at room temperature. The resulting mixture was stirred for 6 h at 60° C. The resulting mixture was concentrated under reduced pressure. The residue was purified by reverse flash chromatography with the following conditions: column, C18 silica gel; mobile phase, ACN in water (10 mM NH₄HCO₃), 25% to 45% gradient in 20 min; detector, UV 254 nm. to afford 6-[[7-(4-chlorophenyl)-1-methyl-8-(2-methylpyrazol-3-yl)-2,6-dioxopurin-3-yl]methyl]pyridine-3-sulfonamide (34.6 mg) as an off-white solid.

Compound 181 was prepared by the methods and scheme described for Compound 182 using appropriate reagents.

### Example 45. Preparation of Compounds 176 and 177

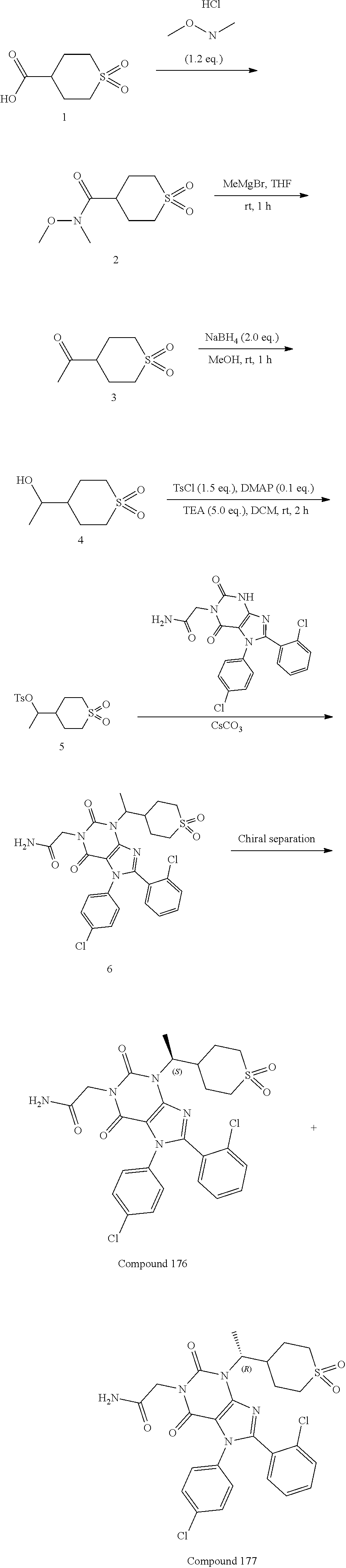

N-methoxy-N-methyl-1,1-dioxo-1λ⁶-thiane-4-carboxamide. To a stirred solution of 1,1-dioxo-1λ⁶-thiane-4-carboxylic acid (5.00 g, 28.058 mmol, 1.00 equiv) and HATU (12.80 g, 33.670 mmol, 1.20 equiv) in DMF (30.00 mL) were added N,O-dimethylhydroxylamine (2.06 g, 33.670 mmol, 1.20 equiv) and TEA (8.52 g, 84.175 mmol, 3.00 equiv) at room temperature. The resulting mixture was stirred for 16 h at room temperature. The resulting mixture was concentrated under reduced pressure. The residue was purified by silica gel column chromatography, eluted with PE/EtOAc (1:2) to afford N-methoxy-N-methyl-1,1-dioxo-1λ⁶-thiane-4-carboxamide (4 g, 64.43%) as a white solid.

4-acetyl-1λ⁶-thiane-1,1-dione. To a stirred solution/mixture of N-methoxy-N-methyl-1,1-dioxo-1λ⁶-thiane-4-carboxamide (3.00 g, 13.558 mmol, 1.00 equiv) in THF (30.00 mL) was added 1M MeMgBr (2.43 g, 20.337 mmol, 1.50 equiv) in portions at room temperature under nitrogen atmosphere. The resulting mixture was stirred for 1 h at room temperature under nitrogen atmosphere. The reaction was quenched with sat. NaHCO₃ (aq.) at room temperature. The resulting mixture was diluted with water (50 mL). The resulting mixture was extracted with EtOAc (3×50 mL), dried over anhydrous Na₂SO₄. After filtration, the filtrate was concentrated under reduced pressure. The residue was purified by silica gel column chromatography, eluted with PE/EtOAc (1:1) to afford 4-acetyl-1λ⁶-thiane-1,1-dione (1.9 g, 79.52%) as a colorless oil. 4-(1-hydroxyethyl)-1λ⁶-thiane-1,1-dione. To a stirred solution of 4-acetyl-1λ⁶-thiane-1,1-dione (1.90 g, 10.781 mmol, 1.00 equiv) in MeOH (20.00 mL) was added NaBH₄ (0.82 g, 21.563 mmol, 2.00 equiv) in portions at room temperature. The resulting mixture was stirred for 1 h at room temperature. The reaction was quenched with sat. NH₄Cl (aq.) at room temperature. The resulting mixture was concentrated under vacuum. The resulting mixture was diluted with water (30 mL). The resulting mixture was extracted with EtOAc (3×30 mL), dried over anhydrous Na₂SO₄. After filtration, the filtrate was concentrated under reduced pressure to afford 4-(1-hydroxyethyl)-1λ⁶-thiane-1,1-dione (1.7 g, 88.46%) as a colorless oil.

1-(1,1-dioxo-1λ⁶-thian-4-yl)ethyl 4-methylbenzenesulfonate. To a stirred solution of 4-(1-hydroxyethyl)-1λ⁶-thiane-1,1-dione (1.70 g, 9.537 mmol, 1.00 equiv) and TEA (4.83 g, 47.686 mmol, 5.00 equiv) in DCM (30.00 mL) were added DMAP (0.12 g, 0.954 mmol, 0.10 equiv) and TsCl (2.73 g, 14.306 mmol, 1.50 equiv) at room temperature. The resulting mixture was stirred for 2 h at room temperature. The resulting mixture was concentrated under reduced pressure. The residue was purified by reverse flash chromatography with the following conditions: column, C18 silica gel; mobile phase, ACN in water (10 mM NH₄HCO₃), 20% to 40% gradient in 20 min; detector, UV 254 nm to afford 1-(1,1-dioxo-1λ⁶-thian-4-yl)ethyl 4-methylbenzenesulfonate (2.5 g, 78.85%) as a white solid.

2-[8-(2-chlorophenyl)-7-(4-chlorophenyl)-3-[(1S)-1-(1,1-dioxo-1λ⁶-thian-4-yl)ethyl]-2,6-dioxopurin-1-yl]acetamide and 2-[8-(2-chlorophenyl)-7-(4-chlorophenyl)-3-[(1R)-1-(1,1-dioxo-1λ⁶-thian-4-yl)ethyl]-2,6-dioxopurin-1-yl]acetamide. To a stirred solution of 2-[8-(2-chlorophenyl)-7-(4-chlorophenyl)-2,6-dioxo-3H-purin-1-yl]acetamide (100.00 mg, 0.232 mmol, 1.00 equiv) and Cs₂CO₃ (227.18 mg, 0.697 mmol, 3.00 equiv) in DMF (10.00 mL) was added 1-(1,1-dioxo-1λ⁶-thian-4-yl)ethyl 4-methylbenzenesulfonate (115.90 mg, 0.349 mmol, 1.50 equiv) at room temperature. The resulting mixture was stirred for 30 h at 40° C. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 40% B to 70% B in 40 min; 254/220 nm. The fractions containing the desired product were collected at 55% B and concentrated under reduced pressure. The crude product was purified by Prep-HPLC to afford 2-[8-(2-chlorophenyl)-7-(4-chlorophenyl)-3-[(1S)-1-(1,1-dioxo-1λ⁶-thian-4-yl)ethyl]-2,6-dioxopurin-1-yl]acetamide (70 mg, 51.01%) and 2-[8-(2-chlorophenyl)-7-(4-chlorophenyl)-3-[(1R)-1-(1,1-dioxo-1λ⁶-thian-4-yl)ethyl]-2,6-dioxopurin-1-yl]acetamide (70 mg, 51.01%) as a white solid.

### Example 46. Preparation of Compound 172

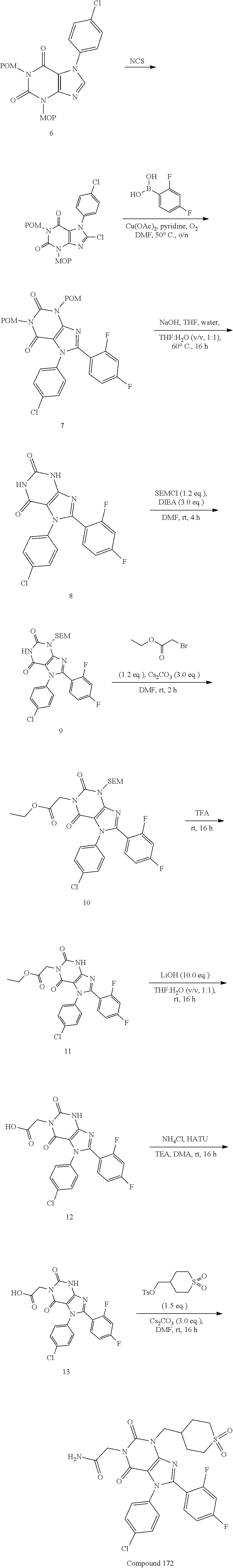

[8-chloro-7-(4-chlorophenyl)-3-[[(2,2-dimethylpropanoyl)oxy]methyl]-2,6-dioxopurin-1-yl]methyl 2,2-dimethylpropanoate. To a stirred solution of [7-(4-chlorophenyl)-3-[[(2,2-dimethylpropanoyl)oxy]methyl]-2,6-dioxopurin-1-yl]methyl 2,2-dimethylpropanoate (5.00 g, 10.185 mmol, 1.00 equiv) and NCS (2.04 g, 15.277 mmol, 1.50 equiv) in DMF (20.00 mL) at room temperature. The resulting mixture was stirred for overnight at room temperature. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 50% B to 80% B in 40 min; 254/220 nm. The fractions containing the desired product were collected at 70% B and concentrated under reduced pressure. To afford [8-chloro-7-(4-chlorophenyl)-3-[[(2,2-dimethylpropanoyl)oxy]methyl]-2,6-dioxopurin-1-yl]methyl 2,2-dimethylpropanoate (4.2 g, 78.49%) as a light yellow solid.

[7-(4-chlorophenyl)-8-(2,4-difluorophenyl)-3-[[(2,2-dimethylpropanoyl)oxy]methyl]-2,6-dioxopurin-1-yl]methyl 2,2-dimethylpropanoate. To a stirred solution of [8-chloro-7-(4-chlorophenyl)-3-[[(2,2-dimethylpropanoyl)oxy]methyl]-2,6-dioxopurin-1-yl]methyl 2,2-dimethylpropanoate (1.00 g, 1.903 mmol, 1.00 equiv) and Cu(AcO)₂ (345.72 mg, 1.903 mmol, 1.00 equiv) in DMF (20.00 mL) were added 2,4-difluorophenylboronic acid (691.30 mg, 4.378 mmol, 2.30 equiv) and Pyridine (602.23 mg, 7.614 mmol, 4.00 equiv) at room temperature under O₂ atmosphere. The resulting mixture was stirred for overnight at 50° C. under 02 atmosphere. The reaction was monitored by LCMS. The mixture was allowed to cool down to room temperature. The resulting mixture was extracted with EtOAc (3×50 mL). The combined organic layers were washed with brine (3×100 mL), dried over anhydrous Na₂SO₄. After filtration, the filtrate was concentrated under reduced pressure. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 50% B to 80% B in 40 min; 254/220 nm. The fractions containing the desired product were collected at 68% B and concentrated under reduced pressure. To afford [7-(4-chlorophenyl)-8-(2,4-difluorophenyl)-3-[[(2,2-dimethylpropanoyl)oxy]methyl]-2,6-dioxopurin-1-yl]methyl 2,2-dimethylpropanoate (600 mg, 52.27%) as an light yellow solid.

7-(4-chlorophenyl)-8-(2,4-difluorophenyl)-1,3-dihydropurine-2,6-dione. To a stirred solution of [7-(4-chlorophenyl)-8-(2,4-difluorophenyl)-3-[[(2,2-dimethylpropanoyl)oxy]methyl]-2,6-dioxopurin-1-yl]methyl 2,2-dimethylpropanoate (500.00 mg, 0.829 mmol, 1.00 equiv) and H₂O (6.00 mL) in THF (6.00 mL) was added NaOH (132.66 mg, 3.317 mmol, 4.00 equiv) at room temperature. The resulting mixture was stirred for overnight at 60° C. The reaction was monitored by LCMS. The mixture was allowed to cool down to room temperature. The mixture was acidified to pH 5 with HCl (aq.). The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 20% B to 50% B in 40 min; 254/220 nm. The fractions containing the desired product were collected at 35% B and concentrated under reduced pressure to afford 7-(4-chlorophenyl)-8-(2,4-difluorophenyl)-1,3-dihydropurine-2,6-dione (250 mg, 80.46%) as a white solid.

7-(4-chlorophenyl)-8-(2,4-difluorophenyl)-3-[[2-(trimethylsilyl)ethoxy]methyl]-1H-purine-2,6-dione. To a stirred solution of 7-(4-chlorophenyl)-8-(2,4-difluorophenyl)-1,3-dihydropurine-2,6-dione (250.00 mg, 0.667 mmol, 1.00 equiv) and DIEA (258.67 mg, 2.001 mmol, 3.00 equiv) in DMF (5.00 mL) were added SEM-Cl (133.47 mg, 0.801 mmol, 1.20 equiv) at room temperature. The resulting mixture was stirred for 4 h at room temperature. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 40% B to 70% B in 40 min; 254/220 nm. The fractions containing the desired product were collected at 65% B and concentrated under reduced pressure. To afford 7-(4-chlorophenyl)-8-(2,4-difluorophenyl)-3-[[2-(trimethylsilyl)ethoxy]methyl]-1H-purine-2,6-dione (250 mg, 74.21%) as a light yellow solid.

Ethyl 2-[7-(4-chlorophenyl)-8-(2,4-difluorophenyl)-2,6-dioxo-3-[[2-(trimethylsilyl)ethoxy]methyl]purin-1-yl]acetate. To a stirred solution of 7-(4-chlorophenyl)-8-(2,4-difluorophenyl)-3-[[2-(trimethylsilyl)ethoxy]methyl]-1H-purine-2,6-dione (250.00 mg, 0.495 mmol, 1.00 equiv) and Cs₂CO₃ (483.90 mg, 1.485 mmol, 3.00 equiv) in DMF (6.00 mL) was added ethyl bromoacetate (165.35 mg, 0.990 mmol, 2.00 equiv) at room temperature. The resulting mixture was stirred for 2 h at room temperature. The resulting mixture was extracted with EtOAc (3×50 mL). The combined organic layers were washed with brine (3×50 mL), dried over anhydrous Na₂SO₄F. After filtration, the filtrate was concentrated under reduced pressure. The crude product was used in the next step directly without further purification.

Ethyl 2-[7-(4-chlorophenyl)-8-(2,4-difluorophenyl)-2,6-dioxo-3H-purin-1-yl]acetate. To a stirred solution of ethyl 2-[7-(4-chlorophenyl)-8-(2,4-difluorophenyl)-2,6-dioxo-3-[[2-(trimethylsilyl)ethoxy]methyl]purin-1-yl]acetate (250.00 mg, 0.423 mmol, 1.00 equiv) in DCM (8.00 mL) was added TFA (2.00 mL) at room temperature. The resulting mixture was stirred for 16 h at room temperature. The reaction was monitored by LCMS. The mixture was basified to pH 7 with saturated NaHCO₃ (aq.). The residue was purified by reverse flash chromatography with the following conditions: Column: C18 Column 120 g; Mobile Phase A: Water (10 mM NH₄HCO₃), Mobile Phase B: ACN; Flow rate: 80 mL/min; Gradient: 30% B to 70% B in 40 min; 254/220 nm. The fractions containing the desired product were collected at 42% B and concentrated under reduced pressure. To afford ethyl 2-[7-(4-chlorophenyl)-8-(2,4-difluorophenyl)-2,6-dioxo-3H-purin-1-yl]acetate (150 mg, 76.96%) as a white solid.

[7-(4-chlorophenyl)-8-(2,4-difluorophenyl)-2,6-dioxo-3H-purin-1-yl]acetic acid. To a stirred solution of ethyl 2-[7-(4-chlorophenyl)-8-(2,4-difluorophenyl)-2,6-dioxo-3H-purin-1-yl]acetate (150.00 mg, 0.326 mmol, 1.00 equiv) and LiOH (77.95 mg, 3.255 mmol, 10 equiv) in THF (5.00 mL) was added H₂O (5.00 mL) at room temperature. The resulting mixture was stirred for 16 h at room temperature. The reaction was monitored by LCMS. The mixture was acidified to pH 5 with HCl (aq.). The residue was purified by reverse flash chromatography with the following conditions: Column: C18 Column 120 g; Mobile Phase A: Water (10 mM NH₄HCO₃), Mobile Phase B: ACN; Flow rate: 80 mL/min; Gradient: 20% B to 50% B in 40 min; 254/220 nm. The fractions containing the desired product were collected at 35% B and concentrated under reduced pressure. To afford [7-(4-chlorophenyl)-8-(2,4-difluorophenyl)-2,6-dioxo-3H-purin-1-yl]acetic acid (120 mg, 85.19%) as a white solid.

2-[7-(4-chlorophenyl)-8-(2,4-difluorophenyl)-2,6-dioxo-3H-purin-1-yl]acetamide. A solution of [7-(4-chlorophenyl)-8-(2,4-difluorophenyl)-2,6-dioxo-3H-purin-1-yl]acetic acid (120.00 mg, 0.277 mmol, 1.00 equiv) and HATU (158.15 mg, 0.416 mmol, 1.5 equiv) in DMA (5.00 mL) was stirred for 30 min at room temperature. To the above mixture was added NH₄Cl (44.50 mg, 0.832 mmol, 3 equiv) at room temperature. The resulting mixture was stirred for additional 30 min at room temperature. To the above mixture was added TEA (84.17 mg, 0.832 mmol, 3 equiv) at room temperature. The resulting mixture was stirred for additional 16 h at room temperature. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 20% B to 50% B in 40 min; 254/220 nm. The fractions containing the desired product were collected at 35% B and concentrated under reduced pressure. To afford 2-[7-(4-chlorophenyl)-8-(2,4-difluorophenyl)-2,6-dioxo-3H-purin-1-yl]acetamide (100 mg, 83.52%) as a white solid. 2-[7-(4-chlorophenyl)-8-(2,4-difluorophenyl)-3-[(1,1-dioxo-1λ⁶-thian-4-yl)methyl]-2,6-dioxopurin-1-yl]acetamide. To a stirred solution of 2-[7-(4-chlorophenyl)-8-(2,4-difluorophenyl)-2,6-dioxo-3H-purin-1-yl]acetamide (110.00 mg, 0.255 mmol, 1.00 equiv) and Cs₂CO₃ (249.02 mg, 0.764 mmol, 3.00 equiv) in DMF (6.00 mL) was added (1,1-dioxo-1λ⁶-thian-4-yl)methyl 4-methylbenzenesulfonate (121.67 mg, 0.382 mmol, 1.50 equiv) at room temperature. The resulting mixture was stirred for 16 h at room temperature. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 80 mL/min; Gradient: 30% B to 50% B in 40 min; 254/220 nm. The fractions containing the desired product were collected at 36% B and concentrated under reduced pressure. To afford 2-[7-(4-chlorophenyl)-8-(2,4-difluorophenyl)-3-[(1,1-dioxo-1 lambda6-thian-4-yl)methyl]-2,6-dioxopurin-1-yl]acetamide (100 mg, 67.91%) as a white solid. Compound 173 was prepared by the methods and scheme described for Compound 172 using the appropriate reagents.

### Example 47. Preparation of Compound 171

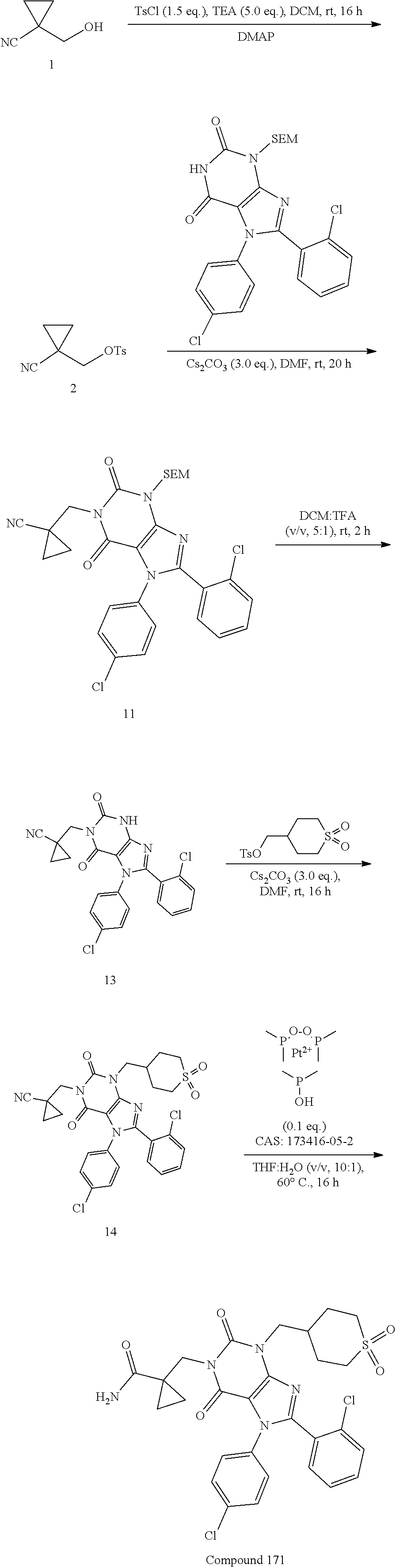

(1-cyanocyclopropyl)methyl 4-methylbenzenesulfonate. To a stirred solution of 1-(hydroxymethyl)cyclopropane-1-carbonitrile (2.00 g, 20.594 mmol, 1.00 equiv) and DMAP (251.59 mg, 2.059 mmol, 0.10 equiv) in DCM (30.00 mL) were added TEA (10.42 g, 102.969 mmol, 5.00 equiv) and TsCl (5.89 g, 30.891 mmol, 1.50 equiv) at 0° C. The resulting mixture was stirred for overnight at room temperature. The reaction was monitored by LCMS. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 20% B to 50% B in 40 min; 254/220 nm. The fractions containing the desired product were collected at 45% B and concentrated under reduced pressure. To afford (1-cyanocyclopropyl)methyl 4-methylbenzenesulfonate (4 g, 77.29%) as an off-white solid.

1-[[8-(2-chlorophenyl)-7-(4-chlorophenyl)-2,6-dioxo-3-[[2-(trimethylsilyl)ethoxy]methyl]purin-1-yl]methyl]cyclopropane-1-carbonitrile. To a stirred solution of 8-(2-chlorophenyl)-7-(4-chlorophenyl)-3-[[2-(trimethylsilyl)ethoxy]methyl]-1H-purine-2,6-dione (150.00 mg, 0.298 mmol, 1.00 equiv) and Cs₂CO₃ (291.22 mg, 0.894 mmol, 3.00 equiv) in DMF (5.00 mL) was added (1-cyanocyclopropyl)methyl 4-methylbenzenesulfonate (112.31 mg, 0.447 mmol, 1.50 equiv) at room temperature. The resulting mixture was stirred for 20 h at room temperature. The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 50% B to 80% B in 40 min; 254/220 nm. The fractions containing the desired product were collected at 72% B and concentrated under reduced pressure. To afford 1-[[8-(2-chlorophenyl)-7-(4-chlorophenyl)-2,6-dioxo-3-[[2-(trimethylsilyl)ethoxy]methyl]purin-1-yl]methyl]cyclopropane-1-carbonitrile (160 mg, 92.18%) as a white solid.

1-[[8-(2-chlorophenyl)-7-(4-chlorophenyl)-2,6-dioxo-3H-purin-1-yl]methyl]cyclopropane-1-carbonitrile. To a stirred solution of 1-[[8-(2-chlorophenyl)-7-(4-chlorophenyl)-2,6-dioxo-3-[[2-(trimethylsilyl)ethoxy]methyl]purin-1-yl]methyl]cyclopropane-1-carbonitrile (160.00 mg, 0.275 mmol, 1.00 equiv) in DCM (5.00 mL) was added TFA (1.00 mL) at room temperature. The resulting mixture was stirred for 2 h at room temperature. The reaction was monitored by LCMS. The mixture was basified to pH 6 with saturated NaHCO₃ (aq.). The residue was purified by reverse flash chromatography with the following conditions: Column: C18 Column 330 g; Mobile Phase A: Water (10 mM NH₄HCO₃), Mobile Phase B: ACN; Flow rate: 80 mL/min; Gradient: 40% B to 60% B in 40 min; 254/220 nm. The fractions containing the desired product were collected at 47% B and concentrated under reduced pressure. To afford 1-[[8-(2-chlorophenyl)-7-(4-chlorophenyl)-2,6-dioxo-3H-purin-1-yl]methyl]cyclopropane-1-carbonitrile (120 mg, 96.60%) as a white solid.

1-[[8-(2-chlorophenyl)-7-(4-chlorophenyl)-3-[(1,1-dioxo-1λ⁶-thian-4-yl)methyl]-2,6-dioxopurin-1-yl]methyl]cyclopropane-1-carbonitrile. To a stirred solution of 1-[[8-(2-chlorophenyl)-7-(4-chlorophenyl)-2,6-dioxo-3H-purin-1-yl]methyl]cyclopropane-1-carbonitrile (0.120 g, 0.265 mmol) and (1,1-dioxo-1λ⁶-thian-4-yl)methyl 4-methylbenzenesulfonate (0.127 g, 0.398 mmol) was added cesium carbonate (0.259 g, 0.796 mmol) in N,N-dimethylformamide (5.00 mL) at ambient temperature. The resulting mixture was stirred at ambient temperature for 16 h. The reaction mixture was diluted by water (10.0 mL) and extracted with ethyl acetate (3×10 mL). The combined organic fractions were washed with brine (2×10 mL), dried with anhydrous sodium sulfate and filtered. The filtrate was concentrated under reduced pressure. The residue was purified by reversed phase flash chromatography with the following conditions: Column: C18, 20-40 μm, 120 g; Mobile Phase A: Water (plus 10 mM NH₄HCO₃); Mobile Phase B: ACN; Flow rate: 80 mL/min; Gradient (B): 30% to 60% in 40 min; Detector: UV 220 nm/254 nm. The fractions containing desired product were collected at 47% B and concentrated under reduced pressure to afford 1-[[8-(2-chlorophenyl)-7-(4-chlorophenyl)-3-[(1,1-dioxo-1λ⁶-thian-4-yl)methyl]-2,6-dioxopurin-1-yl]methyl]cyclopropane-1-carbonitrile (0.100 g, 63%) as a white solid

1-[[8-(2-chlorophenyl)-7-(4-chlorophenyl)-3-[(1,1-dioxo-1λ⁶-thian-4-yl)methyl]-2,6-dioxopurin-1-yl]methyl]cyclopropane-1-carboxamide. To a stirred solution of 1-[[8-(2-chlorophenyl)-7-(4-chlorophenyl)-3-[(1,1-dioxo-1λ⁶-thian-4-yl)methyl]-2,6-dioxopurin-1-yl]methyl]cyclopropane-1-carbonitrile (100.00 mg, 0.167 mmol, 1.00 equiv) and platinum (2+) dimethylphosphinous acid didimethylphosphinite (7.14 mg, 0.017 mmol, 0.10 equiv) in THF (5.00 mL) was added H₂O (0.50 mL) at room temperature. The resulting mixture was stirred for 16 h at 60° C. The reaction was monitored by LCMS. The residue was purified by reverse flash chromatography with the following conditions: Column: C18 Column 120 g; Mobile Phase A: Water (10 mM NH₄HCO₃), Mobile Phase B: ACN; Flow rate: 80 mL/min; Gradient: 30% B to 60% B in 40 min; 254/220 nm. The fractions containing the desired product were collected at 48% B and concentrated under reduced pressure. To afford 1-[[8-(2-chlorophenyl)-7-(4-chlorophenyl)-3-[(1,1-dioxo-1λ⁶-thian-4-yl)methyl]-2,6-dioxopurin-1-yl]methyl]cyclopropane-1-carboxamide (70 mg, 67.96%) as a white solid.

### Example 48. Preparation of Compound 149

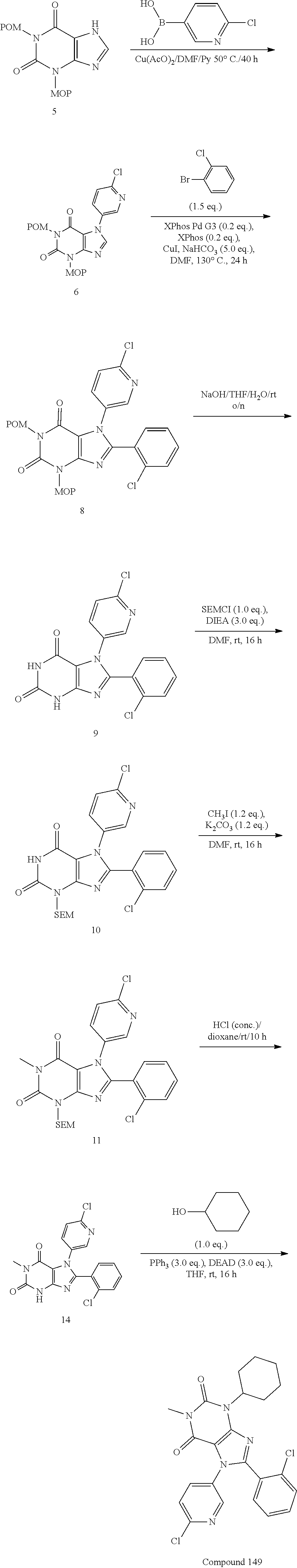

[7-(6-chloropyridin-3-yl)-3-[[(2,2-dimethylpropanoyl)oxy]methyl]-2,6-dioxopurin-1-yl]methyl 2,2-dimethylpropanoate. To a stirred solution of (3-[[(2,2-dimethylpropanoyl)oxy]methyl]-2,6-dioxo-7H-purin-1-yl)methyl 2,2-dimethylpropanoate (10.00 g, 26.288 mmol, 1.00 equiv) and Cu(AcO)₂ (4.77 g, 26.288 mmol, 1.00 equiv) in DMF (500.00 mL) were added 6-chloropyridin-3-ylboronic acid (9.51 g, 60.463 mmol, 2.30 equiv) and Pyridine (6.24 g, 78.864 mmol, 3.00 equiv) at room temperature. The resulting mixture was stirred for 40 h at 50° C. under 02 atmosphere. The reaction was monitored by LCMS. The resulting mixture was extracted with EtOAc (3×2 L). The combined organic layers were washed with brine (3×5 L), dried over anhydrous Na₂SO₄. After filtration, the filtrate was concentrated under reduced pressure. The residue was purified by trituration with MeCN (400 mL). to afford [7-(6-chloropyridin-3-yl)-3-[[(2,2-dimethylpropanoyl)oxy]methyl]-2,6-dioxopurin-1-yl]methyl 2,2-dimethylpropanoate (3.5 g, 27.06%) as a white solid.

[8-(2-chlorophenyl)-7-(6-chloropyridin-3-yl)-3-[[(2,2-dimethylpropanoyl)oxy]methyl]-2,6-dioxopurin-1-yl]methyl 2,2-dimethylpropanoate. To a stirred solution of [7-(6-chloropyridin-3-yl)-3-[[(2,2-dimethylpropanoyl)oxy]methyl]-2,6-dioxopurin-1-yl]methyl 2,2-dimethylpropanoate (3.50 g, 7.115 mmol, 1.00 equiv) and NaHCO₃ (2.99 g, 35.574 mmol, 5.00 equiv) in DMF (80.00 mL) were added 2-bromochlorobenzene (2.04 g, 10.672 mmol, 1.50 equiv) X-Phos (0.68 g, 1.423 mmol, 0.20 equiv) XPhos Pd G3 (1.20 g, 1.423 mmol, 0.20 equiv) and CuI (5.42 g, 28.459 mmol, 4 equiv) at 130° C. under nitrogen atmosphere. The resulting mixture was stirred at 130° C. for 24 h under nitrogen atmosphere. The reaction was monitored by LCMS. The resulting mixture was extracted with EtOAc (3×200 mL). The combined organic layers were washed with brine (3×600 mL), dried over anhydrous Na₂SO₄. After filtration, the filtrate was concentrated under reduced pressure. The residue was purified by silica gel column chromatography, Column: silica-CS Column 330 g; Mobile Phase A: PE, Mobile Phase B: EA; Flow rate: 70 mL/min; Gradient: 20% B to 40% B in 40 min; 254/280 nm. The fractions containing the desired product were collected at 28% B and concentrated under reduced pressure. To afford [8-(2-chlorophenyl)-7-(6-chloropyridin-3-yl)-3-[[(2,2-dimethylpropanoyl)oxy]methyl]-2,6-dioxopurin-1-yl]methyl 2,2-dimethylpropanoate (1.2 g, 28.00%) as a white solid.

8-(2-chlorophenyl)-7-(6-chloropyridin-3-yl)-1,3-dihydropurine-2,6-dione. To a stirred solution of [8-(2-chlorophenyl)-7-(6-chloropyridin-3-yl)-3-[[(2,2-dimethylpropanoyl)oxy]methyl]-2,6-dioxopurin-1-yl]methyl 2,2-dimethylpropanoate (1.00 g, 1.660 mmol, 1.00 equiv) in THF (5.00 mL) and H₂O (5.00 mL) was added caustic soda (199.17 mg, 4.980 mmol, 3.00 equiv) at room temperature. The resulting mixture was stirred for approx. 10 h at room temperature. The reaction was monitored by LCMS. The mixture was acidified to pH 6 with HCl (aq. 1N). The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18 Column 120 g; Mobile Phase A: Water (10 mM AcOH), Mobile Phase B: ACN; Flow rate: 60 mL/min; Gradient: 55% B to 80% B in 20 min; 254/220 nm). The fractions containing the desired product were collected at 59% B and concentrated under reduced pressure to afford 8-(2-chlorophenyl)-7-(6-chloropyridin-3-yl)-1,3-dihydropurine-2,6-dione (256 mg, 41.22%) as a white solid.

8-(2-chlorophenyl)-7-(6-chloropyridin-3-yl)-3-[[2-(trimethylsilyl)ethoxy]methyl]-1H-purine-2,6-dione. To a stirred solution of 8-(2-chlorophenyl)-7-(6-chloropyridin-3-yl)-1,3-dihydropurine-2,6-dione (256.00 mg, 0.684 mmol, 1.00 equiv) in DMF (10.00 mL) were added DIEA (265.27 mg, 2.052 mmol, 3.00 equiv) and [2-(chloromethoxy)ethyl]trimethylsilane (114.06 mg, 0.684 mmol, 1.00 equiv). The resulting mixture was stirred for 16 h at room temperature. The reaction was monitored by LCMS. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18 Column 120 g; Mobile Phase A: Water (10 mM NH₄HCO₃), Mobile Phase B: ACN; Flow rate: 60 mL/min; Gradient: 60% B to 80% B in 25 min; 254/220 nm Column). The fractions containing the desired product were collected at 67% B and concentrated under reduced pressure to afford 8-(2-chlorophenyl)-7-(6-chloropyridin-3-yl)-3-[[2-(trimethylsilyl)ethoxy]methyl]-1H-purine-2,6-dione (216 mg, 62.59%) as a white solid.

8-(2-chlorophenyl)-7-(6-chloropyridin-3-yl)-1-methyl-3-[[2-(trimethylsilyl)ethoxy]methyl]purine-2,6-dione. To a stirred solution of 8-(2-chlorophenyl)-7-(6-chloropyridin-3-yl)-3-[[2-(trimethylsilyl)ethoxy]methyl]-1H-purine-2,6-dione (216.00 mg, 0.428 mmol, 1.00 equiv) in DMF (5.00 mL) was added K₂CO₃ (71.02 mg, 0.514 mmol, 1.20 equiv) and methyl iodide (73.00 mg, 1.20 equiv) at room temperature. The resulting mixture was stirred for 16 h at room temperature. The reaction was monitored by LCMS. The resulting mixture was extracted with EtOAc (3×50 mL). The combined organic layers were washed with brine (1×100 mL), dried over anhydrous Na₂SO₄. After filtration, the filtrate was concentrated under reduced pressure. The crude product was used in the next step without further purification.

8-(2-chlorophenyl)-7-(6-chloropyridin-3-yl)-1-methyl-3H-purine-2,6-dione. A solution of 8-(2-chlorophenyl)-7-(6-chloropyridin-3-yl)-1-methyl-3-[[2-(trimethylsilyl)ethoxy]methyl]purine-2,6-dione (220.00 mg, 0.424 mmol, 1.00 equiv) and hydrogen chloride (10 mL) in 1,4-dioxane (5.00 mL) was stirred for 10 h at room temperature. The reaction was monitored by LCMS. The mixture was basified to pH 6 with saturated NaHCO₃ (aq.). The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18 Column 120 g; Mobile Phase A: Water (10 mM NH₄HCO₃), Mobile Phase B: ACN; Flow rate: 60 mL/min; Gradient: 35% B to 60% B in 30 min; 254/220 nm). The fractions containing the desired product were collected at 39% B and concentrated under reduced pressure to afford 8-(2-chlorophenyl)-7-(6-chloropyridin-3-yl)-1-methyl-3H-purine-2,6-dione (121 mg, 73.45%) as a white solid.

8-(2-chlorophenyl)-7-(6-chloropyridin-3-yl)-3-cyclohexyl-1-methylpurine-2,6-dione. To a stirred solution of 8-(2-chlorophenyl)-7-(6-chloropyridin-3-yl)-1-methyl-3H-purine-2,6-dione (121.00 mg, 1 equiv) and PPh₃ (245.00 mg, 3.00 equiv) in THF were added cyclohexanol (31.00 mg, 1.00 equiv) and DEAD (163.00 mg, 3.00 equiv) dropwise at 0° C. under nitrogen atmosphere. The resulting mixture was stirred for 16 h at room temperature under nitrogen atmosphere. The reaction was monitored by LCMS. The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by reverse phase flash with the following conditions (Column: C18 Column 120 g; Mobile Phase A: Water (10 mM AcOH), Mobile Phase B: ACN; Flow rate: 60 mL/min; Gradient: 60% B to 80% B in 25 min; 254/220 nm). The fractions containing the desired product were collected at 68% B and concentrated under reduced pressure to afford 8-(2-chlorophenyl)-7-(6-chloropyridin-3-yl)-3-cyclohexyl-1-methylpurine-2,6-dione (50 mg, 34.11%) as a white solid.

### Example 49. Preparation of Compounds 189 and 190

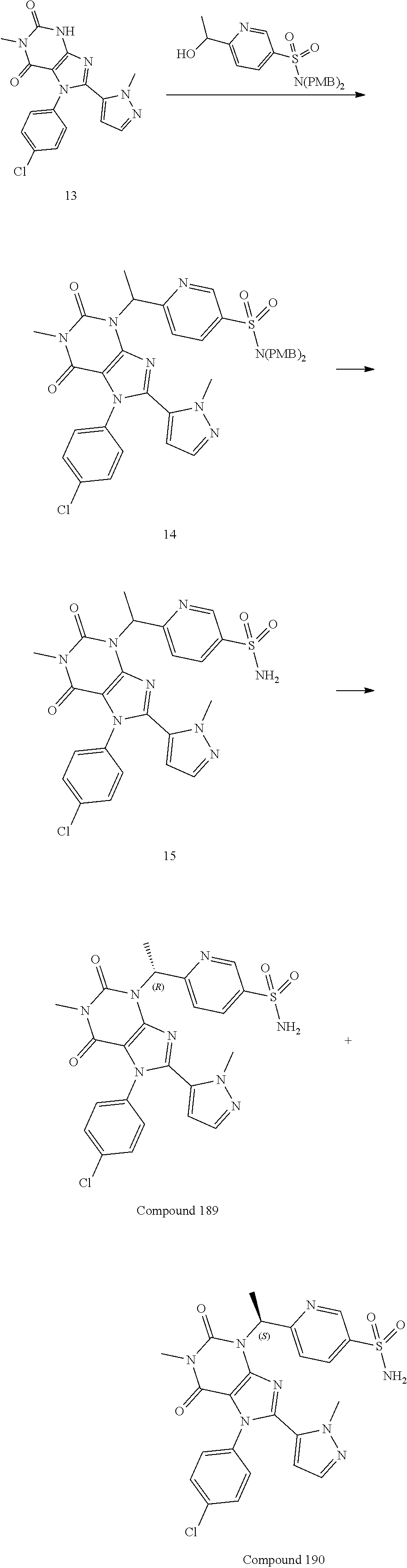

6-[1-[7-(4-chlorophenyl)-1-methyl-8-(2-methylpyrazol-3-yl)-2,6-dioxopurin-3-yl]ethyl]-N,N-bis[(4-methoxyphenyl)methyl]pyridine-3-sulfonamide. To a stirred solution of 7-(4-chlorophenyl)-1-methyl-8-(2-methylpyrazol-3-yl)-3H-purine-2,6-dione (360.00 mg, 1.009 mmol, 1.00 equiv) and 6-(1-hydroxyethyl)-N,N-bis[(4-methoxyphenyl)methyl]pyridine-3-sulfonamide (535.84 mg, 1.211 mmol, 1.20 equiv) in THF (5.00 mL) were added DIAD (408.08 mg, 2.018 mmol, 2.00 equiv) and PPh₃ (529.32 mg, 2.018 mmol, 2.00 equiv) at room temperature under nitrogen atmosphere. The resulting mixture was stirred for 16 h at room temperature under nitrogen atmosphere. The resulting mixture was concentrated under reduced pressure to afford 6-[1-[7-(4-chlorophenyl)-1-methyl-8-(2-methylpyrazol-3-yl)-2,6-dioxopurin-3-yl]ethyl]-N,N-bis[(4-methoxyphenyl)methyl]pyridine-3-sulfonamide (420 mg, crude) as a white solid.

6-[(1S)-1-[7-(4-chlorophenyl)-1-methyl-8-(2-methylpyrazol-3-yl)-2,6-dioxopurin-3-yl]ethyl]pyridine-3-sulfonamide and 6-[(1R)-1-[7-(4-chlorophenyl)-1-methyl-8-(2-methylpyrazol-3-yl)-2,6-dioxopurin-3-yl]ethyl]pyridine-3-sulfonamide. Into a 50 mL round-bottom flask were added 6-[1-[7-(4-chlorophenyl)-1-methyl-8-(2-methylpyrazol-3-yl)-2,6-dioxopurin-3-yl]ethyl]-N,N-bis[(4-methoxyphenyl)methyl]pyridine-3-sulfonamide (420.00 mg, 0.538 mmol, 1.00 equiv) and TFA (10 mL) at room temperature. The resulting mixture was stirred for 1 h at 60° C. The resulting mixture was concentrated under reduced pressure. The residue was purified by silica gel column chromatography, eluted with PE/EtOAc (1:2) to afford a crude product. The crude product (240 mg) was purified by Prep-HPLC with the following conditions (Column: CHIRALPAK IC, 2*25 cm, 5 um; Mobile Phase A: Hex (10 mM NH₃), Mobile Phase B: EtOH:DCM=1:1—HPLC; Flow rate: 16 mL/min; Gradient: 70 B to 70 B in 25 min; 220/254 nm; RT1: 7.955; RT2: 21.868; Injection Volume: 2.667 mL; Number Of Runs: 3) to afford 6-[(1S)-1-[7-(4-chlorophenyl)-1-methyl-8-(2-methylpyrazol-3-yl)-2,6-dioxopurin-3-yl]ethyl]pyridine-3-sulfonamide (82 mg) as an off-white solid and 6-[(1R)-1-[7-(4-chlorophenyl)-1-methyl-8-(2-methylpyrazol-3-yl)-2,6-dioxopurin-3-yl]ethyl]pyridine-3-sulfonamide (81.7 mg) as an off-white solid.

The following compounds were prepared using the methods described above:

| # | Structure | NMR | MS |
|---|---|---|---|
| 100 | 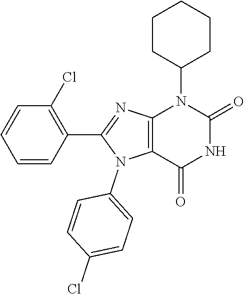 [SMILES: O=c1[nH]c(=O)n(C2CCCCC2)c2nc(-c3ccccc3Cl)n(-c3ccc(Cl)cc3)c12] | ¹H NMR (400 MHz, Chloroform-d) chemical shifts 7.93 (s, 1H), 7.45-7.38 (m, 3H), 7.36- 7.29 (m, 3H), 7.21-7.13 (m, 2H), 4.86-4.79 (m, 1H), 2.65-2.55 (m, 2H), 1.94-1.83 (m, 4H), 1.70 (d, J = 12.9 Hz, 1H), 1.51-1.29 (m, 2H). | 455 |
| 101 | 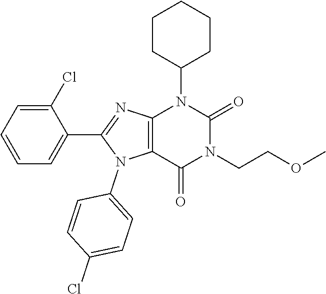 [SMILES: COCCn1c(=O)c2c(nc(-c3ccccc3Cl)n2-c2ccc(Cl)cc2)n(C2CCCCC2)c1=O] | ¹H NMR (400 MHz, Methanol-d4) chemical shifts 7.55-7.53 (m, 1H), 7.50-7.44 (m, 2H), 7.42-7.35 (m, 3H), 7.33-7.29 (m, 2H), 4.88- 4.80 (m, 1H), 4.20 (t, J = 6.0 Hz, 2H), 3.62 (t, J = 6.0 Hz, 2H), 3.35 (s, 3H), 2.69-2.59 (m, 2H), 1.93 (d, J = 13.2 Hz, 2H), 1.84 (d, J = 12.3 Hz, 2H), 1.73 (d, J = 13.0 Hz, 1H), 1.53- 1.41 (m, 2H), 1.38-1.28 (s, 1H). | 513 |
| 102 | 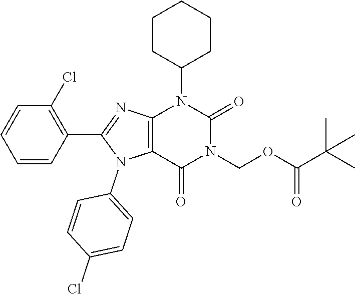 [SMILES: CC(C)(C)C(=O)OCn1c(=O)c2c(nc(-c3ccccc3Cl)n2-c2ccc(Cl)cc2)n(C2CCCCC2)c1=O] | ¹H NMR (400 MHz, Chloroform-d) chemical shifts 7.47-7.31 (m, 6H), 7.21 (d, J = 8.3 Hz, 2H), 6.21 (s, 2H), 4.89-4.83 (m, 1H), 2.53- 2.40 (m, 2H), 1.84 (d, J = 12.8 Hz, 2H), 1.66- 1.64 (m, 2H), 1.47-1.24 (m, 14H). | 569 |
| 103 | 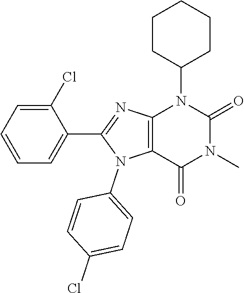 [SMILES: Cn1c(=O)c2c(nc(-c3ccccc3Cl)n2-c2ccc(Cl)cc2)n(C2CCCCC2)c1=O] | ¹NMR (400 MHz, Chloroform-d) chemical shifts 7.43-7.36 (m, 3H), 7.34-7.30 (m, 3H), 7.20-717 (m, 2H), 4.92-4.85 (m, 1H), 3.40 (s, 3H), 2.68-2.56 (m, 2H), 1.94-1.83 (m, 4H), 1.70-1.65 (m, 1H), 1.47 (q, J = 12.9 Hz, 2H), 1.39-1.25 (m, 1H). | 469 |
| 104 | 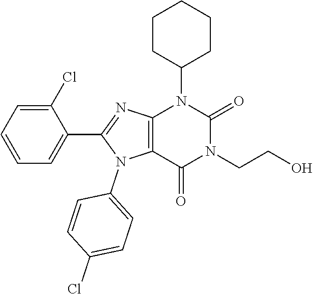 [SMILES: O=c1c2c(nc(-c3ccccc3Cl)n2-c2ccc(Cl)cc2)n(C2CCCCC2)c(=O)n1CCO] | ¹H NMR (400 MHz, Methanol-d4) chemical shifts 7.56-7.51 (m, 1H), 7.51-7.44 (m, 2H), 7.43-7.38 (m, 1H), 7.36 (d, J = 8.8 Hz, 2H), 7.30 (d, J = 8.8 Hz, 2H), 4.92-4.85 (m, 1H), 4.15 (t, J = 6.1 Hz, 2H), 3.75 (t, J = 6.2 Hz, 2H), 2.69-2.60 (m, 2H), 1.93 (d, J = 13.4 Hz, 2H), 1.85 (d, J = 12.1 Hz, 2H), 1.73 (d, J = 12.5 Hz, 1H), 1.48 (q, J = 13.3 Hz, 2H), 1.35- 1.30 (m, 1H). | 499 |
| 105 | 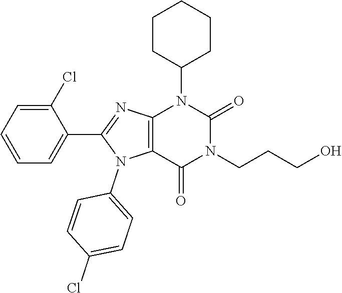 [SMILES: O=c1c2c(nc(-c3ccccc3Cl)n2-c2ccc(Cl)cc2)n(C2CCCCC2)c(=O)n1CCCO] | ¹H NMR (400 MHz, Methanol-d4) chemical shifts 7.53 (d, J = 7.5 Hz, 1H), 7.49-7.45 (m, 2H), 7.41 (d, J = 8.2 Hz, 1H), 7.37 (d, J = 8.8 Hz, 2H), 7.31 (d, J = 8.8 Hz, 2H), 4.90-4.82 (m, 1H), 4.08 (t, J = 7.0 Hz, 2H), 3.60 (t, J = 6.3 Hz, 2H), 2.71-2.61 (m, 2H), 1.91-1.95 (m, 2H), 1.87-1.82 (m, 4H), 1.74-1.71 (m, 1H). 1.53-1.43 (m, 2H), 1.40-1.29 (m, 1H). | 513 |
| 106 | 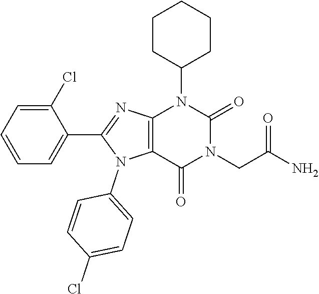 [SMILES: NC(=O)Cn1c(=O)c2c(nc(-c3ccccc3Cl)n2-c2ccc(Cl)cc2)n(C2CCCCC2)c1=O] | ¹H NMR (400 MHz, DMSO-d6) chemical shifts 7.65 (d, J = 7.5 Hz, 1H), 7.55 (s, 1H), 7.52-7.50 (m, 2H), 7.46-7.41 (m, 3H), 7.39- 7.31 (m, 2H), 7.07 (s, 1H), 4.77-4.71 (m, 1H), 4.39 (s, 2H), 1.84 (d, J = 12.7 Hz, 2H), 1.77- 1.63 (m, 4H), 1.42-1.15 (m, 4H). | 512 |
| 107 | 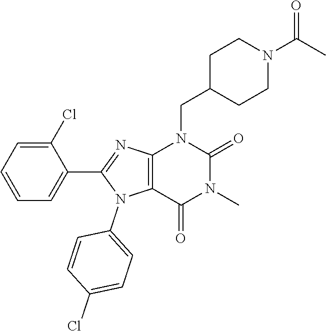 [SMILES: CC(=O)N1CCC(Cn2c(=O)n(C)c(=O)c3c2nc(-c2ccccc2Cl)n3-c2ccc(Cl)cc2)CC1] | ¹H NMR (400 MHz, Chloroform-d) chemical shifts 7.46-7.37 (m, 2H), 7.40-7.29 (m, 4H), 7.19 (d, J = 8.7 Hz, 2H), 4.64 (s, 1H), 4.16 (d, J = 7.1 Hz, 2H), 3.90 (s, 1H), 3.43 (s, 3H), 3.05 (s, 1H), 2.67 (s, 1H), 2.33 (s, 1H), 2.15 (s, 2H), 1.81 (s, 2H), 1.41 (d, J = 12.3 Hz, 2H). | 526 |
| 108 | 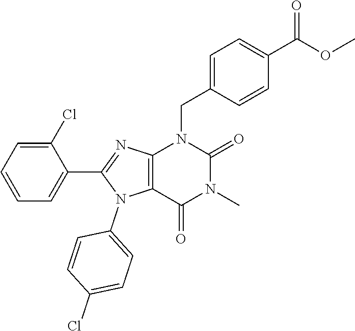 [SMILES: COC(=O)c1ccc(Cn2c(=O)n(C)c(=O)c3c2nc(-c2ccccc2Cl)n3-c2ccc(Cl)cc2)cc1] | ¹H NMR (400 MHz, Chloroform-d) chemical shifts 8.07-8.00 (m, 2H), 7.72-7.66 (m, 2H), 7.45-7.37 (m, 2H), 7.39-7.28 (m, 4H), 7.23- 7.15 (m, 2H), 5.44 (s, 2H), 3.93 (s, 3H), 3.41 (s, 3H). | 535 |
| 109 | 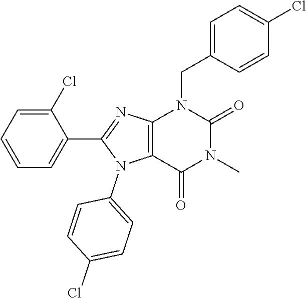 [SMILES: Cn1c(=O)c2c(nc(-c3ccccc3Cl)n2-c2ccc(Cl)cc2)n(Cc2ccc(Cl)cc2)c1=O] | ¹H NMR (400 MHz, Chloroform-d) chemical shifts 7.61 (d, J = 8.1 Hz, 2H), 7.43 (d, J = 3.7 Hz, 2H), 7.38-7.30 (m, 6H), 7.21-7.15 (m, 2H), 5.36 (s, 2H), 3.40 (s, 3H). | 513 |
| 110 | 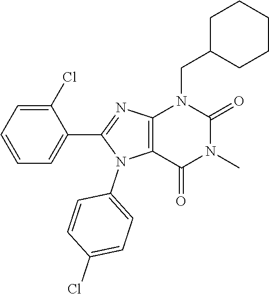 [SMILES: Cn1c(=O)c2c(nc(-c3ccccc3Cl)n2-c2ccc(Cl)cc2)n(CC2CCCCC2)c1=O] | ¹H NMR (400 MHz, Methanol-d4) chemical shifts 7.54 (d, J = 7.5 Hz, 1H), 7.48 (td, J = 8.2, 7.7, 3.8 Hz, 2H), 7.43-7.35 (m, 3H), 7.33 (d, J = 8.5 Hz, 2H), 4.05 (d, J = 7.4 Hz, 2H), 3.36 (s, 3H), 2.05 (s, 1H), 2.00 (s, 0H), 1.73 (d, J = 26.2 Hz, 5H), 1.44-1.22 (m, 3H), 1.15 (q, J = 11.7 Hz, 2H). | 483 |
| 111 | 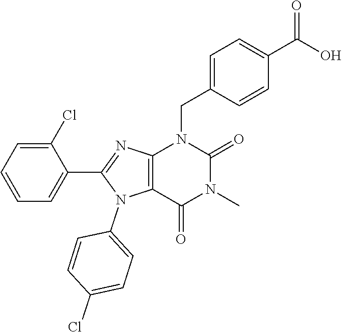 [SMILES: Cn1c(=O)c2c(nc(-c3ccccc3Cl)n2-c2ccc(Cl)cc2)n(Cc2ccc(C(=O)O)cc2)c1=O] | ¹H NMR (400 MHz, Chloroform-d) chemical shifts 8.10 (d, J = 8.3 Hz, 2H), 7.72 (d, J = 8.3 Hz, 2H), 7.46-7.37 (m, 2H), 7.40-7.32 (m, 2H), 7.36-7.28 (m, 2H), 7.24-7.15 (m, 2H), 5.47 (s, 2H), 3.42 (s, 3H). | 521 |
| 112 | 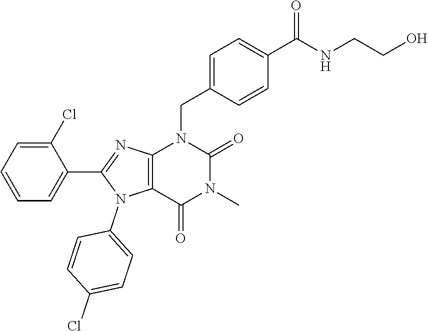 [SMILES: Cn1c(=O)c2c(nc(-c3ccccc3Cl)n2-c2ccc(Cl)cc2)n(Cc2ccc(C(=O)NCCO)cc2)c1=O] | ¹H NMR (400 MHz, Chloroform-d) chemical shifts 7.78 (d, J = 7.9 Hz, 2H), 7.70 (d, J = 7.9 Hz, 2H), 7.42 (d, J = 3.7 Hz, 2H), 7.39-7.28 (m, 4H), 7.22-7.15 (m, 2H), 5.43 (s, 2H), 3.85 (d, J = 5.6 Hz, 2H), 3.65 (d, J = 5.3 Hz, 2H), 3.40 (s, 3H) | 564 |
| 113 | 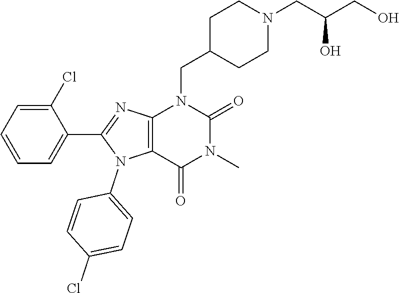 [SMILES: Cn1c(=O)c2c(nc(-c3ccccc3Cl)n2-c2ccc(Cl)cc2)n(CC2CCN(C[C@H](O)CO)CC2)c1=O] | ¹H NMR (400 MHz, Methanol-d4) chemical shifts 7.55-7.44 (m, 3H), 7.43-7.36 (m, 3H), 7.35-7.30 (m, 2H), 4.12 (d, J = 7.3 Hz, 2H), 3.82 (p, J = 5.2 Hz, 1H), 3.58-3.46 (m, 2H), 3.36 (s, 3H), 3.10-3.00 (m, 2H), 2.53-2.40 (m, 2H), 2.20-2.05 (m, 3H), 1.75 (d, J = 13.2 Hz, 2H), 1.56-1.46 (m, 2H) | 558 |
| 114 | 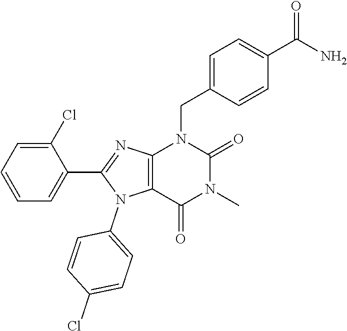 [SMILES: Cn1c(=O)c2c(nc(-c3ccccc3Cl)n2-c2ccc(Cl)cc2)n(Cc2ccc(C(N)=O)cc2)c1=O] | ¹H NMR (400 MHz, Chloroform-d) chemical shifts 7.82 (d, J = 8.0 Hz, 2H), 7.72 (d, J = 8.0 Hz, 2H), 7.42 (d, J = 3.8 Hz, 2H), 7.37-7.31 (m, 4H), 7.22-7.15 (m, 2H), 6.26 (s, 2H), 5.45 (s, 2H), 3.41 (s,3H). | 520 |
| 115 | 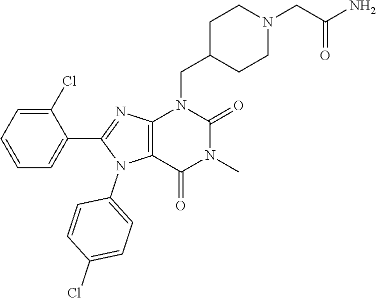 [SMILES: Cn1c(=O)c2c(nc(-c3ccccc3Cl)n2-c2ccc(Cl)cc2)n(CC2CCN(CC(N)=O)CC2)c1=O] | ¹H NMR (400 MHz, Methanol-d4) chemical shifts 7.57-7.45 (m, 3H), 7.45-7.35 (m, 3H), 7.35-7.28 (m, 2H), 4.18 (d, J = 7.0 Hz, 2H), 3.93 (s, 2H), 3.66 (d, J = 12.0 Hz, 2H), 3.37 (s, 3H), 3.05 (t, J = 12.8 Hz, 2H), 2.36 (s, 1H), 2.06 (d, J = 14.1 Hz, 2H), 1.80 (t, J = 13.3 Hz, 2H) | 541 |
| 116 | 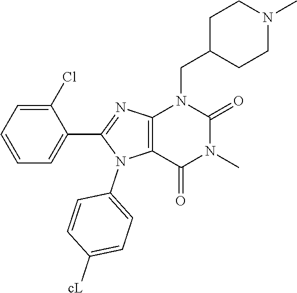 | ¹H NMR (400 MHz, Methanol-d4) chemical shifts 7.54-7.46 (m, 3H), 7.44-7.36 (m, 3H), 7.31 (d, J = 8.7 Hz, 2H), 4.16 (d, J = 7.2 Hz, 2H), 3.55 (d, J = 12.3 Hz, 2H), 3.37 (s, 3H), 2.99 (t, J = 12.7 Hz, 2H), 2.87 (s, 3H), 2.38- 2.28 (m, 1H), 2.09-1.97 (m, 2H), 1.77-1.57 (m, 2H) | 498 |
| 117 | 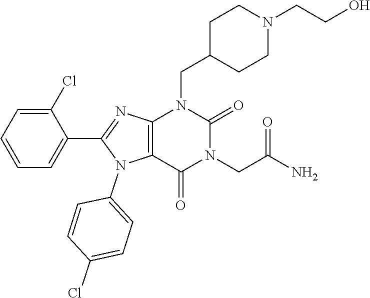 [SMILES: NC(=O)Cn1c(=O)c2c(nc(-c3ccccc3Cl)n2-c2ccc(Cl)cc2)n(CC2CCN(CCO)CC2)c1=O] | ¹H NMR (400 MHz, Methanol-d4) chemical shifts 7.57-7.45 (m, 3H), 7.44-7.35 (m, 3H), 7.34-7.30 (m, 2H), 4.68 (s, 2H), 4.17 (d, J = 7.1 Hz, 2H), 3.91-3.84 (m, 2H), 3.68 (d, J = 12.4 Hz, 2H), 3.27-3.19 (m, 2H), 3.00 (t, J = 12.4 Hz, 2H), 2.37-2.35 (m, 1H), 2.09 (d, J = 14.5 Hz, 2H), 1.72 (q, J = 15.2, 13.4 Hz, 2H). | 571 |
| 118 | 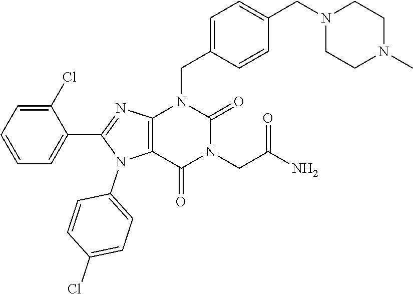 [SMILES: CN1CCN(Cc2ccc(Cn3c(=O)n(CC(N)=O)c(=O)c4c3nc(-c3ccccc3Cl)n4-c3ccc(Cl)cc3)cc2)CC1] | ¹H NMR (400 MHz, Methanol-d4) chemical shifts 7.58-7.44 (m, 5H), 7.43-7.29 (m, 7H), 5.38 (s, 2H), 4.67 (s, 2H), 3.75-3.73 (m, 2H), 3.31-3.23 (m, 4H), 2.93-2.70 (m, 7H) | 632 |
| 119 | 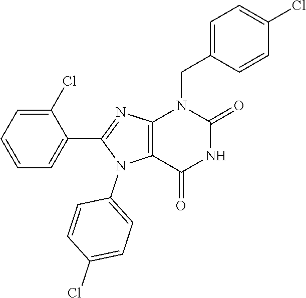 [SMILES: O=c1[nH]c(=O)n(Cc2ccc(Cl)cc2)c2nc(-c3ccccc3Cl)n(-c3ccc(Cl)cc3)c12] | ¹H NMR (400 MHz, DMSO-d6) chemical shifts 11.33 (s, 1H), 7.60 (d, J = 7.4 Hz, 1H), 7.51-7.49 (d, J = 3.6 Hz, 2H), 7.48-7.39 (m, 7H), 7.39-7.35 (m, 2H), 5.18 (s, 2H). | 497 |
| 120 | 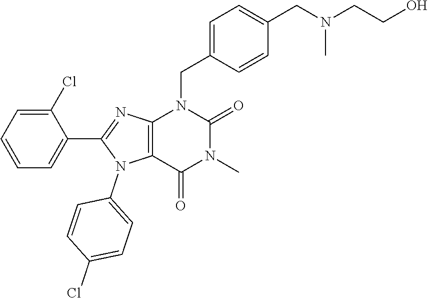 [SMILES: CN(CCO)Cc1ccc(Cn2c(=O)n(C)c(=O)c3c2nc(-c2ccccc2Cl)n3-c2ccc(Cl)cc2)cc1] | ¹H NMR (400 MHz, Methanol-d4) chemical shifts 7.55-7.44 (m, 5H), 7.42-7.30 (m, 7H), 5.37 (s, 2H), 3.68 (t, J = 6.1 Hz, 2H), 3.59 (s, 2H), 3.36 (s, 3H), 2.57 (t, J = 6.1 Hz, 2H), 2.26 (s, 3H). | 564 |
| 121 | 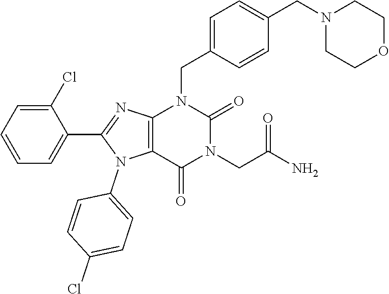 [SMILES: NC(=O)Cn1c(=O)c2c(nc(-c3ccccc3Cl)n2-c2ccc(Cl)cc2)n(Cc2ccc(CN3CCOCC3)cc2)c1=O] | ¹H NMR (400 MHz, Methanol-d4) chemical shifts 7.66 (d, J = 7.9 Hz, 2H), 7.54-7.45 (m, 5H), 7.42-7.35 (m, 3H), 7.34-7.29 (m, 2H), 5.43 (s, 2H), 4.68 (s, 2H), 4.36 (s, 2H), 4.05 (brs, 2H), 3.71 (brs, 2H), 3.47 (brs, 2H), 3.24 (brs, 2H). | 619 |
| 122 | 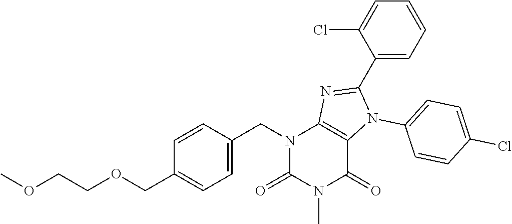 [SMILES: COCCOCc1ccc(Cn2c(=O)n(C)c(=O)c3c2nc(-c2ccccc2Cl)n3-c2ccc(Cl)cc2)cc1] | ¹H NMR (400 MHz, Methanol-d4) chemical shifts 7.54-7.51 (m, 3H), 7.50-7.44 (m, 2H), 7.41-7.35 (m, 4H), 7.34-7.30 (m, 3H), 5.37 (s, 2H), 4.54 (s, 2H), 3.65-3.59 (m, 2H), 3.59- 3.53 (m, 2H), 3.36 (d, J = 3.5 Hz, 6H). | 565 |
| 123 | 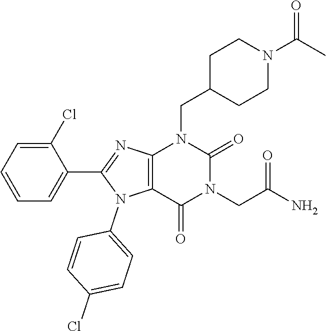 [SMILES: CC(=O)N1CCC(Cn2c(=O)n(CC(N)=O)c(=O)c3c2nc(-c2ccccc2Cl)n3-c2ccc(Cl)cc2)CC1] | ¹H NMR (400 MHz, Methanol-d4) chemical shifts 7.58-7.29 (m, 8H), 4.67 (s, 2H), 4.52 (d, J = 13.5 Hz, 1H), 4.13 (d, J = 7.3 Hz, 2H), 3.95 (d, J = 13.5 Hz, 1H), 3.17-3.06 (m, 1H), 2.72-2.61 (m, 1H), 2.35-2.28 (m 1H), 2.11 (s, 3H), 1.88-1.79 (m, 2H), 1.46-1.25 (m, 2H). | 569 |
| 124 | 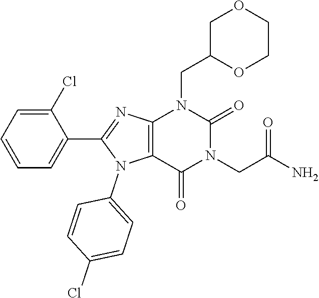 [SMILES: NC(=O)Cn1c(=O)c2c(nc(-c3ccccc3Cl)n2-c2ccc(Cl)cc2)n(CC2COCCO2)c1=O] | ¹H NMR (400 MHz, Methanol-d4) chemical shifts 7.56 (dd, J = 7.6, 1.6 Hz, 1H), 7.53- 7.47 (m, 2H), 7.46-7.40 (m, 1H), 7.40-7.36 (m, 1H), 7.36-7.31 (m, 3H), 4.66 (d, J = 1.8 Hz, 2H), 4.44-4.33 (m, 1H), 4.17-4.06 (m, 2H), 3.92-3.78 (m, 2H), 3.75-3.57 (m, 3H), 3.51 (dd, J = 11.6, 8.7 Hz, 1H). | 530 |
| 125 | 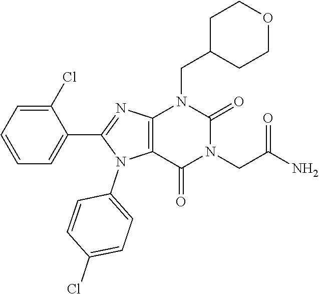 [SMILES: NC(=O)Cn1c(=O)c2c(nc(-c3ccccc3Cl)n2-c2ccc(Cl)cc2)n(CC2CCOCC2)c1=O] | ¹H NMR (400 MHz, Methanol-d4) chemical shifts 7.55 (dd, J = 7.7, 1.6 Hz, 1H), 7.53- 7.45 (m, 2H), 7.42 (dd, J = 7.5, 1.9 Hz, 1H), 7.39-7.30 (m, 4H), 4.67 (s, 2H), 4.11 (d, J = 7.3 Hz, 2H), 3.96 (dd. J = 11.6, 2.0 Hz, 2H), 3.40 (dd, J = 13.0 , 2.1 Hz, 2H), 2.30 (dd, J = 11.4,, 3.8 Hz, 1H), 1.74-1.65 (m, 2H), 1.49 (qd, J = 12.3, 4.4 Hz, 2H). | 528 |
| 126 | 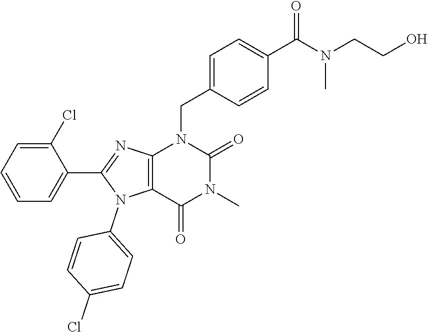 [SMILES: CN(CCO)C(=O)c1ccc(Cn2c(=O)n(C)c(=O)c3c2nc(-c2ccccc2Cl)n3-c2ccc(Cl)cc2)cc1] | ¹H NMR (400 MHz, Methanol-d4) chemical shifts 7.61 (d, J = 6.6 Hz, 2H), 7.56-7.35 (m, 8H), 7.32 (d, J = 8.8 Hz, 2H), 5.42 (s, 2H), 3.83 (t, J = 5.6 Hz, 1H), 3.68 (t, J = 5.9 Hz, 1H), 3.63 (t, J = 5.9 Hz, 1H), 3.42 (t, J = 5.6 Hz, 1H), 3.37 (s, 3H), 3.12-3.05 (m, 3H). | 578 |
| 127 | 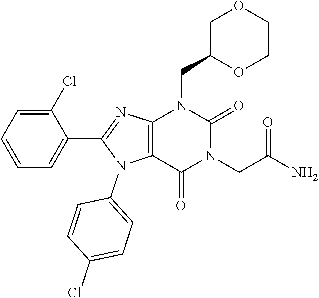 [SMILES: NC(=O)Cn1c(=O)c2c(nc(-c3ccccc3Cl)n2-c2ccc(Cl)cc2)n(C[C@H]2COCCO2)c1=O] | ¹H NMR (400 MHz, Methanol-d4) chemical shifts 7.55 (dd, J = 7.6, 1.6 Hz, 1H), 7.51- 7.45 (m, 2H), 7.42 (dd, J = 7.2, 1.8 Hz, 1H), 7.39-7.29 (m, 4H), 4.66 (d, J = 1.8 Hz, 2H), 4.44-4.33 (m, 1H), 4.17-4.06 (m, 2H), 3.92- 3.78 (m, 2H), 3.72-3.57 (m, 3H), 3.54-3.49 (m, 1H). | 530 |
| 128 | 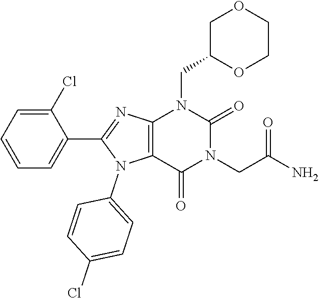 [SMILES: NC(=O)Cn1c(=O)c2c(nc(-c3ccccc3Cl)n2-c2ccc(Cl)cc2)n(C[C@@H]2COCCO2)c1=O] | ¹H NMR (400 MHz, Methanol-d4) chemical shifts 7.55 (dd, J = 7.6, 1.6 Hz, 1H), 7.53- 7.44 (m, 2H), 7.42 (dd, J = 7.2, 1.8 Hz, 1H), 7.40-7.31 (m, 4H), 4.66 (d, J = 1.8 Hz, 2H), 4.44-4.30 (m, 1H), 4.17- 4.06 (m, 2H), 3.92- 3.78 (m, 2H), 3.72-3.57 (m, 3H), 3.54-3.49 (m, 1H). | 530 |
| 129 | 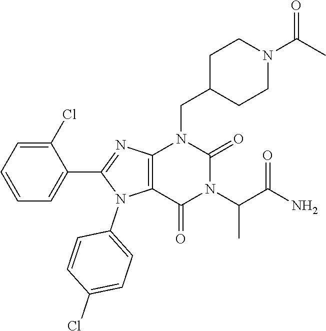 [SMILES: CC(=O)N1CCC(Cn2c(=O)n(C(C)C(N)=O)c(=O)c3c2nc(-c2ccccc2Cl)n3-c2ccc(Cl)cc2)CC1] | ¹H NMR (400 MHz, DMSO-d6) chemical shifts 7.60 (d, J = 7.5 Hz, 1H), 7.55-7.39 (m, 5H), 7.38-7.30 (m, 2H), 7.28 (s, 1H), 6.90 (s, 1H), 5.25 (q, J = 6.7 Hz, 1H), 4.33 (d, J = 13.0 Hz, 1H), 3.97 (d, J = 7.1 Hz, 2H), 3.80 (d, J = 13.7 Hz, 1H), 3.00 (t, J = 13.0 Hz, 1H), 2.55- 2.49 (m, 1H), 2.17-2.12 (m, 1H), 1.98 (s, 3H), 1.80-1.63 (m, 2H), 1.42 (d, J = 6.9 Hz, 3H), 1.28-1.10 (m, 2H). | 583 |
| 130 | 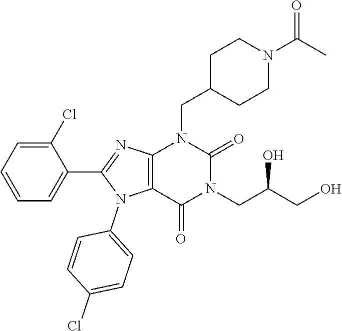 [SMILES: CC(=O)N1CCC(Cn2c(=O)n(C[C@@H](O)CO)c(=O)c3c2nc(-c2ccccc2Cl)n3-c2ccc(Cl)cc2)CC1] | ¹H NMR (400 MHz, DMSO-d6) chemical shifts 7.61 (d, J = 7.7 Hz, 1H), 7.54-7.39 (m, 5H), 7.39-7.32 (m, 2H), 4.33 (d, J = 13.1 Hz, 1H), 4.04-3.99 (m, 3H), 3.85-3.76 (m, 3H), 3.34 (d, J = 5.1 Hz, 2H), 3.00 (t, J = 12.5 Hz, 1H), 2.56-2.50 (m, 1H), 2.27-2.12 (m, 1H), 1.98 (s, 3H), 1.73-1.64 (m, 2H), 1.31-1.09 (m,2H). | 586 |
| 131 | 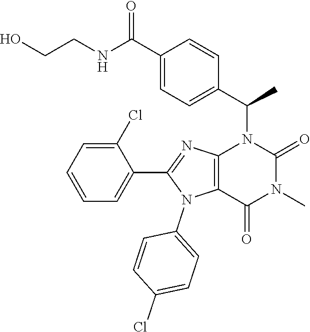 [SMILES: C[C@H](c1ccc(C(=O)NCCO)cc1)n1c(=O)n(C)c(=O)c2c1nc(-c1ccccc1Cl)n2-c1ccc(Cl)cc1] | ¹H NMR (400 MHz, DMSO-d6) chemical shifts 8.39 (t, J = 5.6 Hz, 1H), 7.81 (d, J = 8.2 Hz, 2H), 7.59-7.33 (m, 10H), 6.25 (q, J = 7.1 Hz, 1H), 4.71 (t, J = 5.6 Hz, 1H), 3.52-3.49 (m, 2H), 3.37-3.28 (m, 2H), 3.21 (s, 3H), 2.00 (d, J = 7.1 Hz, 3H). | 578 |
| 132 | 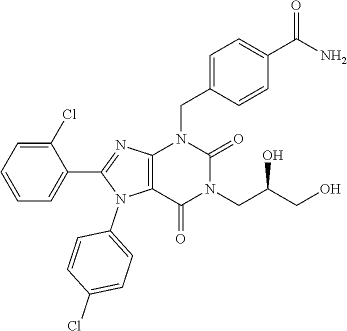 [SMILES: NC(=O)c1ccc(Cn2c(=O)n(C[C@@H](O)CO)c(=O)c3c2nc(-c2ccccc2Cl)n3-c2ccc(Cl)cc2)cc1] | ¹H NMR (400 MHz, DMSO-d6) chemical shifts 7.94 (s, 1H), 7.88-7.81 (m, 2H), 7.64- 7.57 (m, 1H), 7.54-7.31 (m, 10H), 5.31 (s, 2H), 4.61 (br, 2H), 4.07-3.97 (m, 1H), 3.83- 3.78 (m, 2H), 3.39-3.27 (m, 2H). | 580 |
| 133 | 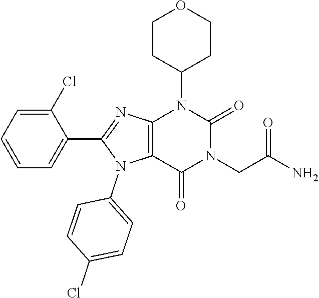 [SMILES: NC(=O)Cn1c(=O)c2c(nc(-c3ccccc3Cl)n2-c2ccc(Cl)cc2)n(C2CCOCC2)c1=O] | ¹H NMR (400 MHz, DMSO-d6) chemical shifts 7.65 (d, J = 7.5 Hz, 1H), 7.55 (s, 1H), 7.53-7.51 (m, 2H), 7.48-7.40 (m, 3H), 7.39- 7.32 (m, 2H), 7.08 (s, 1H), 5.06-4.95 (m, 1H), 4.40 (s, 2H), 3.99 (dd, J = 11.8, 4.4 Hz, 2H), 3.45 (t, J = 11.8 Hz, 2H), 2.83-2.73 (m, 2H), 1.72-1.63 (m, 2H). | 514 |
| 134 | 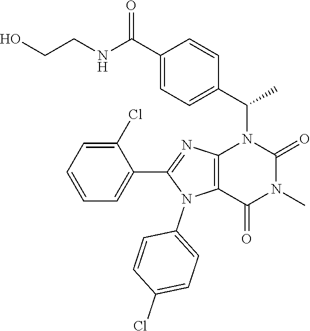 [SMILES: C[C@@H](c1ccc(C(=O)NCCO)cc1)n1c(=O)n(C)c(=O)c2c1nc(-c1ccccc1Cl)n2-c1ccc(Cl)cc1] | ¹H NMR (400 MHz, DMSO-d6) chemical shifts 8.39 (t, J = 5.7 Hz, 1H), 7.81 (d, J = 8.1 Hz, 2H), 7.58-7.34 (m, 10H), 6.25 (q, J = 7.0 Hz, 1H), 4.71 (t, J = 5.6 Hz, 1H), 3.52-3.49 (m, 2H), 3.35-3.28 (m, 2H), 3.21 (s, 3H), 2.00 (d, J = 7.2 Hz, 3H). | 578 |
| 135 | 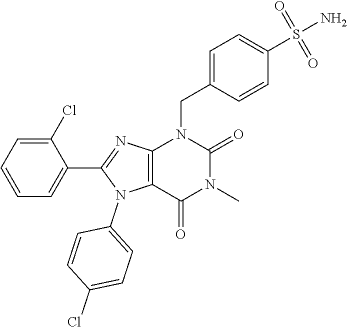 [SMILES: Cn1c(=O)c2c(nc(-c3ccccc3Cl)n2-c2ccc(Cl)cc2)n(Cc2ccc(S(N)(=O)=O)cc2)c1=O] | ¹H NMR (400 MHz, DMSO-d6) chemical shifts 7.80 (d, J = 8.1 Hz, 2H), 7.64-7.59 (m, 3H), 7.53-7.44 (m, 4H), 7.44-7.40 (m, 1H), 7.40-7.33 (m, 4H), 5.34 (s, 2H), 3.24 (s, 3H). | 556 |
| 136 | 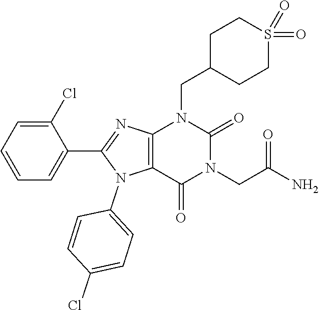 [SMILES: NC(=O)Cn1c(=O)c2c(nc(-c3ccccc3Cl)n2-c2ccc(Cl)cc2)n(CC2CCS(=O)(=O)CC2)c1=O] | ¹H NMR (400 MHz, DMSO-d6) chemical shifts 7.67-7.60 (m, 1H), 7.57-7.50 (m, 3H), 7.50-7.40 (m, 3H), 7.40-7.31 (m, 2H), 7.08 (s, 1H), 4.41 (s, 2H), 4.04 (d, J = 7.3 Hz, 2H), 3.16 (d, J = 13.4 Hz, 1H), 3.09 (s, 2H), 2.37- 2.25 (m, 1H), 2.07 (d, J = 14.1 Hz, 2H), 1.78 (q, J = 12.1 Hz, 2H). | 576 |
| 137 | 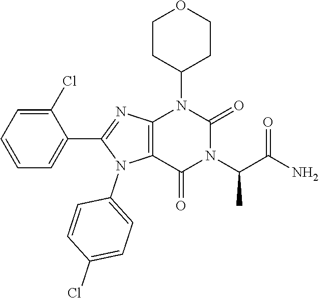 [SMILES: C[C@H](C(N)=O)n1c(=O)c2c(nc(-c3ccccc3Cl)n2-c2ccc(Cl)cc2)n(C2CCOCC2)c1=O] | ¹H NMR (400 MHz, DMSO-d6) chemical shifts 7.61 (d, J = 7.4 Hz, 1H), 7.58-7.49 (m, 2H), 7.49-7.40 (m, 3H), 7.38-7.31 (m, 2H), 7.29 (s, 1H), 6.89 (s, 1H), 5.25 (q, J = 6.8 Hz, 1H), 5.05-4.94 (m, 1H), 3.98 (dd, J = 11.7, 4.4 Hz, 2H), 3.44 (t, J = 11.8 Hz, 2H), 2.77 (tt, J = 12.2, 6.3 Hz, 2H), 1.72-1.65 (m, 2H), 1.42 (d, J = 7.0 Hz, 3H). | 528 |
| 138 | 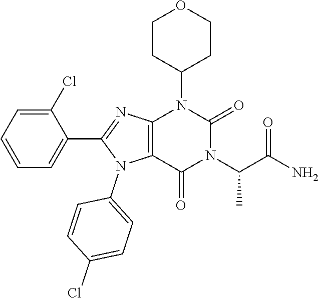 [SMILES: C[C@@H](C(N)=O)n1c(=O)c2c(nc(-c3ccccc3Cl)n2-c2ccc(Cl)cc2)n(C2CCOCC2)c1=O] | ¹H NMR (400 MHz, DMSO-d6) chemical shifts 7.61 (d, J = 7.3 Hz, 1H), 7.55-7.49 (m, 2H), 7.49-7.40 (m, 3H), 7.34 (d, J = 8.7 Hz, 2H), 7.29 (s, 1H), 6.88 (s, 1H), 5.25 (q, J = 7.0 Hz, 1H), 4.98 (d, J = 12.7 Hz, 1H), 3.98 (d, J = 8.4 Hz, 2H). 3.43 (q, J = 10.4, 8.7 Hz, 2H), 2.84-2.71 (m,2H), 1.68 (s, 2H), 1.42 (d, J = 7.0 Hz, 3H). | 528 |
| 139 | 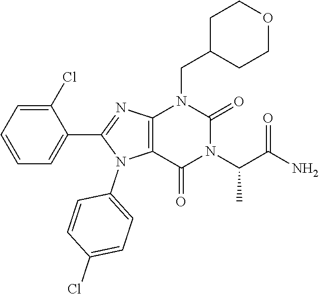 [SMILES: C[C@@H](C(N)=O)n1c(=O)c2c(nc(-c3ccccc3Cl)n2-c2ccc(Cl)cc2)n(CC2CCOCC2)c1=O] | ¹H NMR (400 MHz, DMSO-d6) chemical shifts 7.64-7.57 (m, 1H), 7.57-7.40 (m, 5H), 7.38-7.27 (m, 2H), 7.28 (s, 1H), 6.90 (s, 1H), 5.25 (q, J = 6.9 Hz, 1H), 3.97 (d, J = 7.2 Hz, 2H), 3.86 (d, J = 11.4 Hz, 2H), 3.32-3.21 (m, 2H), 2.26-1.98 (m, 1H), 1.62 (d, J = 13.0 Hz, 2H), 1.43 (d, J = 6.9 Hz, 3H), 1.34 (q, J = 10.7, 10.3 Hz, 2H). | 542 |
| 140 | 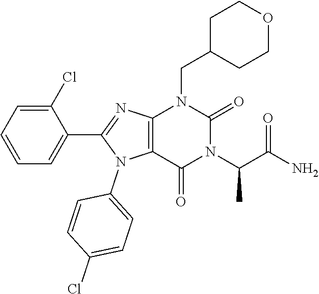 [SMILES: C[C@H](C(N)=O)n1c(=O)c2c(nc(-c3ccccc3Cl)n2-c2ccc(Cl)cc2)n(CC2CCOCC2)c1=O] | ¹H NMR (400 MHz, DMSO-d6) chemical shifts 7.64-7.57 (m, 1H), 7.57-7.50 (m, 2H), 7.49-7.41 (m, 3H), 7.38-7.30 (m, 2H), 7.28 (s, 1H), 6.90 (s, 1H), 5.25 (q, J = 6.9 Hz, 1H), 3.97 (d, J = 7.2 Hz, 2H), 3.86 (d, J = 11.5 Hz, 2H), 3.32-3.22 (m, 2H), 2.17-2.12 (m, 1H), 1.62 (d, J = 13.2 Hz, 2H), 1.43 (d, J = 6.9 Hz, 3H), 1.34 (q, J = 10.7, 10.3 Hz, 2H). | 542 |
| 141 | 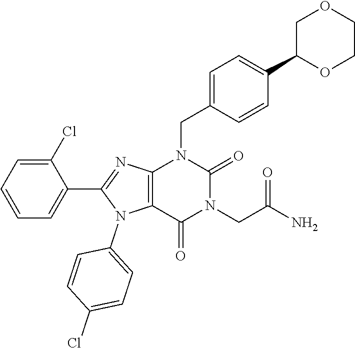 [SMILES: NC(=O)Cn1c(=O)c2c(nc(-c3ccccc3Cl)n2-c2ccc(Cl)cc2)n(Cc2ccc([C@H]3COCCO3)cc2)c1=O] | ¹H NMR (400 MHz, DMSO-d6) chemical shifts 7.63 (d, J = 7.5 Hz, 1H), 7.56 (s, 1H), 7.51 (d, J = 3.9 Hz, 2H), 7.49-7.38 (m, 5H), 7.35 (t, J = 8.1 Hz, 4H), 7.08 (s, 1H), 5.25 (s, 2H), 4.56 (dd, J = 10.2, 2.7 Hz, 1H), 4.41 (s, 2H), 3.88 (dd, J = 11.3, 2.6 Hz, 1H), 3.82- 3.76 (m, 1H), 3.76-3.69 (m, 2H), 3.57 (td, J = 11.5, 2.9 Hz, 1H), 3.28 (dd, J = 11.5, 10.1 Hz, 1H). | 606 |
| 142 | 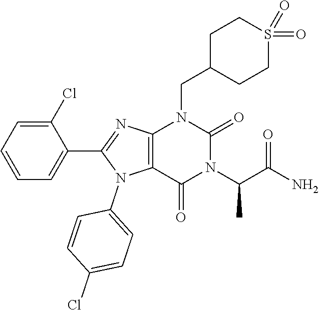 [SMILES: C[C@H](C(N)=O)n1c(=O)c2c(nc(-c3ccccc3Cl)n2-c2ccc(Cl)cc2)n(CC2CCS(=O)(=O)CC2)c1=O] | ¹H NMR (400 MHz, DMSO-d6) chemical shifts 7.62-7.56 (m, 1H), 7.56-7.41 (m, 5H), 7.36-7.31 (m, 2H), 7.29 (s, 1H), 6.90 (s, 1H), 5.24 (q, J = 6.9 Hz, 1H), 4.02 (d, J = 7.3 Hz, 2H), 3.20-3.05 (m, 4H), 2.27 (s, 1H), 2.13- 2.05 (m, 2H), 1.84-1.70 (m, 2H), 1.42 (d, J = 6.9 Hz, 3H). | 590 |
| 143 | 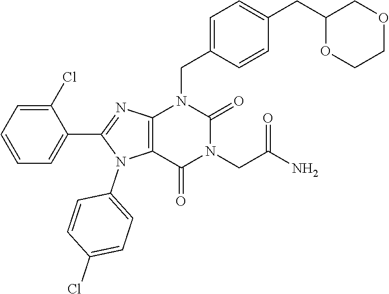 [SMILES: NC(=O)Cn1c(=O)c2c(nc(-c3ccccc3Cl)n2-c2ccc(Cl)cc2)n(Cc2ccc(CC3COCCO3)cc2)c1=O] | ¹H NMR (400 MHz, DMSO-d6) chemical shifts 7.63 (d, J = 7.5 Hz, 1H), 7.56 (s, 1H), 7.51 (d, J = 3.9 Hz, 2H), 7.49-7.40 (m, 3H), 7.39-7.32 (m, 4H), 7.19 (d, J = 7.9 Hz, 2H), 7.09 (s, 1H), 5.23 (s, 2H), 4.41 (s, 2H), 3.71- 3.57 (m, 4H), 3.46 (pd, J = 11.0, 2.2 Hz, 2H), 3.22 (dd, J = 11.8, 10.2 Hz, 1H), 2.60 (dd, J = 6.4, 4.4 Hz, 2H). | 620 |
| 144 | 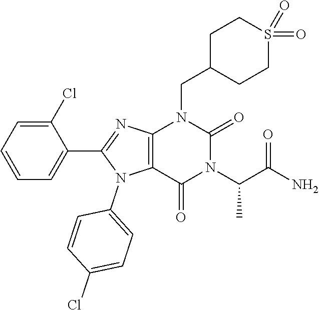 [SMILES: C[C@@H](C(N)=O)n1c(=O)c2c(nc(-c3ccccc3Cl)n2-c2ccc(Cl)cc2)n(CC2CCS(=O)(=O)CC2)c1=O] | ¹H NMR (400 MHz, DMSO-d6) chemical shifts 7.62-7.56 (m, 1H), 7.56-7.40 (m, 5H), 7.37-7.29 (m, 2H), 7.29 (s, 1H), 6.90 (s, 1H), 5.24 (q, J = 6.9 Hz, 1H), 4.02 (d, J = 7.3 Hz, 2H), 3.20-2.95 (m, 4H), 2.27 (s, 1H), 2.13- 2.04 (m, 2H), 1.78 (q, J = 12.4, 11.7 Hz, 2H), 1.42 (d, J = 6.9 Hz, 3H). | 590 |
| 145 | 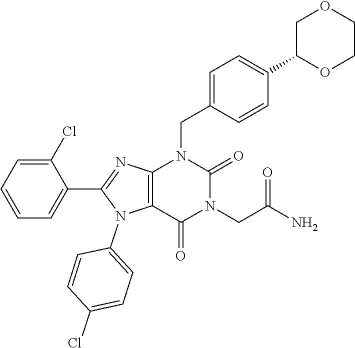 [SMILES: NC(=O)Cn1c(=O)c2c(nc(-c3ccccc3Cl)n2-c2ccc(Cl)cc2)n(Cc2ccc([C@@H]3COCCO3)cc2)c1=O] | ¹H NMR (400 MHz, DMSO-d6) chemical shifts 7.63 (dt, J = 7.5, 1.2 Hz, 1H), 7.56 (s, 1H), 7.53-7.49 (m, 2H), 7.48-7.39 (m, 5H), 7.35 (t, J = 8.1 Hz, 4H), 7.08 (s, 1H), 5.25 (s, 2H), 4.56 (dd, J = 10.2, 2.7 Hz, 1H), 4.41 (s, 2H), 3.88 (dd, J = 11.2, 2.6 Hz, 1H), 3.82- 3.76 (m, 1H), 3.75-3.70 (m, 2H), 3.57 (td, J = 11.3, 10.8, 2.7 Hz, 1H), 3.28 (dd, J = 11.5, 10.2 Hz, 1H). | 606 |
| 146 | 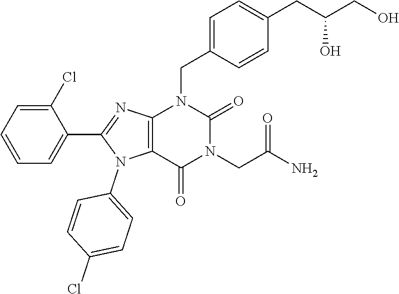 [SMILES: NC(=O)Cn1c(=O)c2c(nc(-c3ccccc3Cl)n2-c2ccc(Cl)cc2)n(Cc2ccc(C[C@@H](O)CO)cc2)c1=O] | ¹H NMR (400 MHz, DMSO-d6) chemical shifts 7.64 (d, J = 7.5 Hz, 1H), 7.56 (s, 1H), 7.51 (d, J = 4.0 Hz, 2H), 7.48-7.40 (m, 3H), 7.39-7.32 (m, 4H), 7.18 (d, J = 7.9 Hz, 2H), 7.08 (s, 1H), 5.22 (s, 2H), 4.59-4.51 (m, 2H), 4.41 (s, 2H), 3.60 (dt, J = 7.9, 5.1 Hz, 1H), 3.27 (dt, J = 7.3, 5.5 Hz, 2H), 2.75 (dd, J = 13.6, 4.6 Hz, 1H), 2.47 (d, J = 7.7 Hz, 1H). | 594 |
| 147 | 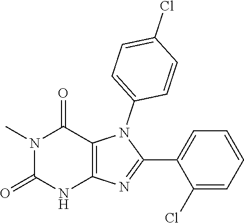 [SMILES: Cn1c(=O)[nH]c2nc(-c3ccccc3Cl)n(-c3ccc(Cl)cc3)c2c1=O] | ¹H NMR (400 MHz, DMSO-d6) chemical shifts 11.46 (s, 1H), 7.60 (d, J = 7.5 Hz, 1H), 7.49 (d, J = 4.1 Hz, 2H), 7.47-7.37 (m, 3H), 7.33 (d, J = 8.3 Hz, 2H), 3.17 (s, 3H). | 387 |
| 148 | 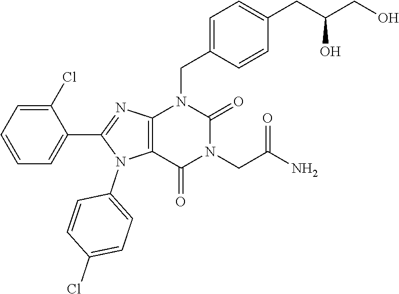 [SMILES: NC(=O)Cn1c(=O)c2c(nc(-c3ccccc3Cl)n2-c2ccc(Cl)cc2)n(Cc2ccc(C[C@H](O)CO)cc2)c1=O] | ¹H NMR (400 MHz, DMSO-d6) chemical shifts 7.64 (d, J = 7.5 Hz, 1H), 7.56 (s, 1H), 7.51 (d, J = 4.0 Hz, 2H), 7.48-7.40 (m, 3H), 7.40-7.32 (m, 4H), 7.18 (d, J = 7.9 Hz, 2H), 7.08 (s, 1H), 5.22 (s, 2H), 4.59-4.51 (m, 2H), 4.41 (s, 2H), 3.60 (dt, J = 7.9, 5.1 Hz, 1H), 3.26 (dq, J = 10.9, 5.8 Hz, 2H), 2.75 (dd, J = 13.6, 4.6 Hz, 1H), 2.47 (d, J = 7.8 Hz, 1H). | 594 |
| 149 | 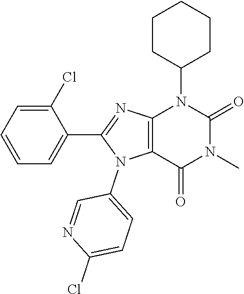 [SMILES: Cn1c(=O)c2c(nc(-c3ccccc3Cl)n2-c2ccc(Cl)nc2)n(C2CCCCC2)c1=O] | ¹H NMR (400 MHz, DMSO-d6) chemical shifts 8.40 (d, J = 2.8 Hz, 1H), 7.91 (dd, J = 8.5, 2.8 Hz, 1H), 7.71 (dd, J = 7.5, 1.7 Hz, 1H), 7.61 (d, J = 8.5 Hz, 1H), 7.59-7.44 (m, 3H), 4.80-4.74 (m, 1H), 3.23 (s, 3H), 2.55- 2.46 (m, 2H), 1.85 (d, J = 13.0 Hz, 2H), 1.77 (d, J = 12.1 Hz, 2H), 1.72-1.62 (m, 1H), 1.38 (q, J = 13.3 Hz, 2H), 1.23-1.13 (m, 1H). | 470 |
| 150 | 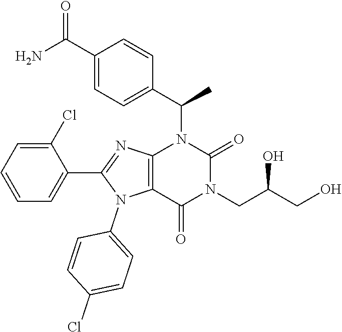 [SMILES: C[C@H](c1ccc(C(N)=O)cc1)n1c(=O)n(C[C@@H](O)CO)c(=O)c2c1nc(-c1ccccc1Cl)n2-c1ccc(Cl)cc1] | ¹H NMR (400 MHz, DMSO-d6) chemical shifts 7.94 (s, 1H), 7.87-7.80 (m, 2H), 7.57- 7.46 (m, 5H), 7.50-7.41 (m, 2H), 7.44-7.32 (m, 3H), 7.33 (s, 1H), 6.24 (q, J = 7.0 Hz, 1H), 4.65 (d, J = 5.4 Hz, 1H), 4.50 (t, J = 5.7 Hz, 1H), 4.04-3.94 (m, 1H), 3.83-3.72 (m, 2H), 3.30 (d, J = 2.6 Hz, 2H), 2.00 (d, J = 7.1 Hz, 3H). | 594 |
| 151 | 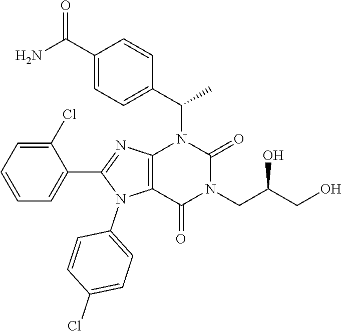 [SMILES: C[C@@H](c1ccc(C(N)=O)cc1)n1c(=O)n(C[C@@H](O)CO)c(=O)c2c1nc(-c1ccccc1Cl)n2-c1ccc(Cl)cc1] | ¹H NMR (400 MHz, DMSO-d6) chemical shifts 7.94 (s, 1H), 7.87-7.80 (m, 2H), 7.57- 7.44 (m, 6H), 7.48-7.36 (m, 2H), 7.36 (td, J = 6.5, 2.0 Hz, 2H), 7.33 (s, 1H), 6.24 (q, J = 7.1 Hz, 1H), 4.65 (d, J = 5.4 Hz, 1H), 4.51 (t, J = 5.7 Hz, 1H), 4.05-3.95 (m, 1H), 3.78 (dq, J = 10.1, 5.3 Hz, 2H), 3.33 (s, 1H), 3.30 (s, 1H), 2.00 (d, J = 7.2 Hz, 3H). | 594 |
| 152 | 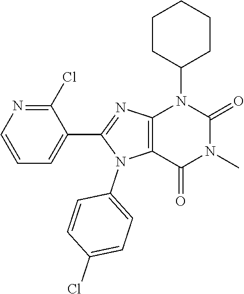 [SMILES: Cn1c(=O)c2c(nc(-c3cccnc3Cl)n2-c2ccc(Cl)cc2)n(C2CCCCC2)c1=O] | ¹H NMR (400 MHz, DMSO-d6) chemical shifts 8.53 (dd, J = 4.9, 1.9 Hz, 1H), 8.17 (dd, J = 7.6, 2.0 Hz, 1H), 7.56 (dd, J = 7.6, 4.8 Hz, 1H), 7.5-7.44 (m, 2H), 7.46-7.36 (m, 2H), 4.77 (ddt, J = 12.3, 8.6, 3.8 Hz, 1H), 3.22 (s, 3H), 2.52 (s, 1H), 2.48 (s, 1H), 1.85 (d, J = 13.0 Hz, 2H), 1.81-1.73 (m, 2H), 1.65 (d, J = 12.6 Hz, 1H), 1.36 (dd, J = 14.8, 11.4 Hz, 2H), 1.26-1.10 (m, 1H). | 470 |
| 153 | 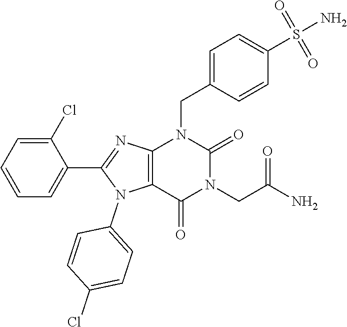 [SMILES: NC(=O)Cn1c(=O)c2c(nc(-c3ccccc3Cl)n2-c2ccc(Cl)cc2)n(Cc2ccc(S(N)(=O)=O)cc2)c1=O] | ¹H NMR (400 MHz, DMSO-d6) chemical shifts 7.83-7.77 (m, 2H), 7.66-7.56 (m, 4H), 7.53-7.43 (m, 5H), 7.43-7.32 (m, 4H), 7.08 (s, 1H), 5.34 (s, 2H), 4.42 (s, 2H). | 599 |
| 154 | 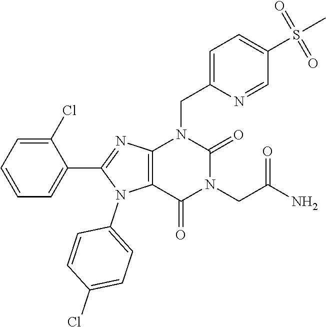 [SMILES: CS(=O)(=O)c1ccc(Cn2c(=O)n(CC(N)=O)c(=O)c3c2nc(-c2ccccc2Cl)n3-c2ccc(Cl)cc2)nc1] | ¹H NMR (400 MHz, DMSO-d6) chemical shifts 9.01 (d, J = 2.3 Hz, 1H), 8.32 (dd, J = 8.3, 2.4 Hz, 1H), 7.70 (d, J = 8.3 Hz, 1H), 7.63- 7.57 (m, 1H), 7.55 (s, 1H), 7.52-7.45 (m, 4H), 7.44-7.34 (m, 3H), 7.09 (s, 1H), 5.50 (s, 2H), 4.42 (s, 2H), 3.33 (s,3H). | 599 |
| 155 | 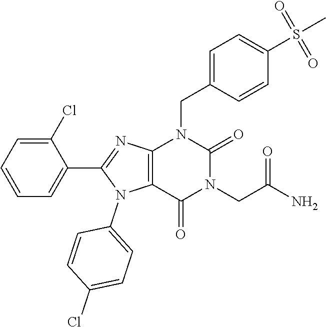 [SMILES: CS(=O)(=O)c1ccc(Cn2c(=O)n(CC(N)=O)c(=O)c3c2nc(-c2ccccc2Cl)n3-c2ccc(Cl)cc2)cc1] | ¹H NMR (400 MHz, DMSO-d6) chemical shifts 7.95-7.89 (m, 2H), 7.71 (d, J = 8.3 Hz, 2H), 7.63 (d, J = 7.3 Hz, 1H), 7.57 (s, 1H), 7.52-7.49 (m, 2H), 7.48 (d, J = 2.0 Hz, 1H), 7.47-7.40 (m, 2H), 7.39-7.34 (m, 2H), 7.10 (s, 1H), 5.38 (s, 2H), 4.42 (s, 2H), 3.21 (s, 3H). | 598 |
| 156 | 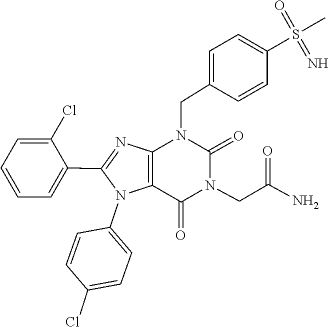 [SMILES: CS(=N)(=O)c1ccc(Cn2c(=O)n(CC(N)=O)c(=O)c3c2nc(-c2ccccc2Cl)n3-c2ccc(Cl)cc2)cc1] | ¹H NMR (400 MHz, Methanol-d4) chemical shifts 7.99 (d, J = 8.3 Hz, 2H), 7.75 (d, J = 8.1 Hz, 2H), 7.55-7.43 (m, 3H), 7.42-7.29 (m, 5H), 5.48 (s, 2H), 4.68 (s, 2H), 3.14 (s, 3H). | 597 |
| 157 | 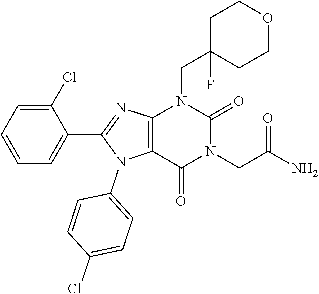 [SMILES: NC(=O)Cn1c(=O)c2c(nc(-c3ccccc3Cl)n2-c2ccc(Cl)cc2)n(CC2(F)CCOCC2)c1=O] | ¹H NMR (400 MHz, DMSO-d6) chemical shifts 7.62 (d, J = 7.5 Hz, 1H), 7.56 (s, 1H), 7.51 (d, J = 3.2 Hz, 2H), 7.49-7.41 (m, 3H), 7.39-7.32 (m, 2H), 7.08 (s, 1H), 4.41 (s, 2H), 4.34 (d, J = 18.6 Hz, 2H), 3.80-3.71 (m, 2H), 3.59-3.49 (m, 2H), 1.89 (d, J = 25.2 Hz, 2H), 1.77 (q, J = 14.7, 12.7 Hz, 2H). | 546 |
| 158 | 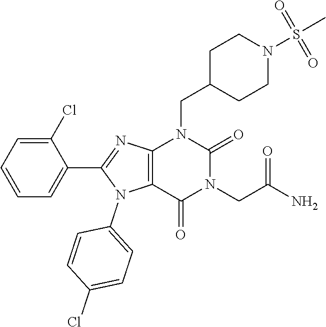 [SMILES: CS(=O)(=O)N1CCC(Cn2c(=O)n(CC(N)=O)c(=O)c3c2nc(-c2ccccc2Cl)n3-c2ccc(Cl)cc2)CC1] | ¹H NMR (400 MHz, DMSO-d6) chemical shifts 7.67-7.60 (m, 1H), 7.58 (s, 1H), 7.55- 4.40 (s, 2H), 4.02 (d, J = 7.1 Hz, 2H), 3.54 (d, J = 11.9 Hz, 3H), 2.83 (s, 3H), 2.74-2.60 (m, 1H), 2.02 (d, J = 12.2 Hz, 1H), 1.78 (d, J = 12.7 Hz, 2H), 1.40-1.27 (m, 2H). | 605 |
| 159 | 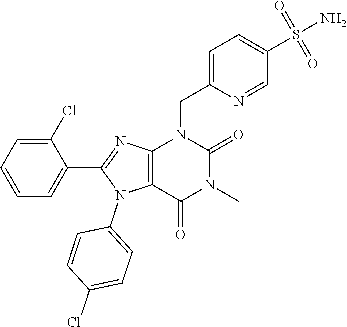 [SMILES: Cn1c(=O)c2c(nc(-c3ccccc3Cl)n2-c2ccc(Cl)cc2)n(Cc2ccc(S(N)(=O)=O)cn2)c1=O] | ¹H NMR (400 MHz, DMSO-d6) chemical shifts 8.95 (dd, J = 2.4, 0.8 Hz, 1H), 8.26 (dd, J = 8.3, 2.4 Hz, 1H), 7.63 (dd, J = 8.3, 0.8 Hz, 1H), 7.52-7.30 (m, 8H), 5.59 (s, 2H), 3.38 (s, 3H). | 557 |
| 160 | 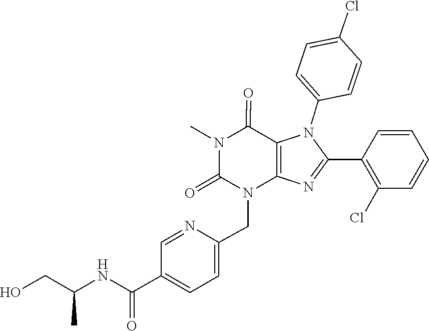 [SMILES: C[C@@H](CO)NC(=O)c1ccc(Cn2c(=O)n(C)c(=O)c3c2nc(-c2ccccc2Cl)n3-c2ccc(Cl)cc2)nc1] | ¹H NMR (500 MHz, Chloroform-d) δ ppm 1.22-1.35 (m, 3 H) 1.26-1.33 (m, 1 H) 3.40 (s, 3 H) 3.65 (dd, J = 10.97, 5.37 Hz, 1 H) 3.78 (dd, J = 10.97, 3.60 Hz, 1 H) 4.19-4.33 (m, 1 H) 5.57 (s, 2 H) 6.33 (br d, J = 7.37 Hz, 1 H) 7.12-7.22 (m, 2 H) 7.22-7.25 (m, 1 H) 7.27- 7.37 (m, 5 H) 7.42 (d, J = 8.17 Hz, 1 H) 8.08 (dd, J = 8.17, 2.24 Hz, 1 H) 8.89 (d, J = 1.92 Hz, 1 H) | 579 |
| 161 | 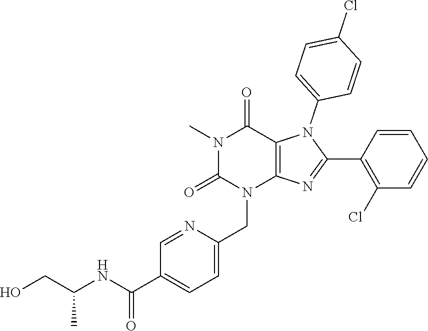 [SMILES: C[C@H](CO)NC(=O)c1ccc(Cn2c(=O)n(C)c(=O)c3c2nc(-c2ccccc2Cl)n3-c2ccc(Cl)cc2)nc1] | ¹H NMR (400 MHz, Chloroform-d) δ ppm 1.29 (d, J = 6.82 Hz, 4 H) 1.47-1.62 (m, 2 H) 3.40 (s, 3 H) 3.65 (dd, J = 10.86, 5.31 Hz, 1 H) 3.79 (dd, J = 10.99, 3.66 Hz, 1 H) 4.18-4.36 (m, 1 H) 4.28 (br d, J = 1.77 Hz, 1 H) 5.57 (s, 2 H) 6.34 (br d, J = 7.33 Hz, 1 H) 7.17-7.25 (m, 2 H) 7.27-7.37 (m, 6 H) 7.42 (d, J = 8.34 Hz, 1 H) 8.08 (dd, J = 8.08, 2.27 Hz, 1 H) 8.90 (d, J = 1.77 Hz, 1H) | 579 |
| 162 | 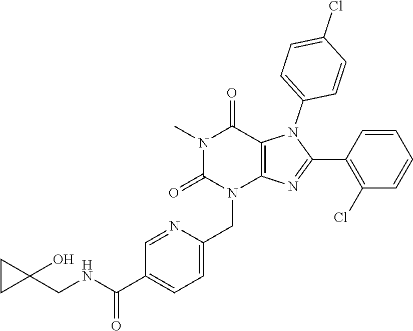 [SMILES: Cn1c(=O)c2c(nc(-c3ccccc3Cl)n2-c2ccc(Cl)cc2)n(Cc2ccc(C(=O)NCC3(O)CC3)cn2)c1=O] | ¹H NMR (400 MHz, Chloroform-d) δ ppm 0.66 (s, 1 H) 0.66-0.69 (m, 1 H) 0.87 (br d, J = 12.38 Hz, 1 H) 0.86-0.89 (m, 1 H) 3.35- 3.47 (m, 1 H) 3.41 (s, 2 H) 3.51-3.63 (m, 1 H) 3.59 (d, J = 5.56 Hz, 1 H) 5.50-5.64 (m, 1 H) 5.58 (s, 1 H) 7.13-7.25 (m, 1 H) 7.19 (d, J = 7.86 Hz, 1 H) 7.27-7.36 (m, 4 H) 7.31- 7.53 (m, 1 H) 7.42 (s, 1 H) 7.44 (s, 1 H) 8.03- 8.15 (m, 1 H) 8.11 (dd, J = 8.08, 2.27 Hz, 1 H) 8.88-8.98 (in, 1 H) 8.93 (d, J = 1.52 Hz, 1 H) | 591 |
| 163 | 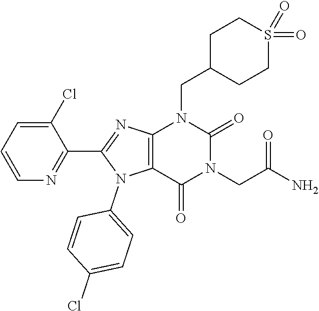 [SMILES: NC(=O)Cn1c(=O)c2c(nc(-c3ncccc3Cl)n2-c2ccc(Cl)cc2)n(CC2CCS(=O)(=O)CC2)c1=O] | ¹H NMR (400 MHz, DMSO-d6) chemical shifts 8.56 (dd, J = 4.7, 1.4 Hz, 1H), 8.10 (dd, J = 8.3, 1.4 Hz, 1H), 7.58 (dd, J = 8.3, 4.7 Hz, 1H), 7.54 (s, 1H), 7.52-7.42 (m, 2H), 7.34- 7.26 (m, 2H), 7.08 (s, 1H), 4.40 (s, 2H), 4.04 (d, J = 7.3 Hz, 2H), 3.19-3.04 (m, 4H), 2.31- 2.23 (m, 1H), 2.07 (d, J = 13.8 Hz, 2H), 1.77 (q, J = 12.1 Hz, 2H). | 577 |
| 164 | 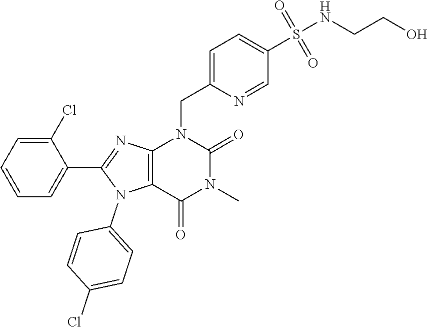 [SMILES: Cn1c(=O)c2c(nc(-c3ccccc3Cl)n2-c2ccc(Cl)cc2)n(Cc2ccc(S(=O)(=O)NCCO)cn2)c1=O] | ¹H NMR (400 MHz, Methanol-d4) chemical shifts 8.91 (d, J = 2.3 Hz, 1H), 8.23 (dd, J = 8.3, 2.3 Hz, 1H), 7.65 (d, J = 8.3 Hz, 1H), 7.52- 7.31 (m, 8H), 5.60 (s, 2H), 3.56 (t, J = 5.8 Hz, 2H), 3.39 (s, 3H), 3.02 (t, J = 5.8 Hz, 2H). | 600 |
| 165 | 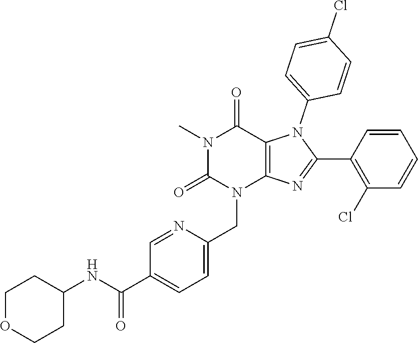 [SMILES: Cn1c(=O)c2c(nc(-c3ccccc3Cl)n2-c2ccc(Cl)cc2)n(Cc2ccc(C(=O)NC3CCOCC3)cn2)c1=O] | ¹H NMR (400 MHz, Chloroform-d) δ ppm 1.19-1.34 (m, 1 H) 1.25 (s, 2 H) 1.38-1.88 (m, 1 H) 1.66 (td, J = 12.00, 4.04 Hz, 6 H) 1.93- 2.06 (m, 1 H) 1.99 (br dd, J = 12.25, 1.89 Hz, 2 H) 2.75-2.83 (m, 1 H) 2.81 (s, 1 H) 3.37- 3.42 (m, 1 H) 3.38-3.41 (m, 2 H) 3.47-3.55 (m, 2 H) 3.47-3.58 (m, 1 H) 3.95-4.07 (m, 1 H) 3.98-4.03 (m, 1 H) 5.67 (br s, 1 H) 5.71 (s, 1 H) 7.10-7.23 (m, 1 H) 7.18 (s, 1 H) 7.20 (s, 1 H) 7.25 (s, 1 H) 7.26-7.38 (m, 5 H) 7.28- 7.41 (m, 1 H) 7.51-7.63 (m, 1 H) 7.52-7.55 (m, 1 H) 7.53-7.59 (m, 1 H) 7.57 (d, J = 8.34 Hz, 1 H) | 605 |
| 166 | 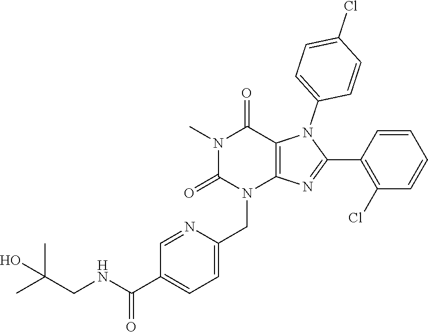 [SMILES: Cn1c(=O)c2c(nc(-c3ccccc3Cl)n2-c2ccc(Cl)cc2)n(Cc2ccc(C(=O)NCC(C)(C)O)cn2)c1=O] | ¹H NMR (400 MHz, DMSO-d6 ) δ ppm 0.98- 1.15 (m, 8 H) 1.27 (br s, 1 H) 3.21-3.26 (m, 1 H) 3.21-3.26 (m, 1 H) 3.23-3.44 (m, 242 H) 3.24-3.24 (m, 1 H) 3.38-3.40 (m, 1 H) 3.39- 3.39 (m, 1 H) 3.43-3.46 (m, 1 H) 3.43-3.45 (m, 1 H) 3.43-3.44 (m, 1 H) 5.39-5.46 (m, 2 H) 7.34-7.50 (m, 8 H) 7.52-7.68 (m, 2 H) 8.32-8.55 (m, 1 H) | 593 |
| 167 | 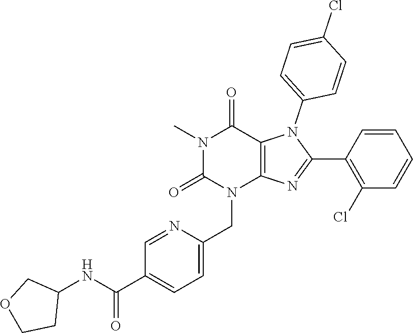 [SMILES: Cn1c(=O)c2c(nc(-c3ccccc3Cl)n2-c2ccc(Cl)cc2)n(Cc2ccc(C(=O)NC3CCOC3)cn2)c1=O] | ¹H NMR (400 MHz, DMSO-d6 ) δ ppm 1.01- 1.01 (m, 1 H) 1.22 (s, 1 H) 1.20-1.25 (m, 1 H) 1.21-1.25 (m, 1 H) 1.78-1.95 (m, 1 H) 2.09- 2.18 (m, 1 H) 2.38-2.44 (m, 1 H) 2.51-2.55 (m, 2 H) 2.66-2.68 (m, 2 H) 2.72 (s, 1 H) 2.88 (s, 1 H) 3.14-3.29 (m, 10 H) 3.21-3.23 (m, 1 H) 3.22-3.23 (m, 1 H) 3.37-3.44 (m, 6 H) 3.49-3.59 (m, 1 H) 3.69 (td, J = 8.15, 5.94 Hz, 1 H) 3.80-3.86 (m, 2 H) 4.40-4.47 (m, 1 H) 5.41 (s, 2 H) 7.32-7.50 (m, 10 H) 7.54 (d, J = 7.48 Hz, 1 H) 7.94 (s, 1 H) 8.15 (dd, J = 8.34, 2.27 Hz, 1 H) 8.67 (d, J = 6.32 Hz, 1 H) 8.81- 8.90 (m, 1 H) | 591 |
| 168 | 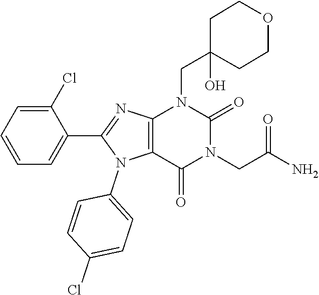 [SMILES: NC(=O)Cn1c(=O)c2c(nc(-c3ccccc3Cl)n2-c2ccc(Cl)cc2)n(CC2(O)CCOCC2)c1=O] | ¹H NMR (400 MHz, DMSO-d6) chemical shifts 7.61 (d, J = 7.5 Hz, 1H), 7.56-7.50 (m, 3H), 7.49-7.40 (m, 3H), 7.39-7.31 (m, 2H), 7.08 (s, 1H), 4.68 (s, 1H), 4.41 (s, 2H), 4.12 (s, 2H), 3.64-3.58 (m, 4H), 1.73-1.65 (m, 2H), 1.49-1.46 (m, 2H). | 544 |
| 169 | 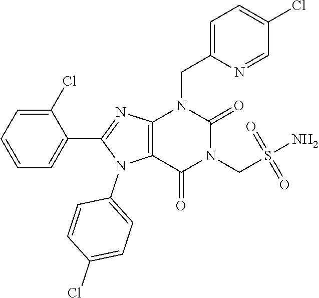 [SMILES: NS(=O)(=O)Cn1c(=O)c2c(nc(-c3ccccc3Cl)n2-c2ccc(Cl)cc2)n(Cc2ccc(Cl)cn2)c1=O] | ¹H NMR (400 MHz, DMSO-d6) chemical shifts 8.57 (d, J = 2.5 Hz, 1H), 7.94 (dd, J = 8.4, 2.5 Hz, 1H), 7.63-7.56 (m, 1H), 7.55- 7.45 (m, 5H), 7.48-7.37 (m, 3H), 7.06 (s, 2H), 5.39 (s, 2H), 5.22 (s, 2H). | 591 |
| 170 | 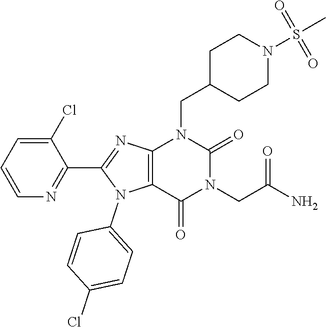 [SMILES: CS(=O)(=O)N1CCC(Cn2c(=O)n(CC(N)=O)c(=O)c3c2nc(-c2ncccc2Cl)n3-c2ccc(Cl)cc2)CC1] | ¹H NMR (400 MHz, DMSO-d6) chemical shifts 8.56 (dd, J = 4.6, 1.4 Hz, 1H), 8.11 (dd, J = 8.3, 1.4 Hz, 1H), 7.58 (dd, J = 8.3, 4.7 Hz, 1H), 7.49 (d, J = 8.7 Hz, 2H), 7.37-7.30 (m, 2H), 4.87 (s, 2H), 4.28 (d, J = 12.7 Hz, 1H), 4.03 (d, J = 7.3 Hz, 2H), 3.86 (d, J = 13.2 Hz, 1H), 3.31-3.23 (m, 4H), 2.93 (d, J = 12.9 Hz, 1H), 2.38-2.22 (m, 1H), 1.84 (t, J = 13.3 Hz, 2H), 1.31 (d, J = 14.7 Hz, 2H). | 606 |
| 171 | 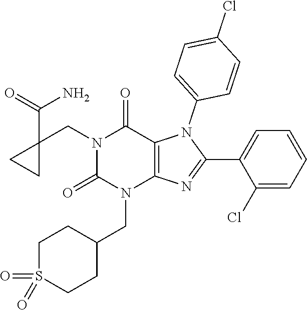 [SMILES: NC(=O)C1(Cn2c(=O)c3c(nc(-c4ccccc4Cl)n3-c3ccc(Cl)cc3)n(CC3CCS(=O)(=O)CC3)c2=O)CC1] | ¹H NMR (400 MHz, DMSO-d6) chemical shifts 7.60 (d, J = 7.5 Hz, 1H), 7.51 (dd, J = 4.8, 1.5 Hz, 2H), 7.49-7.39 (m, 3H), 7.39- 7.32 (m, 3H), 6.96 (s, 1H), 4.28 (s, 2H), 4.04 (d, J = 7.4 Hz, 2H), 3.12 (d, J = 14.0 Hz, 4H), 2.33 (s, 1H), 2.11-2.00 (m, 2H), 1.77 (d, J = 12.6 Hz, 2H), 0.94-0.88 (m, 2H), 0.72-0.66 (m, 2H). | 616 |
| 172 | 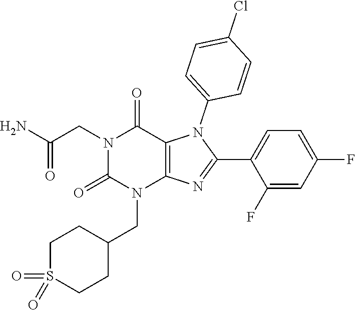 [SMILES: NC(=O)Cn1c(=O)c2c(nc(-c3ccc(F)cc3F)n2-c2ccc(Cl)cc2)n(CC2CCS(=O)(=O)CC2)c1=O] | ¹H NMR (400 MHz, DMSO-d6) chemical shifts 7.70 (td, J = 8.4, 6.4 Hz, 1H), 7.57-7.47 (m, 3H), 7.37 (dd, J = 9.2, 7.0 Hz, 3H), 7.35- 7.22 (m, 1H), 7.10 (s, 1H), 4.40 (s, 2H), 4.04 (d, J = 7.3 Hz, 2H), 3.21-3.03 (m, 4H), 2.29 (s, 1H), 2.07 (d, J = 14.5 Hz, 2H), 1.77 (q, J = 12.5 Hz, 2H). | 578 |
| 173 | 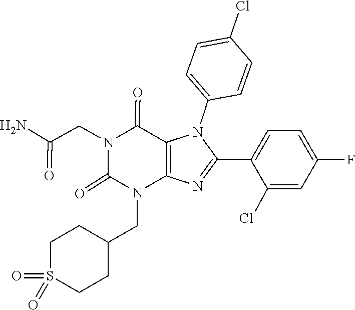 [SMILES: NC(=O)Cn1c(=O)c2c(nc(-c3ccc(F)cc3Cl)n2-c2ccc(Cl)cc2)n(CC2CCS(=O)(=O)CC2)c1=O] | ¹H NMR (400 MHz, DMSO-d6) chemical shifts 7.72 (dd, J = 8.7, 6.1 Hz, 1H), 7.57 (dd, J = 8.8, 2.5 Hz, 2H), 7.53-7.43 (m, 2H), 7.43- 7.27 (m, 3H), 7.10 (s, 1H), 4.40 (s, 2H), 4.03 (d, J = 7.3 Hz, 2H), 3.11 (dt, J = 20.1, 13.2 Hz, 4H), 2.38-2.20 (m, 1H), 2.05 (d, J = 12.8 Hz, 2H), 1.77 (q, J = 12.4 Hz, 2H). | 596 |
| 174 | 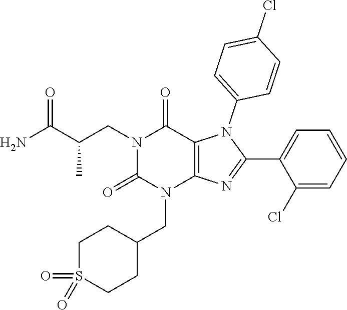 [SMILES: C[C@@H](Cn1c(=O)c2c(nc(-c3ccccc3Cl)n2-c2ccc(Cl)cc2)n(CC2CCS(=O)(=O)CC2)c1=O)C(N)=O] | ¹H NMR (400 MHz, DMSO-d6) chemical shifts 7.63-7.57 (m, 1H), 7.52 (dd, J = 4.9, 1.4 Hz, 2H), 7.50-7.39 (m, 3H), 7.39-7.31 (m, 2H), 7.29 (s, 1H), 6.78 (s, 1H), 4.03 (d, J = 7.4 Hz, 2H), 3.93 (d, J = 7.1 Hz, 2H), 3.12 (d, J = 14.5 Hz, 4H), 2.69-2.61 (m, 1H), 2.28 (s, 1H), 2.05 (d, J = 14.0 Hz, 2H), 1.83-1.70 (m, 2H), 0.97 (d, J = 7.0 Hz, 3H). | 604 |
| 175 | 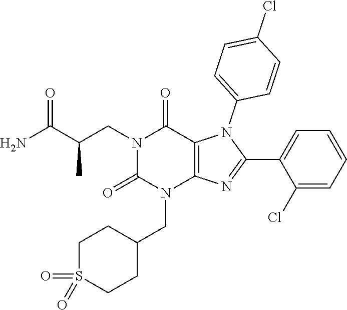 [SMILES: C[C@H](Cn1c(=O)c2c(nc(-c3ccccc3Cl)n2-c2ccc(Cl)cc2)n(CC2CCS(=O)(=O)CC2)c1=O)C(N)=O] | ¹H NMR (400 MHz, DMSO-d6) chemical shifts 7.63-7.57 (m, 1H), 7.57-7.39 (m, 5H), 7.39-7.31 (m, 2H), 7.31-7.26 (m, 1H), 6.78 (s, 1H), 4.03 (d, J = 7.4 Hz, 2H), 3.93 (d, J = 7.1 Hz, 2H), 3.19-3.08 (m, 4H), 2.69-2.66 (m, 1H), 2.64 (d, J = 7.1 Hz, 1H), 2.05 (d, J = 14.0 Hz, 2H), 1.83-1.73 (m, 2H), 0.97 (d, J = 7.0 Hz, 3H). | 604 |
| 176 | 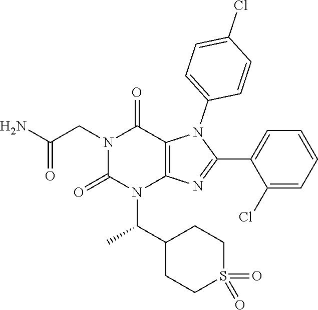 [SMILES: C[C@@H](C1CCS(=O)(=O)CC1)n1c(=O)n(CC(N)=O)c(=O)c2c1nc(-c1ccccc1Cl)n2-c1ccc(Cl)cc1] | 1H NMR (400 MHz, DMSO-d6) chemical shifts 7.63 (d, J = 7.5 Hz, 1H), 7.57-7.39 (m, 6H), 7.39-7.31 (m, 2H), 7.07 (s, 1H), 4.77 (s, 1H), 4.40 (s, 2H), 3.22 (d, J = 12.3 Hz, 1H), 3.09 (s, 1H), 3.03 (d, J = 16.4 Hz, 3H), 2.24 (d, J = 13.8 Hz, 1H), 2.00 (d, J = 65.9 Hz, 1H), 1.77 (t, J = 12.0 Hz, 1H), 1.58 (d, J = 6.5 Hz, 4H). | 590 |
| 177 | 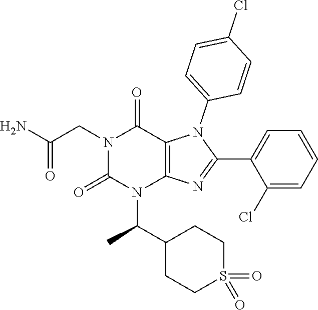 [SMILES: C[C@H](C1CCS(=O)(=O)CC1)n1c(=O)n(CC(N)=O)c(=O)c2c1nc(-c1ccccc1Cl)n2-c1ccc(Cl)cc1] | ¹H NMR (400 MHz, DMSO-d6) chemical shifts 7.63 (d, J = 7.5 Hz, 1H), 7.57-7.39 (m, 6H), 7.39-7.31 (m, 2H), 7.07 (s, 1H), 4.77 (s, 1H), 4.40 (s, 2H), 3.22 (d, J = 12.3 Hz, 1H), 3.09 (s, 1H), 3.03 (d, J = 16.4 Hz, 3H), 2.24 (d, J = 13.8 Hz, 1H), 2.00 (d, J = 65.9 Hz, 1H), 1.77 (t, J = 12.0 Hz, 1H), 1.58 (d, J = 6.5 Hz, 4H). | 590 |
| 179 | 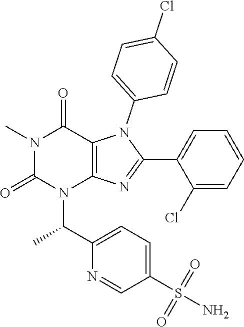 [SMILES: C[C@@H](c1ccc(S(N)(=O)=O)cn1)n1c(=O)n(C)c(=O)c2c1nc(-c1ccccc1Cl)n2-c1ccc(Cl)cc1] | ¹H NMR (400 MHz, DMSO-d6) chemical shifts 8.88 (d, J = 2.3 Hz, 1H), 8.16 (dd, J = 8.4, 2.4 Hz, 1H), 7.70 (d, J = 8.4 Hz, 1H), 7.64- 7.48 (m, 3H), 7.48-7.44 (m, 4H), 7.43-7.33 (m, 3H), 6.23 (q, J = 6.9 Hz, 1H), 3.20 (s, 3H), 1.99 (d, J = 7.1 Hz, 3H). | 571 |
| 180 | 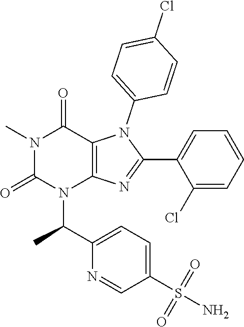 [SMILES: C[C@H](c1ccc(S(N)(=O)=O)cn1)n1c(=O)n(C)c(=O)c2c1nc(-c1ccccc1Cl)n2-c1ccc(Cl)cc1] | ¹H NMR (400 MHz, DMSO-d6) chemical shifts 8.88 (d, J = 2.4 Hz, 1H), 8.16 (dd, J = 8.4, 2.4 Hz, 1H), 7.70 (d, J = 8.4 Hz, 1H), 7.66- 7.48 (m, 3H), 7.49-7.45 (m, 4H), 7.38 (td, J = 6.6, 6.0, 2.7 Hz, 3H), 6.23 (q, J = 7.0 Hz, 1H), 3.20 (s, 3H), 1.99 (d, J = 7.0 Hz, 3H). | 571 |
| 181 | 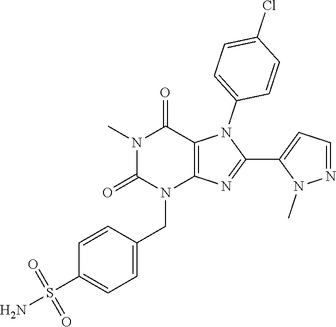 [SMILES: Cn1nccc1-c1nc2c(c(=O)n(C)c(=O)n2Cc2ccc(S(N)(=O)=O)cc2)n1-c1ccc(Cl)cc1] | ¹H NMR (400 MHz, DMSO-d6) chemical shifts 7.81 (d, J = 8.1 Hz, 2H), 7.66 (d, J = 8.2 Hz, 2H), 7.61 (d, J = 8.7 Hz, 2H), 7.55 (d, J = 8.8 Hz, 2H), 7.42-7.31 (m, 2H), 5.70 (d, J = 2.1 Hz, 1H), 5.37 (s, 2H), 4.07 (s, 3H), 3.22 (s, 3H). | 526 |
| 182 | 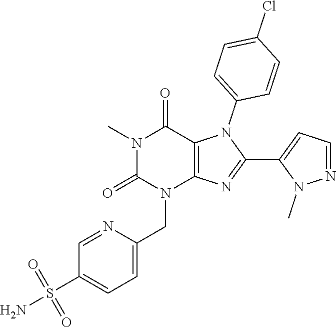 [SMILES: Cn1nccc1-c1nc2c(c(=O)n(C)c(=O)n2Cc2ccc(S(N)(=O)=O)cn2)n1-c1ccc(Cl)cc1] | ¹H NMR (400 MHz, DMSO-d6) chemical shifts 8.89 (d, J = 2.3 Hz, 1H), 8.18 (dd, J = 8.3, 2.3 Hz, 1H), 7.69 (d, J = 8.3 Hz, 1H), 7.65- 7.54 (m, 6H), 7.37 (d, J = 2.1 Hz, 1H), 5.71 (d, J = 2.1 Hz, 1H), 5.50 (s, 2H), 3.94 (s, 3H), 3.23 (s, 3H). | 527 |
| 183 | 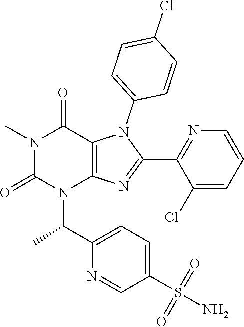 [SMILES: C[C@@H](c1ccc(S(N)(=O)=O)cn1)n1c(=O)n(C)c(=O)c2c1nc(-c1ncccc1Cl)n2-c1ccc(Cl)cc1] | ¹H MR (400 MHz, DMSO-d6) 8.88 (d, J = 2.4 Hz, 1H), 8.49 (dd, J = 4.6, 1.4 Hz, 1H), 8.15 (dd, J = 8.4, 2.4 Hz, 1H), 8.04 (dd, J = 8.3, 1.4 Hz, 1H), 7.71 (d, J = 8.4 Hz, 1H), 7.59 (s, 2H), 7.52 (dd, J = 8.3, 4.7 Hz, 1H), 7.49- 7.43 (m, 2H), 7.34-7.29 (m, 2H), 6.24 (q, J = 6.8 Hz, 1H), 3.22 (s, 3H), 2.00 (d, J = 7.1 Hz, 3H). | 572 |
| 184 | 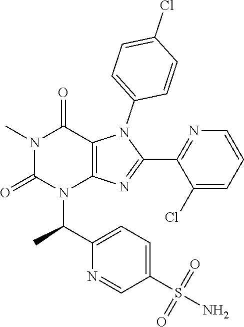 [SMILES: C[C@H](c1ccc(S(N)(=O)=O)cn1)n1c(=O)n(C)c(=O)c2c1nc(-c1ncccc1Cl)n2-c1ccc(Cl)cc1] | ¹H NMR (400 MHz, DMSO-d6) 8.88 (d, J = 2.3 Hz, 1H), 8.49 (dd, J = 4.6, 1.4 Hz, 1H), 8.15 (dd, J = 8.4, 2.4 Hz, 1H), 8.04 (dd, J = 8.2, 1.4 Hz, 1H), 7.71 (d, J = 8.4 Hz, 1H), 7.59 (s, 2H), 7.52 (dd, J = 8.3, 4.7 Hz, 1H), 7.48- 7.42 (m, 2H), 7.34-7.28 (m, 2H), 6.24 (q, J = 7.0 Hz, 1H), 3.22 (s, 3H), 2.00 (d, J = 7.1 Hz, 3H). | 572 |
| 185 | 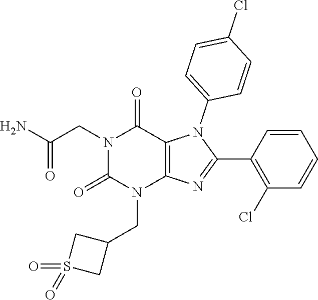 [SMILES: NC(=O)Cn1c(=O)c2c(nc(-c3ccccc3Cl)n2-c2ccc(Cl)cc2)n(CC2CS(=O)(=O)C2)c1=O] | ¹H NMR (400 MHz, DMSO-d6 ) δ ppm 1.22 (s, 1 H) 1.36 (d, J = 4.40 Hz, 1 H) 1.94 (s, 1 H) 2.50-2.64 (m, 1 H) 2.67-2.84 (m, 1 H) 2.93 (s, 1 H) 3.04 (br s, 1 H) 3.16-3.27 (m, 1 H) 4.12 (dd, J = 14.18, 5.87 Hz, 2 H) 4.26-4.42 (m, 4 H) 7.07 (s, 1 H) 7.26-7.35 (m, 2 H) 7.39- 7.54 (m, 4 H) 7.61 (d, J = 7.34 Hz, 1 H) | 548 |
| 186 | 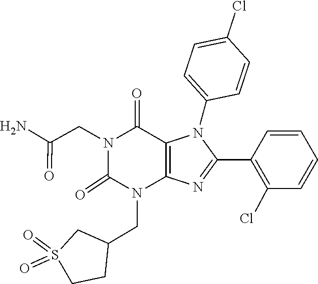 [SMILES: NC(=O)Cn1c(=O)c2c(nc(-c3ccccc3Cl)n2-c2ccc(Cl)cc2)n(CC2CCS(=O)(=O)C2)c1=O] | ¹H NMR (400 MHz, DMSO-d6 ) δ ppm 1.23 (s, 2 H) 1.88-2.08 (m, 3 H) 2.14-2.37 (m, 2 H) 2.78 (s, 2 H) 2.86-3.13 (m, 5 H) 3.18- 3.29 (m, 2 H) 4.08-4.30 (m, 2 H) 4.39 (s, 2 H) 5.52 (s, 1 H) 5.75 (s, 8 H) 7.08 (s, 2 H) 7.23- 7.37 (m, 2 H) 7.27-7.66 (m, 1 H) 7.39-7.57 (m, 4 H) 7.63 (d, J = 7.83 Hz, 1 H) | 562 |
| 187 | 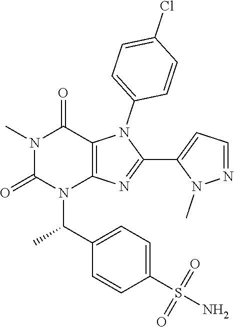 [SMILES: C[C@@H](c1ccc(S(N)(=O)=O)cc1)n1c(=O)n(C)c(=O)c2c1nc(-c1ccnn1C)n2-c1ccc(Cl)cc1] | ¹H NMR (400 MHz, DMSO-d6) 7.80 (d, J = 8.4 Hz, 2H), 7.67 (d, J = 8.3 Hz, 2H), 7.59 (d, J = 8.9 Hz, 2H), 7.53 (d, J = 8.7 Hz, 2H), 7.38- 7.31 (m, 3H) 6.31 (q, J = 7.2 Hz, 1H), 5.62 (d. J = 2.1 Hz, 1H), 3.88 (s, 3H), 3.22 (s, 3H), 2.01 (d, J = 7.1 Hz, 3H). | 540 |
| 188 | 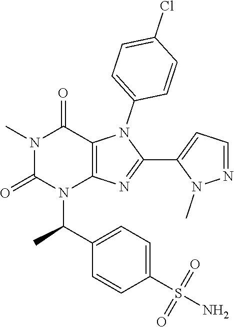 [SMILES: C[C@H](c1ccc(S(N)(=O)=O)cc1)n1c(=O)n(C)c(=O)c2c1nc(-c1ccnn1C)n2-c1ccc(Cl)cc1] | ¹H NMR (400 MHz, DMSO-d6) 7.83-7.78 (m, 2H), 7.67 (d, J = 8.3 Hz, 2H), 7.59 (d, J = 8.8 Hz, 2H), 7.56-7.50 (m, 2H), 7.38-7.33 (m, 3H), 6.31 (d, J = 7.1 Hz, 1H), 5.62 (d, J = 2.1 Hz, 1H), 3.88 (s, 3H), 3.22 (s, 3H), 2.01 (d, J = 7.1 Hz, 3H). | 540 |
| 189 | 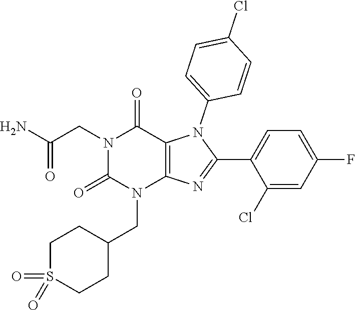 [SMILES: NC(=O)Cn1c(=O)c2c(nc(-c3ccc(F)cc3Cl)n2-c2ccc(Cl)cc2)n(CC2CCS(=O)(=O)CC2)c1=O] | ¹H NMR (400 MHz, DMSO-d6) chemical shifts 8.91 (d, J = 2.3 Hz, 1H), 8.18 (dd, J = 8.4, 2.4 Hz, 1H), 7.76 (d, J = 8.4 Hz, 1H), 7.57 (dd, J = 22.8, 9.3 Hz, 5H), 7.34 (d, J = 2.1 Hz, 1H), 6.30 (q, J = 7.0 Hz, 1H), 5.63 (d, J = 2.1 Hz, 1H), 3.76 (s, 3H), 3.23 (s, 3H), 2.00 (d, J = 7.0 Hz, 3H). | 541 |
| 190 | 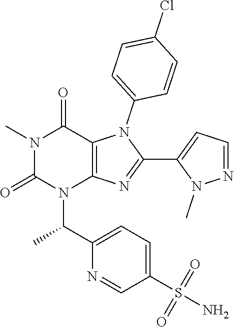 [SMILES: C[C@@H](c1ccc(S(N)(=O)=O)cn1)n1c(=O)n(C)c(=O)c2c1nc(-c1ccnn1C)n2-c1ccc(Cl)cc1] | ¹H NMR (400 MHz, DMSO-d6) chemical shifts 8.91 (d, J = 2.3 Hz, 1H), 8.18 (dd, J = 8.4, 2.4 Hz, 1H), 7.76 (d, J = 8.4 Hz, 1H), 7.62- 7.54 (m, 5H), 7.34 (d, J = 2.1 Hz, 1H), 6.30 (q, J = 7.0 Hz, 1H), 5.63 (d, J = 2.1 Hz, 1H), 3.76 (s, 3H), 3.23 (s, 3H), 2.00 (d, J = 7.0 Hz, 3H). | 541 |

### Example 50. CR1 and CR2 cAMP Antagonist Assay

LANCE Ultra cAMP kit (Perkin Elmer) was used to quantitate the amount of 3′,5′-cyclic adenosine monophosphate (cAMP) produced in Flp-In CHO cells (Invitrogen) stably expressing the CB1 receptor, CB1-CHO cells (Perkin Elmer) stably expressing the CB1 receptor, or CB2-CHO cells (Perkin Elmer) stably expressing the CB2 receptor.

Forskolin was initially titrated to determine the response of the cells. The EC₉₀ of forskolin was used for compound testing. CP55940 was titrated and used with EC₉₀ of forskolin to determine the level of agonist stimulation. EC₉₀ of the agonist was used for subsequent compound testing. For the CB1 assay, Forskolin (Sigma), CP55940 (Cayman Chemicals), and AM251 (MCE) were diluted in 100% DMSO, starting at 100 mM, 1 mM, and 1 mM respectively, in 3-fold serial dilutions. For the CB2 assay, Forskolin (Sigma), CP55940 (Cayman Chemicals), and AM630 (MCE) were diluted in 100% DMSO, starting at 100 mM, 1 mM, and 30 mM respectively, in 3-fold serial dilutions. Test compounds were diluted in 100% DMSO starting from 10 mM, 3-fold dilutions. The cAMP assay buffer contains 1× Hank's Buffered Saline Solution with Ca²⁺ and Mg²⁺ (Invitrogen), 5.3 mM HEPES (Invitrogen), 0.05% BSA, 0.5 mM IBMX (Sigma). For all assays, cells were harvested, counted, and diluted in cAMP assay buffer to 1×10⁵ cells/mL. Only cells with viability >85% were used for the assay. Cells were seeded at 1000 cells/well in 384-well plates and 10 nL/well AM251 (for CB1), AM630 (for CB2) or test compound was added and incubated at 37° C. for 10 min. Then forskolin and agonist were added to reach their EC₉₀ and incubated at 25° C. for 30 min. To detect the amount of cAMP produced, 5 μL of a 100× diluted stock of Eu-cAMP tracer and 5 μL of 200× diluted stock of Ulight-anti-cAMP were added to each well, and the plate incubated at 25° C. for 15 min. The FRET signal was read using an EnVision microplate reader (λₑₓ=320 nm, λₑₘ=615 nm and 665 nm). The results are expressed as % Inhibition, where % inhibition=100−100×(U−C2)/(C1−C2), where U is the FRET ratio (λₑₘ (665 nm)/λₑₘ (615 nm)) of sample, C1 is the average of the high controls (signal with no antagonist added), and C2 is the average of low controls (signal with the highest concentration of AM251 or AM630 antagonist). The IC₅₀ is determined by fitting the percentage of inhibition as a function of compound concentrations with the Hill equation, using a 4-parameter fit in either XLfit or GraphPad Prism.

The following IC₅₀ data were obtained for the compounds using the assays described above (#=compound number; A<100 nM; 100 nM≤B<1 μM; 1 μM≤C<5 μM; D≥5 μM; NT—not tested):

| | CB1 Antagonist | CB2 Antagonist |
|---|---|---|
| # | cAMP IC50 | cAMP IC50 |
| 100 | A | D |
| 101 | A | D |
| 102 | A | NT |
| 103 | A | D |
| 104 | A | D |
| 105 | A | D |
| 106 | A | D |
| 107 | A | NT |
| 108 | A | NT |
| 109 | A | NT |
| 110 | A | NT |
| 111 | C | NT |
| 112 | A | D |
| 113 | D | NT |
| 114 | A | D |
| 115 | B | NT |
| 116 | D | NT |
| 117 | D | NT |
| 118 | C | NT |
| 119 | A | NT |
| 120 | B | NT |
| 121 | A | NT |
| 122 | A | NT |
| 123 | B | NT |
| 124 | A | NT |
| 125 | A | D |
| 126 | B | NT |
| 127 | B | NT |
| 128 | B | NT |
| 129 | B | NT |
| 130 | B | NT |
| 131 | A | D |
| 132 | A | D |
| 133 | A | NT |
| 134 | B | NT |
| 135 | A | D |
| 136 | A | D |
| 137 | B | NT |
| 138 | B | NT |
| 139 | A | NT |
| 140 | A | NT |
| 141 | A | NT |
| 142 | A | NT |
| 143 | A | NT |
| 144 | A | NT |
| 145 | A | NT |
| 146 | B | NT |
| 147 | C | NT |
| 148 | B | NT |
| 149 | A | NT |
| 150 | A | D |
| 151 | B | NT |
| 152 | A | NT |
| 153 | A | NT |
| 154 | A | NT |
| 155 | A | NT |
| 156 | A | NT |
| 157 | A | NT |
| 158 | A | NT |
| 159 | A | D |
| 160 | A | NT |
| 161 | A | NT |
| 162 | A | NT |
| 163 | C | NT |
| 164 | A | D |
| 165 | A | D |
| 166 | A | NT |
| 167 | A | NT |
| 168 | A | NT |
| 169 | A | NT |
| 170 | A | D |
| 171 | NT | NT |
| 172 | B | NT |
| 173 | A | NT |
| 174 | A | NT |
| 175 | A | NT |
| 176 | NT | NT |
| 177 | NT | NT |
| 178 | NT | NT |
| 179 | A | NT |
| 180 | A | NT |
| 181 | A | NT |
| 182 | B | NT |
| 183 | NT | NT |
| 184 | NT | NT |
| 185 | NT | NT |
| 186 | NT | NT |
| 187 | NT | NT |
| 188 | NT | NT |
| 189 | C | NT |
| 190 | C | NT |

1. A compound having structural formula I: 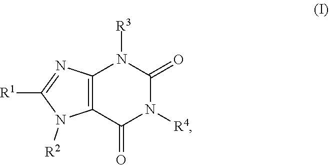
or a pharmaceutically acceptable salt thereof, wherein:
R¹ is aryl, or a 5- to 6-membered heteroaryl that is optionally benzofused, wherein R¹ is optionally substituted;
R² is aryl or a 5- to 6-membered heteroaryl that is optionally benzofused, wherein R² is optionally substituted;
R³ is hydrogen, —(C(R⁵)₂)₀₋₂-carbocyclyl, —(C(R⁵)₂)₀₋₂-heterocyclyl, —(C(R⁵)₂)₁₋₂-pyridinyl, or —(C(R⁵)₂)₁₋₂-phenyl, wherein each R⁵ is independently hydrogen or C₁-C₃ alkyl optionally substituted with one or more substituents independently selected from —OH and halo, and wherein each carbocyclyl, heterocyclyl, pyridinyl and phenyl is optionally substituted with up to two substituents independently selected from halo, —CN, or C₁-C₄ alkyl optionally substituted with halo or hydroxy; and
R⁴ is hydrogen, —C₁-C₄ alkyl optionally substituted with 1 to 3 hydroxyls, —C₁-C₄ alkylene-C(O)—NR⁶R⁷, —C₁-C₄ alkylene-S(O)₂—NR⁶R⁷, —C₁-C₄ alkylene-O—C(O)—C₁-C₄ alkyl, —C₁-C₄ alkylene-O—C₁-C₄ alkyl, —(C(R⁵)₂)₀₋₂-cycloalkyl, or —(C(R⁵)₂)₀₋₂-saturated heterocyclyl, wherein each of R⁶ and R⁷ is independently selected from hydrogen and C₁-C₄ alkyl, and wherein any two methylene units in any alkyl or alkylene portion of R⁴ are optionally taken together with any intervening methylene unit or units to form a cycloalkyl, oxetanyl, tetrahydropyranyl, tetrahydrofuranyl, pyrrolidinyl; and
when R³ is hydrogen, R⁴ is not hydrogen or methyl.

2. The compound or salt of claim 1, wherein:
R¹ is optionally substituted with up to 3 substituents independently selected from —CN, —CF₃, halo, or methyl; and
R² is optionally substituted with up to 3 substituents independently selected from —CN, —CF₃, halo, or methyl.

3. The compound or salt of claim 1, wherein at least one of R¹ or R² is phenyl optionally substituted with one or more halo.

4. The compound or salt of claim 1, wherein R¹ is phenyl, pyridin-2-yl, pyridin-3-yl, or pyrazol-5-yl, and wherein R¹ is optionally substituted with up to two substituents independently selected from methyl and halo.

5. The compound or salt of claim 4, wherein R¹ is 3-chloropyridin-2-yl, 2-chloropyridin-3-yl, 2-chlorophenyl, 2,4-difluorophenyl, 2-chloro-4-fluorophenyl, or 1-methylpyrazol-5-yl.

6. The compound or salt of claim 1, wherein R² is 4-chlorophenyl or 6-chloropyridin-3-yl.

7. The compound or salt of claim 1, wherein R³ is hydrogen, —(CHR⁵)₀₋₁-piperidin-4-yl, —(CHR⁵)₀₋₁-pyridin-2-yl, —(CHR⁵)₀₋₁-tetrahydropyran-4-yl, —(CHR⁵)₀₋₁-tetrahydrothiopyran-4-yl, —(CHR⁵)₀₋₁-phenyl, —(CHR⁵)₀₋₁-cyclohexyl, —(CHR⁵)₀₋₁-1,4-dioxan-2-yl, —(CHR⁵)₀₋₁-thietan-3-yl, or —(CHR⁵)₀₋₁-tetrahydrothiofuran-3-yl, —(CHR⁵)₀₋₁—, wherein R³ is optionally substituted on any ring with one or more of halo, oxo, —OH, —C₁-C₄ alkyl, —CH₂—O—(CH₂)₂O—CH₃, —C(═O)—O—C₁-C₄ alkyl, —C(═O)OH, —C(═O)—C₁-C₄ alkyl, —C(═O)N(R⁶)₂, —C(═O)N(R⁶)—CH₂-cyclopropyl, —S(═O)₂N(R⁶)₂, —S(═O)₂—C₁-C₄ alkyl, —S(═O)(═NH)—C₁-C₄ alkyl, 4-methylpiperazin-1-yl, morpholin-4-ylmethyl, 1,4-dioxan-2-yl, 1,4-dioxan-2-ylmethyl, tetrahydropyran-4-ylcarbamyl, or tetrahydrofuran-3-ylcarbamyl.

8. The compound or salt of claim 7, wherein R³ is hydrogen, 1-methylsulfonylpiperidin-4-ylmethyl, 5-chloropyridin-2-ylmethyl, 4-hydroxytetrahydropyran-4-ylmethyl, 5-(tetrahydrofuran-2-ylcarbamyl)pyridin-2-ylmethyl, 5-(2-hydroxy-2-methylpropan-1-ylcarbamyl)pyridin-2-ylmethyl, 5-(tetrahydropyran-4-ylcarbamyl)pyridin-2-ylmethyl, 5-(2-hydroxyethan-1-ylaminosulfonyl)pyridin-2-yl-methyl, 1,1-dioxothiopyran-4-ylmethyl, 5-((1-hydroxycycloprop-1-ylmethyl)carbamyl)pyridin-2-ylmethyl, 5-(3-hydroxypropan-2-ylcarbamyl)pyridin-2-ylmethyl, 5-(aminosulfonyl)pyridin-2-ylmethyl, 4-fluorotetrahydropyran-4-ylmethyl, 4-(methylsulfonimidoyl)phenylmethyl, 4-(methylsulfonyl)phenylmethyl, 5-(methylsulfonyl)pyridin-2-ylmethyl, 4-(aminosulfonyl)phenylmethyl, cyclohexyl, 4-(carbamyl)phenylethan-2-yl, 4-(2,3-dihydroxylpropan-1-yl)phenylmethyl, 4-(1,4-dioxan-2-yl)phenylmethyl, 4-(1,4-dioxan-2-ylmethyl)phenylmethyl, tetrahydropyran-4-ylmethyl, tetrahydropyran-4-yl, 4-(2-hydroxyethan-1-ylcarbamyl)phenylethan-2-yl, 4-(carbamyl)phenylmethyl, 1-acetylpiperidin-4-ylmethyl, 1,4-dioxan-2-ylmethyl, 4-(2-hydroxyethan-1-ylmethylcarbamyl)phenylmethyl, 4-(2-methoxyethan-1-oxymethyl)phenylmethyl, 4-(morpholin-4-ylemthyl)phenylmethyl, 4-(2-hydroxyethan-1-ylmethylaminomethyl)phenylmethyl, 4-chlorophenylmethyl, 4-(4-methylpiperazin-1-ylmethyl)phenylmethyl, 1-(2-hydroxyethan-1-yl)piperidin-4-ylmethyl, 1-methylpiperidin-4-ylmethyl, 1-(carbamylmethyl)piperidin-4-ylmethyl, 1-(2,3-dihydroxypropan-1-yl)piperidin-4-ylmethyl, 4-(2-hydroxyethan-1-ylcarbamyl)phenylmethyl, 4-carboxyphenylmethyl, cyclohexylmethyl, 1-(1,1-dioxotetrahydrothiopyran-4-yl)ethan-1-yl, (1,1-dioxo-4-fluorotetrahydrothiopyran-4-yl)methyl, 1-(5-aminosulfonylpyridin-2-yl)ethan-1-yl, 4-(methylcarboxy)phenylmethyl, 1,1-dioxothietane-3-ylmethyl, 1,1-dioxotetrahydrofuran-3-ylmethyl, 1-(4-aminosulfonylphenyl)ethan-1-yl, (1,1-dioxo-tetrahydrothiopyran-4-yl)methyl, or 1-(5-aminosulfonylpyridin-2-yl)ethan-1-yl.

9. The compound of salt of claim 1, wherein R⁴ is hydrogen, methyl, 2,3-dihydroxypropan-1-yl, 3-hydroxyproan-1-yl, 2-hydroxyethan-1-yl, carbamylmethyl, 1-carbamylcycloprop-1-ylmethyl, aminosulfonylmethyl, 2-(carbamyl)ethan-2-yl, 2-carbamylpropan-1-yl, tetrabutylcarboxymethyl, or 2-methoxyethan-1-yl.

10. The compound of claim 1 selected from any one of the following structures:

| # | Structure |
|---|---|
| 100 | 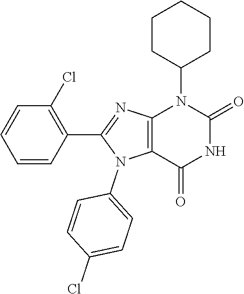 [SMILES: O=c1[nH]c(=O)n(C2CCCCC2)c2nc(-c3ccccc3Cl)n(-c3ccc(Cl)cc3)c12] |
| 101 | 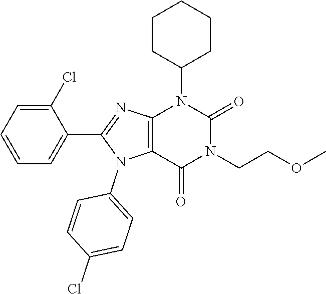 [SMILES: COCCn1c(=O)c2c(nc(-c3ccccc3Cl)n2-c2ccc(Cl)cc2)n(C2CCCCC2)c1=O] |
| 102 | 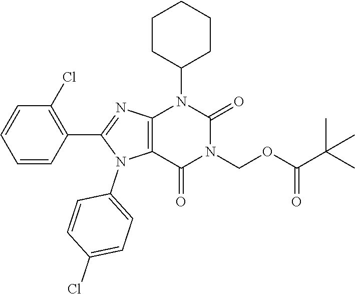 [SMILES: CC(C)(C)C(=O)OCn1c(=O)c2c(nc(-c3ccccc3Cl)n2-c2ccc(Cl)cc2)n(C2CCCCC2)c1=O] |
| 103 | 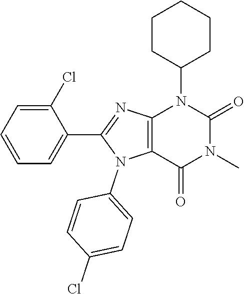 [SMILES: Cn1c(=O)c2c(nc(-c3ccccc3Cl)n2-c2ccc(Cl)cc2)n(C2CCCCC2)c1=O] |
| 104 | 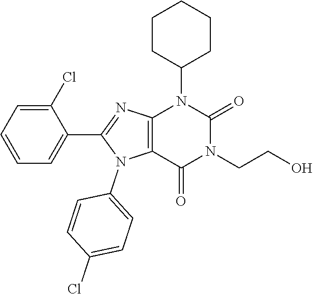 [SMILES: O=c1c2c(nc(-c3ccccc3Cl)n2-c2ccc(Cl)cc2)n(C2CCCCC2)c(=O)n1CCO] |
| 105 | 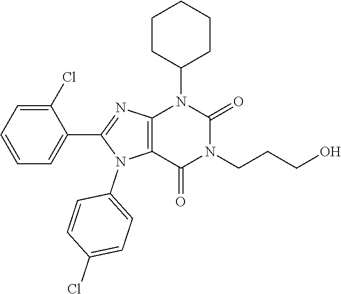 [SMILES: O=c1c2c(nc(-c3ccccc3Cl)n2-c2ccc(Cl)cc2)n(C2CCCCC2)c(=O)n1CCCO] |
| 106 | 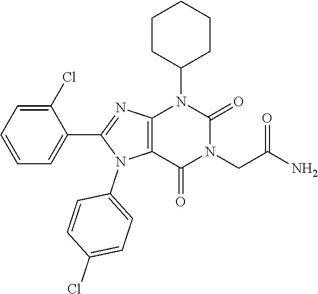 [SMILES: NC(=O)Cn1c(=O)c2c(nc(-c3ccccc3Cl)n2-c2ccc(Cl)cc2)n(C2CCCCC2)c1=O] |
| 107 | 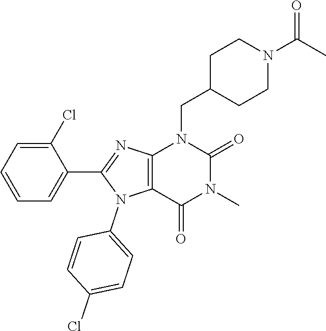 [SMILES: CC(=O)N1CCC(Cn2c(=O)n(C)c(=O)c3c2nc(-c2ccccc2Cl)n3-c2ccc(Cl)cc2)CC1] |
| 108 | 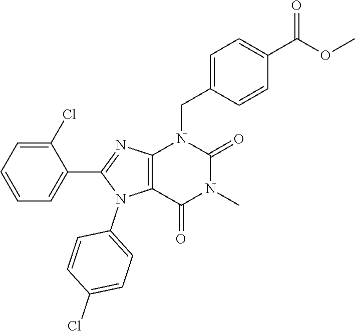 [SMILES: COC(=O)c1ccc(Cn2c(=O)n(C)c(=O)c3c2nc(-c2ccccc2Cl)n3-c2ccc(Cl)cc2)cc1] |
| 109 | 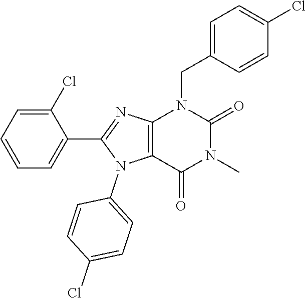 [SMILES: Cn1c(=O)c2c(nc(-c3ccccc3Cl)n2-c2ccc(Cl)cc2)n(Cc2ccc(Cl)cc2)c1=O] |
| 110 | 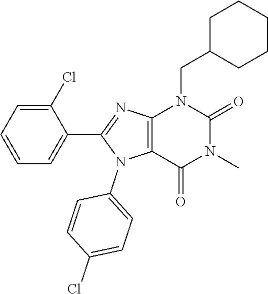 [SMILES: Cn1c(=O)c2c(nc(-c3ccccc3Cl)n2-c2ccc(Cl)cc2)n(CC2CCCCC2)c1=O] |
| 111 | 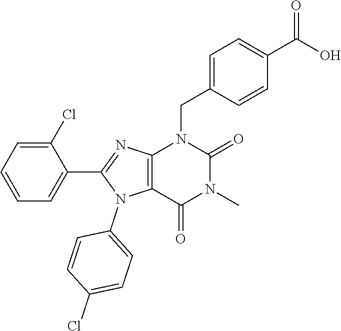 [SMILES: Cn1c(=O)c2c(nc(-c3ccccc3Cl)n2-c2ccc(Cl)cc2)n(Cc2ccc(C(=O)O)cc2)c1=O] |
| 112 | 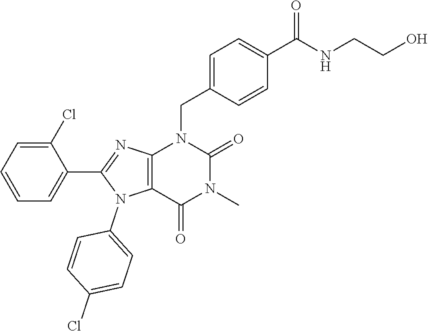 [SMILES: Cn1c(=O)c2c(nc(-c3ccccc3Cl)n2-c2ccc(Cl)cc2)n(Cc2ccc(C(=O)NCCO)cc2)c1=O] |
| 113 | 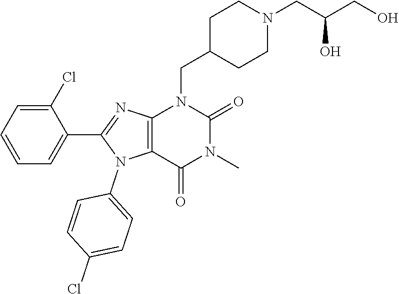 [SMILES: Cn1c(=O)c2c(nc(-c3ccccc3Cl)n2-c2ccc(Cl)cc2)n(CC2CCN(C[C@H](O)CO)CC2)c1=O] |
| 114 | 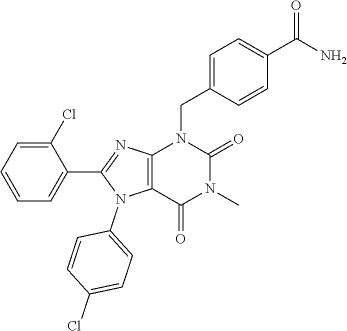 [SMILES: Cn1c(=O)c2c(nc(-c3ccccc3Cl)n2-c2ccc(Cl)cc2)n(Cc2ccc(C(N)=O)cc2)c1=O] |
| 115 | 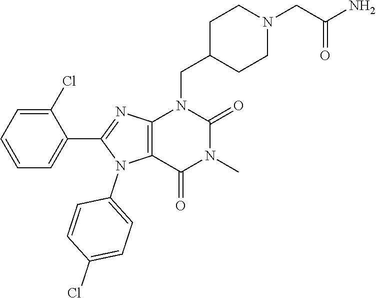 [SMILES: Cn1c(=O)c2c(nc(-c3ccccc3Cl)n2-c2ccc(Cl)cc2)n(CC2CCN(CC(N)=O)CC2)c1=O] |
| 116 | 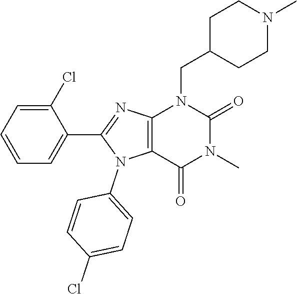 [SMILES: CN1CCC(Cn2c(=O)n(C)c(=O)c3c2nc(-c2ccccc2Cl)n3-c2ccc(Cl)cc2)CC1] |
| 117 | 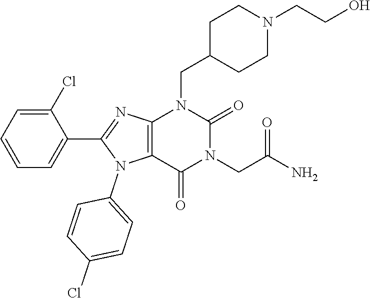 [SMILES: NC(=O)Cn1c(=O)c2c(nc(-c3ccccc3Cl)n2-c2ccc(Cl)cc2)n(CC2CCN(CCO)CC2)c1=O] |
| 118 | 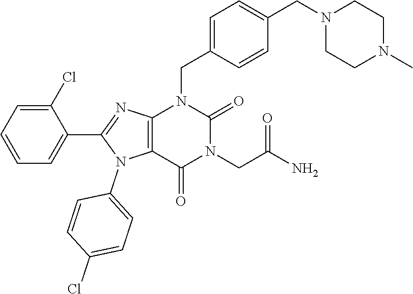 [SMILES: CN1CCN(Cc2ccc(Cn3c(=O)n(CC(N)=O)c(=O)c4c3nc(-c3ccccc3Cl)n4-c3ccc(Cl)cc3)cc2)CC1] |
| 119 | 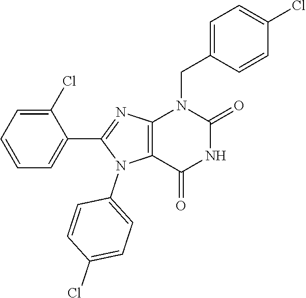 [SMILES: O=c1[nH]c(=O)n(Cc2ccc(Cl)cc2)c2nc(-c3ccccc3Cl)n(-c3ccc(Cl)cc3)c12] |
| 120 | 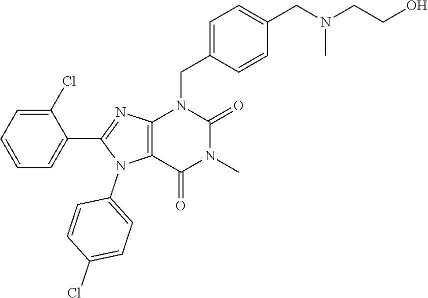 [SMILES: CN(CCO)Cc1ccc(Cn2c(=O)n(C)c(=O)c3c2nc(-c2ccccc2Cl)n3-c2ccc(Cl)cc2)cc1] |
| 121 | 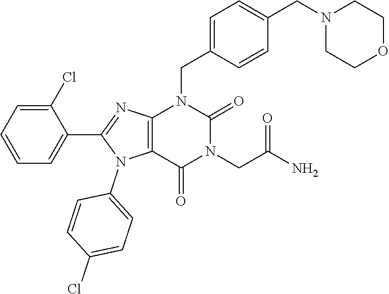 [SMILES: NC(=O)Cn1c(=O)c2c(nc(-c3ccccc3Cl)n2-c2ccc(Cl)cc2)n(Cc2ccc(CN3CCOCC3)cc2)c1=O] |
| 122 | 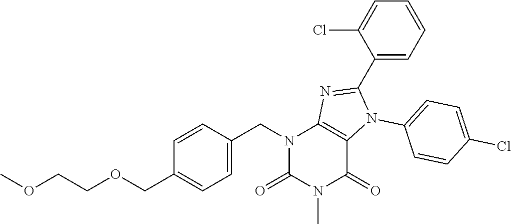 [SMILES: COCCOCc1ccc(Cn2c(=O)n(C)c(=O)c3c2nc(-c2ccccc2Cl)n3-c2ccc(Cl)cc2)cc1] |
| 123 | 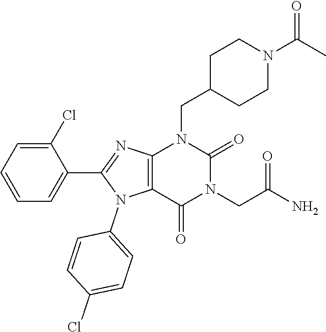 [SMILES: CC(=O)N1CCC(Cn2c(=O)n(CC(N)=O)c(=O)c3c2nc(-c2ccccc2Cl)n3-c2ccc(Cl)cc2)CC1] |
| 124 | 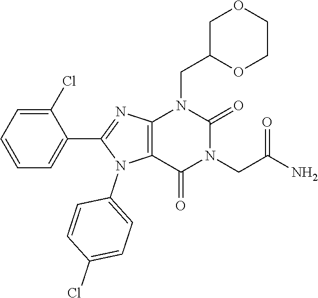 [SMILES: NC(=O)Cn1c(=O)c2c(nc(-c3ccccc3Cl)n2-c2ccc(Cl)cc2)n(CC2COCCO2)c1=O] |
| 125 | 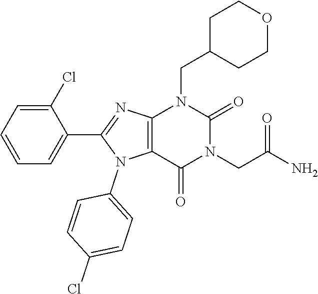 [SMILES: NC(=O)Cn1c(=O)c2c(nc(-c3ccccc3Cl)n2-c2ccc(Cl)cc2)n(CC2CCOCC2)c1=O] |
| 126 | 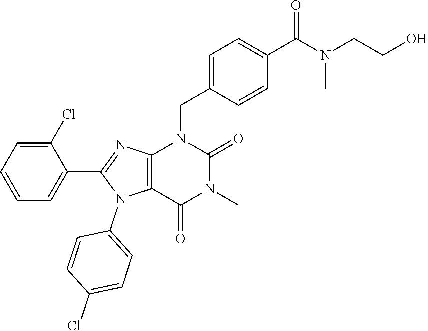 [SMILES: CN(CCO)C(=O)c1ccc(Cn2c(=O)n(C)c(=O)c3c2nc(-c2ccccc2Cl)n3-c2ccc(Cl)cc2)cc1] |
| 127 | 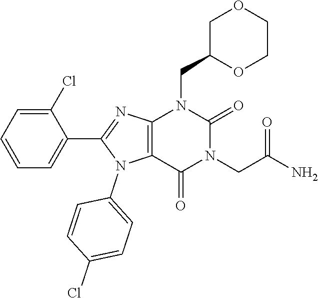 [SMILES: NC(=O)Cn1c(=O)c2c(nc(-c3ccccc3Cl)n2-c2ccc(Cl)cc2)n(C[C@H]2COCCO2)c1=O] |
| 128 | 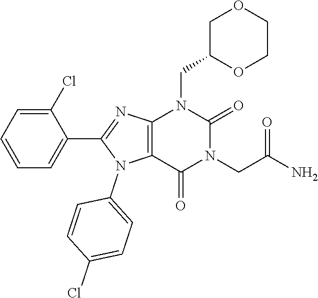 [SMILES: NC(=O)Cn1c(=O)c2c(nc(-c3ccccc3Cl)n2-c2ccc(Cl)cc2)n(C[C@@H]2COCCO2)c1=O] |
| 129 | 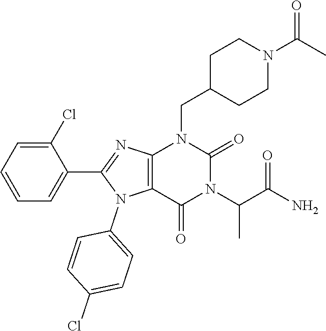 [SMILES: CC(=O)N1CCC(Cn2c(=O)n(C(C)C(N)=O)c(=O)c3c2nc(-c2ccccc2Cl)n3-c2ccc(Cl)cc2)CC1] |
| 130 | 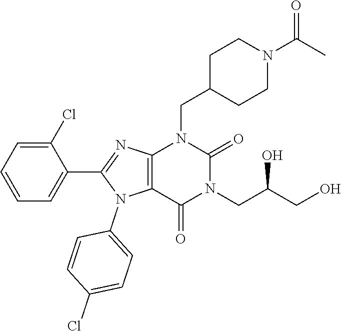 [SMILES: CC(=O)N1CCC(Cn2c(=O)n(C[C@@H](O)CO)c(=O)c3c2nc(-c2ccccc2Cl)n3-c2ccc(Cl)cc2)CC1] |
| 131 | 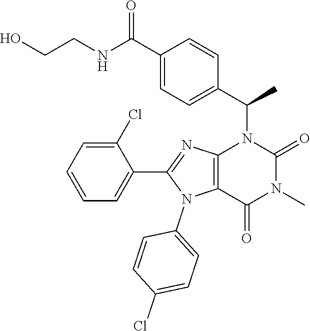 [SMILES: C[C@H](c1ccc(C(=O)NCCO)cc1)n1c(=O)n(C)c(=O)c2c1nc(-c1ccccc1Cl)n2-c1ccc(Cl)cc1] |
| 132 | 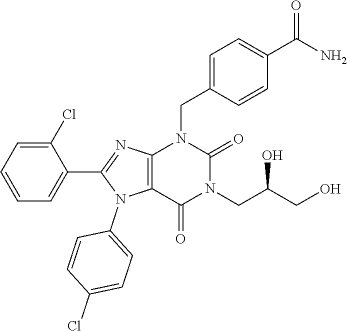 [SMILES: NC(=O)c1ccc(Cn2c(=O)n(C[C@@H](O)CO)c(=O)c3c2nc(-c2ccccc2Cl)n3-c2ccc(Cl)cc2)cc1] |
| 133 | 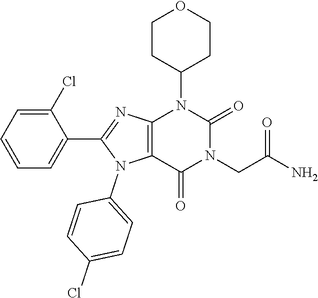 [SMILES: NC(=O)Cn1c(=O)c2c(nc(-c3ccccc3Cl)n2-c2ccc(Cl)cc2)n(C2CCOCC2)c1=O] |
| 134 | 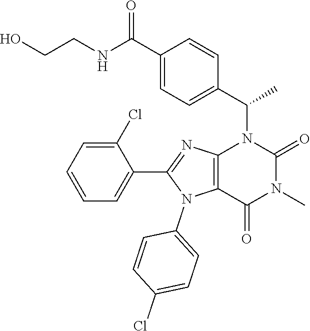 [SMILES: C[C@@H](c1ccc(C(=O)NCCO)cc1)n1c(=O)n(C)c(=O)c2c1nc(-c1ccccc1Cl)n2-c1ccc(Cl)cc1] |
| 135 | 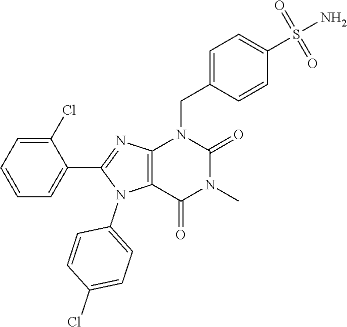 [SMILES: Cn1c(=O)c2c(nc(-c3ccccc3Cl)n2-c2ccc(Cl)cc2)n(Cc2ccc(S(N)(=O)=O)cc2)c1=O] |
| 136 | 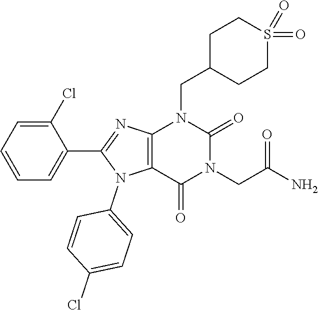 [SMILES: NC(=O)Cn1c(=O)c2c(nc(-c3ccccc3Cl)n2-c2ccc(Cl)cc2)n(CC2CCS(=O)(=O)CC2)c1=O] |
| 137 | 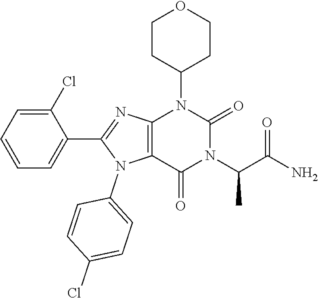 [SMILES: C[C@H](C(N)=O)n1c(=O)c2c(nc(-c3ccccc3Cl)n2-c2ccc(Cl)cc2)n(C2CCOCC2)c1=O] |
| 138 | 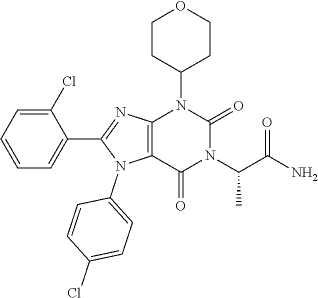 [SMILES: C[C@@H](C(N)=O)n1c(=O)c2c(nc(-c3ccccc3Cl)n2-c2ccc(Cl)cc2)n(C2CCOCC2)c1=O] |
| 139 | 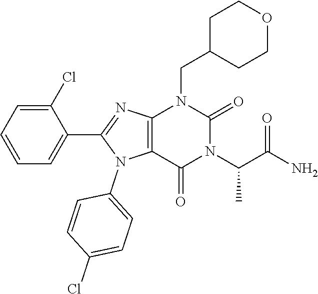 [SMILES: C[C@@H](C(N)=O)n1c(=O)c2c(nc(-c3ccccc3Cl)n2-c2ccc(Cl)cc2)n(CC2CCOCC2)c1=O] |
| 140 | 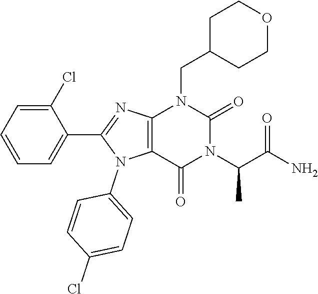 [SMILES: C[C@H](C(N)=O)n1c(=O)c2c(nc(-c3ccccc3Cl)n2-c2ccc(Cl)cc2)n(CC2CCOCC2)c1=O] |
| 141 | 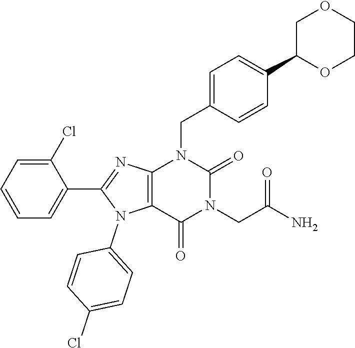 [SMILES: NC(=O)Cn1c(=O)c2c(nc(-c3ccccc3Cl)n2-c2ccc(Cl)cc2)n(Cc2ccc([C@H]3COCCO3)cc2)c1=O] |
| 142 | 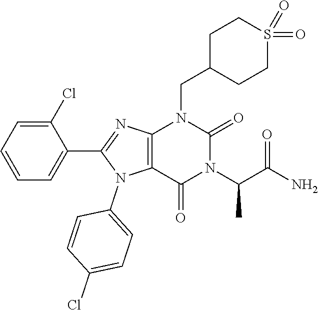 [SMILES: C[C@H](C(N)=O)n1c(=O)c2c(nc(-c3ccccc3Cl)n2-c2ccc(Cl)cc2)n(CC2CCS(=O)(=O)CC2)c1=O] |
| 143 | 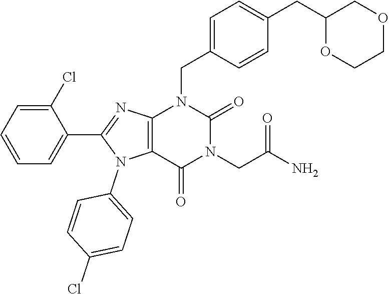 [SMILES: NC(=O)Cn1c(=O)c2c(nc(-c3ccccc3Cl)n2-c2ccc(Cl)cc2)n(Cc2ccc(CC3COCCO3)cc2)c1=O] |
| 144 | 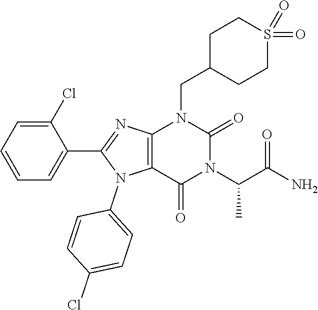 [SMILES: C[C@@H](C(N)=O)n1c(=O)c2c(nc(-c3ccccc3Cl)n2-c2ccc(Cl)cc2)n(CC2CCS(=O)(=O)CC2)c1=O] |
| 145 | 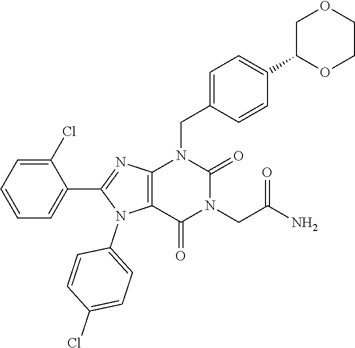 [SMILES: NC(=O)Cn1c(=O)c2c(nc(-c3ccccc3Cl)n2-c2ccc(Cl)cc2)n(Cc2ccc([C@@H]3COCCO3)cc2)c1=O] |
| 146 | 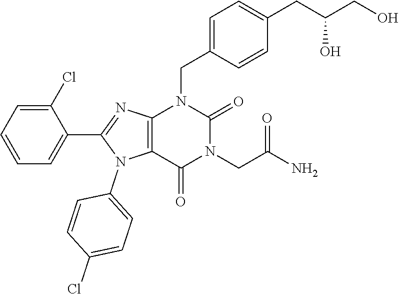 [SMILES: NC(=O)Cn1c(=O)c2c(nc(-c3ccccc3Cl)n2-c2ccc(Cl)cc2)n(Cc2ccc(C[C@@H](O)CO)cc2)c1=O] |
| 147 | 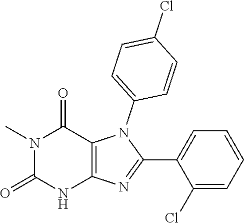 [SMILES: Cn1c(=O)[nH]c2nc(-c3ccccc3Cl)n(-c3ccc(Cl)cc3)c2c1=O] |
| 148 | 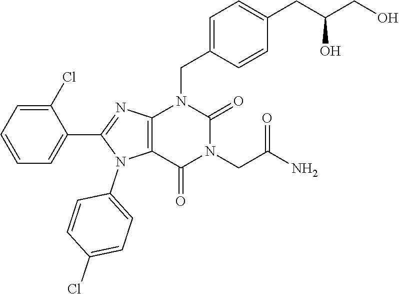 [SMILES: NC(=O)Cn1c(=O)c2c(nc(-c3ccccc3Cl)n2-c2ccc(Cl)cc2)n(Cc2ccc(C[C@H](O)CO)cc2)c1=O] |
| 149 | 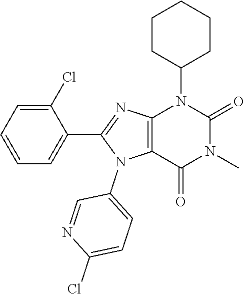 [SMILES: Cn1c(=O)c2c(nc(-c3ccccc3Cl)n2-c2ccc(Cl)nc2)n(C2CCCCC2)c1=O] |
| 150 | 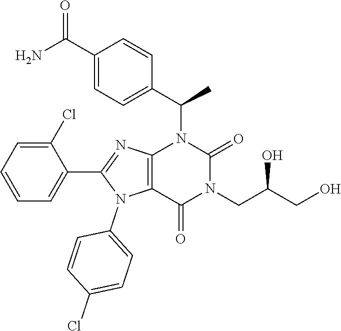 [SMILES: C[C@H](c1ccc(C(N)=O)cc1)n1c(=O)n(C[C@@H](O)CO)c(=O)c2c1nc(-c1ccccc1Cl)n2-c1ccc(Cl)cc1] |
| 151 | 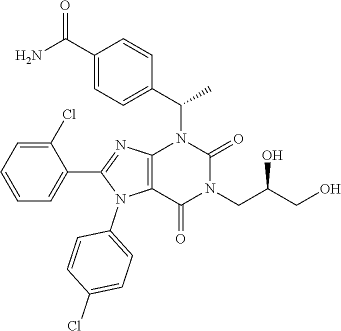 [SMILES: C[C@@H](c1ccc(C(N)=O)cc1)n1c(=O)n(C[C@@H](O)CO)c(=O)c2c1nc(-c1ccccc1Cl)n2-c1ccc(Cl)cc1] |
| 152 | 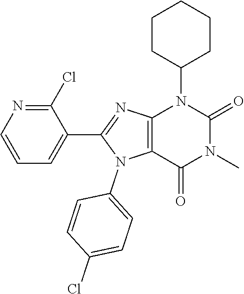 [SMILES: Cn1c(=O)c2c(nc(-c3cccnc3Cl)n2-c2ccc(Cl)cc2)n(C2CCCCC2)c1=O] |
| 153 | 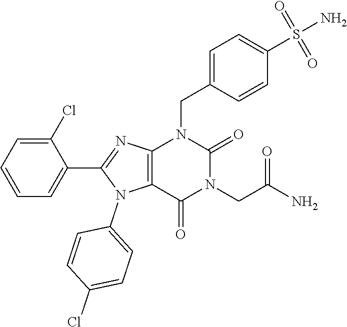 [SMILES: NC(=O)Cn1c(=O)c2c(nc(-c3ccccc3Cl)n2-c2ccc(Cl)cc2)n(Cc2ccc(S(N)(=O)=O)cc2)c1=O] |
| 154 | 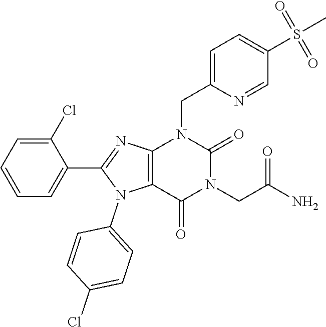 [SMILES: CS(=O)(=O)c1ccc(Cn2c(=O)n(CC(N)=O)c(=O)c3c2nc(-c2ccccc2Cl)n3-c2ccc(Cl)cc2)nc1] |
| 155 | 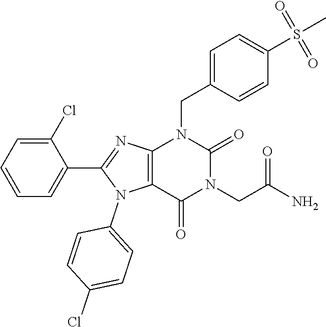 [SMILES: CS(=O)(=O)c1ccc(Cn2c(=O)n(CC(N)=O)c(=O)c3c2nc(-c2ccccc2Cl)n3-c2ccc(Cl)cc2)cc1] |
| 156 | 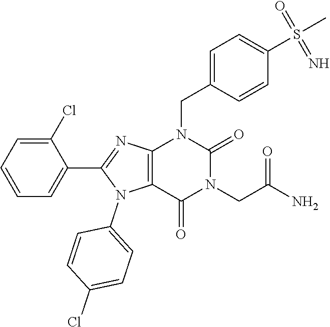 [SMILES: CS(=N)(=O)c1ccc(Cn2c(=O)n(CC(N)=O)c(=O)c3c2nc(-c2ccccc2Cl)n3-c2ccc(Cl)cc2)cc1] |
| 157 | 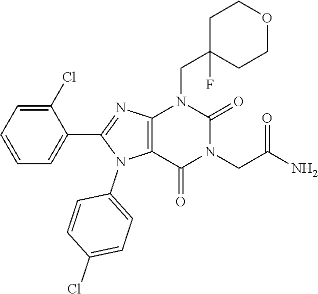 [SMILES: NC(=O)Cn1c(=O)c2c(nc(-c3ccccc3Cl)n2-c2ccc(Cl)cc2)n(CC2(F)CCOCC2)c1=O] |
| 158 | 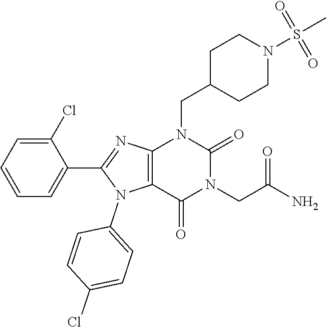 [SMILES: CS(=O)(=O)N1CCC(Cn2c(=O)n(CC(N)=O)c(=O)c3c2nc(-c2ccccc2Cl)n3-c2ccc(Cl)cc2)CC1] |
| 159 | 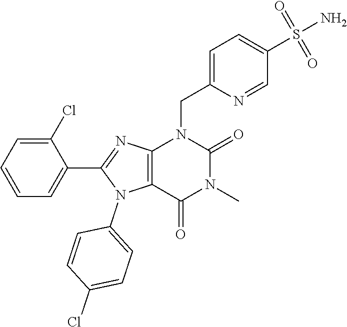 [SMILES: Cn1c(=O)c2c(nc(-c3ccccc3Cl)n2-c2ccc(Cl)cc2)n(Cc2ccc(S(N)(=O)=O)cn2)c1=O] |
| 160 | 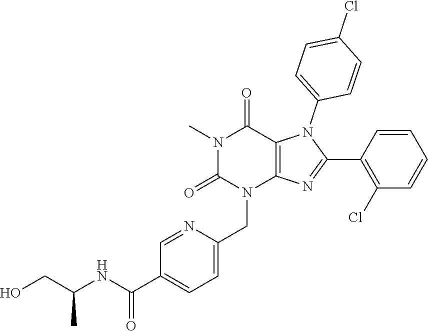 [SMILES: C[C@@H](CO)NC(=O)c1ccc(Cn2c(=O)n(C)c(=O)c3c2nc(-c2ccccc2Cl)n3-c2ccc(Cl)cc2)nc1] |
| 161 | 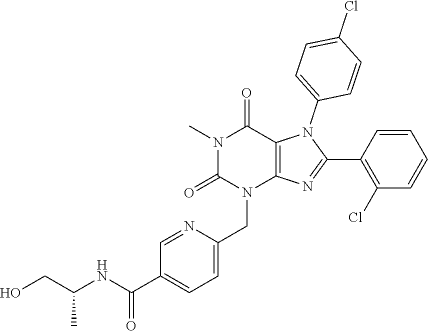 [SMILES: C[C@H](CO)NC(=O)c1ccc(Cn2c(=O)n(C)c(=O)c3c2nc(-c2ccccc2Cl)n3-c2ccc(Cl)cc2)nc1] |
| 162 | 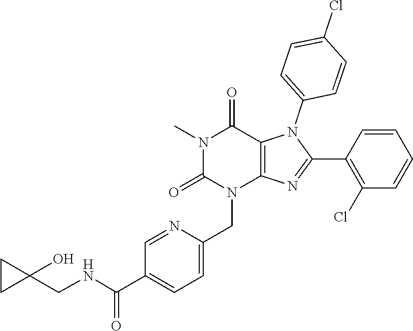 [SMILES: Cn1c(=O)c2c(nc(-c3ccccc3Cl)n2-c2ccc(Cl)cc2)n(Cc2ccc(C(=O)NCC3(O)CC3)cn2)c1=O] |
| 163 | 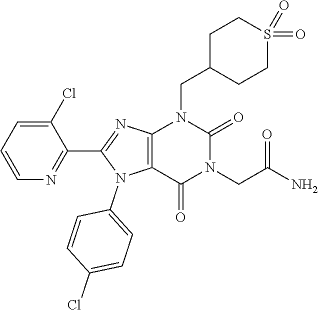 [SMILES: NC(=O)Cn1c(=O)c2c(nc(-c3ncccc3Cl)n2-c2ccc(Cl)cc2)n(CC2CCS(=O)(=O)CC2)c1=O] |
| 164 | 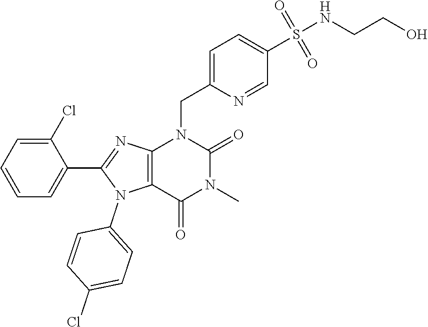 [SMILES: Cn1c(=O)c2c(nc(-c3ccccc3Cl)n2-c2ccc(Cl)cc2)n(Cc2ccc(S(=O)(=O)NCCO)cn2)c1=O] |
| 165 | 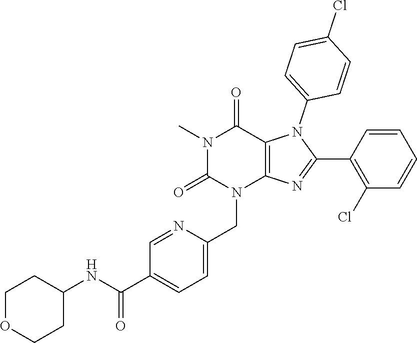 [SMILES: Cn1c(=O)c2c(nc(-c3ccccc3Cl)n2-c2ccc(Cl)cc2)n(Cc2ccc(C(=O)NC3CCOCC3)cn2)c1=O] |
| 166 | 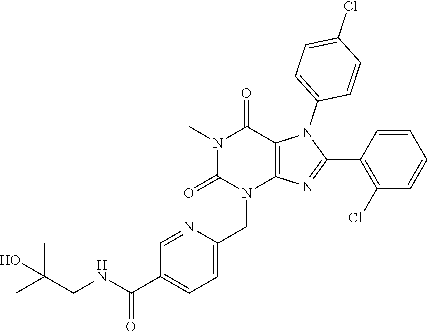 [SMILES: Cn1c(=O)c2c(nc(-c3ccccc3Cl)n2-c2ccc(Cl)cc2)n(Cc2ccc(C(=O)NCC(C)(C)O)cn2)c1=O] |
| 167 | 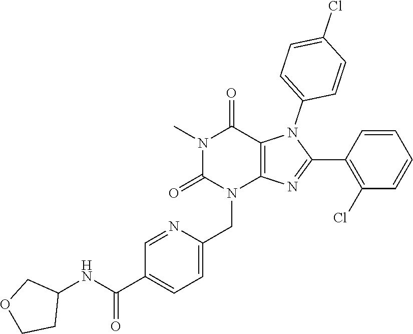 [SMILES: Cn1c(=O)c2c(nc(-c3ccccc3Cl)n2-c2ccc(Cl)cc2)n(Cc2ccc(C(=O)NC3CCOC3)cn2)c1=O] |
| 168 | 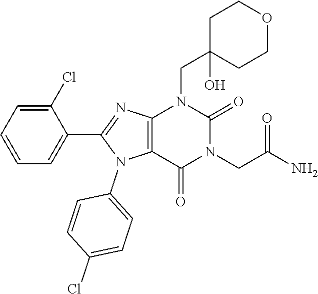 [SMILES: NC(=O)Cn1c(=O)c2c(nc(-c3ccccc3Cl)n2-c2ccc(Cl)cc2)n(CC2(O)CCOCC2)c1=O] |
| 169 | 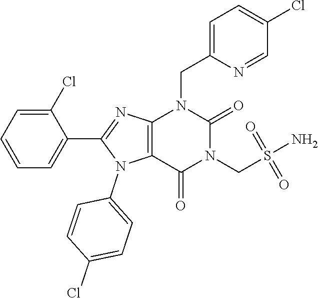 [SMILES: NS(=O)(=O)Cn1c(=O)c2c(nc(-c3ccccc3Cl)n2-c2ccc(Cl)cc2)n(Cc2ccc(Cl)cn2)c1=O] |
| 170 | 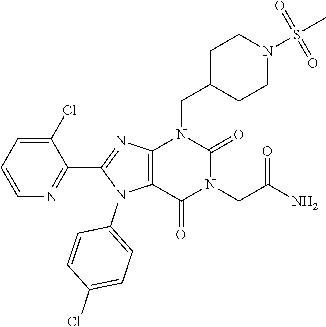 [SMILES: CS(=O)(=O)N1CCC(Cn2c(=O)n(CC(N)=O)c(=O)c3c2nc(-c2ncccc2Cl)n3-c2ccc(Cl)cc2)CC1] |
| 171 | 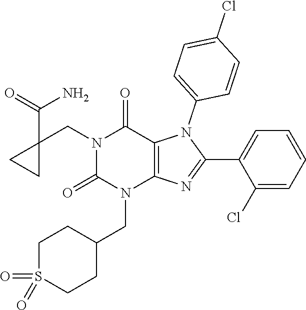 [SMILES: NC(=O)C1(Cn2c(=O)c3c(nc(-c4ccccc4Cl)n3-c3ccc(Cl)cc3)n(CC3CCS(=O)(=O)CC3)c2=O)CC1] |
| 172 | 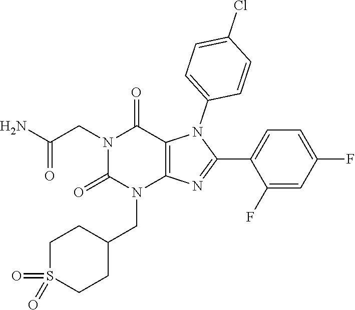 [SMILES: NC(=O)Cn1c(=O)c2c(nc(-c3ccc(F)cc3F)n2-c2ccc(Cl)cc2)n(CC2CCS(=O)(=O)CC2)c1=O] |
| 173 | 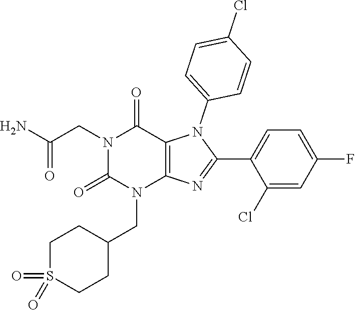 [SMILES: NC(=O)Cn1c(=O)c2c(nc(-c3ccc(F)cc3Cl)n2-c2ccc(Cl)cc2)n(CC2CCS(=O)(=O)CC2)c1=O] |
| 174 | 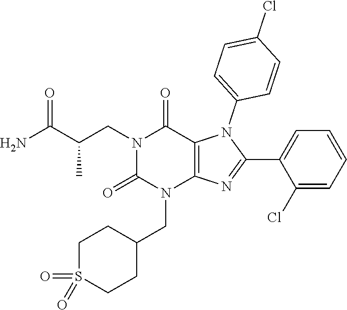 [SMILES: C[C@@H](Cn1c(=O)c2c(nc(-c3ccccc3Cl)n2-c2ccc(Cl)cc2)n(CC2CCS(=O)(=O)CC2)c1=O)C(N)=O] |
| 175 | 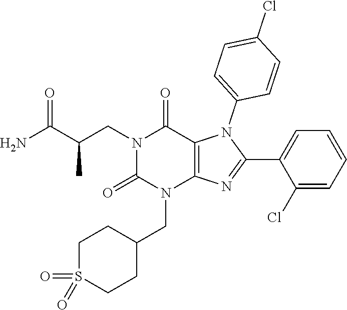 [SMILES: C[C@H](Cn1c(=O)c2c(nc(-c3ccccc3Cl)n2-c2ccc(Cl)cc2)n(CC2CCS(=O)(=O)CC2)c1=O)C(N)=O] |
| 176 | 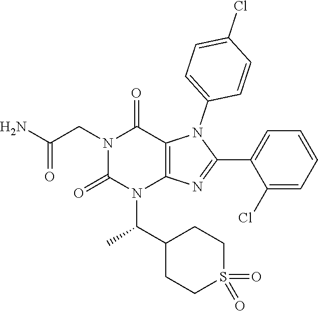 [SMILES: C[C@@H](C1CCS(=O)(=O)CC1)n1c(=O)n(CC(N)=O)c(=O)c2c1nc(-c1ccccc1Cl)n2-c1ccc(Cl)cc1] |
| 177 | 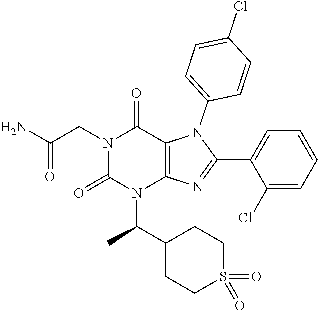 [SMILES: C[C@H](C1CCS(=O)(=O)CC1)n1c(=O)n(CC(N)=O)c(=O)c2c1nc(-c1ccccc1Cl)n2-c1ccc(Cl)cc1] |
| 178 | 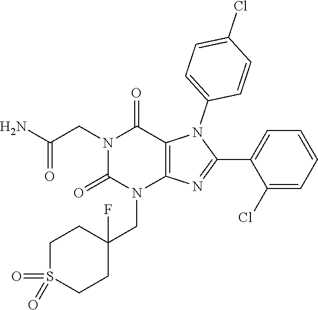 [SMILES: NC(=O)Cn1c(=O)c2c(nc(-c3ccccc3Cl)n2-c2ccc(Cl)cc2)n(CC2(F)CCS(=O)(=O)CC2)c1=O] |
| 179 | 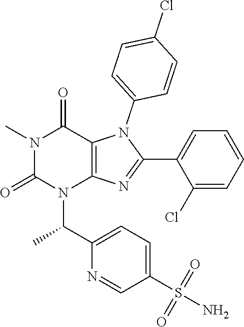 [SMILES: C[C@@H](c1ccc(S(N)(=O)=O)cn1)n1c(=O)n(C)c(=O)c2c1nc(-c1ccccc1Cl)n2-c1ccc(Cl)cc1] |
| 180 | 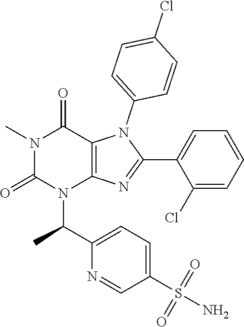 [SMILES: C[C@H](c1ccc(S(N)(=O)=O)cn1)n1c(=O)n(C)c(=O)c2c1nc(-c1ccccc1Cl)n2-c1ccc(Cl)cc1] |
| 181 | 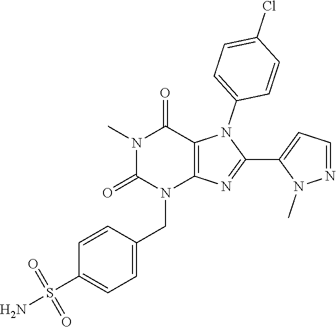 [SMILES: Cn1nccc1-c1nc2c(c(=O)n(C)c(=O)n2Cc2ccc(S(N)(=O)=O)cc2)n1-c1ccc(Cl)cc1] |
| 182 | 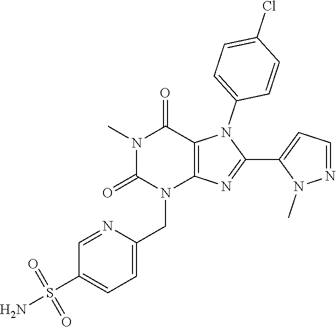 [SMILES: Cn1nccc1-c1nc2c(c(=O)n(C)c(=O)n2Cc2ccc(S(N)(=O)=O)cn2)n1-c1ccc(Cl)cc1] |
| 183 | 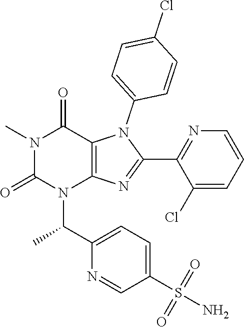 [SMILES: C[C@@H](c1ccc(S(N)(=O)=O)cn1)n1c(=O)n(C)c(=O)c2c1nc(-c1ncccc1Cl)n2-c1ccc(Cl)cc1] |
| 184 | 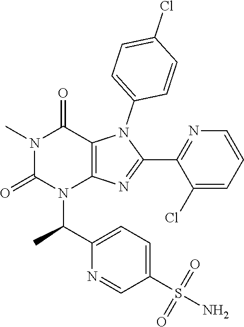 [SMILES: C[C@H](c1ccc(S(N)(=O)=O)cn1)n1c(=O)n(C)c(=O)c2c1nc(-c1ncccc1Cl)n2-c1ccc(Cl)cc1] |
| 185 | 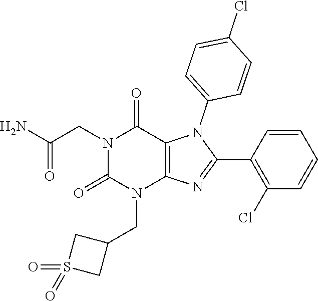 [SMILES: NC(=O)Cn1c(=O)c2c(nc(-c3ccccc3Cl)n2-c2ccc(Cl)cc2)n(CC2CS(=O)(=O)C2)c1=O] |
| 186 | 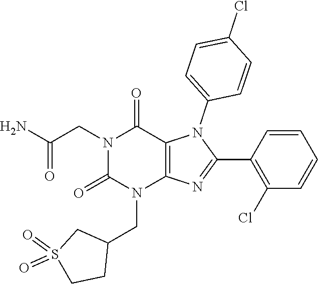 [SMILES: NC(=O)Cn1c(=O)c2c(nc(-c3ccccc3Cl)n2-c2ccc(Cl)cc2)n(CC2CCS(=O)(=O)C2)c1=O] |
| 187 | 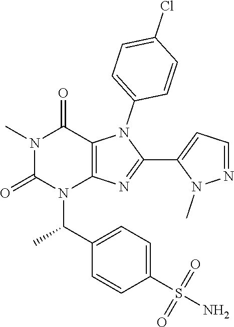 [SMILES: C[C@@H](c1ccc(S(N)(=O)=O)cc1)n1c(=O)n(C)c(=O)c2c1nc(-c1ccnn1C)n2-c1ccc(Cl)cc1] |
| 188 | 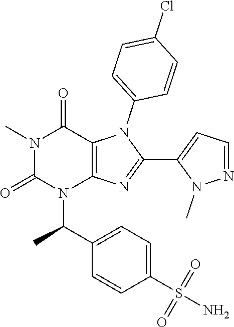 [SMILES: C[C@H](c1ccc(S(N)(=O)=O)cc1)n1c(=O)n(C)c(=O)c2c1nc(-c1ccnn1C)n2-c1ccc(Cl)cc1] |
| 189 | 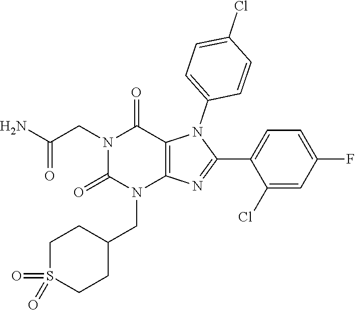 [SMILES: NC(=O)Cn1c(=O)c2c(nc(-c3ccc(F)cc3Cl)n2-c2ccc(Cl)cc2)n(CC2CCS(=O)(=O)CC2)c1=O] |
| 190 | 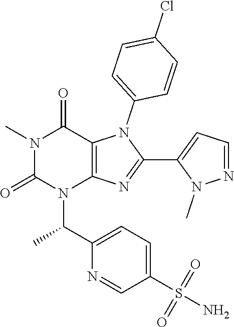 [SMILES: C[C@@H](c1ccc(S(N)(=O)=O)cn1)n1c(=O)n(C)c(=O)c2c1nc(-c1ccnn1C)n2-c1ccc(Cl)cc1] |

11. A composition, comprising a compound of claim 1; and a pharmaceutically acceptable carrier.

12. A method of treating a disease or condition characterized by aberrant CB1 activity, the method comprising the step of administering to a subject in need thereof a compound of claim 1.

13. The method of claim 12, wherein the disease or condition is diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

14. The method of claim 13, wherein the disease or condition is diabetic nephropathy.

15. The method of claim 13, wherein the disease or condition is focal segmental glomerular sclerosis.

16. The method of claim 13, wherein the disease or conditions is nonalcoholic steatohepatitis.
